SEQUENCE LISTING (46 sequences)

SEQ ID NO: 1 - DNA, 668 bp, Artificial Sequence
  >0.7UCOE
cgcgtgtggcatctgaagcaccaccagcgagcgagagctagagagaaggaaagccaccgacttcaccgcctccgagctgctccgggtcgcgggtctgcagcgtctccggccctccgcgcctacagctcaagccacatccgaagggggagggagccgggagctgcgcgcggggccgctggggggaggggtggcaccgcccacgccgggcggccacgaagggcggggcagcgggcgcgcgcccggcggggggaggggccgcgcgccgcgcccgctgggaattggggccctagggggagggcggaggcgccgacgaccgcggcacttaccgttcgcggcgtggcgcccggtggtccccaaggggagggaagggggaggcggggcgaggacagtgaccggagtctcctcagcggtggcttttctgcttggcagcctcagcggctggcgccaaaaccggactccgcccacttcctcgcccctgcggtgcgagggtgtggaatcctccagacgctgggggagggggagttgggagcttaaaaactagtacccctttgggaccactttcagcagcgaactctcctgtacaccaggggtcagttccacagacgcgggccaggggtgggtcattgcggcgtgaacaataatttgactagaagttgattcgggtgttt

SEQ ID NO: 2 - DNA, 668 bp, Artificial Sequence
  >0.7UCOEfwd
cgcaaacacccgaatcaacttctagtcaaattattgttcacgccgcaatgacccacccctggcccgcgtctgtggaactgacccctggtgtacaggagagttcgctgctgaaagtggtcccaaaggggtactagtttttaagctcccaactccccctcccccagcgtctggaggattccacaccctcgcaccgcaggggcgaggaagtgggcggagtccggttttggcgccagccgctgaggctgccaagcagaaaagccaccgctgaggagactccggtcactgtcctcgccccgcctcccccttccctccccttggggaccaccgggcgccacgccgcgaacggtaagtgccgcggtcgtcggcgcctccgccctccccctagggccccaattcccagcgggcgcggcgcgcggcccctccccccgccgggcgcgcgcccgctgccccgcccttcgtggccgcccggcgtgggcggtgccacccctccccccagcggccccgcgcgcagctcccggctccctcccccttcggatgtggcttgagctgtaggcgcggagggccggagacgctgcagacccgcgacccggagcagctcggaggcggtgaagtcggtggctttccttctctctagctctcgctcgctggtggtgcttcagatgccacac

SEQ ID NO: 3 - DNA, 455 bp, Artificial Sequence
  >DHS4
aattctatcatagtgtgtcttgtctatgataactgcattgagaaagatgctctgcttgttgagtgagcatttcacttccttctggttctgactatctgtctaatagtggtcatgtgggttgaaaagatagaaaaggggagtagtattaggaagttcagtatgaggaagacttattagacttatgcataaacctaaattctgttgtaatctggaagagctgaagtgccacatatgcatctgtttaggagagcaagaactacaaatttggtcttcagtttggcttgcttacatcctgagaactctgtaggccacatgtcgtgaatatagcagcctctgcaacagtgaaagccagaaaaggaagtggaaagtctcaggggagggggctttctgtcatggatttatgagcacagcaagactaacaagcaaaaagaaaaatgtaaaaggatcttgttcgt

SEQ ID NO: 4 - DNA, 2404 bp, Artificial Sequence
  >IE4-5
acatctttgagcttcagtttcctcatctgtaaataggggaataatacatacttcttaaggctactgcaaagatcaaataagtaatacatttgaagcacttgggacagagcctgctacatagtaagtgctcattaagtgttagttatcattgttgttgtttttaggccaaggttgttgtgaaaattaaatgagataatatataaaaggtatttagctcaatatctggcacatagcaataattgaatagatgatccttcatcttcgttcctcctgttccctttcagtttgaaagacttggctaatataattttgaccaaccaaacttgcattcaagggagtgtacaaggctggtatagccagccagtgagtatcagaatctaaatgtttattaagacaaagggctgtcatgcaataacccaaccataccattatcagtctgccatccttcctgtttctctaggcagcctttcctgatgtcaactcaaccagttaatctctcagtcacttgacatgtggctatatatacacacaaatatgtgtgcatgcatcctgtgctgcaagcatttacagtcaagtttatctgaacacactgtatggttgatgtgaaatgctgaaactgttcaagtttaggtcctcacaaagcaaggaatatgaaatatttccttgggaaatatttatccacaacaaagagatgtacagtgctttcgtatacagtgatttacagttttccatgtgcttttacatgtattattacttcatttgatccttacaacaaccccagaggtagatgtggcatgaattaccattattctcctttgagaagaagaaactgagcatcaaagaagcttgttggccttcttgccagaaatcacccagtttgtaaatggtaaaagagggcttgaaaccaggttctctgactctgacttcaagcactctcatacatcatctatttaattttttggagctaggtattttatacttaggattctaaatattgcataaccattgaatgccacaccacccttgtattcagtgcaaaaaatgggacttttcttaataaatagagaaatggaggtgcctaaaattacaaaattgcactagagagatagtgatagaactgggaaactcttagtctaatattttatcttttattcatatgatggaatactaagctcaatggcagaatcttcagtcagcaatggtgttcaggttatgtgaactatctgaaggattcctgaactcttcatatctaggaatgtagcgtttaaaagctcttagaatttttcatactcttaggtcctcctgacttgtgcttcaattcatgcataaacttattttataaggtctccgtctgcccttgctggagataacatttttgtttatccaacaaagggtattttatcttattattaaattctgactttgtatagaagagaaatgaagtgataatctatataaattaagtcttgattagtacatatgggttattcacttggataatatggagtaaaattttaattcatggctaattacctccacctccactacctagtggcctcccctcaccaatattagccaaaataaatcaaatttggaactacaaacctacttcaaaaagggtaaggtatataataagcaatatcatcaagtcaaatagtatttttttaaccatgtacaaggcatcatgctaggtgttacgaagatgcatgaaatatataagatgtggttccaaccctcacagagtttatagacatcacataataaattctgaagtcaaatataaattaatttaaaattatctgctgttcagcattgttcactagtgcagccaaacaatgtcatcttgttgaaaggcattggtagtaaaaactgtgtctgaaatacccctctttcaaaggtttcgtaaatttgatatagtcagggacacgaacaagatccttttacatttttctttttgcttgttagtcttgctgtgctcataaatccatgacagaaagccccctcccctgagactttccacttccttttctggctttcactgttgcagaggctgctatattcacgacatgtggcctacagagttctcaggatgtaagcaagccaaactgaagaccaaatttgtagttcttgctctcctaaacagatgcatatgtggcacttcagctcttccagattacaacagaatttaggtttatgcataagtctaataagtcttcctcatactgaacttcctaatactactccccttttctatcttttcaacccacatgaccactattagacagatagtcagaaccagaaggaagtgaaatgctcactcaacaagcagagcatctttctcaatgcagttatcatagacaagacacactatgatagaattggtgctgctatgtattctttttga

SEQ ID NO: 5 - DNA, 837 bp, Artificial Sequence
  >BTKp
tgcatttcctaggagaatccctgggggaatcattgcagttggagcataatgtagggggcccctgagaaaacctccaggcttcaagtgacatacctagtctgctttaccggtttacaggactcaagagaaaggtggacattgagagttaatccctgaggccaaatcttaaatggagaaagtcaacatccacagaaaatggggaagggcacaagtatttctgtgggcttatattccgacatttttatctgtaggggaaaaatgctttcttagaaaatgactcagcacggggaagtcttgtctctacctctgtcttgttttgtcctttggggtcccttcactatcaagttcaactgtgtgtccctgagactcctctgccccggaggacaggagactcgaaaaacgctcttcctggccagtctctttgctctgtgtctgccagcccccagcatctctcctctttcctgtaagcccctctccctgtgctgactgtcttcatagtactttaggtatgttgtccctttacctctgggaggatagcttgatgacctgtctgctcaggccagccccatctagagtctcagtggccccagtcatgttgagaaaggttctttcaaagatagactcaagatagtagtgtcagaggtcccaagcaaatgaagggcggggacagttgagggggtggaatagggacggcagcagggaaccagatagcatgctgctgagaagaaaaaaagacattggtttaggtcaggaagcaaaaaaagggaactgagtggctgtgaaagggtggggtttgctcagactgtccttcctctctggactgtaagaattagtctc

SEQ ID NO: 6 - DNA, 1980 bp, Artificial Sequence
  >coBTK
atggccgctgtgatcctggagagcattttcctgaagaggtcccagcagaaaaagaaaacctctcccctgaactttaagaaaagactgttcctgctgacagtgcacaagctgtcttactatgagtacgactttgagcggggccgccgaggatcaaaaaaggggagcatcgatgtggagaagattacatgcgtggagaccgtggtccctgaaaagaatccaccccctgagaggcagatcccaagacggggcgaggagtcctctgagatggagcagattagtatcattgagcgcttcccctatccttttcaggtggtgtacgacgagggaccactgtatgtgttctcacccacagaggagctgagaaagaggtggattcaccagctgaagaacgtgattagatacaatagcgatctggtgcagaagtatcacccttgtttttggatcgacgggcagtacctgtgctgttcccagacagctaagaacgctatgggatgccagattctggaaaatcggaacggatctctgaaaccagggagttcacaccgcaagaccaaaaagcccctgcctccaacacccgaggaggatcagatcctgaaaaagcctctgccacccgagcctgctgcagccccagtcagcacttccgaactgaaaaaggtggtggctctgtatgactacatgcccatgaatgctaacgatctgcagctgagaaagggcgacgagtatttcattctggaagagtctaatctgccttggtggagggccagagataagaacggacaggaggggtacatcccatctaattatgtgaccgaggctgaggactctattgagatgtacgagtggtatagcaagcacatgacacggtcccaggctgagcagctgctgaagcaggagggcaaagagggagggtttatcgtgcgcgattctagtaaggccggcaaatacactgtgtcagtgttcgctaagagcaccggagacccccagggcgtgatcagacactatgtggtgtgttccacacctcagtctcagtactatctggctgagaagcacctgtttagtacaatcccagagctgattaactaccaccagcacaattctgccggcctgatcagcaggctgaagtatcccgtctcccagcagaacaaaaatgctccttctaccgctggactggggtacggcagttgggagattgatccaaaggacctgacattcctgaaggagctgggaactgggcagtttggcgtggtgaagtatggaaaatggagagggcagtacgatgtggccatcaagatgatcaaggagggctcaatgagcgaggacgagttcatcgaggaggctaaggtcatgatgaacctgtcccacgagaaactggtgcagctgtatggagtgtgcaccaagcagcggcccatttttatcattacagagtacatggctaatgggtgtctgctgaactatctgcgcgagatgagacacagattccagacacagcagctgctggaaatgtgcaaggatgtgtgtgaggctatggagtacctggagtctaagcagtttctgcaccgggacctggctgctcgcaattgcctggtgaacgatcagggcgtggtgaaggtgagtgacttcggactgtcaaggtatgtgctggatgacgagtacaccagctccgtgggctctaagtttcctgtgagatggtctccacccgaggtgctgatgtatagcaagttctcctctaagagcgatatctgggcctttggcgtgctgatgtgggaaatctacagcctgggcaagatgccttacgagcggttcacaaattccgagacagctgagcacatcgcccagggcctgcgcctgtaccggccacatctggcctctgagaaggtgtacaccatcatgtacagctgttggcacgagaaggccgacgagagacccacattcaagatcctgctgtccaacattctagatgtgatggacgaggagagctga

SEQ ID NO: 7 - DNA, 1980 bp, Artificial Sequence
  >co2BTK
atggccgccgtgatcctggaaagcatcttcctgaagcggagccagcagaagaagaaaaccagccccctgaacttcaagaagcggctgttcctgctgaccgtgcacaagctgtcctactacgagtacgacttcgagcggggcagacggggcagcaagaagggcagcatcgacgtcgagaagatcacctgcgtggagaccgtggtgcccgagaagaacccccctcccgagcggcagatccccagacggggcgaggaaagcagcgagatggaacagatcagcatcatcgagcggttcccttacccattccaagtggtgtacgacgagggccccctgtacgtgttcagccccaccgaggaactgcggaagcggtggattcaccagctgaagaacgtgatccggtacaacagcgacctggtgcagaagtaccacccctgcttttggatcgacggccagtacctgtgctgcagccagaccgccaagaacgctatgggctgccagattctggaaaaccggaacggcagcctgaagcccggcagcagccacagaaagaccaagaagcccctgccccccacccccgaagaggaccagatcctgaagaagcctctgcctcccgagcccgccgctgcacctgtgagcaccagcgagctgaagaaagtggtggccctgtacgactacatgcccatgaacgccaacgacctgcagctgcggaagggcgacgagtacttcatcctggaagaaagcaacctgccctggtggcgggccagggacaagaacggccaggaaggctacatccccagcaactacgtgaccgaggccgaggactccatcgagatgtacgagtggtacagcaagcacatgaccagaagccaggccgaacagctgctgaagcaggaaggcaaagagggcggcttcatcgtccgggacagcagcaaggccggcaagtacaccgtgagcgtgttcgccaagagcaccggcgacccccagggcgtgatccggcactacgtggtgtgcagcaccccccagagccagtactacctggccgagaagcacctgttcagcaccatccccgagctgatcaactatcaccagcacaacagcgctggactgatttctcggctgaagtaccccgtgtcccagcagaacaaaaacgcccccagcacagccggcctgggctacggcagctgggagatcgaccccaaggacctgaccttcctgaaagagctgggcaccggccagttcggcgtggtgaagtacggcaagtggaggggccagtacgacgtggccatcaagatgatcaaggaaggcagcatgagcgaggacgagttcatcgaggaagccaaagtgatgatgaacctgagccacgagaagctggtgcagctgtacggcgtgtgcaccaagcagcggcccatcttcatcatcaccgagtacatggccaacggctgcctgctgaactacctgcgggagatgcggcacaggttccagacacagcagctgctcgaaatgtgcaaggacgtgtgcgaggctatggaatacctggaatccaagcagttcctgcaccgggacctggccgccagaaactgcctggtgaacgaccagggggtggtgaaggtgtccgacttcggcctgagcagatacgtgctggacgacgagtacaccagcagcgtgggcagcaagttccccgtgcggtggagcccccctgaggtgctgatgtacagcaagttcagcagcaagagcgacatctgggccttcggcgtgctgatgtgggagatctacagcctgggcaagatgccctacgagcggttcaccaacagcgagaccgccgagcacatcgcccagggcctgcggctgtacaggccccacctggccagcgagaaggtgtacaccatcatgtacagctgctggcacgagaaggccgacgagaggcccaccttcaagatcctgctgtccaacatcctggacgtgatggacgaggaaagctga

SEQ ID NO: 8 - DNA, 720 bp, Artificial Sequence
  >GFP
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 9 - DNA, 1227 bp, Artificial Sequence
  >Intron 4
ctcaaaaagaatacatagcagcaccaattctatcatagtgtgtcttgtctatgataactgcattgagaaagatgctctgcttgttgagtgagcatttcacttccttctggttctgactatctgtctaatagtggtcatgtgggttgaaaagatagaaaaggggagtagtattaggaagttcagtatgaggaagacttattagacttatgcataaacctaaattctgttgtaatctggaagagctgaagtgccacatatgcatctgtttaggagagcaagaactacaaatttggtcttcagtttggcttgcttacatcctgagaactctgtaggccacatgtcgtgaatatagcagcctctgcaacagtgaaagccagaaaaggaagtggaaagtctcaggggagggggctttctgtcatggatttatgagcacagcaagactaacaagcaaaaagaaaaatgtaaaaggatcttgttcgtgtccctgactatatcaaatttacgaaacctttgaaagaggggtatttcagacacagtttttactaccaatgcctttcaacaagatgacattgtttggctgcactagtgaacaatgctgaacagcagataattttaaattaatttatatttgacttcagaatttattatgtgatgtctataaactctgtgagggttggaaccacatcttatatatttcatgcatcttcgtaacacctagcatgatgccttgtacatggttaaaaaaatactatttgacttgatgatattgcttattatataccttaccctttttgaagtaggtttgtagttccaaatttgatttattttggctaatattggtgaggggaggccactaggtagtggaggtggaggtaattagccatgaattaaaattttactccatattatccaagtgaataacccatatgtactaatcaagacttaatttatatagattatcacttcatttctcttctatacaaagtcagaatttaataataagataaaataccctttgttggataaacaaaaatgttatctccagcaagggcagacggagaccttataaaataagtttatgcatgaattgaagcacaagtcaggaggacctaagagtatgaaaaattctaagagcttttaaacgctacattcctagatatgaagagttcaggaatccttcagatagttcacataacctgaacaccattgctgactgaagattctgcc

SEQ ID NO: 10 - DNA, 1163 bp, Artificial Sequence
  >Intron 5
ccatcatatgaataaaagataaaatattagactaagagtttcccagttctatcactatctctctagtgcaattttgtaattttaggcacctccatttctctatttattaagaaaagtcccattttttgcactgaatacaagggtggtgtggcattcaatggttatgcaatatttagaatcctaagtataaaatacctagctccaaaaaattaaatagatgatgtatgagagtgcttgaagtcagagtcagagaacctggtttcaagccctcttttaccatttacaaactgggtgatttctggcaagaaggccaacaagcttctttgatgctcagtttcttcttctcaaaggagaataatggtaattcatgccacatctacctctggggttgttgtaaggatcaaatgaagtaataatacatgtaaaagcacatggaaaactgtaaatcactgtatacgaaagcactgtacatctctttgttgtggataaatatttcccaaggaaatatttcatattccttgctttgtgaggacctaaacttgaacagtttcagcatttcacatcaaccatacagtgtgttcagataaacttgactgtaaatgcttgcagcacaggatgcatgcacacatatttgtgtgtatatatagccacatgtcaagtgactgagagattaactggttgagttgacatcaggaaaggctgcctagagaaacaggaaggatggcagactgataatggtatggttgggttattgcatgacagccctttgtcttaataaacatttagattctgatactcactggctggctataccagccttgtacactcccttgaatgcaagtttggttggtcaaaattatattagccaagtctttcaaactgaaagggaacaggaggaacgaagatgaaggatcatctattcaattattgctatgtgccagatattgagctaaataccttttatatattatctcatttaattttcacaacaaccttggcctaaaaacaacaacaatgataactaacacttaatgagcacttactatgtagcaggctctgtcccaagtgcttcaaatgtattacttatttgatctttgcagtagccttaagaagtatgtattattcccctatttacagatgaggaaactgaagctcaaagatgt

SEQ ID NO: 11 - DNA, 526 bp, Artificial Sequence
  >Intron 13
ggtagggtggactggccagttgcaaaaactacctttgctggccttgccttagggagtgtccttgaggtacactgttctgcagcagctgcctcaaggacgctcaagacagatccaagcaaaagttattcactgattttcttcctctagtggctacgactgggactgcaaaaacatagattcataaagggctttgtcgttgtcttgggtctttttgtcttttatttttaattgtgggaaaattttcagtactatccctgagttcattaactaccatcactaacataatcataaagggatttggggaggttgcttagtctatcttcttgccttatggccaccttgaacctaaaattcccagattcctctaaccaatgaatcccgtttctgagattgacttaagcaaagacagattagtacttctaaaaatttcccttttactagttttcctatttctaccccagtagggatttttgtctattgtaagaattatacattcatgaccccaaagaatcacaccaagacttta

SEQ ID NO: 12-13 - skipped (2 entries)

SEQ ID NO: 14 - DNA, 2947 bp, Artificial Sequence
  >1Kb
tcaaaaagaatacatagcagcaccaattctatcatagtgtgtcttgtctatgataactgcattgagaaagatgctctgcttgttgagtgagcatttcacttccttctggttctgactatctgtctaatagtggtcatgtgggttgaaaagatagaaaaggggagtagtattaggaagttcagtatgaggaagacttattagacttatgcataaacctaaattctgttgtaatctggaagagctgaagtgccacatatgcatctgtttaggagagcaagaactacaaatttggtcttcagtttggcttgcttacatcctgagaactctgtaggccacatgtcgtgaatatagcagcctctgcaacagtgaaagccagaaaaggaagtggaaagtctcaggggagggggctttctgtcatggatttatgagcacagcaagactaacaagcaaaaagaaaaatgtaaaaggatcttgttcgtgtccctgactatatcaaatttacgaaacctttgaaagaggggtatttcagacacagtttttactaccaatgcctttcaacaagatgacattgtttggctgcactagtgaacaatgctgaacagcagataattttaaattaatttatatttgacttcagaatttattatgtgatgtctataaactctgtgagggttggaaccacatcttatatatttcatgcatcttcgtaacacctagcatgatgccttgtacatggttaaaaaaatactatttgacttgatgatattgcttattatataccttaccctttttgaagtaggtttgtagttccaaatttgatttattttggctaatattggtgaggggaggccactaggtagtggaggtggaggtaattagccatgaattaaaattttactccatattatccaagtgaataacccatatgtactaatcaagacttaatttatatagattatcacttcatttctcttctatacaaagtcagaatttaataataagataaaataccctttgttggataaacaaaaatgttatctccagcaagggcagacggagaccttataaaataagtttatgcatgaattgaagcacaagtcaggaggacctaagagtatgaaaaattctaagagcttttaaacgctacattcctagatatgaagagttcaggaatccttcagatagttcacataacctgaacaccattgctgactgaagattctgccattgagcttagtattccatcatatgaataaaagataaaatattagactaagagtttcccagttctatcactatctctctagtgcaattttgtaattttaggcacctccatttctctatttattaagaaaagtcccattttttgcactgaatacaagggtggtgtggcattcaatggttatgcaatatttagaatcctaagtataaaatacctagctccaaaaaattaaatagatgatgtatgagagtgcttgaagtcagagtcagagaacctggtttcaagccctcttttaccatttacaaactgggtgatttctggcaagaaggccaacaagcttctttgatgctcagtttcttcttctcaaaggagaataatggtaattcatgccacatctacctctggggttgttgtaaggatcaaatgaagtaataatacatgtaaaagcacatggaaaactgtaaatcactgtatacgaaagcactgtacatctctttgttgtggataaatatttcccaaggaaatatttcatattccttgctttgtgaggacctaaacttgaacagtttcagcatttcacatcaaccatacagtgtgttcagataaacttgactgtaaatgcttgcagcacaggatgcatgcacacatatttgtgtgtatatatagccacatgtcaagtgactgagagattaactggttgagttgacatcaggaaaggctgcctagagaaacaggaaggatggcagactgataatggtatggttgggttattgcatgacagccctttgtcttaataaacatttagattctgatactcactggctggctataccagccttgtacactcccttgaatgcaagtttggttggtcaaaattatattagccaagtctttcaaactgaaagggaacaggaggaacgaagatgaaggatcatctattcaattattgctatgtgccagatattgagctaaataccttttatatattatctcatttaattttcacaacaaccttggcctaaaaacaacaacaatgataactaacacttaatgagcacttactatgtagcaggctctgtcccaagtgcttcaaatgtattacttatttgatctttgcagtagccttaagaagtatgtattattcccctatttacagatgaggaaactgaagctcaaagatgttatccttatttctctggggtagggtggactggccagttgcaaaaactacctttgctggccttgccttagggagtgtccttgaggtacactgttctgcagcagctgcctcaaggacgctcaagacagatccaagcaaaagttattcactgattttcttcctctagtggctacgactgggactgcaaaaacatagattcataaagggctttgtcgttgtcttgggtctttttgtcttttatttttaattgtgggaaaattttcagtactatccctgagttcattaactaccatcactaacataatcataaagggatttggggaggttgcttagtctatcttcttgccttatggccaccttgaacctaaaattcccagattcctctaaccaatgaatcccgtttctgagattgacttaagcaaagacagattagtacttctaaaaatttcccttttactagttttcctatttctaccccagtagggatttttgtctattgtaagaattatacattcatgaccccaaagaatcacaccaagacttta

SEQ ID NO: 15 - DNA, 2953 bp, Artificial Sequence
  >revCONTIG
taacaataaagtcttggtgtgattctttggggtcatgaatgtataattcttacaatagacaaaaatccctactggggtagaaataggaaaactagtaaaagggaaatttttagaagtactaatctgtctttgcttaagtcaatctcagaaacgggattcattggttagaggaatctgggaattttaggttcaaggtggccataaggcaagaagatagactaagcaacctccccaaatccctttatgattatgttagtgatggtagttaatgaactcagggatagtactgaaaattttcccacaattaaaaataaaagacaaaaagacccaagacaacgacaaagccctttatgaatctatgtttttgcagtcccagtcgtagccactagaggaagaaaatcagtgaataacttttgcttggatctgtcttgagcgtccttgaggcagctgctgcagaacagtgtacctcaaggacactccctaaggcaaggccagcaaaggtagtttttgcaactggccagtccaccctaccccagagaaataaggataacatctttgagcttcagtttcctcatctgtaaataggggaataatacatacttcttaaggctactgcaaagatcaaataagtaatacatttgaagcacttgggacagagcctgctacatagtaagtgctcattaagtgttagttatcattgttgttgtttttaggccaaggttgttgtgaaaattaaatgagataatatataaaaggtatttagctcaatatctggcacatagcaataattgaatagatgatccttcatcttcgttcctcctgttccctttcagtttgaaagacttggctaatataattttgaccaaccaaacttgcattcaagggagtgtacaaggctggtatagccagccagtgagtatcagaatctaaatgtttattaagacaaagggctgtcatgcaataacccaaccataccattatcagtctgccatccttcctgtttctctaggcagcctttcctgatgtcaactcaaccagttaatctctcagtcacttgacatgtggctatatatacacacaaatatgtgtgcatgcatcctgtgctgcaagcatttacagtcaagtttatctgaacacactgtatggttgatgtgaaatgctgaaactgttcaagtttaggtcctcacaaagcaaggaatatgaaatatttccttgggaaatatttatccacaacaaagagatgtacagtgctttcgtatacagtgatttacagttttccatgtgcttttacatgtattattacttcatttgatccttacaacaaccccagaggtagatgtggcatgaattaccattattctcctttgagaagaagaaactgagcatcaaagaagcttgttggccttcttgccagaaatcacccagtttgtaaatggtaaaagagggcttgaaaccaggttctctgactctgacttcaagcactctcatacatcatctatttaattttttggagctaggtattttatacttaggattctaaatattgcataaccattgaatgccacaccacccttgtattcagtgcaaaaaatgggacttttcttaataaatagagaaatggaggtgcctaaaattacaaaattgcactagagagatagtgatagaactgggaaactcttagtctaatattttatcttttattcatatgatggaatactaagctcaatggcagaatcttcagtcagcaatggtgttcaggttatgtgaactatctgaaggattcctgaactcttcatatctaggaatgtagcgtttaaaagctcttagaatttttcatactcttaggtcctcctgacttgtgcttcaattcatgcataaacttattttataaggtctccgtctgcccttgctggagataacatttttgtttatccaacaaagggtattttatcttattattaaattctgactttgtatagaagagaaatgaagtgataatctatataaattaagtcttgattagtacatatgggttattcacttggataatatggagtaaaattttaattcatggctaattacctccacctccactacctagtggcctcccctcaccaatattagccaaaataaatcaaatttggaactacaaacctacttcaaaaagggtaaggtatataataagcaatatcatcaagtcaaatagtatttttttaaccatgtacaaggcatcatgctaggtgttacgaagatgcatgaaatatataagatgtggttccaaccctcacagagtttatagacatcacataataaattctgaagtcaaatataaattaatttaaaattatctgctgttcagcattgttcactagtgcagccaaacaatgtcatcttgttgaaaggcattggtagtaaaaactgtgtctgaaatacccctctttcaaaggtttcgtaaatttgatatagtcagggacacgaacaagatccttttacatttttctttttgcttgttagtcttgctgtgctcataaatccatgacagaaagccccctcccctgagactttccacttccttttctggctttcactgttgcagaggctgctatattcacgacatgtggcctacagagttctcaggatgtaagcaagccaaactgaagaccaaatttgtagttcttgctctcctaaacagatgcatatgtggcacttcagctcttccagattacaacagaatttaggtttatgcataagtctaataagtcttcctcatactgaacttcctaatactactccccttttctatcttttcaacccacatgaccactattagacagatagtcagaaccagaaggaagtgaaatgctcactcaacaagcagagcatctttctcaatgcagttatcatagacaagacacactatgatagaattggtgctgctatgtattctttttga

SEQ ID NO: 16 - DNA, 2282 bp, Artificial Sequence
  >segments ABCD of test intron 1
ctggggtatggcaggggctgggcagcagcagcaatgtaccttgcttgggacccctaaaaaccagagagacagcatggctggtgccatttatcagctagtggaggaggctgacggagggtgggagtgtcatcagcacaaggccctggcagtcccttctggtgattagagaggccgaaagggtcctttccgacaagggctgagggtgggcggaacaggaagagaaaaatgtgacatgaggtgaccatccgaacaggtagcaaatgttagaaaggggtacctctggcaaacttagtggaaaagtaatattgcagggagcagtcagataaaaacaagcccttctgtcaaatagtgcttgaagactcaatagggatacatgggtcaatgaagcctttagaaaaagaaatactaagaggcagattctctgagaacatggtaaaagctcacgctccacgttatgaagttgacctttgtgagctagggaaaggcctggctaggccagggtgtaggctacctgccttgagctgtaccaggccaaatgtcgccagggtcagagctggcttattaaaggactgtgtggaagctgtgccaacctcgtggtaacaatgggtaaaagactgggccaggagaaagcagcctctgcctcagcccagacagtgcggccaacccttgaggttgtggcaaaggtttctcctcttaccattgccctccatgtgcatggcttgcttttctcttgtcttcattatttctcctttcctttcctcggatccacgcgtgaattctttgtaaactccttatggtgcgaactaatgtaactttccatccagttatgggggattggtgcaattttaaattatcactatgatttgctatttccatttgagcaaatttcctatagagtttcctttcagtggactagacccatatcaggaagtgacttaggtataaagggaagatacagctttcgaaaaccaaagtttgggcgttctccaaagagttatcagatacccccttctacacccacaatgatctgattgctgagatctgattgctaactactgaaaataaggaagaactagaattttcagtgacacagtgctcagcaagaagctagaaaagaggccttgacatatttgactccaaagctacttggttatgcatgaagccatctggggaggggaaggaggagggagaactcctctgaggaccctgaaacaattgggccacgtgtgactttcagtttctatggagattcatgtgcagtggctgagggcaatctgagagcattggaaacccagaagctttaaacgcgtaggaaagacagggaatggccagaatcttcctagccaattgagtagtgctttcaaggagaaatcaagagaaaacactacttcttggatattttggctaagtagtcatttgaagtacagttgactggttattttattttaaatcatatctcatagactcttcccaatcataggctggctgtgtagttttctgaattttgctctggtattcttcttcttttttttttttatttttattttttattttttttttgagactccaggctggagtgcagtgtcacgatcttggctcactgcaacctccgcctcccgagttcaagcgattctcctgcctcagcctcctgagttgctgggactacaggcgcctgtcaccacgcccggctaattttttgtattttaatagagacgggatttcaccatgttggccaggctggtctctaactcctgacctcaagtgatccgccagcctcagcctcccaaagtgctgggattacgggcatgagccactgcgcctggacttattattcttaatagtattttatcttatgagcgaagataagagcccaagatggtttagtttactgattctgcaagtgctatttctattaattccttggcatactgcagtttgtatgatggctgcactcttgttaataagcttcgtctttctgaattctgttgctccatagggagctgggaggctgcaaaaggtggccctgtaaaaatctttgcatttataatttaataattacagaccccagtgggacaatgtttgaaaaattatattcaccgtctaggaaattgggaactgaaagtccaatatctgcctcagtggagttctggcacctgcattatcccttctgggtatatcaagatcaacagctgcacagatacttttgcttttcacagattctacacatatcatataaaggtgaatagtgtaaagctacctctacaccttaccaagcacacagg

SEQ ID NO: 17 - DNA, 2282 bp, Artificial Sequence
  >segments DCBA of test intron 1
cctgtgtgcttggtaaggtgtagaggtagctttacactattcacctttatatgatatgtgtagaatctgtgaaaagcaaaagtatctgtgcagctgttgatcttgatatacccagaagggataatgcaggtgccagaactccactgaggcagatattggactttcagttcccaatttcctagacggtgaatataatttttcaaacattgtcccactggggtctgtaattattaaattataaatgcaaagatttttacagggccaccttttgcagcctcccagctccctatggagcaacagaattcagaaagacgaagcttattaacaagagtgcagccatcatacaaactgcagtatgccaaggaattaatagaaatagcacttgcagaatcagtaaactaaaccatcttgggctcttatcttcgctcataagataaaatactattaagaataataagtccaggcgcagtggctcatgcccgtaatcccagcactttgggaggctgaggctggcggatcacttgaggtcaggagttagagaccagcctggccaacatggtgaaatcccgtctctattaaaatacaaaaaattagccgggcgtggtgacaggcgcctgtagtcccagcaactcaggaggctgaggcaggagaatcgcttgaactcgggaggcggaggttgcagtgagccaagatcgtgacactgcactccagcctggagtctcaaaaaaaaaataaaaaataaaaataaaaaaaaaaaagaagaagaataccagagcaaaattcagaaaactacacagccagcctatgattgggaagagtctatgagatatgatttaaaataaaataaccagtcaactgtacttcaaatgactacttagccaaaatatccaagaagtagtgttttctcttgatttctccttgaaagcactactcaattggctaggaagattctggccattccctgtctttcctacgcgtttaaagcttctgggtttccaatgctctcagattgccctcagccactgcacatgaatctccatagaaactgaaagtcacacgtggcccaattgtttcagggtcctcagaggagttctccctcctccttcccctccccagatggcttcatgcataaccaagtagctttggagtcaaatatgtcaaggcctcttttctagcttcttgctgagcactgtgtcactgaaaattctagttcttccttattttcagtagttagcaatcagatctcagcaatcagatcattgtgggtgtagaagggggtatctgataactctttggagaacgcccaaactttggttttcgaaagctgtatcttccctttatacctaagtcacttcctgatatgggtctagtccactgaaaggaaactctataggaaatttgctcaaatggaaatagcaaatcatagtgataatttaaaattgcaccaatcccccataactggatggaaagttacattagttcgcaccataaggagtttacaaagaattcacgcgtggatccgaggaaaggaaaggagaaataatgaagacaagagaaaagcaagccatgcacatggagggcaatggtaagaggagaaacctttgccacaacctcaagggttggccgcactgtctgggctgaggcagaggctgctttctcctggcccagtcttttacccattgttaccacgaggttggcacagcttccacacagtcctttaataagccagctctgaccctggcgacatttggcctggtacagctcaaggcaggtagcctacaccctggcctagccaggcctttccctagctcacaaaggtcaacttcataacgtggagcgtgagcttttaccatgttctcagagaatctgcctcttagtatttctttttctaaaggcttcattgacccatgtatccctattgagtcttcaagcactatttgacagaagggcttgtttttatctgactgctccctgcaatattacttttccactaagtttgccagaggtacccctttctaacatttgctacctgttcggatggtcacctcatgtcacatttttctcttcctgttccgcccaccctcagcccttgtcggaaaggaccctttcggcctctctaatcaccagaagggactgccagggccttgtgctgatgacactcccaccctccgtcagcctcctccactagctgataaatggcaccagccatgctgtctctctggtttttaggggtcccaagcaaggtacattgctgctgctgcccagcccctgccataccccag

SEQ ID NO: 18 - DNA, 1304 bp, Artificial Sequence
  >segments AB of test intron 1
ctggggtatggcaggggctgggcagcagcagcaatgtaccttgcttgggacccctaaaaaccagagagacagcatggctggtgccatttatcagctagtggaggaggctgacggagggtgggagtgtcatcagcacaaggccctggcagtcccttctggtgattagagaggccgaaagggtcctttccgacaagggctgagggtgggcggaacaggaagagaaaaatgtgacatgaggtgaccatccgaacaggtagcaaatgttagaaaggggtacctctggcaaacttagtggaaaagtaatattgcagggagcagtcagataaaaacaagcccttctgtcaaatagtgcttgaagactcaatagggatacatgggtcaatgaagcctttagaaaaagaaatactaagaggcagattctctgagaacatggtaaaagctcacgctccacgttatgaagttgacctttgtgagctagggaaaggcctggctaggccagggtgtaggctacctgccttgagctgtaccaggccaaatgtcgccagggtcagagctggcttattaaaggactgtgtggaagctgtgccaacctcgtggtaacaatgggtaaaagactgggccaggagaaagcagcctctgcctcagcccagacagtgcggccaacccttgaggttgtggcaaaggtttctcctcttaccattgccctccatgtgcatggcttgcttttctcttgtcttcattatttctcctttcctttcctcggatccacgcgtgaattctttgtaaactccttatggtgcgaactaatgtaactttccatccagttatgggggattggtgcaattttaaattatcactatgatttgctatttccatttgagcaaatttcctatagagtttcctttcagtggactagacccatatcaggaagtgacttaggtataaagggaagatacagctttcgaaaaccaaagtttgggcgttctccaaagagttatcagatacccccttctacacccacaatgatctgattgctgagatctgattgctaactactgaaaataaggaagaactagaattttcagtgacacagtgctcagcaagaagctagaaaagaggccttgacatatttgactccaaagctacttggttatgcatgaagccatctggggaggggaaggaggagggagaactcctctgaggaccctgaaacaattgggccacgtgtgactttcagtttctatggagattcatgtgcagtggctgagggcaatctgagagcattggaaacccagaagctttaa

SEQ ID NO: 19 - DNA, 761 bp, Artificial Sequence
  >segment A of test intron 1
ctggggtatggcaggggctgggcagcagcagcaatgtaccttgcttgggacccctaaaaaccagagagacagcatggctggtgccatttatcagctagtggaggaggctgacggagggtgggagtgtcatcagcacaaggccctggcagtcccttctggtgattagagaggccgaaagggtcctttccgacaagggctgagggtgggcggaacaggaagagaaaaatgtgacatgaggtgaccatccgaacaggtagcaaatgttagaaaggggtacctctggcaaacttagtggaaaagtaatattgcagggagcagtcagataaaaacaagcccttctgtcaaatagtgcttgaagactcaatagggatacatgggtcaatgaagcctttagaaaaagaaatactaagaggcagattctctgagaacatggtaaaagctcacgctccacgttatgaagttgacctttgtgagctagggaaaggcctggctaggccagggtgtaggctacctgccttgagctgtaccaggccaaatgtcgccagggtcagagctggcttattaaaggactgtgtggaagctgtgccaacctcgtggtaacaatgggtaaaagactgggccaggagaaagcagcctctgcctcagcccagacagtgcggccaacccttgaggttgtggcaaaggtttctcctcttaccattgccctccatgtgcatggcttgcttttctcttgtcttcattatttctcctttcctttcctc

SEQ ID NO: 20 - DNA, 531 bp, Artificial Sequence
  >segment B of test intron 1
gaattctttgtaaactccttatggtgcgaactaatgtaactttccatccagttatgggggattggtgcaattttaaattatcactatgatttgctatttccatttgagcaaatttcctatagagtttcctttcagtggactagacccatatcaggaagtgacttaggtataaagggaagatacagctttcgaaaaccaaagtttgggcgttctccaaagagttatcagatacccccttctacacccacaatgatctgattgctgagatctgattgctaactactgaaaataaggaagaactagaattttcagtgacacagtgctcagcaagaagctagaaaagaggccttgacatatttgactccaaagctacttggttatgcatgaagccatctggggaggggaaggaggagggagaactcctctgaggaccctgaaacaattgggccacgtgtgactttcagtttctatggagattcatgtgcagtggctgagggcaatctgagagcattggaaacccagaagctttaa

SEQ ID NO: 21 - DNA, 1645 bp, Artificial Sequence
  >BTKe
tccatcacctactagatatatcagtgcagtgaaaacttcgctaaactaacgctatacctatatcatgaagtgtgtggactagagacaagtgcatatccttacggcaattaactgggaaacgtcaaatagtaactaccactcacctttttccggaaaatcggcttagtttgcccaccatagccactctgcttcctgtcataacgccgctttcctgggaaaacgaattggtatttgttataaaatactgaagatcagcaagtaagtcttacaggttttatcttaatttcgcagcagaaatattaacgctcaagccaggcgtggagggagagagacccggactcgtatgttattctacaacacaaatgtcacattaacaccaaattatgcggaatccatcttaccctgggcgtacagagaatccttgcccttcttgtactgtgtcactttatggggttggtgcttgccacacttcttacagaaagtccggcgggttttagggacgttaacctagtaaagaaacagttcagaacgtgcaatgttatttgaccacaatggcacaacgccctaccttacccagctaaagctgaggcactccaggaggactcctcattacttgctacctctgactacagggtgggccagccccatgtgcttcaagcagagcttcctccctccgtcgagccccaaagagggaagagacctcattaactccacccccggctaactctacctctttgaacccatcacttcaattcctggccccgtagcccggtccctttagggttgatcccggcaagattgggttgctctgatatatcgagtccacacaggagcctggacccatcccggcatagcacgggcgacgaagggggggaaagattaagctggatgttactcggcccccaccagcaagtcctaccatgcttgcgtgagcgctatcggcgcggaaagaaagaaaccgcgaggcaaacggaagtatataggaggttcccgatcgcacttcctcatgggagtcggtaggagcaatcatagagtgtaaggctcagcgcagcgccctcgggcggctgagaggactcagttcggagccgcgggcgggagcttaaggaaggactccgcctaaagggtggtccactcaccccgacttcctcccgccccgcagctttcaacgtttcgtcactttatctcttttggtggactctgctacgtagtggcgttcagtgaagggagcagtgtttttcccagatcctctggcctccccgtccccgagggaagccaggactagggtcgaatgaaggggtcctccacctccacgttccattcctgttccacctcaaggtcactgggaacacctttcgcagcaaactgctaattcaatgaagacctggagggagccaattgttccagttcatctatcacatggccagttggtccattcaacaaatggttattggatgcccattatgtggcaggcactgttccgggggagaggtacagtaatctaataggcttataaatgtgcaattatgaactaagtactttgaagaaaaggaacaatgattggcattaaagcagcacccttctgttgagggagtaagtcagcagctctaggttctgaaaagtgacaatgaaattgtttggctcctgt

SEQ ID NO: 22 - DNA, 756 bp, Artificial Sequence
  >BTKeMyc
tgttactcggcccccaccagcaagtcctaccatgcttgcgtgagcgctatcggcgcggaaagaaagaaaccgcgaggcaaacggaagtatataggaggttcccgatcgcacttcctcatgggagtcggtaggagcaatcatagagtgtaaggctcagcgcagcgccctcgggcggctgagaggactcagttcggagccgcgggcgggagcttaaggaaggactccgcctaaagggtggtccactcaccccgacttcctcccgccccgcagctttcaacgtttcgtcactttatctcttttggtggactctgctacgtagtggcgttcagtgaagggagcagtgtttttcccagatcctctggcctccccgtccccgagggaagccaggactagggtcgaatgaaggggtcctccacctccacgttccattcctgttccacctcaaggtcactgggaacacctttcgcagcaaactgctaattcaatgaagacctggagggagccaattgttccagttcatctatcacatggccagttggtccattcaacaaatggttattggatgcccattatgtggcaggcactgttccgggggagaggtacagtaatctaataggcttataaatgtgcaattatgaactaagtactttgaagaaaaggaacaatgattggcattaaagcagcacccttctgttgagggagtaagtcagcagctctaggttctgaaaagtgacaatgaaattgtttggctcctgt

SEQ ID NO: 23 - DNA, 1565 bp, Artificial Sequence
  >BTKp.GFP
gcatttcctaggagaatccctgggggaatcattgcagttggagcataatgtagggggcccctgagaaaacctccaggcttcaagtgacatacctagtctgctttaccggtttacaggactcaagagaaaggtggacattgagagttaatccctgaggccaaatcttaaatggagaaagtcaacatccacagaaaatggggaagggcacaagtatttctgtgggcttatattccgacatttttatctgtaggggaaaaatgctttcttagaaaatgactcagcacggggaagtcttgtctctacctctgtcttgttttgtcctttggggtcccttcactatcaagttcaactgtgtgtccctgagactcctctgccccggaggacaggagactcgaaaaacgctcttcctggccagtctctttgctctgtgtctgccagcccccagcatctctcctctttcctgtaagcccctctccctgtgctgactgtcttcatagtactttaggtatgttgtccctttacctctgggaggatagcttgatgacctgtctgctcaggccagccccatctagagtctcagtggccccagtcatgttgagaaaggttctttcaaagatagactcaagatagtagtgtcagaggtcccaagcaaatgaagggcggggacagttgagggggtggaatagggacggcagcagggaaccagatagcatgctgctgagaagaaaaaaagacattggtttaggtcaggaagcaaaaaaagggaactgagtggctgtgaaagggtggggtttgctcagactgtccttcctctctggactgtaagaattagtctcgaggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 24 - DNA, 2798 bp, Artificial Sequence
  INT4.BTKp.GFP
gcatttcctaggagaatccctgggggaatcattgcagttggagcataatgtagggggcccctcaaaaagaatacatagcagcaccaattctatcatagtgtgtcttgtctatgataactgcattgagaaagatgctctgcttgttgagtgagcatttcacttccttctggttctgactatctgtctaatagtggtcatgtgggttgaaaagatagaaaaggggagtagtattaggaagttcagtatgaggaagacttattagacttatgcataaacctaaattctgttgtaatctggaagagctgaagtgccacatatgcatctgtttaggagagcaagaactacaaatttggtcttcagtttggcttgcttacatcctgagaactctgtaggccacatgtcgtgaatatagcagcctctgcaacagtgaaagccagaaaaggaagtggaaagtctcaggggagggggctttctgtcatggatttatgagcacagcaagactaacaagcaaaaagaaaaatgtaaaaggatcttgttcgtgtccctgactatatcaaatttacgaaacctttgaaagaggggtatttcagacacagtttttactaccaatgcctttcaacaagatgacattgtttggctgcactagtgaacaatgctgaacagcagataattttaaattaatttatatttgacttcagaatttattatgtgatgtctataaactctgtgagggttggaaccacatcttatatatttcatgcatcttcgtaacacctagcatgatgccttgtacatggttaaaaaaatactatttgacttgatgatattgcttattatataccttaccctttttgaagtaggtttgtagttccaaatttgatttattttggctaatattggtgaggggaggccactaggtagtggaggtggaggtaattagccatgaattaaaattttactccatattatccaagtgaataacccatatgtactaatcaagacttaatttatatagattatcacttcatttctcttctatacaaagtcagaatttaataataagataaaataccctttgttggataaacaaaaatgttatctccagcaagggcagacggagaccttataaaataagtttatgcatgaattgaagcacaagtcaggaggacctaagagtatgaaaaattctaagagcttttaaacgctacattcctagatatgaagagttcaggaatccttcagatagttcacataacctgaacaccattgctgactgaagattctgccgggcccctgagaaaacctccaggcttcaagtgacatacctagtctgctttaccggtttacaggactcaagagaaaggtggacattgagagttaatccctgaggccaaatcttaaatggagaaagtcaacatccacagaaaatggggaagggcacaagtatttctgtgggcttatattccgacatttttatctgtaggggaaaaatgctttcttagaaaatgactcagcacggggaagtcttgtctctacctctgtcttgttttgtcctttggggtcccttcactatcaagttcaactgtgtgtccctgagactcctctgccccggaggacaggagactcgaaaaacgctcttcctggccagtctctttgctctgtgtctgccagcccccagcatctctcctctttcctgtaagcccctctccctgtgctgactgtcttcatagtactttaggtatgttgtccctttacctctgggaggatagcttgatgacctgtctgctcaggccagccccatctagagtctcagtggccccagtcatgttgagaaaggttctttcaaagatagactcaagatagtagtgtcagaggtcccaagcaaatgaagggcggggacagttgagggggtggaatagggacggcagcagggaaccagatagcatgctgctgagaagaaaaaaagacattggtttaggtcaggaagcaaaaaaagggaactgagtggctgtgaaagggtggggtttgctcagactgtccttcctctctggactgtaagaattagtctcgaggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 25 - DNA, 2735 bp, Artificial Sequence
  INT5.BTKp.GFP
gcatttcctaggagaatccctgggggaatcattgcagttggagcataatgtagggggccccatcatatgaataaaagataaaatattagactaagagtttcccagttctatcactatctctctagtgcaattttgtaattttaggcacctccatttctctatttattaagaaaagtcccattttttgcactgaatacaagggtggtgtggcattcaatggttatgcaatatttagaatcctaagtataaaatacctagctccaaaaaattaaatagatgatgtatgagagtgcttgaagtcagagtcagagaacctggtttcaagccctcttttaccatttacaaactgggtgatttctggcaagaaggccaacaagcttctttgatgctcagtttcttcttctcaaaggagaataatggtaattcatgccacatctacctctggggttgttgtaaggatcaaatgaagtaataatacatgtaaaagcacatggaaaactgtaaatcactgtatacgaaagcactgtacatctctttgttgtggataaatatttcccaaggaaatatttcatattccttgctttgtgaggacctaaacttgaacagtttcagcatttcacatcaaccatacagtgtgttcagataaacttgactgtaaatgcttgcagcacaggatgcatgcacacatatttgtgtgtatatatagccacatgtcaagtgactgagagattaactggttgagttgacatcaggaaaggctgcctagagaaacaggaaggatggcagactgataatggtatggttgggttattgcatgacagccctttgtcttaataaacatttagattctgatactcactggctggctataccagccttgtacactcccttgaatgcaagtttggttggtcaaaattatattagccaagtctttcaaactgaaagggaacaggaggaacgaagatgaaggatcatctattcaattattgctatgtgccagatattgagctaaataccttttatatattatctcatttaattttcacaacaaccttggcctaaaaacaacaacaatgataactaacacttaatgagcacttactatgtagcaggctctgtcccaagtgcttcaaatgtattacttatttgatctttgcagtagccttaagaagtatgtattattcccctatttacagatgaggaaactgaagctcaaagatgttagggcccctgagaaaacctccaggcttcaagtgacatacctagtctgctttaccggtttacaggactcaagagaaaggtggacattgagagttaatccctgaggccaaatcttaaatggagaaagtcaacatccacagaaaatggggaagggcacaagtatttctgtgggcttatattccgacatttttatctgtaggggaaaaatgctttcttagaaaatgactcagcacggggaagtcttgtctctacctctgtcttgttttgtcctttggggtcccttcactatcaagttcaactgtgtgtccctgagactcctctgccccggaggacaggagactcgaaaaacgctcttcctggccagtctctttgctctgtgtctgccagcccccagcatctctcctctttcctgtaagcccctctccctgtgctgactgtcttcatagtactttaggtatgttgtccctttacctctgggaggatagcttgatgacctgtctgctcaggccagccccatctagagtctcagtggccccagtcatgttgagaaaggttctttcaaagatagactcaagatagtagtgtcagaggtcccaagcaaatgaagggcggggacagttgagggggtggaatagggacggcagcagggaaccagatagcatgctgctgagaagaaaaaaagacattggtttaggtcaggaagcaaaaaaagggaactgagtggctgtgaaagggtggggtttgctcagactgtccttcctctctggactgtaagaattagtctcgaggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 26 - DNA, 2097 bp, Artificial Sequence
  INT13.BTKp.GFP
gcatttcctaggagaatccctgggggaatcattgcagttggagcataatgtagggggcccggtagggtggactggccagttgcaaaaactacctttgctggccttgccttagggagtgtccttgaggtacactgttctgcagcagctgcctcaaggacgctcaagacagatccaagcaaaagttattcactgattttcttcctctagtggctacgactgggactgcaaaaacatagattcataaagggctttgtcgttgtcttgggtctttttgtcttttatttttaattgtgggaaaattttcagtactatccctgagttcattaactaccatcactaacataatcataaagggatttggggaggttgcttagtctatcttcttgccttatggccaccttgaacctaaaattcccagattcctctaaccaatgaatcccgtttctgagattgacttaagcaaagacagattagtacttctaaaaatttcccttttactagttttcctatttctaccccagtagggatttttgtctattgtaagaattatacattcatgaccccaaagaatcacaccaagactttagggcccctgagaaaacctccaggcttcaagtgacatacctagtctgctttaccggtttacaggactcaagagaaaggtggacattgagagttaatccctgaggccaaatcttaaatggagaaagtcaacatccacagaaaatggggaagggcacaagtatttctgtgggcttatattccgacatttttatctgtaggggaaaaatgctttcttagaaaatgactcagcacggggaagtcttgtctctacctctgtcttgttttgtcctttggggtcccttcactatcaagttcaactgtgtgtccctgagactcctctgccccggaggacaggagactcgaaaaacgctcttcctggccagtctctttgctctgtgtctgccagcccccagcatctctcctctttcctgtaagcccctctccctgtgctgactgtcttcatagtactttaggtatgttgtccctttacctctgggaggatagcttgatgacctgtctgctcaggccagccccatctagagtctcagtggccccagtcatgttgagaaaggttctttcaaagatagactcaagatagtagtgtcagaggtcccaagcaaatgaagggcggggacagttgagggggtggaatagggacggcagcagggaaccagatagcatgctgctgagaagaaaaaaagacattggtttaggtcaggaagcaaaaaaagggaactgagtggctgtgaaagggtggggtttgctcagactgtccttcctctctggactgtaagaattagtctcgaggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 27 - DNA, 2596 bp, Artificial Sequence
  1kb.BTKp.GFP
gcatttcctaggagaatccctgggggaatcattgcagttggagcataatgtagggggcccaaactgtagtctgttagctcaccctagaagaaggaaattctagagtcaagagaagttaagttttagacaagctggaagttggaatatactggagtttaagatcattgactcaatgaaaaaaaaaaaagatttgtctgcagtaaaaccagagttttaaaaatgattagaaatggaccagaaaagagataaggataatgagaaagggggctctctaaaactatttggaactgaaattgggacaatgtttctggaaactattttggcagtatgtatcaaaatgcaaaatgcacatacttttttgactctggaaatgatgcttgtatagagatcctaataacatgggaaaataattaggaataatgttaaataagaacattagatctttttttggaaaaaggtaccttgaacggatggatggatggatagatacatacatacatgcatacatacatacatagatgttacgaaaaataggtaaattaactgaatgtgcaatatgatctcaaatattataagaaacacacacagaaaaaaatggaaggaatatgccgatatattaatgcctctggatgagtttatgaatgattttttcttttttatatccctgtacttgacatattttctacaataagcatgttttattttactatattttgttttattttgagatgggggtctccctatgtttctcaggctgaccttgaactcttgggctcaagcaatcctccaatatcagcctcctgagtagctgggactacaggtgcaccccactgcacccagctgcgtgtgttattttgatatcgatggaaaaaataaataaaatgtttaagccaaggaaaacaaaaactaggttgaaaaagaaggccaaaagggcacacaagtccagagtgaaagacagacaccccagcagtcaccctcagagcagagggagaatattgaaagtattaccactgatctgatctggcactgactataaacttgcagattggctctctggctctccttccatcatcagttggccagaagggcccctgagaaaacctccaggcttcaagtgacatacctagtctgctttaccggtttacaggactcaagagaaaggtggacattgagagttaatccctgaggccaaatcttaaatggagaaagtcaacatccacagaaaatggggaagggcacaagtatttctgtgggcttatattccgacatttttatctgtaggggaaaaatgctttcttagaaaatgactcagcacggggaagtcttgtctctacctctgtcttgttttgtcctttggggtcccttcactatcaagttcaactgtgtgtccctgagactcctctgccccggaggacaggagactcgaaaaacgctcttcctggccagtctctttgctctgtgtctgccagcccccagcatctctcctctttcctgtaagcccctctccctgtgctgactgtcttcatagtactttaggtatgttgtccctttacctctgggaggatagcttgatgacctgtctgctcaggccagccccatctagagtctcagtggccccagtcatgttgagaaaggttctttcaaagatagactcaagatagtagtgtcagaggtcccaagcaaatgaagggcggggacagttgagggggtggaatagggacggcagcagggaaccagatagcatgctgctgagaagaaaaaaagacattggtttaggtcaggaagcaaaaaaagggaactgagtggctgtgaaagggtggggtttgctcagactgtccttcctctctggactgtaagaattagtctcgaggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 28 - DNA, 4519 bp, Artificial Sequence
  CONTIG.BTKp.GFP
gcatttcctaggagaatccctgggggaatcattgcagttggagcataatgtagggggcccctcaaaaagaatacatagcagcaccaattctatcatagtgtgtcttgtctatgataactgcattgagaaagatgctctgcttgttgagtgagcatttcacttccttctggttctgactatctgtctaatagtggtcatgtgggttgaaaagatagaaaaggggagtagtattaggaagttcagtatgaggaagacttattagacttatgcataaacctaaattctgttgtaatctggaagagctgaagtgccacatatgcatctgtttaggagagcaagaactacaaatttggtcttcagtttggcttgcttacatcctgagaactctgtaggccacatgtcgtgaatatagcagcctctgcaacagtgaaagccagaaaaggaagtggaaagtctcaggggagggggctttctgtcatggatttatgagcacagcaagactaacaagcaaaaagaaaaatgtaaaaggatcttgttcgtgtccctgactatatcaaatttacgaaacctttgaaagaggggtatttcagacacagtttttactaccaatgcctttcaacaagatgacattgtttggctgcactagtgaacaatgctgaacagcagataattttaaattaatttatatttgacttcagaatttattatgtgatgtctataaactctgtgagggttggaaccacatcttatatatttcatgcatcttcgtaacacctagcatgatgccttgtacatggttaaaaaaatactatttgacttgatgatattgcttattatataccttaccctttttgaagtaggtttgtagttccaaatttgatttattttggctaatattggtgaggggaggccactaggtagtggaggtggaggtaattagccatgaattaaaattttactccatattatccaagtgaataacccatatgtactaatcaagacttaatttatatagattatcacttcatttctcttctatacaaagtcagaatttaataataagataaaataccctttgttggataaacaaaaatgttatctccagcaagggcagacggagaccttataaaataagtttatgcatgaattgaagcacaagtcaggaggacctaagagtatgaaaaattctaagagcttttaaacgctacattcctagatatgaagagttcaggaatccttcagatagttcacataacctgaacaccattgctgactgaagattctgccattgagcttagtattccatcatatgaataaaagataaaatattagactaagagtttcccagttctatcactatctctctagtgcaattttgtaattttaggcacctccatttctctatttattaagaaaagtcccattttttgcactgaatacaagggtggtgtggcattcaatggttatgcaatatttagaatcctaagtataaaatacctagctccaaaaaattaaatagatgatgtatgagagtgcttgaagtcagagtcagagaacctggtttcaagccctcttttaccatttacaaactgggtgatttctggcaagaaggccaacaagcttctttgatgctcagtttcttcttctcaaaggagaataatggtaattcatgccacatctacctctggggttgttgtaaggatcaaatgaagtaataatacatgtaaaagcacatggaaaactgtaaatcactgtatacgaaagcactgtacatctctttgttgtggataaatatttcccaaggaaatatttcatattccttgctttgtgaggacctaaacttgaacagtttcagcatttcacatcaaccatacagtgtgttcagataaacttgactgtaaatgcttgcagcacaggatgcatgcacacatatttgtgtgtatatatagccacatgtcaagtgactgagagattaactggttgagttgacatcaggaaaggctgcctagagaaacaggaaggatggcagactgataatggtatggttgggttattgcatgacagccctttgtcttaataaacatttagattctgatactcactggctggctataccagccttgtacactcccttgaatgcaagtttggttggtcaaaattatattagccaagtctttcaaactgaaagggaacaggaggaacgaagatgaaggatcatctattcaattattgctatgtgccagatattgagctaaataccttttatatattatctcatttaattttcacaacaaccttggcctaaaaacaacaacaatgataactaacacttaatgagcacttactatgtagcaggctctgtcccaagtgcttcaaatgtattacttatttgatctttgcagtagccttaagaagtatgtattattcccctatttacagatgaggaaactgaagctcaaagatgttatccttatttctctggggtagggtggactggccagttgcaaaaactacctttgctggccttgccttagggagtgtccttgaggtacactgttctgcagcagctgcctcaaggacgctcaagacagatccaagcaaaagttattcactgattttcttcctctagtggctacgactgggactgcaaaaacatagattcataaagggctttgtcgttgtcttgggtctttttgtcttttatttttaattgtgggaaaattttcagtactatccctgagttcattaactaccatcactaacataatcataaagggatttggggaggttgcttagtctatcttcttgccttatggccaccttgaacctaaaattcccagattcctctaaccaatgaatcccgtttctgagattgacttaagcaaagacagattagtacttctaaaaatttcccttttactagttttcctatttctaccccagtagggatttttgtctattgtaagaattatacattcatgaccccaaagaatcacaccaagactttagggcccgtgagaaaacctccaggcttcaagtgacatacctagtctgctttaccggtttacaggactcaagagaaaggtggacattgagagttaatccctgaggccaaatcttaaatggagaaagtcaacatccacagaaaatggggaagggcacaagtatttctgtgggcttatattccgacatttttatctgtaggggaaaaatgctttcttagaaaatgactcagcacggggaagtcttgtctctacctctgtcttgttttgtcctttggggtcccttcactatcaagttcaactgtgtgtccctgagactcctctgccccggaggacaggagactcgaaaaacgctcttcctggccagtctctttgctctgtgtctgccagcccccagcatctctcctctttcctgtaagcccctctccctgtgctgactgtcttcatagtactttaggtatgttgtccctttacctctgggaggatagcttgatgacctgtctgctcaggccagccccatctagagtctcagtggccccagtcatgttgagaaaggttctttcaaagatagactcaagatagtagtgtcagaggtcccaagcaaatgaagggcggggacagttgagggggtggaatagggacggcagcagggaaccagatagcatgctgctgagaagaaaaaaagacattggtttaggtcaggaagcaaaaaaagggaactgagtggctgtgaaagggtggggtttgctcagactgtccttcctctctggactgtaagaattagtctcgaggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 29 - DNA, 4499 bp, Artificial Sequence
  3kb.BTKp.GFP
gcatttcctaggagaatccctgggggaatcattgcagttggagcataatgtagggggcccaaactgtagtctgttagctcaccctagaagaaggaaattctagagtcaagagaagttaagttttagacaagctggaagttggaatatactggagtttaagatcattgactcaatgaaaaaaaaaaaagatttgtctgcagtaaaaccagagttttaaaaatgattagaaatggaccagaaaagagataaggataatgagaaagggggctctctaaaactatttggaactgaaattgggacaatgtttctggaaactattttggcagtatgtatcaaaatgcaaaatgcacatacttttttgactctggaaatgatgcttgtatagagatcctaataacatgggaaaataattaggaataatgttaaataagaacattagatctttttttggaaaaaggtaccttgaacggatggatggatggatagatacatacatacatgcatacatacatacatagatgttacgaaaaataggtaaattaactgaatgtgcaatatgatctcaaatattataagaaacacacacagaaaaaaatggaaggaatatgccgatatattaatgcctctggatgagtttatgaatgattttttcttttttatatccctgtacttgacatattttctacaataagcatgttttattttactatattttgttttattttgagatgggggtctccctatgtttctcaggctgaccttgaactcttgggctcaagcaatcctccaatatcagcctcctgagtagctgggactacaggtgcaccccactgcacccagctgcgtgtgttattttgatatcgatggaaaaaataaataaaatgtttaagccaaggaaaacaaaaactaggttgaaaaagaaggccaaaagggcacacaagtccagagtgaaagacagacaccccagcagtcaccctcagagcagagggagaatattgaaagtattaccactgatctgatctggcactgactataaacttgcagattggctctctggctctccttccatcatcagttggccagaatatattgtcgctaatgcaagacaaaaagagatgatgaatcacagagacctagaagtttccaaaaccacagaagaaaaatgtaaaatgctaaaaacataaggccaggcgcggtggctcaagcctgtaatcccagcattttgggaggccgaggcaggtgtcatggtgtgcggctgtagtccaagctactcaggaggctgaggcaagagaattgcttgaaactgggaggtagcgattgcagtgagccaagattgcgctactgtaccgaagcctggatgacagagcgagactctgtctcaaaaataaataaataaataaataaataaataaaacatgaactgctgtggaagaaccagagagccctaaggcctcctgtaaatgactacagcaggagagtttccctggtcagcatgcagctttcgaaaattgagtttggcttgatgccttcatcacaaatttggtcccctgtagccttgactatgaagcttatcataaaataattgggtttcactggggcccgaacaaaagcatcttgggaatttaacaagattaaaggttgctttgttgctgtgggtatgatttttttttcttttttctttttttttttttttgagacggagtctgactctgtcacccaggctggagtgcagtggcacgatctcggctcactgcaacctccgcctcctgggttcaagcaattctcctgcctcagcctcctgagtaactgggattacaggtgcgcgccaccacgccaggctaatttttgtatttttagtagagacagggtttctccatgttggtcaggctggctgctctcaaactcctgaccttgccatccagccgcctcagcctcccaaagtgctgggattacaggcatgagctaccacacgtggccatatgtaaattttaaacatgtaaaccaatactgtatatcttttatagatatttacatctgtaatgataatatgaaaacgtacctaggaatgataaacaccaaattcaagatattggtaatctctaaggagggagaagactaagattttggagtacataaaggggtcttgattatatagatagtatttcagttcttaacacgttctcttctggtcccaaggccaaggaattctcaattgaaatctcagttagaacgtgaaggatgcttcttgggactgggaagagattttccgtaggccaaatttaacctcccactagagatagttgataattctaccacatttcatagtgaagcatcaggaagacaggactgaaaaggcaagaaggaatgatgcaaattgcatagtccaaaagataagctggacctaggtttgccagtttgtctaagtaactgagaattcattctgagatcaaaaaatcaaaataatgttcacagcagcattgtttatcactgaaagcaaacaacctaaatgtccatcaacaggagaatgaataaatatattggaatgtattcatcatatagaatattttatgcatataaaaatgaattaaaacatatgaagtagaactagccagataaataaatcctaacatattatattgaggggaaaaaagcaagttgctggagaatgtgtaccatatatatatatataaattttctttttttttttttttttttttcgcgaccgagtttcactcttgttgcccaggctggagtgcaatggcaagatctcggctcactgcaacctctgcctcccgggttcaagggattctcccgcctcagcctcctgagtagctgggagagtagcgttggcccagctagtttttgtatttttagtagagacagggtttctccatgttggtcaggctggctgctctcaaactcctgaccttgtcatccacccgcctcagcctcccaaagtgctgggattacaggcctgagttaccgcacgggcccctgagaaaacctccaggcttcaagtgacatacctagtctgctttaccggtttacaggactcaagagaaaggtggacattgagagttaatccctgaggccaaatcttaaatggagaaagtcaacatccacagaaaatggggaagggcacaagtatttctgtgggcttatattccgacatttttatctgtaggggaaaaatgctttcttagaaaatgactcagcacggggaagtcttgtctctacctctgtcttgttttgtcctttggggtcccttcactatcaagttcaactgtgtgtccctgagactcctctgccccggaggacaggagactcgaaaaacgctcttcctggccagtctctttgctctgtgtctgccagcccccagcatctctcctctttcctgtaagcccctctccctgtgctgactgtcttcatagtactttaggtatgttgtccctttacctctgggaggatagcttgatgacctgtctgctcaggccagccccatctagagtctcagtggccccagtcatgttgagaaaggttctttcaaagatagactcaagatagtagtgtcagaggtcccaagcaaatgaagggcggggacagttgagggggtggaatagggacggcagcagggaaccagatagcatgctgctgagaagaaaaaaagacattggtttaggtcaggaagcaaaaaaagggaactgagtggctgtgaaagggtggggtttgctcagactgtccttcctctctggactgtaagaattagtctcgaggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 30 - DNA, 4519 bp, Artificial Sequence
  revCONTIG.BTKp.GFP
gcatttcctaggagaatccctgggggaatcattgcagttggagcataatgtagggggccctaaagtcttggtgtgattctttggggtcatgaatgtataattcttacaatagacaaaaatccctactggggtagaaataggaaaactagtaaaagggaaatttttagaagtactaatctgtctttgcttaagtcaatctcagaaacgggattcattggttagaggaatctgggaattttaggttcaaggtggccataaggcaagaagatagactaagcaacctccccaaatccctttatgattatgttagtgatggtagttaatgaactcagggatagtactgaaaattttcccacaattaaaaataaaagacaaaaagacccaagacaacgacaaagccctttatgaatctatgtttttgcagtcccagtcgtagccactagaggaagaaaatcagtgaataacttttgcttggatctgtcttgagcgtccttgaggcagctgctgcagaacagtgtacctcaaggacactccctaaggcaaggccagcaaaggtagtttttgcaactggccagtccaccctaccccagagaaataaggataacatctttgagcttcagtttcctcatctgtaaataggggaataatacatacttcttaaggctactgcaaagatcaaataagtaatacatttgaagcacttgggacagagcctgctacatagtaagtgctcattaagtgttagttatcattgttgttgtttttaggccaaggttgttgtgaaaattaaatgagataatatataaaaggtatttagctcaatatctggcacatagcaataattgaatagatgatccttcatcttcgttcctcctgttccctttcagtttgaaagacttggctaatataattttgaccaaccaaacttgcattcaagggagtgtacaaggctggtatagccagccagtgagtatcagaatctaaatgtttattaagacaaagggctgtcatgcaataacccaaccataccattatcagtctgccatccttcctgtttctctaggcagcctttcctgatgtcaactcaaccagttaatctctcagtcacttgacatgtggctatatatacacacaaatatgtgtgcatgcatcctgtgctgcaagcatttacagtcaagtttatctgaacacactgtatggttgatgtgaaatgctgaaactgttcaagtttaggtcctcacaaagcaaggaatatgaaatatttccttgggaaatatttatccacaacaaagagatgtacagtgctttcgtatacagtgatttacagttttccatgtgcttttacatgtattattacttcatttgatccttacaacaaccccagaggtagatgtggcatgaattaccattattctcctttgagaagaagaaactgagcatcaaagaagcttgttggccttcttgccagaaatcacccagtttgtaaatggtaaaagagggcttgaaaccaggttctctgactctgacttcaagcactctcatacatcatctatttaattttttggagctaggtattttatacttaggattctaaatattgcataaccattgaatgccacaccacccttgtattcagtgcaaaaaatgggacttttcttaataaatagagaaatggaggtgcctaaaattacaaaattgcactagagagatagtgatagaactgggaaactcttagtctaatattttatcttttattcatatgatggaatactaagctcaatggcagaatcttcagtcagcaatggtgttcaggttatgtgaactatctgaaggattcctgaactcttcatatctaggaatgtagcgtttaaaagctcttagaatttttcatactcttaggtcctcctgacttgtgcttcaattcatgcataaacttattttataaggtctccgtctgcccttgctggagataacatttttgtttatccaacaaagggtattttatcttattattaaattctgactttgtatagaagagaaatgaagtgataatctatataaattaagtcttgattagtacatatgggttattcacttggataatatggagtaaaattttaattcatggctaattacctccacctccactacctagtggcctcccctcaccaatattagccaaaataaatcaaatttggaactacaaacctacttcaaaaagggtaaggtatataataagcaatatcatcaagtcaaatagtatttttttaaccatgtacaaggcatcatgctaggtgttacgaagatgcatgaaatatataagatgtggttccaaccctcacagagtttatagacatcacataataaattctgaagtcaaatataaattaatttaaaattatctgctgttcagcattgttcactagtgcagccaaacaatgtcatcttgttgaaaggcattggtagtaaaaactgtgtctgaaatacccctctttcaaaggtttcgtaaatttgatatagtcagggacacgaacaagatccttttacatttttctttttgcttgttagtcttgctgtgctcataaatccatgacagaaagccccctcccctgagactttccacttccttttctggctttcactgttgcagaggctgctatattcacgacatgtggcctacagagttctcaggatgtaagcaagccaaactgaagaccaaatttgtagttcttgctctcctaaacagatgcatatgtggcacttcagctcttccagattacaacagaatttaggtttatgcataagtctaataagtcttcctcatactgaacttcctaatactactccccttttctatcttttcaacccacatgaccactattagacagatagtcagaaccagaaggaagtgaaatgctcactcaacaagcagagcatctttctcaatgcagttatcatagacaagacacactatgatagaattggtgctgctatgtattctttttgaggggcccctgagaaaacctccaggcttcaagtgacatacctagtctgctttaccggtttacaggactcaagagaaaggtggacattgagagttaatccctgaggccaaatcttaaatggagaaagtcaacatccacagaaaatggggaagggcacaagtatttctgtgggcttatattccgacatttttatctgtaggggaaaaatgctttcttagaaaatgactcagcacggggaagtcttgtctctacctctgtcttgttttgtcctttggggtcccttcactatcaagttcaactgtgtgtccctgagactcctctgccccggaggacaggagactcgaaaaacgctcttcctggccagtctctttgctctgtgtctgccagcccccagcatctctcctctttcctgtaagcccctctccctgtgctgactgtcttcatagtactttaggtatgttgtccctttacctctgggaggatagcttgatgacctgtctgctcaggccagccccatctagagtctcagtggccccagtcatgttgagaaaggttctttcaaagatagactcaagatagtagtgtcagaggtcccaagcaaatgaagggcggggacagttgagggggtggaatagggacggcagcagggaaccagatagcatgctgctgagaagaaaaaaagacattggtttaggtcaggaagcaaaaaaagggaactgagtggctgtgaaagggtggggtttgctcagactgtccttcctctctggactgtaagaattagtctcgaggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 31 - DNA, 3974 bp, Artificial Sequence
  IE.BTKp.GFP
gcatttcctaggagaatccctgggggaatcattgcagttggagcataatgtagggggcccacatctttgagcttcagtttcctcatctgtaaataggggaataatacatacttcttaaggctactgcaaagatcaaataagtaatacatttgaagcacttgggacagagcctgctacatagtaagtgctcattaagtgttagttatcattgttgttgtttttaggccaaggttgttgtgaaaattaaatgagataatatataaaaggtatttagctcaatatctggcacatagcaataattgaatagatgatccttcatcttcgttcctcctgttccctttcagtttgaaagacttggctaatataattttgaccaaccaaacttgcattcaagggagtgtacaaggctggtatagccagccagtgagtatcagaatctaaatgtttattaagacaaagggctgtcatgcaataacccaaccataccattatcagtctgccatccttcctgtttctctaggcagcctttcctgatgtcaactcaaccagttaatctctcagtcacttgacatgtggctatatatacacacaaatatgtgtgcatgcatcctgtgctgcaagcatttacagtcaagtttatctgaacacactgtatggttgatgtgaaatgctgaaactgttcaagtttaggtcctcacaaagcaaggaatatgaaatatttccttgggaaatatttatccacaacaaagagatgtacagtgctttcgtatacagtgatttacagttttccatgtgcttttacatgtattattacttcatttgatccttacaacaaccccagaggtagatgtggcatgaattaccattattctcctttgagaagaagaaactgagcatcaaagaagcttgttggccttcttgccagaaatcacccagtttgtaaatggtaaaagagggcttgaaaccaggttctctgactctgacttcaagcactctcatacatcatctatttaattttttggagctaggtattttatacttaggattctaaatattgcataaccattgaatgccacaccacccttgtattcagtgcaaaaaatgggacttttcttaataaatagagaaatggaggtgcctaaaattacaaaattgcactagagagatagtgatagaactgggaaactcttagtctaatattttatcttttattcatatgatggaatactaagctcaatggcagaatcttcagtcagcaatggtgttcaggttatgtgaactatctgaaggattcctgaactcttcatatctaggaatgtagcgtttaaaagctcttagaatttttcatactcttaggtcctcctgacttgtgcttcaattcatgcataaacttattttataaggtctccgtctgcccttgctggagataacatttttgtttatccaacaaagggtattttatcttattattaaattctgactttgtatagaagagaaatgaagtgataatctatataaattaagtcttgattagtacatatgggttattcacttggataatatggagtaaaattttaattcatggctaattacctccacctccactacctagtggcctcccctcaccaatattagccaaaataaatcaaatttggaactacaaacctacttcaaaaagggtaaggtatataataagcaatatcatcaagtcaaatagtatttttttaaccatgtacaaggcatcatgctaggtgttacgaagatgcatgaaatatataagatgtggttccaaccctcacagagtttatagacatcacataataaattctgaagtcaaatataaattaatttaaaattatctgctgttcagcattgttcactagtgcagccaaacaatgtcatcttgttgaaaggcattggtagtaaaaactgtgtctgaaatacccctctttcaaaggtttcgtaaatttgatatagtcagggacacgaacaagatccttttacatttttctttttgcttgttagtcttgctgtgctcataaatccatgacagaaagccccctcccctgagactttccacttccttttctggctttcactgttgcagaggctgctatattcacgacatgtggcctacagagttctcaggatgtaagcaagccaaactgaagaccaaatttgtagttcttgctctcctaaacagatgcatatgtggcacttcagctcttccagattacaacagaatttaggtttatgcataagtctaataagtcttcctcatactgaacttcctaatactactccccttttctatcttttcaacccacatgaccactattagacagatagtcagaaccagaaggaagtgaaatgctcactcaacaagcagagcatctttctcaatgcagttatcatagacaagacacactatgatagaattggtgctgctatgtattctttttgaggcccctgagaaaacctccaggcttcaagtgacatacctagtctgctttaccggtttacaggactcaagagaaaggtggacattgagagttaatccctgaggccaaatcttaaatggagaaagtcaacatccacagaaaatggggaagggcacaagtatttctgtgggcttatattccgacatttttatctgtaggggaaaaatgctttcttagaaaatgactcagcacggggaagtcttgtctctacctctgtcttgttttgtcctttggggtcccttcactatcaagttcaactgtgtgtccctgagactcctctgccccggaggacaggagactcgaaaaacgctcttcctggccagtctctttgctctgtgtctgccagcccccagcatctctcctctttcctgtaagcccctctccctgtgctgactgtcttcatagtactttaggtatgttgtccctttacctctgggaggatagcttgatgacctgtctgctcaggccagccccatctagagtctcagtggccccagtcatgttgagaaaggttctttcaaagatagactcaagatagtagtgtcagaggtcccaagcaaatgaagggcggggacagttgagggggtggaatagggacggcagcagggaaccagatagcatgctgctgagaagaaaaaaagacattggtttaggtcaggaagcaaaaaaagggaactgagtggctgtgaaagggtggggtttgctcagactgtccttcctctctggactgtaagaattagtctcgaggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 32 - DNA, 4647 bp, Artificial Sequence
  0.7UCOE.IE.BTKp.GFP
gcatttcctaggagaatccctgggggaatcattgcagttggagcataatgtagggggccgcgtgtggcatctgaagcaccaccagcgagcgagagctagagagaaggaaagccaccgacttcaccgcctccgagctgctccgggtcgcgggtctgcagcgtctccggccctccgcgcctacagctcaagccacatccgaagggggagggagccgggagctgcgcgcggggccgccggggggaggggtggcaccgcccacgccgggcggccacgaagggcggggcagcgggcgcgcgcccggcggggggaggggccgcgcgccgcgcccgctgggaattggggccctagggggagggcggaggcgccgacgaccgcggcacttaccgttcgcggcgtggcgcccggtggtccccaaggggagggaagggggaggcggggcgaggacagtgaccggagtctcctcagcggtggcttttctgcttggcagcctcagcggctggcgccaaaaccggactccgcccacttcctcgcccctgcggtgcgagggtgtggaatcctccagacgctgggggagggggagttgggagcttaaaaactagtacccctttgggaccactttcagcagcgaactctcctgtacaccaggggtcagttccacagacgcgggccaggggtgggtcattgcggcgtgaacaataatttgactagaagttgattcgggtgtttgcggcccacatctttgagcttcagtttcctcatctgtaaataggggaataatacatacttcttaaggctactgcaaagatcaaataagtaatacatttgaagcacttgggacagagcctgctacatagtaagtgctcattaagtgttagttatcattgttgttgtttttaggccaaggttgttgtgaaaattaaatgagataatatataaaaggtatttagctcaatatctggcacatagcaataattgaatagatgatccttcatcttcgttcctcctgttccctttcagtttgaaagacttggctaatataattttgaccaaccaaacttgcattcaagggagtgtacaaggctggtatagccagccagtgagtatcagaatctaaatgtttattaagacaaagggctgtcatgcaataacccaaccataccattatcagtctgccatccttcctgtttctctaggcagcctttcctgatgtcaactcaaccagttaatctctcagtcacttgacatgtggctatatatacacacaaatatgtgtgcatgcatcctgtgctgcaagcatttacagtcaagtttatctgaacacactgtatggttgatgtgaaatgctgaaactgttcaagtttaggtcctcacaaagcaaggaatatgaaatatttccttgggaaatatttatccacaacaaagagatgtacagtgctttcgtatacagtgatttacagttttccatgtgcttttacatgtattattacttcatttgatccttacaacaaccccagaggtagatgtggcatgaattaccattattctcctttgagaagaagaaactgagcatcaaagaagcttgttggccttcttgccagaaatcacccagtttgtaaatggtaaaagagggcttgaaaccaggttctctgactctgacttcaagcactctcatacatcatctatttaattttttggagctaggtattttatacttaggattctaaatattgcataaccattgaatgccacaccacccttgtattcagtgcaaaaaatgggacttttcttaataaatagagaaatggaggtgcctaaaattacaaaattgcactagagagatagtgatagaactgggaaactcttagtctaatattttatcttttattcatatgatggaatactaagctcaatggcagaatcttcagtcagcaatggtgttcaggttatgtgaactatctgaaggattcctgaactcttcatatctaggaatgtagcgtttaaaagctcttagaatttttcatactcttaggtcctcctgacttgtgcttcaattcatgcataaacttattttataaggtctccgtctgcccttgctggagataacatttttgtttatccaacaaagggtattttatcttattattaaattctgactttgtatagaagagaaatgaagtgataatctatataaattaagtcttgattagtacatatgggttattcacttggataatatggagtaaaattttaattcatggctaattacctccacctccactacctagtggcctcccctcaccaatattagccaaaataaatcaaatttggaactacaaacctacttcaaaaagggtaaggtatataataagcaatatcatcaagtcaaatagtatttttttaaccatgtacaaggcatcatgctaggtgttacgaagatgcatgaaatatataagatgtggttccaaccctcacagagtttatagacatcacataataaattctgaagtcaaatataaattaatttaaaattatctgctgttcagcattgttcactagtgcagccaaacaatgtcatcttgttgaaaggcattggtagtaaaaactgtgtctgaaatacccctctttcaaaggtttcgtaaatttgatatagtcagggacacgaacaagatccttttacatttttctttttgcttgttagtcttgctgtgctcataaatccatgacagaaagccccctcccctgagactttccacttccttttctggctttcactgttgcagaggctgctatattcacgacatgtggcctacagagttctcaggatgtaagcaagccaaactgaagaccaaatttgtagttcttgctctcctaaacagatgcatatgtggcacttcagctcttccagattacaacagaatttaggtttatgcataagtctaataagtcttcctcatactgaacttcctaatactactccccttttctatcttttcaacccacatgaccactattagacagatagtcagaaccagaaggaagtgaaatgctcactcaacaagcagagcatctttctcaatgcagttatcatagacaagacacactatgatagaattggtgctgctatgtattctttttgaggcccctgagaaaacctccaggcttcaagtgacatacctagtctgctttaccggtttacaggactcaagagaaaggtggacattgagagttaatccctgaggccaaatcttaaatggagaaagtcaacatccacagaaaatggggaagggcacaagtatttctgtgggcttatattccgacatttttatctgtaggggaaaaatgctttcttagaaaatgactcagcacggggaagtcttgtctctacctctgtcttgttttgtcctttggggtcccttcactatcaagttcaactgtgtgtccctgagactcctctgccccggaggacaggagactcgaaaaacgctcttcctggccagtctctttgctctgtgtctgccagcccccagcatctctcctctttcctgtaagcccctctccctgtgctgactgtcttcatagtactttaggtatgttgtccctttacctctgggaggatagcttgatgacctgtctgctcaggccagccccatctagagtctcagtggccccagtcatgttgagaaaggttctttcaaagatagactcaagatagtagtgtcagaggtcccaagcaaatgaagggcggggacagttgagggggtggaatagggacggcagcagggaaccagatagcatgctgctgagaagaaaaaaagacattggtttaggtcaggaagcaaaaaaagggaactgagtggctgtgaaagggtggggtttgctcagactgtccttcctctctggactgtaagaattagtctcgaggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 33 - DNA, 3541 bp, Artificial Sequence
  0.7UCOE.BTKp.coBTK
ccgcgtgtggcatctgaagcaccaccagcgagcgagagctagagagaaggaaagccaccgacttcaccgcctccgagctgctccgggtcgcgggtctgcagcgtctccggccctccgcgcctacagctcaagccacatccgaagggggagggagccgggagctgcgcgcggggccgctggggggaggggtggcaccgcccacgccgggcggccacgaagggcggggcagcgggcgcgcgcccggcggggggaggggccgcgcgccgcgcccgctgggaattggggccctagggggagggcggaggcgccgacgaccgcggcacttaccgttcgcggcgtggcgcccggtggtccccaaggggagggaagggggaggcggggcgaggacagtgaccggagtctcctcagcggtggcttttctgcttggcagcctcagcggctggcgccaaaaccggactccgcccacttcctcgcccctgcggtgcgagggtgtggaatcctccagacgctgggggagggggagttgggagcttaaaaactagtacccctttgggaccactttcagcagcgaactctcctgtacaccaggggtcagttccacagacgcgggccaggggtgggtcattgcggcgtgaacaataatttgactagaagttgattcgggtgtttgcggccggggctagctacgacgcgttccggaattcgcccttgcatttcctaggagaatccctgggggaatcattgcagttggagcataatgtagggggcccctgagaaaacctccaggcttcaagtgacatacctagtctgctttaccggtttacaggactcaagagaaaggtggacattgagagttaatccctgaggccaaatcttaaatggagaaagtcaacatccacagaaaatggggaagggcacaagtatttctgtgggcttatattccgacatttttatctgtaggggaaaaatgctttcttagaaaatgactcagcacggggaagtcttgtctctacctctgtcttgttttgtcctttggggtcccttcactatcaagttcaactgtgtgtccctgagactcctctgccccggaggacaggagactcgaaaaacgctcttcctggccagtctctttgctctgtgtctgccagcccccagcatctctcctctttcctgtaagcccctctccctgtgctgactgtcttcatagtactttaggtatgttgtccctttacctctgggaggatagcttgatgacctgtctgctcaggccagccccatctagagtctcagtggccccagtcatgttgagaaaggttctttcaaagatagactcaagatagtagtgtcagaggtcccaagcaaatgaagggcggggacagttgagggggtggaatagggacggcagcagggaaccagatagcatgctgctgagaagaaaaaaagacattggtttaggtcaggaagcaaaaaaagggaactgagtggctgtgaaagggtggggtttgctcagactgtccttcctctctggactgtaagaattagtctcgagaaagaagccaccatggccgctgtgatcctggagagcattttcctgaagaggtcccagcagaaaaagaaaacctctcccctgaactttaagaaaagactgttcctgctgacagtgcacaagctgtcttactatgagtacgactttgagcggggccgccgaggatcaaaaaaggggagcatcgatgtggagaagattacatgcgtggagaccgtggtccctgaaaagaatccaccccctgagaggcagatcccaagacggggcgaggagtcctctgagatggagcagattagtatcattgagcgcttcccctatccttttcaggtggtgtacgacgagggaccactgtatgtgttctcacccacagaggagctgagaaagaggtggattcaccagctgaagaacgtgattagatacaatagcgatctggtgcagaagtatcacccttgtttttggatcgacgggcagtacctgtgctgttcccagacagctaagaacgctatgggatgccagattctggaaaatcggaacggatctctgaaaccagggagttcacaccgcaagaccaaaaagcccctgcctccaacacccgaggaggatcagatcctgaaaaagcctctgccacccgagcctgctgcagccccagtcagcacttccgaactgaaaaaggtggtggctctgtatgactacatgcccatgaatgctaacgatctgcagctgagaaagggcgacgagtatttcattctggaagagtctaatctgccttggtggagggccagagataagaacggacaggaggggtacatcccatctaattatgtgaccgaggctgaggactctattgagatgtacgagtggtatagcaagcacatgacacggtcccaggctgagcagctgctgaagcaggagggcaaagagggagggtttatcgtgcgcgattctagtaaggccggcaaatacactgtgtcagtgttcgctaagagcaccggagacccccagggcgtgatcagacactatgtggtgtgttccacacctcagtctcagtactatctggctgagaagcacctgtttagtacaatcccagagctgattaactaccaccagcacaattctgccggcctgatcagcaggctgaagtatcccgtctcccagcagaacaaaaatgctccttctaccgctggactggggtacggcagttgggagattgatccaaaggacctgacattcctgaaggagctgggaactgggcagtttggcgtggtgaagtatggaaaatggagagggcagtacgatgtggccatcaagatgatcaaggagggctcaatgagcgaggacgagttcatcgaggaggctaaggtcatgatgaacctgtcccacgagaaactggtgcagctgtatggagtgtgcaccaagcagcggcccatttttatcattacagagtacatggctaatgggtgtctgctgaactatctgcgcgagatgagacacagattccagacacagcagctgctggaaatgtgcaaggatgtgtgtgaggctatggagtacctggagtctaagcagtttctgcaccgggacctggctgctcgcaattgcctggtgaacgatcagggcgtggtgaaggtgagtgacttcggactgtcaaggtatgtgctggatgacgagtacaccagctccgtgggctctaagtttcctgtgagatggtctccacccgaggtgctgatgtatagcaagttctcctctaagagcgatatctgggcctttggcgtgctgatgtgggaaatctacagcctgggcaagatgccttacgagcggttcacaaattccgagacagctgagcacatcgcccagggcctgcgcctgtaccggccacatctggcctctgagaaggtgtacaccatcatgtacagctgttggcacgagaaggccgacgagagacccacattcaagatcctgctgtccaacattctagatgtgatggacgaggagagctga

SEQ ID NO: 34 - DNA, 5240 bp, Artificial Sequence
  IE.BTKp.coBTK
gcatttcctaggagaatccctgggggaatcattgcagttggagcataatgtagggggcccacatctttgagcttcagtttcctcatctgtaaataggggaataatacatacttcttaaggctactgcaaagatcaaataagtaatacatttgaagcacttgggacagagcctgctacatagtaagtgctcattaagtgttagttatcattgttgttgtttttaggccaaggttgttgtgaaaattaaatgagataatatataaaaggtatttagctcaatatctggcacatagcaataattgaatagatgatccttcatcttcgttcctcctgttccctttcagtttgaaagacttggctaatataattttgaccaaccaaacttgcattcaagggagtgtacaaggctggtatagccagccagtgagtatcagaatctaaatgtttattaagacaaagggctgtcatgcaataacccaaccataccattatcagtctgccatccttcctgtttctctaggcagcctttcctgatgtcaactcaaccagttaatctctcagtcacttgacatgtggctatatatacacacaaatatgtgtgcatgcatcctgtgctgcaagcatttacagtcaagtttatctgaacacactgtatggttgatgtgaaatgctgaaactgttcaagtttaggtcctcacaaagcaaggaatatgaaatatttccttgggaaatatttatccacaacaaagagatgtacagtgctttcgtatacagtgatttacagttttccatgtgcttttacatgtattattacttcatttgatccttacaacaaccccagaggtagatgtggcatgaattaccattattctcctttgagaagaagaaactgagcatcaaagaagcttgttggccttcttgccagaaatcacccagtttgtaaatggtaaaagagggcttgaaaccaggttctctgactctgacttcaagcactctcatacatcatctatttaattttttggagctaggtattttatacttaggattctaaatattgcataaccattgaatgccacaccacccttgtattcagtgcaaaaaatgggacttttcttaataaatagagaaatggaggtgcctaaaattacaaaattgcactagagagatagtgatagaactgggaaactcttagtctaatattttatcttttattcatatgatggaatactaagctcaatggcagaatcttcagtcagcaatggtgttcaggttatgtgaactatctgaaggattcctgaactcttcatatctaggaatgtagcgtttaaaagctcttagaatttttcatactcttaggtcctcctgacttgtgcttcaattcatgcataaacttattttataaggtctccgtctgcccttgctggagataacatttttgtttatccaacaaagggtattttatcttattattaaattctgactttgtatagaagagaaatgaagtgataatctatataaattaagtcttgattagtacatatgggttattcacttggataatatggagtaaaattttaattcatggctaattacctccacctccactacctagtggcctcccctcaccaatattagccaaaataaatcaaatttggaactacaaacctacttcaaaaagggtaaggtatataataagcaatatcatcaagtcaaatagtatttttttaaccatgtacaaggcatcatgctaggtgttacgaagatgcatgaaatatataagatgtggttccaaccctcacagagtttatagacatcacataataaattctgaagtcaaatataaattaatttaaaattatctgctgttcagcattgttcactagtgcagccaaacaatgtcatcttgttgaaaggcattggtagtaaaaactgtgtctgaaatacccctctttcaaaggtttcgtaaatttgatatagtcagggacacgaacaagatccttttacatttttctttttgcttgttagtcttgctgtgctcataaatccatgacagaaagccccctcccctgagactttccacttccttttctggctttcactgttgcagaggctgctatattcacgacatgtggcctacagagttctcaggatgtaagcaagccaaactgaagaccaaatttgtagttcttgctctcctaaacagatgcatatgtggcacttcagctcttccagattacaacagaatttaggtttatgcataagtctaataagtcttcctcatactgaacttcctaatactactccccttttctatcttttcaacccacatgaccactattagacagatagtcagaaccagaaggaagtgaaatgctcactcaacaagcagagcatctttctcaatgcagttatcatagacaagacacactatgatagaattggtgctgctatgtattctttttgaggcccctgagaaaacctccaggcttcaagtgacatacctagtctgctttaccggtttacaggactcaagagaaaggtggacattgagagttaatccctgaggccaaatcttaaatggagaaagtcaacatccacagaaaatggggaagggcacaagtatttctgtgggcttatattccgacatttttatctgtaggggaaaaatgctttcttagaaaatgactcagcacggggaagtcttgtctctacctctgtcttgttttgtcctttggggtcccttcactatcaagttcaactgtgtgtccctgagactcctctgccccggaggacaggagactcgaaaaacgctcttcctggccagtctctttgctctgtgtctgccagcccccagcatctctcctctttcctgtaagcccctctccctgtgctgactgtcttcatagtactttaggtatgttgtccctttacctctgggaggatagcttgatgacctgtctgctcaggccagccccatctagagtctcagtggccccagtcatgttgagaaaggttctttcaaagatagactcaagatagtagtgtcagaggtcccaagcaaatgaagggcggggacagttgagggggtggaatagggacggcagcagggaaccagatagcatgctgctgagaagaaaaaaagacattggtttaggtcaggaagcaaaaaaagggaactgagtggctgtgaaagggtggggtttgctcagactgtccttcctctctggactgtaagaattagtctcgagaaagaagccaccatggccgctgtgatcctggagagcattttcctgaagaggtcccagcagaaaaagaaaacctctcccctgaactttaagaaaagactgttcctgctgacagtgcacaagctgtcttactatgagtacgactttgagcggggccgccgaggatcaaaaaaggggagcatcgatgtggagaagattacatgcgtggagaccgtggtccctgaaaagaatccaccccctgagaggcagatcccaagacggggcgaggagtcctctgagatggagcagattagtatcattgagcgcttcccctatccttttcaggtggtgtacgacgagggaccactgtatgtgttctcacccacagaggagctgagaaagaggtggattcaccagctgaagaacgtgattagatacaatagcgatctggtgcagaagtatcacccttgtttttggatcgacgggcagtacctgtgctgttcccagacagctaagaacgctatgggatgccagattctggaaaatcggaacggatctctgaaaccagggagttcacaccgcaagaccaaaaagcccctgcctccaacacccgaggaggatcagatcctgaaaaagcctctgccacccgagcctgctgcagccccagtcagcacttccgaactgaaaaaggtggtggctctgtatgactacatgcccatgaatgctaacgatctgcagctgagaaagggcgacgagtatttcattctggaagagtctaatctgccttggtggagggccagagataagaacggacaggaggggtacatcccatctaattatgtgaccgaggctgaggactctattgagatgtacgagtggtatagcaagcacatgacacggtcccaggctgagcagctgctgaagcaggagggcaaagagggagggtttatcgtgcgcgattctagtaaggccggcaaatacactgtgtcagtgttcgctaagagcaccggagacccccagggcgtgatcagacactatgtggtgtgttccacacctcagtctcagtactatctggctgagaagcacctgtttagtacaatcccagagctgattaactaccaccagcacaattctgccggcctgatcagcaggctgaagtatcccgtctcccagcagaacaaaaatgctccttctaccgctggactggggtacggcagttgggagattgatccaaaggacctgacattcctgaaggagctgggaactgggcagtttggcgtggtgaagtatggaaaatggagagggcagtacgatgtggccatcaagatgatcaaggagggctcaatgagcgaggacgagttcatcgaggaggctaaggtcatgatgaacctgtcccacgagaaactggtgcagctgtatggagtgtgcaccaagcagcggcccatttttatcattacagagtacatggctaatgggtgtctgctgaactatctgcgcgagatgagacacagattccagacacagcagctgctggaaatgtgcaaggatgtgtgtgaggctatggagtacctggagtctaagcagtttctgcaccgggacctggctgctcgcaattgcctggtgaacgatcagggcgtggtgaaggtgagtgacttcggactgtcaaggtatgtgctggatgacgagtacaccagctccgtgggctctaagtttcctgtgagatggtctccacccgaggtgctgatgtatagcaagttctcctctaagagcgatatctgggcctttggcgtgctgatgtgggaaatctacagcctgggcaagatgccttacgagcggttcacaaattccgagacagctgagcacatcgcccagggcctgcgcctgtaccggccacatctggcctctgagaaggtgtacaccatcatgtacagctgttggcacgagaaggccgacgagagacccacattcaagatcctgctgtccaacattctagatgtgatggacgaggagagctga

SEQ ID NO: 35 - DNA, 5941 bp, Artificial Sequence
  0.7UCOE.IE.BTKp.coBTK
ccgcgtgtggcatctgaagcaccaccagcgagcgagagctagagagaaggaaagccaccgacttcaccgcctccgagctgctccgggtcgcgggtctgcagcgtctccggccctccgcgcctacagctcaagccacatccgaagggggagggagccgggagctgcgcgcggggccgctggggggaggggtggcaccgcccacgccgggcggccacgaagggcggggcagcgggcgcgcgcccggcggggggaggggccgcgcgccgcgcccgctgggaattggggccctagggggagggcggaggcgccgacgaccgcggcacttaccgttcgcggcgtggcgcccggtggtccccaaggggagggaagggggaggcggggcgaggacagtgaccggagtctcctcagcggtggcttttctgcttggcagcctcagcggctggcgccaaaaccggactccgcccacttcctcgcccctgcggtgcgagggtgtggaatcctccagacgctgggggagggggagttgggagcttaaaaactagtacccctttgggaccactttcagcagcgaactctcctgtacaccaggggtcagttccacagacgcgggccaggggtgggtcattgcggcgtgaacaataatttgactagaagttgattcgggtgtttgcggccggggctagcacatctttgagcttcagtttcctcatctgtaaataggggaataatacatacttcttaaggctactgcaaagatcaaataagtaatacatttgaagcacttgggacagagcctgctacatagtaagtgctcattaagtgttagttatcattgttgttgtttttaggccaaggttgttgtgaaaattaaatgagataatatataaaaggtatttagctcaatatctggcacatagcaataattgaatagatgatccttcatcttcgttcctcctgttccctttcagtttgaaagacttggctaatataattttgaccaaccaaacttgcattcaagggagtgtacaaggctggtatagccagccagtgagtatcagaatctaaatgtttattaagacaaagggctgtcatgcaataacccaaccataccattatcagtctgccatccttcctgtttctctaggcagcctttcctgatgtcaactcaaccagttaatctctcagtcacttgacatgtggctatatatacacacaaatatgtgtgcatgcatcctgtgctgcaagcatttacagtcaagtttatctgaacacactgtatggttgatgtgaaatgctgaaactgttcaagtttaggtcctcacaaagcaaggaatatgaaatatttccttgggaaatatttatccacaacaaagagatgtacagtgctttcgtatacagtgatttacagttttccatgtgcttttacatgtattattacttcatttgatccttacaacaaccccagaggtagatgtggcatgaattaccattattctcctttgagaagaagaaactgagcatcaaagaagcttgttggccttcttgccagaaatcacccagtttgtaaatggtaaaagagggcttgaaaccaggttctctgactctgacttcaagcactctcatacatcatctatttaattttttggagctaggtattttatacttaggattctaaatattgcataaccattgaatgccacaccacccttgtattcagtgcaaaaaatgggacttttcttaataaatagagaaatggaggtgcctaaaattacaaaattgcactagagagatagtgatagaactgggaaactcttagtctaatattttatcttttattcatatgatggaatactaagctcaatggcagaatcttcagtcagcaatggtgttcaggttatgtgaactatctgaaggattcctgaactcttcatatctaggaatgtagcgtttaaaagctcttagaatttttcatactcttaggtcctcctgacttgtgcttcaattcatgcataaacttattttataaggtctccgtctgcccttgctggagataacatttttgtttatccaacaaagggtattttatcttattattaaattctgactttgtatagaagagaaatgaagtgataatctatataaattaagtcttgattagtacatatgggttattcacttggataatatggagtaaaattttaattcatggctaattacctccacctccactacctagtggcctcccctcaccaatattagccaaaataaatcaaatttggaactacaaacctacttcaaaaagggtaaggtatataataagcaatatcatcaagtcaaatagtatttttttaaccatgtacaaggcatcatgctaggtgttacgaagatgcatgaaatatataagatgtggttccaaccctcacagagtttatagacatcacataataaattctgaagtcaaatataaattaatttaaaattatctgctgttcagcattgttcactagtgcagccaaacaatgtcatcttgttgaaaggcattggtagtaaaaactgtgtctgaaatacccctctttcaaaggtttcgtaaatttgatatagtcagggacacgaacaagatccttttacatttttctttttgcttgttagtcttgctgtgctcataaatccatgacagaaagccccctcccctgagactttccacttccttttctggctttcactgttgcagaggctgctatattcacgacatgtggcctacagagttctcaggatgtaagcaagccaaactgaagaccaaatttgtagttcttgctctcctaaacagatgcatatgtggcacttcagctcttccagattacaacagaatttaggtttatgcataagtctaataagtcttcctcatactgaacttcctaatactactccccttttctatcttttcaacccacatgaccactattagacagatagtcagaaccagaaggaagtgaaatgctcactcaacaagcagagcatctttctcaatgcagttatcatagacaagacacactatgatagaattggtgctgctatgtattctttttgaacgcgttccggaattcgcccttgcatttcctaggagaatccctgggggaatcattgcagttggagcataatgtagggggcccctgagaaaacctccaggcttcaagtgacatacctagtctgctttaccggtttacaggactcaagagaaaggtggacattgagagttaatccctgaggccaaatcttaaatggagaaagtcaacatccacagaaaatggggaagggcacaagtatttctgtgggcttatattccgacatttttatctgtaggggaaaaatgctttcttagaaaatgactcagcacggggaagtcttgtctctacctctgtcttgttttgtcctttggggtcccttcactatcaagttcaactgtgtgtccctgagactcctctgccccggaggacaggagactcgaaaaacgctcttcctggccagtctctttgctctgtgtctgccagcccccagcatctctcctctttcctgtaagcccctctccctgtgctgactgtcttcatagtactttaggtatgttgtccctttacctctgggaggatagcttgatgacctgtctgctcaggccagccccatctagagtctcagtggccccagtcatgttgagaaaggttctttcaaagatagactcaagatagtagtgtcagaggtcccaagcaaatgaagggcggggacagttgagggggtggaatagggacggcagcagggaaccagatagcatgctgctgagaagaaaaaaagacattggtttaggtcaggaagcaaaaaaagggaactgagtggctgtgaaagggtggggtttgctcagactgtccttcctctctggactgtaagaattagtctcgagaaagaagccaccatggccgctgtgatcctggagagcattttcctgaagaggtcccagcagaaaaagaaaacctctcccctgaactttaagaaaagactgttcctgctgacagtgcacaagctgtcttactatgagtacgactttgagcggggccgccgaggatcaaaaaaggggagcatcgatgtggagaagattacatgcgtggagaccgtggtccctgaaaagaatccaccccctgagaggcagatcccaagacggggcgaggagtcctctgagatggagcagattagtatcattgagcgcttcccctatccttttcaggtggtgtacgacgagggaccactgtatgtgttctcacccacagaggagctgagaaagaggtggattcaccagctgaagaacgtgattagatacaatagcgatctggtgcagaagtatcacccttgtttttggatcgacgggcagtacctgtgctgttcccagacagctaagaacgctatgggatgccagattctggaaaatcggaacggatctctgaaaccagggagttcacaccgcaagaccaaaaagcccctgcctccaacacccgaggaggatcagatcctgaaaaagcctctgccacccgagcctgctgcagccccagtcagcacttccgaactgaaaaaggtggtggctctgtatgactacatgcccatgaatgctaacgatctgcagctgagaaagggcgacgagtatttcattctggaagagtctaatctgccttggtggagggccagagataagaacggacaggaggggtacatcccatctaattatgtgaccgaggctgaggactctattgagatgtacgagtggtatagcaagcacatgacacggtcccaggctgagcagctgctgaagcaggagggcaaagagggagggtttatcgtgcgcgattctagtaaggccggcaaatacactgtgtcagtgttcgctaagagcaccggagacccccagggcgtgatcagacactatgtggtgtgttccacacctcagtctcagtactatctggctgagaagcacctgtttagtacaatcccagagctgattaactaccaccagcacaattctgccggcctgatcagcaggctgaagtatcccgtctcccagcagaacaaaaatgctccttctaccgctggactggggtacggcagttgggagattgatccaaaggacctgacattcctgaaggagctgggaactgggcagtttggcgtggtgaagtatggaaaatggagagggcagtacgatgtggccatcaagatgatcaaggagggctcaatgagcgaggacgagttcatcgaggaggctaaggtcatgatgaacctgtcccacgagaaactggtgcagctgtatggagtgtgcaccaagcagcggcccatttttatcattacagagtacatggctaatgggtgtctgctgaactatctgcgcgagatgagacacagattccagacacagcagctgctggaaatgtgcaaggatgtgtgtgaggctatggagtacctggagtctaagcagtttctgcaccgggacctggctgctcgcaattgcctggtgaacgatcagggcgtggtgaaggtgagtgacttcggactgtcaaggtatgtgctggatgacgagtacaccagctccgtgggctctaagtttcctgtgagatggtctccacccgaggtgctgatgtatagcaagttctcctctaagagcgatatctgggcctttggcgtgctgatgtgggaaatctacagcctgggcaagatgccttacgagcggttcacaaattccgagacagctgagcacatcgcccagggcctgcgcctgtaccggccacatctggcctctgagaaggtgtacaccatcatgtacagctgttggcacgagaaggccgacgagagacccacattcaagatcctgctgtccaacattctagatgtgatggacgaggagagctga

SEQ ID NO: 36 - DNA, 3856 bp, Artificial Sequence
  ABCD.BTKp.GFP
tttcctaggagaatccctgggggaatcattgcagttggagcataatgtagggggcccggatccctggggtatggcaggggctgggcagcagcagcaatgtaccttgcttgggacccctaaaaaccagagagacagcatggctggtgccatttatcagctagtggaggaggctgacggagggtgggagtgtcatcagcacaaggccctggcagtcccttctggtgattagagaggccgaaagggtcctttccgacaagggctgagggtgggcggaacaggaagagaaaaatgtgacatgaggtgaccatccgaacaggtagcaaatgttagaaaggggtacctctggcaaacttagtggaaaagtaatattgcagggagcagtcagataaaaacaagcccttctgtcaaatagtgcttgaagactcaatagggatacatgggtcaatgaagcctttagaaaaagaaatactaagaggcagattctctgagaacatggtaaaagctcacgctccacgttatgaagttgacctttgtgagctagggaaaggcctggctaggccagggtgtaggctacctgccttgagctgtaccaggccaaatgtcgccagggtcagagctggcttattaaaggactgtgtggaagctgtgccaacctcgtggtaacaatgggtaaaagactgggccaggagaaagcagcctctgcctcagcccagacagtgcggccaacccttgaggttgtggcaaaggtttctcctcttaccattgccctccatgtgcatggcttgcttttctcttgtcttcattatttctcctttcctttcctcggatccacgcgtgaattctttgtaaactccttatggtgcgaactaatgtaactttccatccagttatgggggattggtgcaattttaaattatcactatgatttgctatttccatttgagcaaatttcctatagagtttcctttcagtggactagacccatatcaggaagtgacttaggtataaagggaagatacagctttcgaaaaccaaagtttgggcgttctccaaagagttatcagatacccccttctacacccacaatgatctgattgctgagatctgattgctaactactgaaaataaggaagaactagaattttcagtgacacagtgctcagcaagaagctagaaaagaggccttgacatatttgactccaaagctacttggttatgcatgaagccatctggggaggggaaggaggagggagaactcctctgaggaccctgaaacaattgggccacgtgtgactttcagtttctatggagattcatgtgcagtggctgagggcaatctgagagcattggaaacccagaagctttaaacgcgtaggaaagacagggaatggccagaatcttcctagccaattgagtagtgctttcaaggagaaatcaagagaaaacactacttcttggatattttggctaagtagtcatttgaagtacagttgactggttattttattttaaatcatatctcatagactcttcccaatcataggctggctgtgtagttttctgaattttgctctggtattcttcttcttttttttttttatttttattttttattttttttttgagactccaggctggagtgcagtgtcacgatcttggctcactgcaacctccgcctcccgagttcaagcgattctcctgcctcagcctcctgagttgctgggactacaggcgcctgtcaccacgcccggctaattttttgtattttaatagagacgggatttcaccatgttggccaggctggtctctaactcctgacctcaagtgatccgccagcctcagcctcccaaagtgctgggattacgggcatgagccactgcgcctggacttattattcttaatagtattttatcttatgagcgaagataagagcccaagatggtttagtttactgattctgcaagtgctatttctattaattccttggcatactgcagtttgtatgatggctgcactcttgttaataagcttcgtctttctgaattctgttgctccatagggagctgggaggctgcaaaaggtggccctgtaaaaatctttgcatttataatttaataattacagaccccagtgggacaatgtttgaaaaattatattcaccgtctaggaaattgggaactgaaagtccaatatctgcctcagtggagttctggcacctgcattatcccttctgggtatatcaagatcaacagctgcacagatacttttgcttttcacagattctacacatatcatataaaggtgaatagtgtaaagctacctctacaccttaccaagcacacagggggcccctgagaaaacctccaggcttcaagtgacatacctagtctgctttaccggtttacaggactcaagagaaaggtggacattgagagttaatccctgaggccaaatcttaaatggagaaagtcaacatccacagaaaatggggaagggcacaagtatttctgtgggcttatattccgacatttttatctgtaggggaaaaatgctttcttagaaaatgactcagcacggggaagtcttgtctctacctctgtcttgttttgtcctttggggtcccttcactatcaagttcaactgtgtgtccctgagactcctctgccccggaggacaggagactcgaaaaacgctcttcctggccagtctctttgctctgtgtctgccagcccccagcatctctcctctttcctgtaagcccctctccctgtgctgactgtcttcatagtactttaggtatgttgtccctttacctctgggaggatagcttgatgacctgtctgctcaggccagccccatctagagtctcagtggccccagtcatgttgagaaaggttctttcaaagatagactcaagatagtagtgtcagaggtcccaagcaaatgaagggcggggacagttgagggggtggaatagggacggcagcagggaaccagatagcatgctgctgagaagaaaaaaagacattggtttaggtcaggaagcaaaaaaagggaactgagtggctgtgaaagggtggggtttgctcagactgtccttcctctctggactgtaagaattagtctcgaggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 37 - DNA, 3856 bp, Artificial Sequence
  DBCA.BTKp.GFP
tttcctaggagaatccctgggggaatcattgcagttggagcataatgtagggggccccctgtgtgcttggtaaggtgtagaggtagctttacactattcacctttatatgatatgtgtagaatctgtgaaaagcaaaagtatctgtgcagctgttgatcttgatatacccagaagggataatgcaggtgccagaactccactgaggcagatattggactttcagttcccaatttcctagacggtgaatataatttttcaaacattgtcccactggggtctgtaattattaaattataaatgcaaagatttttacagggccaccttttgcagcctcccagctccctatggagcaacagaattcagaaagacgaagcttattaacaagagtgcagccatcatacaaactgcagtatgccaaggaattaatagaaatagcacttgcagaatcagtaaactaaaccatcttgggctcttatcttcgctcataagataaaatactattaagaataataagtccaggcgcagtggctcatgcccgtaatcccagcactttgggaggctgaggctggcggatcacttgaggtcaggagttagagaccagcctggccaacatggtgaaatcccgtctctattaaaatacaaaaaattagccgggcgtggtgacaggcgcctgtagtcccagcaactcaggaggctgaggcaggagaatcgcttgaactcgggaggcggaggttgcagtgagccaagatcgtgacactgcactccagcctggagtctcaaaaaaaaaataaaaaataaaaataaaaaaaaaaaagaagaagaataccagagcaaaattcagaaaactacacagccagcctatgattgggaagagtctatgagatatgatttaaaataaaataaccagtcaactgtacttcaaatgactacttagccaaaatatccaagaagtagtgttttctcttgatttctccttgaaagcactactcaattggctaggaagattctggccattccctgtctttcctacgcgtttaaagcttctgggtttccaatgctctcagattgccctcagccactgcacatgaatctccatagaaactgaaagtcacacgtggcccaattgtttcagggtcctcagaggagttctccctcctccttcccctccccagatggcttcatgcataaccaagtagctttggagtcaaatatgtcaaggcctcttttctagcttcttgctgagcactgtgtcactgaaaattctagttcttccttattttcagtagttagcaatcagatctcagcaatcagatcattgtgggtgtagaagggggtatctgataactctttggagaacgcccaaactttggttttcgaaagctgtatcttccctttatacctaagtcacttcctgatatgggtctagtccactgaaaggaaactctataggaaatttgctcaaatggaaatagcaaatcatagtgataatttaaaattgcaccaatcccccataactggatggaaagttacattagttcgcaccataaggagtttacaaagaattcacgcgtggatccgaggaaaggaaaggagaaataatgaagacaagagaaaagcaagccatgcacatggagggcaatggtaagaggagaaacctttgccacaacctcaagggttggccgcactgtctgggctgaggcagaggctgctttctcctggcccagtcttttacccattgttaccacgaggttggcacagcttccacacagtcctttaataagccagctctgaccctggcgacatttggcctggtacagctcaaggcaggtagcctacaccctggcctagccaggcctttccctagctcacaaaggtcaacttcataacgtggagcgtgagcttttaccatgttctcagagaatctgcctcttagtatttctttttctaaaggcttcattgacccatgtatccctattgagtcttcaagcactatttgacagaagggcttgtttttatctgactgctccctgcaatattacttttccactaagtttgccagaggtacccctttctaacatttgctacctgttcggatggtcacctcatgtcacatttttctcttcctgttccgcccaccctcagcccttgtcggaaaggaccctttcggcctctctaatcaccagaagggactgccagggccttgtgctgatgacactcccaccctccgtcagcctcctccactagctgataaatggcaccagccatgctgtctctctggtttttaggggtcccaagcaaggtacattgctgctgctgcccagcccctgccataccccagggatccgggcccctgagaaaacctccaggcttcaagtgacatacctagtctgctttaccggtttacaggactcaagagaaaggtggacattgagagttaatccctgaggccaaatcttaaatggagaaagtcaacatccacagaaaatggggaagggcacaagtatttctgtgggcttatattccgacatttttatctgtaggggaaaaatgctttcttagaaaatgactcagcacggggaagtcttgtctctacctctgtcttgttttgtcctttggggtcccttcactatcaagttcaactgtgtgtccctgagactcctctgccccggaggacaggagactcgaaaaacgctcttcctggccagtctctttgctctgtgtctgccagcccccagcatctctcctctttcctgtaagcccctctccctgtgctgactgtcttcatagtactttaggtatgttgtccctttacctctgggaggatagcttgatgacctgtctgctcaggccagccccatctagagtctcagtggccccagtcatgttgagaaaggttctttcaaagatagactcaagatagtagtgtcagaggtcccaagcaaatgaagggcggggacagttgagggggtggaatagggacggcagcagggaaccagatagcatgctgctgagaagaaaaaaagacattggtttaggtcaggaagcaaaaaaagggaactgagtggctgtgaaagggtggggtttgctcagactgtccttcctctctggactgtaagaattagtctcgaggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 38 - DNA, 2886 bp, Artificial Sequence
  AB.BTKp.GFP
tttcctaggagaatccctgggggaatcattgcagttggagcataatgtagggggcccggatccctggggtatggcaggggctgggcagcagcagcaatgtaccttgcttgggacccctaaaaaccagagagacagcatggctggtgccatttatcagctagtggaggaggctgacggagggtgggagtgtcatcagcacaaggccctggcagtcccttctggtgattagagaggccgaaagggtcctttccgacaagggctgagggtgggcggaacaggaagagaaaaatgtgacatgaggtgaccatccgaacaggtagcaaatgttagaaaggggtacctctggcaaacttagtggaaaagtaatattgcagggagcagtcagataaaaacaagcccttctgtcaaatagtgcttgaagactcaatagggatacatgggtcaatgaagcctttagaaaaagaaatactaagaggcagattctctgagaacatggtaaaagctcacgctccacgttatgaagttgacctttgtgagctagggaaaggcctggctaggccagggtgtaggctacctgccttgagctgtaccaggccaaatgtcgccagggtcagagctggcttattaaaggactgtgtggaagctgtgccaacctcgtggtaacaatgggtaaaagactgggccaggagaaagcagcctctgcctcagcccagacagtgcggccaacccttgaggttgtggcaaaggtttctcctcttaccattgccctccatgtgcatggcttgcttttctcttgtcttcattatttctcctttcctttcctcggatccacgcgtgaattctttgtaaactccttatggtgcgaactaatgtaactttccatccagttatgggggattggtgcaattttaaattatcactatgatttgctatttccatttgagcaaatttcctatagagtttcctttcagtggactagacccatatcaggaagtgacttaggtataaagggaagatacagctttcgaaaaccaaagtttgggcgttctccaaagagttatcagatacccccttctacacccacaatgatctgattgctgagatctgattgctaactactgaaaataaggaagaactagaattttcagtgacacagtgctcagcaagaagctagaaaagaggccttgacatatttgactccaaagctacttggttatgcatgaagccatctggggaggggaaggaggagggagaactcctctgaggaccctgaaacaattgggccacgtgtgactttcagtttctatggagattcatgtgcagtggctgagggcaatctgagagcattggaaacccagaagctttaaacgcgtaggggcccctgagaaaacctccaggcttcaagtgacatacctagtctgctttaccggtttacaggactcaagagaaaggtggacattgagagttaatccctgaggccaaatcttaaatggagaaagtcaacatccacagaaaatggggaagggcacaagtatttctgtgggcttatattccgacatttttatctgtaggggaaaaatgctttcttagaaaatgactcagcacggggaagtcttgtctctacctctgtcttgttttgtcctttggggtcccttcactatcaagttcaactgtgtgtccctgagactcctctgccccggaggacaggagactcgaaaaacgctcttcctggccagtctctttgctctgtgtctgccagcccccagcatctctcctctttcctgtaagcccctctccctgtgctgactgtcttcatagtactttaggtatgttgtccctttacctctgggaggatagcttgatgacctgtctgctcaggccagccccatctagagtctcagtggccccagtcatgttgagaaaggttctttcaaagatagactcaagatagtagtgtcagaggtcccaagcaaatgaagggcggggacagttgagggggtggaatagggacggcagcagggaaccagatagcatgctgctgagaagaaaaaaagacattggtttaggtcaggaagcaaaaaaagggaactgagtggctgtgaaagggtggggtttgctcagactgtccttcctctctggactgtaagaattagtctcgaggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 39 - DNA, 2349 bp, Artificial Sequence
  A.BTKp.GFP
tttcctaggagaatccctgggggaatcattgcagttggagcataatgtagggggcccggatccctggggtatggcaggggctgggcagcagcagcaatgtaccttgcttgggacccctaaaaaccagagagacagcatggctggtgccatttatcagctagtggaggaggctgacggagggtgggagtgtcatcagcacaaggccctggcagtcccttctggtgattagagaggccgaaagggtcctttccgacaagggctgagggtgggcggaacaggaagagaaaaatgtgacatgaggtgaccatccgaacaggtagcaaatgttagaaaggggtacctctggcaaacttagtggaaaagtaatattgcagggagcagtcagataaaaacaagcccttctgtcaaatagtgcttgaagactcaatagggatacatgggtcaatgaagcctttagaaaaagaaatactaagaggcagattctctgagaacatggtaaaagctcacgctccacgttatgaagttgacctttgtgagctagggaaaggcctggctaggccagggtgtaggctacctgccttgagctgtaccaggccaaatgtcgccagggtcagagctggcttattaaaggactgtgtggaagctgtgccaacctcgtggtaacaatgggtaaaagactgggccaggagaaagcagcctctgcctcagcccagacagtgcggccaacccttgaggttgtggcaaaggtttctcctcttaccattgccctccatgtgcatggcttgcttttctcttgtcttcattatttctcctttcctttcctcggatccacgcgtaggggcccctgagaaaacctccaggcttcaagtgacatacctagtctgctttaccggtttacaggactcaagagaaaggtggacattgagagttaatccctgaggccaaatcttaaatggagaaagtcaacatccacagaaaatggggaagggcacaagtatttctgtgggcttatattccgacatttttatctgtaggggaaaaatgctttcttagaaaatgactcagcacggggaagtcttgtctctacctctgtcttgttttgtcctttggggtcccttcactatcaagttcaactgtgtgtccctgagactcctctgccccggaggacaggagactcgaaaaacgctcttcctggccagtctctttgctctgtgtctgccagcccccagcatctctcctctttcctgtaagcccctctccctgtgctgactgtcttcatagtactttaggtatgttgtccctttacctctgggaggatagcttgatgacctgtctgctcaggccagccccatctagagtctcagtggccccagtcatgttgagaaaggttctttcaaagatagactcaagatagtagtgtcagaggtcccaagcaaatgaagggcggggacagttgagggggtggaatagggacggcagcagggaaccagatagcatgctgctgagaagaaaaaaagacattggtttaggtcaggaagcaaaaaaagggaactgagtggctgtgaaagggtggggtttgctcagactgtccttcctctctggactgtaagaattagtctcgaggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 40 - DNA, 2119 bp, Artificial Sequence
  B.BTKp.GFP
tttcctaggagaatccctgggggaatcattgcagttggagcataatgtagggggcccggatccacgcgtgaattctttgtaaactccttatggtgcgaactaatgtaactttccatccagttatgggggattggtgcaattttaaattatcactatgatttgctatttccatttgagcaaatttcctatagagtttcctttcagtggactagacccatatcaggaagtgacttaggtataaagggaagatacagctttcgaaaaccaaagtttgggcgttctccaaagagttatcagatacccccttctacacccacaatgatctgattgctgagatctgattgctaactactgaaaataaggaagaactagaattttcagtgacacagtgctcagcaagaagctagaaaagaggccttgacatatttgactccaaagctacttggttatgcatgaagccatctggggaggggaaggaggagggagaactcctctgaggaccctgaaacaattgggccacgtgtgactttcagtttctatggagattcatgtgcagtggctgagggcaatctgagagcattggaaacccagaagctttaaacgcgtaggggcccctgagaaaacctccaggcttcaagtgacatacctagtctgctttaccggtttacaggactcaagagaaaggtggacattgagagttaatccctgaggccaaatcttaaatggagaaagtcaacatccacagaaaatggggaagggcacaagtatttctgtgggcttatattccgacatttttatctgtaggggaaaaatgctttcttagaaaatgactcagcacggggaagtcttgtctctacctctgtcttgttttgtcctttggggtcccttcactatcaagttcaactgtgtgtccctgagactcctctgccccggaggacaggagactcgaaaaacgctcttcctggccagtctctttgctctgtgtctgccagcccccagcatctctcctctttcctgtaagcccctctccctgtgctgactgtcttcatagtactttaggtatgttgtccctttacctctgggaggatagcttgatgacctgtctgctcaggccagccccatctagagtctcagtggccccagtcatgttgagaaaggttctttcaaagatagactcaagatagtagtgtcagaggtcccaagcaaatgaagggcggggacagttgagggggtggaatagggacggcagcagggaaccagatagcatgctgctgagaagaaaaaaagacattggtttaggtcaggaagcaaaaaaagggaactgagtggctgtgaaagggtggggtttgctcagactgtccttcctctctggactgtaagaattagtctcgaggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 41 - DNA, 4809 bp, Artificial Sequence
  0.7UCOE.AB.BTKp.coBTK
tttcctaggagaatccctgggggaatcattgcagttggagcataatgtagggggccgcgtgtggcatctgaagcaccaccagcgagcgagagctagagagaaggaaagccaccgacttcaccgcctccgagctgctccgggtcgcgggtctgcagcgtctccggccctccgcgcctacagctcaagccacatccgaagggggagggagccgggagctgcgcgcggggccgccggggggaggggtggcaccgcccacgccgggcggccacgaagggcggggcagcgggcgcgcgcccggcggggggaggggccgcgcgccgcgcccgctgggaattggggccctagggggagggcggaggcgccgacgaccgcggcacttaccgttcgcggcgtggcgcccggtggtccccaaggggagggaagggggaggcggggcgaggacagtgaccggagtctcctcagcggtggcttttctgcttggcagcctcagcggctggcgccaaaaccggactccgcccacttcctcgcccctgcggtgcgagggtgtggaatcctccagacgctgggggagggggagttgggagcttaaaaactagtacccctttgggaccactttcagcagcgaactctcctgtacaccaggggtcagttccacagacgcgggccaggggtgggtcattgcggcgtgaacaataatttgactagaagttgattcgggtgtttgcggccctggggtatggcaggggctgggcagcagcagcaatgtaccttgcttgggacccctaaaaaccagagagacagcatggctggtgccatttatcagctagtggaggaggctgacggagggtgggagtgtcatcagcacaaggccctggcagtcccttctggtgattagagaggccgaaagggtcctttccgacaagggctgagggtgggcggaacaggaagagaaaaatgtgacatgaggtgaccatccgaacaggtagcaaatgttagaaaggggtacctctggcaaacttagtggaaaagtaatattgcagggagcagtcagataaaaacaagcccttctgtcaaatagtgcttgaagactcaatagggatacatgggtcaatgaagcctttagaaaaagaaatactaagaggcagattctctgagaacatggtaaaagctcacgctccacgttatgaagttgacctttgtgagctagggaaaggcctggctaggccagggtgtaggctacctgccttgagctgtaccaggccaaatgtcgccagggtcagagctggcttattaaaggactgtgtggaagctgtgccaacctcgtggtaacaatgggtaaaagactgggccaggagaaagcagcctctgcctcagcccagacagtgcggccaacccttgaggttgtggcaaaggtttctcctcttaccattgccctccatgtgcatggcttgcttttctcttgtcttcattatttctcctttcctttcctcggatccacgcgtgaattctttgtaaactccttatggtgcgaactaatgtaactttccatccagttatgggggattggtgcaattttaaattatcactatgatttgctatttccatttgagcaaatttcctatagagtttcctttcagtggactagacccatatcaggaagtgacttaggtataaagggaagatacagctttcgaaaaccaaagtttgggcgttctccaaagagttatcagatacccccttctacacccacaatgatctgattgctgagatctgattgctaactactgaaaataaggaagaactagaattttcagtgacacagtgctcagcaagaagctagaaaagaggccttgacatatttgactccaaagctacttggttatgcatgaagccatctggggaggggaaggaggagggagaactcctctgaggaccctgaaacaattgggccacgtgtgactttcagtttctatggagattcatgtgcagtggctgagggcaatctgagagcattggaaacccagaagctttaaggcccctgagaaaacctccaggcttcaagtgacatacctagtctgctttaccggtttacaggactcaagagaaaggtggacattgagagttaatccctgaggccaaatcttaaatggagaaagtcaacatccacagaaaatggggaagggcacaagtatttctgtgggcttatattccgacatttttatctgtaggggaaaaatgctttcttagaaaatgactcagcacggggaagtcttgtctctacctctgtcttgttttgtcctttggggtcccttcactatcaagttcaactgtgtgtccctgagactcctctgccccggaggacaggagactcgaaaaacgctcttcctggccagtctctttgctctgtgtctgccagcccccagcatctctcctctttcctgtaagcccctctccctgtgctgactgtcttcatagtactttaggtatgttgtccctttacctctgggaggatagcttgatgacctgtctgctcaggccagccccatctagagtctcagtggccccagtcatgttgagaaaggttctttcaaagatagactcaagatagtagtgtcagaggtcccaagcaaatgaagggcggggacagttgagggggtggaatagggacggcagcagggaaccagatagcatgctgctgagaagaaaaaaagacattggtttaggtcaggaagcaaaaaaagggaactgagtggctgtgaaagggtggggtttgctcagactgtccttcctctctggactgtaagaattagtctcgagaaagaagccaccatggccgctgtgatcctggagagcattttcctgaagaggtcccagcagaaaaagaaaacctctcccctgaactttaagaaaagactgttcctgctgacagtgcacaagctgtcttactatgagtacgactttgagcggggccgccgaggatcaaaaaaggggagcatcgatgtggagaagattacatgcgtggagaccgtggtccctgaaaagaatccaccccctgagaggcagatcccaagacggggcgaggagtcctctgagatggagcagattagtatcattgagcgcttcccctatccttttcaggtggtgtacgacgagggaccactgtatgtgttctcacccacagaggagctgagaaagaggtggattcaccagctgaagaacgtgattagatacaatagcgatctggtgcagaagtatcacccttgtttttggatcgacgggcagtacctgtgctgttcccagacagctaagaacgctatgggatgccagattctggaaaatcggaacggatctctgaaaccagggagttcacaccgcaagaccaaaaagcccctgcctccaacacccgaggaggatcagatcctgaaaaagcctctgccacccgagcctgctgcagccccagtcagcacttccgaactgaaaaaggtggtggctctgtatgactacatgcccatgaatgctaacgatctgcagctgagaaagggcgacgagtatttcattctggaagagtctaatctgccttggtggagggccagagataagaacggacaggaggggtacatcccatctaattatgtgaccgaggctgaggactctattgagatgtacgagtggtatagcaagcacatgacacggtcccaggctgagcagctgctgaagcaggagggcaaagagggagggtttatcgtgcgcgattctagtaaggccggcaaatacactgtgtcagtgttcgctaagagcaccggagacccccagggcgtgatcagacactatgtggtgtgttccacacctcagtctcagtactatctggctgagaagcacctgtttagtacaatcccagagctgattaactaccaccagcacaattctgccggcctgatcagcaggctgaagtatcccgtctcccagcagaacaaaaatgctccttctaccgctggactggggtacggcagttgggagattgatccaaaggacctgacattcctgaaggagctgggaactgggcagtttggcgtggtgaagtatggaaaatggagagggcagtacgatgtggccatcaagatgatcaaggagggctcaatgagcgaggacgagttcatcgaggaggctaaggtcatgatgaacctgtcccacgagaaactggtgcagctgtatggagtgtgcaccaagcagcggcccatttttatcattacagagtacatggctaatgggtgtctgctgaactatctgcgcgagatgagacacagattccagacacagcagctgctggaaatgtgcaaggatgtgtgtgaggctatggagtacctggagtctaagcagtttctgcaccgggacctggctgctcgcaattgcctggtgaacgatcagggcgtggtgaaggtgagtgacttcggactgtcaaggtatgtgctggatgacgagtacaccagctccgtgggctctaagtttcctgtgagatggtctccacccgaggtgctgatgtatagcaagttctcctctaagagcgatatctgggcctttggcgtgctgatgtgggaaatctacagcctgggcaagatgccttacgagcggttcacaaattccgagacagctgagcacatcgcccagggcctgcgcctgtaccggccacatctggcctctgagaaggtgtacaccatcatgtacagctgttggcacgagaaggccgacgagagacccacattcaagatcctgctgtccaacattctagatgtgatggacgaggagagctga

SEQ ID NO: 42 - DNA, 4136 bp, Artificial Sequence
  AB.BTKp.coBTK
tttcctaggagaatccctgggggaatcattgcagttggagcataatgtagggggccctggggtatggcaggggctgggcagcagcagcaatgtaccttgcttgggacccctaaaaaccagagagacagcatggctggtgccatttatcagctagtggaggaggctgacggagggtgggagtgtcatcagcacaaggccctggcagtcccttctggtgattagagaggccgaaagggtcctttccgacaagggctgagggtgggcggaacaggaagagaaaaatgtgacatgaggtgaccatccgaacaggtagcaaatgttagaaaggggtacctctggcaaacttagtggaaaagtaatattgcagggagcagtcagataaaaacaagcccttctgtcaaatagtgcttgaagactcaatagggatacatgggtcaatgaagcctttagaaaaagaaatactaagaggcagattctctgagaacatggtaaaagctcacgctccacgttatgaagttgacctttgtgagctagggaaaggcctggctaggccagggtgtaggctacctgccttgagctgtaccaggccaaatgtcgccagggtcagagctggcttattaaaggactgtgtggaagctgtgccaacctcgtggtaacaatgggtaaaagactgggccaggagaaagcagcctctgcctcagcccagacagtgcggccaacccttgaggttgtggcaaaggtttctcctcttaccattgccctccatgtgcatggcttgcttttctcttgtcttcattatttctcctttcctttcctcggatccacgcgtgaattctttgtaaactccttatggtgcgaactaatgtaactttccatccagttatgggggattggtgcaattttaaattatcactatgatttgctatttccatttgagcaaatttcctatagagtttcctttcagtggactagacccatatcaggaagtgacttaggtataaagggaagatacagctttcgaaaaccaaagtttgggcgttctccaaagagttatcagatacccccttctacacccacaatgatctgattgctgagatctgattgctaactactgaaaataaggaagaactagaattttcagtgacacagtgctcagcaagaagctagaaaagaggccttgacatatttgactccaaagctacttggttatgcatgaagccatctggggaggggaaggaggagggagaactcctctgaggaccctgaaacaattgggccacgtgtgactttcagtttctatggagattcatgtgcagtggctgagggcaatctgagagcattggaaacccagaagctttaaggcccctgagaaaacctccaggcttcaagtgacatacctagtctgctttaccggtttacaggactcaagagaaaggtggacattgagagttaatccctgaggccaaatcttaaatggagaaagtcaacatccacagaaaatggggaagggcacaagtatttctgtgggcttatattccgacatttttatctgtaggggaaaaatgctttcttagaaaatgactcagcacggggaagtcttgtctctacctctgtcttgttttgtcctttggggtcccttcactatcaagttcaactgtgtgtccctgagactcctctgccccggaggacaggagactcgaaaaacgctcttcctggccagtctctttgctctgtgtctgccagcccccagcatctctcctctttcctgtaagcccctctccctgtgctgactgtcttcatagtactttaggtatgttgtccctttacctctgggaggatagcttgatgacctgtctgctcaggccagccccatctagagtctcagtggccccagtcatgttgagaaaggttctttcaaagatagactcaagatagtagtgtcagaggtcccaagcaaatgaagggcggggacagttgagggggtggaatagggacggcagcagggaaccagatagcatgctgctgagaagaaaaaaagacattggtttaggtcaggaagcaaaaaaagggaactgagtggctgtgaaagggtggggtttgctcagactgtccttcctctctggactgtaagaattagtctcgagaaagaagccaccatggccgctgtgatcctggagagcattttcctgaagaggtcccagcagaaaaagaaaacctctcccctgaactttaagaaaagactgttcctgctgacagtgcacaagctgtcttactatgagtacgactttgagcggggccgccgaggatcaaaaaaggggagcatcgatgtggagaagattacatgcgtggagaccgtggtccctgaaaagaatccaccccctgagaggcagatcccaagacggggcgaggagtcctctgagatggagcagattagtatcattgagcgcttcccctatccttttcaggtggtgtacgacgagggaccactgtatgtgttctcacccacagaggagctgagaaagaggtggattcaccagctgaagaacgtgattagatacaatagcgatctggtgcagaagtatcacccttgtttttggatcgacgggcagtacctgtgctgttcccagacagctaagaacgctatgggatgccagattctggaaaatcggaacggatctctgaaaccagggagttcacaccgcaagaccaaaaagcccctgcctccaacacccgaggaggatcagatcctgaaaaagcctctgccacccgagcctgctgcagccccagtcagcacttccgaactgaaaaaggtggtggctctgtatgactacatgcccatgaatgctaacgatctgcagctgagaaagggcgacgagtatttcattctggaagagtctaatctgccttggtggagggccagagataagaacggacaggaggggtacatcccatctaattatgtgaccgaggctgaggactctattgagatgtacgagtggtatagcaagcacatgacacggtcccaggctgagcagctgctgaagcaggagggcaaagagggagggtttatcgtgcgcgattctagtaaggccggcaaatacactgtgtcagtgttcgctaagagcaccggagacccccagggcgtgatcagacactatgtggtgtgttccacacctcagtctcagtactatctggctgagaagcacctgtttagtacaatcccagagctgattaactaccaccagcacaattctgccggcctgatcagcaggctgaagtatcccgtctcccagcagaacaaaaatgctccttctaccgctggactggggtacggcagttgggagattgatccaaaggacctgacattcctgaaggagctgggaactgggcagtttggcgtggtgaagtatggaaaatggagagggcagtacgatgtggccatcaagatgatcaaggagggctcaatgagcgaggacgagttcatcgaggaggctaaggtcatgatgaacctgtcccacgagaaactggtgcagctgtatggagtgtgcaccaagcagcggcccatttttatcattacagagtacatggctaatgggtgtctgctgaactatctgcgcgagatgagacacagattccagacacagcagctgctggaaatgtgcaaggatgtgtgtgaggctatggagtacctggagtctaagcagtttctgcaccgggacctggctgctcgcaattgcctggtgaacgatcagggcgtggtgaaggtgagtgacttcggactgtcaaggtatgtgctggatgacgagtacaccagctccgtgggctctaagtttcctgtgagatggtctccacccgaggtgctgatgtatagcaagttctcctctaagagcgatatctgggcctttggcgtgctgatgtgggaaatctacagcctgggcaagatgccttacgagcggttcacaaattccgagacagctgagcacatcgcccagggcctgcgcctgtaccggccacatctggcctctgagaaggtgtacaccatcatgtacagctgttggcacgagaaggccgacgagagacccacattcaagatcctgctgtccaacattctagatgtgatggacgaggagagctga

SEQ ID NO: 43 - DNA, 5782 bp, Artificial Sequence
  BTKe.AB.BTKp.coBTK
tttcctaggagaatccctgggggaatcattgcagttggagcataatgtagggggccctccatcacctactagatatatcagtgcagtgaaaacttcgctaaactaacgctatacctatatcatgaagtgtgtggactagagacaagtgcatatccttacggcaattaactgggaaacgtcaaatagtaactaccactcacctttttccggaaaatcggcttagtttgcccaccatagccactctgcttcctgtcataacgccgctttcctgggaaaacgaattggtatttgttataaaatactgaagatcagcaagtaagtcttacaggttttatcttaatttcgcagcagaaatattaacgctcaagccaggcgtggagggagagagacccggactcgtatgttattctacaacacaaatgtcacattaacaccaaattatgcggaatccatcttaccctgggcgtacagagaatccttgcccttcttgtactgtgtcactttatggggttggtgcttgccacacttcttacagaaagtccggcgggttttagggacgttaacctagtaaagaaacagttcagaacgtgcaatgttatttgaccacaatggcacaacgccctaccttacccagctaaagctgaggcactccaggaggactcctcattacttgctacctctgactacagggtgggccagccccatgtgcttcaagcagagcttcctccctccgtcgagccccaaagagggaagagacctcattaactccacccccggctaactctacctctttgaacccatcacttcaattcctggccccgtagcccggtccctttagggttgatcccggcaagattgggttgctctgatatatcgagtccacacaggagcctggacccatcccggcatagcacgggcgacgaagggggggaaagattaagctggatgttactcggcccccaccagcaagtcctaccatgcttgcgtgagcgctatcggcgcggaaagaaagaaaccgcgaggcaaacggaagtatataggaggttcccgatcgcacttcctcatgggagtcggtaggagcaatcatagagtgtaaggctcagcgcagcgccctcgggcggctgagaggactcagttcggagccgcgggcgggagcttaaggaaggactccgcctaaagggtggtccactcaccccgacttcctcccgccccgcagctttcaacgtttcgtcactttatctcttttggtggactctgctacgtagtggcgttcagtgaagggagcagtgtttttcccagatcctctggcctccccgtccccgagggaagccaggactagggtcgaatgaaggggtcctccacctccacgttccattcctgttccacctcaaggtcactgggaacacctttcgcagcaaactgctaattcaatgaagacctggagggagccaattgttccagttcatctatcacatggccagttggtccattcaacaaatggttattggatgcccattatgtggcaggcactgttccgggggagaggtacagtaatctaataggcttataaatgtgcaattatgaactaagtactttgaagaaaaggaacaatgattggcattaaagcagcacccttctgttgagggagtaagtcagcagctctaggttctgaaaagtgacaatgaaattgtttggctcctgtctggggtatggcaggggctgggcagcagcagcaatgtaccttgcttgggacccctaaaaaccagagagacagcatggctggtgccatttatcagctagtggaggaggctgacggagggtgggagtgtcatcagcacaaggccctggcagtcccttctggtgattagagaggccgaaagggtcctttccgacaagggctgagggtgggcggaacaggaagagaaaaatgtgacatgaggtgaccatccgaacaggtagcaaatgttagaaaggggtacctctggcaaacttagtggaaaagtaatattgcagggagcagtcagataaaaacaagcccttctgtcaaatagtgcttgaagactcaatagggatacatgggtcaatgaagcctttagaaaaagaaatactaagaggcagattctctgagaacatggtaaaagctcacgctccacgttatgaagttgacctttgtgagctagggaaaggcctggctaggccagggtgtaggctacctgccttgagctgtaccaggccaaatgtcgccagggtcagagctggcttattaaaggactgtgtggaagctgtgccaacctcgtggtaacaatgggtaaaagactgggccaggagaaagcagcctctgcctcagcccagacagtgcggccaacccttgaggttgtggcaaaggtttctcctcttaccattgccctccatgtgcatggcttgcttttctcttgtcttcattatttctcctttcctttcctcggatccacgcgtgaattctttgtaaactccttatggtgcgaactaatgtaactttccatccagttatgggggattggtgcaattttaaattatcactatgatttgctatttccatttgagcaaatttcctatagagtttcctttcagtggactagacccatatcaggaagtgacttaggtataaagggaagatacagctttcgaaaaccaaagtttgggcgttctccaaagagttatcagatacccccttctacacccacaatgatctgattgctgagatctgattgctaactactgaaaataaggaagaactagaattttcagtgacacagtgctcagcaagaagctagaaaagaggccttgacatatttgactccaaagctacttggttatgcatgaagccatctggggaggggaaggaggagggagaactcctctgaggaccctgaaacaattgggccacgtgtgactttcagtttctatggagattcatgtgcagtggctgagggcaatctgagagcattggaaacccagaagctttaaggcccctgagaaaacctccaggcttcaagtgacatacctagtctgctttaccggtttacaggactcaagagaaaggtggacattgagagttaatccctgaggccaaatcttaaatggagaaagtcaacatccacagaaaatggggaagggcacaagtatttctgtgggcttatattccgacatttttatctgtaggggaaaaatgctttcttagaaaatgactcagcacggggaagtcttgtctctacctctgtcttgttttgtcctttggggtcccttcactatcaagttcaactgtgtgtccctgagactcctctgccccggaggacaggagactcgaaaaacgctcttcctggccagtctctttgctctgtgtctgccagcccccagcatctctcctctttcctgtaagcccctctccctgtgctgactgtcttcatagtactttaggtatgttgtccctttacctctgggaggatagcttgatgacctgtctgctcaggccagccccatctagagtctcagtggccccagtcatgttgagaaaggttctttcaaagatagactcaagatagtagtgtcagaggtcccaagcaaatgaagggcggggacagttgagggggtggaatagggacggcagcagggaaccagatagcatgctgctgagaagaaaaaaagacattggtttaggtcaggaagcaaaaaaagggaactgagtggctgtgaaagggtggggtttgctcagactgtccttcctctctggactgtaagaattagtctcgagaaagaagccaccatggccgctgtgatcctggagagcattttcctgaagaggtcccagcagaaaaagaaaacctctcccctgaactttaagaaaagactgttcctgctgacagtgcacaagctgtcttactatgagtacgactttgagcggggccgccgaggatcaaaaaaggggagcatcgatgtggagaagattacatgcgtggagaccgtggtccctgaaaagaatccaccccctgagaggcagatcccaagacggggcgaggagtcctctgagatggagcagattagtatcattgagcgcttcccctatccttttcaggtggtgtacgacgagggaccactgtatgtgttctcacccacagaggagctgagaaagaggtggattcaccagctgaagaacgtgattagatacaatagcgatctggtgcagaagtatcacccttgtttttggatcgacgggcagtacctgtgctgttcccagacagctaagaacgctatgggatgccagattctggaaaatcggaacggatctctgaaaccagggagttcacaccgcaagaccaaaaagcccctgcctccaacacccgaggaggatcagatcctgaaaaagcctctgccacccgagcctgctgcagccccagtcagcacttccgaactgaaaaaggtggtggctctgtatgactacatgcccatgaatgctaacgatctgcagctgagaaagggcgacgagtatttcattctggaagagtctaatctgccttggtggagggccagagataagaacggacaggaggggtacatcccatctaattatgtgaccgaggctgaggactctattgagatgtacgagtggtatagcaagcacatgacacggtcccaggctgagcagctgctgaagcaggagggcaaagagggagggtttatcgtgcgcgattctagtaaggccggcaaatacactgtgtcagtgttcgctaagagcaccggagacccccagggcgtgatcagacactatgtggtgtgttccacacctcagtctcagtactatctggctgagaagcacctgtttagtacaatcccagagctgattaactaccaccagcacaattctgccggcctgatcagcaggctgaagtatcccgtctcccagcagaacaaaaatgctccttctaccgctggactggggtacggcagttgggagattgatccaaaggacctgacattcctgaaggagctgggaactgggcagtttggcgtggtgaagtatggaaaatggagagggcagtacgatgtggccatcaagatgatcaaggagggctcaatgagcgaggacgagttcatcgaggaggctaaggtcatgatgaacctgtcccacgagaaactggtgcagctgtatggagtgtgcaccaagcagcggcccatttttatcattacagagtacatggctaatgggtgtctgctgaactatctgcgcgagatgagacacagattccagacacagcagctgctggaaatgtgcaaggatgtgtgtgaggctatggagtacctggagtctaagcagtttctgcaccgggacctggctgctcgcaattgcctggtgaacgatcagggcgtggtgaaggtgagtgacttcggactgtcaaggtatgtgctggatgacgagtacaccagctccgtgggctctaagtttcctgtgagatggtctccacccgaggtgctgatgtatagcaagttctcctctaagagcgatatctgggcctttggcgtgctgatgtgggaaatctacagcctgggcaagatgccttacgagcggttcacaaattccgagacagctgagcacatcgcccagggcctgcgcctgtaccggccacatctggcctctgagaaggtgtacaccatcatgtacagctgttggcacgagaaggccgacgagagacccacattcaagatcctgctgtccaacattctagatgtgatggacgaggagagctga

SEQ ID NO: 44 - DNA, 4478 bp, Artificial Sequence
  BTKe.BTKp.coBTK
tttcctaggagaatccctgggggaatcattgcagttggagcataatgtagggggccctccatcacctactagatatatcagtgcagtgaaaacttcgctaaactaacgctatacctatatcatgaagtgtgtggactagagacaagtgcatatccttacggcaattaactgggaaacgtcaaatagtaactaccactcacctttttccggaaaatcggcttagtttgcccaccatagccactctgcttcctgtcataacgccgctttcctgggaaaacgaattggtatttgttataaaatactgaagatcagcaagtaagtcttacaggttttatcttaatttcgcagcagaaatattaacgctcaagccaggcgtggagggagagagacccggactcgtatgttattctacaacacaaatgtcacattaacaccaaattatgcggaatccatcttaccctgggcgtacagagaatccttgcccttcttgtactgtgtcactttatggggttggtgcttgccacacttcttacagaaagtccggcgggttttagggacgttaacctagtaaagaaacagttcagaacgtgcaatgttatttgaccacaatggcacaacgccctaccttacccagctaaagctgaggcactccaggaggactcctcattacttgctacctctgactacagggtgggccagccccatgtgcttcaagcagagcttcctccctccgtcgagccccaaagagggaagagacctcattaactccacccccggctaactctacctctttgaacccatcacttcaattcctggccccgtagcccggtccctttagggttgatcccggcaagattgggttgctctgatatatcgagtccacacaggagcctggacccatcccggcatagcacgggcgacgaagggggggaaagattaagctggatgttactcggcccccaccagcaagtcctaccatgcttgcgtgagcgctatcggcgcggaaagaaagaaaccgcgaggcaaacggaagtatataggaggttcccgatcgcacttcctcatgggagtcggtaggagcaatcatagagtgtaaggctcagcgcagcgccctcgggcggctgagaggactcagttcggagccgcgggcgggagcttaaggaaggactccgcctaaagggtggtccactcaccccgacttcctcccgccccgcagctttcaacgtttcgtcactttatctcttttggtggactctgctacgtagtggcgttcagtgaagggagcagtgtttttcccagatcctctggcctccccgtccccgagggaagccaggactagggtcgaatgaaggggtcctccacctccacgttccattcctgttccacctcaaggtcactgggaacacctttcgcagcaaactgctaattcaatgaagacctggagggagccaattgttccagttcatctatcacatggccagttggtccattcaacaaatggttattggatgcccattatgtggcaggcactgttccgggggagaggtacagtaatctaataggcttataaatgtgcaattatgaactaagtactttgaagaaaaggaacaatgattggcattaaagcagcacccttctgttgagggagtaagtcagcagctctaggttctgaaaagtgacaatgaaattgtttggctcctgtggcccctgagaaaacctccaggcttcaagtgacatacctagtctgctttaccggtttacaggactcaagagaaaggtggacattgagagttaatccctgaggccaaatcttaaatggagaaagtcaacatccacagaaaatggggaagggcacaagtatttctgtgggcttatattccgacatttttatctgtaggggaaaaatgctttcttagaaaatgactcagcacggggaagtcttgtctctacctctgtcttgttttgtcctttggggtcccttcactatcaagttcaactgtgtgtccctgagactcctctgccccggaggacaggagactcgaaaaacgctcttcctggccagtctctttgctctgtgtctgccagcccccagcatctctcctctttcctgtaagcccctctccctgtgctgactgtcttcatagtactttaggtatgttgtccctttacctctgggaggatagcttgatgacctgtctgctcaggccagccccatctagagtctcagtggccccagtcatgttgagaaaggttctttcaaagatagactcaagatagtagtgtcagaggtcccaagcaaatgaagggcggggacagttgagggggtggaatagggacggcagcagggaaccagatagcatgctgctgagaagaaaaaaagacattggtttaggtcaggaagcaaaaaaagggaactgagtggctgtgaaagggtggggtttgctcagactgtccttcctctctggactgtaagaattagtctcgagaaagaagccaccatggccgctgtgatcctggagagcattttcctgaagaggtcccagcagaaaaagaaaacctctcccctgaactttaagaaaagactgttcctgctgacagtgcacaagctgtcttactatgagtacgactttgagcggggccgccgaggatcaaaaaaggggagcatcgatgtggagaagattacatgcgtggagaccgtggtccctgaaaagaatccaccccctgagaggcagatcccaagacggggcgaggagtcctctgagatggagcagattagtatcattgagcgcttcccctatccttttcaggtggtgtacgacgagggaccactgtatgtgttctcacccacagaggagctgagaaagaggtggattcaccagctgaagaacgtgattagatacaatagcgatctggtgcagaagtatcacccttgtttttggatcgacgggcagtacctgtgctgttcccagacagctaagaacgctatgggatgccagattctggaaaatcggaacggatctctgaaaccagggagttcacaccgcaagaccaaaaagcccctgcctccaacacccgaggaggatcagatcctgaaaaagcctctgccacccgagcctgctgcagccccagtcagcacttccgaactgaaaaaggtggtggctctgtatgactacatgcccatgaatgctaacgatctgcagctgagaaagggcgacgagtatttcattctggaagagtctaatctgccttggtggagggccagagataagaacggacaggaggggtacatcccatctaattatgtgaccgaggctgaggactctattgagatgtacgagtggtatagcaagcacatgacacggtcccaggctgagcagctgctgaagcaggagggcaaagagggagggtttatcgtgcgcgattctagtaaggccggcaaatacactgtgtcagtgttcgctaagagcaccggagacccccagggcgtgatcagacactatgtggtgtgttccacacctcagtctcagtactatctggctgagaagcacctgtttagtacaatcccagagctgattaactaccaccagcacaattctgccggcctgatcagcaggctgaagtatcccgtctcccagcagaacaaaaatgctccttctaccgctggactggggtacggcagttgggagattgatccaaaggacctgacattcctgaaggagctgggaactgggcagtttggcgtggtgaagtatggaaaatggagagggcagtacgatgtggccatcaagatgatcaaggagggctcaatgagcgaggacgagttcatcgaggaggctaaggtcatgatgaacctgtcccacgagaaactggtgcagctgtatggagtgtgcaccaagcagcggcccatttttatcattacagagtacatggctaatgggtgtctgctgaactatctgcgcgagatgagacacagattccagacacagcagctgctggaaatgtgcaaggatgtgtgtgaggctatggagtacctggagtctaagcagtttctgcaccgggacctggctgctcgcaattgcctggtgaacgatcagggcgtggtgaaggtgagtgacttcggactgtcaaggtatgtgctggatgacgagtacaccagctccgtgggctctaagtttcctgtgagatggtctccacccgaggtgctgatgtatagcaagttctcctctaagagcgatatctgggcctttggcgtgctgatgtgggaaatctacagcctgggcaagatgccttacgagcggttcacaaattccgagacagctgagcacatcgcccagggcctgcgcctgtaccggccacatctggcctctgagaaggtgtacaccatcatgtacagctgttggcacgagaaggccgacgagagacccacattcaagatcctgctgtccaacattctagatgtgatggacgaggagagctga

SEQ ID NO: 45 - DNA, 3589 bp, Artificial Sequence
  BTKeMyc.BTKp.coBTK
tttcctaggagaatccctgggggaatcattgcagttggagcataatgtagggggccctgttactcggcccccaccagcaagtcctaccatgcttgcgtgagcgctatcggcgcggaaagaaagaaaccgcgaggcaaacggaagtatataggaggttcccgatcgcacttcctcatgggagtcggtaggagcaatcatagagtgtaaggctcagcgcagcgccctcgggcggctgagaggactcagttcggagccgcgggcgggagcttaaggaaggactccgcctaaagggtggtccactcaccccgacttcctcccgccccgcagctttcaacgtttcgtcactttatctcttttggtggactctgctacgtagtggcgttcagtgaagggagcagtgtttttcccagatcctctggcctccccgtccccgagggaagccaggactagggtcgaatgaaggggtcctccacctccacgttccattcctgttccacctcaaggtcactgggaacacctttcgcagcaaactgctaattcaatgaagacctggagggagccaattgttccagttcatctatcacatggccagttggtccattcaacaaatggttattggatgcccattatgtggcaggcactgttccgggggagaggtacagtaatctaataggcttataaatgtgcaattatgaactaagtactttgaagaaaaggaacaatgattggcattaaagcagcacccttctgttgagggagtaagtcagcagctctaggttctgaaaagtgacaatgaaattgtttggctcctgtggcccctgagaaaacctccaggcttcaagtgacatacctagtctgctttaccggtttacaggactcaagagaaaggtggacattgagagttaatccctgaggccaaatcttaaatggagaaagtcaacatccacagaaaatggggaagggcacaagtatttctgtgggcttatattccgacatttttatctgtaggggaaaaatgctttcttagaaaatgactcagcacggggaagtcttgtctctacctctgtcttgttttgtcctttggggtcccttcactatcaagttcaactgtgtgtccctgagactcctctgccccggaggacaggagactcgaaaaacgctcttcctggccagtctctttgctctgtgtctgccagcccccagcatctctcctctttcctgtaagcccctctccctgtgctgactgtcttcatagtactttaggtatgttgtccctttacctctgggaggatagcttgatgacctgtctgctcaggccagccccatctagagtctcagtggccccagtcatgttgagaaaggttctttcaaagatagactcaagatagtagtgtcagaggtcccaagcaaatgaagggcggggacagttgagggggtggaatagggacggcagcagggaaccagatagcatgctgctgagaagaaaaaaagacattggtttaggtcaggaagcaaaaaaagggaactgagtggctgtgaaagggtggggtttgctcagactgtccttcctctctggactgtaagaattagtctcgagaaagaagccaccatggccgctgtgatcctggagagcattttcctgaagaggtcccagcagaaaaagaaaacctctcccctgaactttaagaaaagactgttcctgctgacagtgcacaagctgtcttactatgagtacgactttgagcggggccgccgaggatcaaaaaaggggagcatcgatgtggagaagattacatgcgtggagaccgtggtccctgaaaagaatccaccccctgagaggcagatcccaagacggggcgaggagtcctctgagatggagcagattagtatcattgagcgcttcccctatccttttcaggtggtgtacgacgagggaccactgtatgtgttctcacccacagaggagctgagaaagaggtggattcaccagctgaagaacgtgattagatacaatagcgatctggtgcagaagtatcacccttgtttttggatcgacgggcagtacctgtgctgttcccagacagctaagaacgctatgggatgccagattctggaaaatcggaacggatctctgaaaccagggagttcacaccgcaagaccaaaaagcccctgcctccaacacccgaggaggatcagatcctgaaaaagcctctgccacccgagcctgctgcagccccagtcagcacttccgaactgaaaaaggtggtggctctgtatgactacatgcccatgaatgctaacgatctgcagctgagaaagggcgacgagtatttcattctggaagagtctaatctgccttggtggagggccagagataagaacggacaggaggggtacatcccatctaattatgtgaccgaggctgaggactctattgagatgtacgagtggtatagcaagcacatgacacggtcccaggctgagcagctgctgaagcaggagggcaaagagggagggtttatcgtgcgcgattctagtaaggccggcaaatacactgtgtcagtgttcgctaagagcaccggagacccccagggcgtgatcagacactatgtggtgtgttccacacctcagtctcagtactatctggctgagaagcacctgtttagtacaatcccagagctgattaactaccaccagcacaattctgccggcctgatcagcaggctgaagtatcccgtctcccagcagaacaaaaatgctccttctaccgctggactggggtacggcagttgggagattgatccaaaggacctgacattcctgaaggagctgggaactgggcagtttggcgtggtgaagtatggaaaatggagagggcagtacgatgtggccatcaagatgatcaaggagggctcaatgagcgaggacgagttcatcgaggaggctaaggtcatgatgaacctgtcccacgagaaactggtgcagctgtatggagtgtgcaccaagcagcggcccatttttatcattacagagtacatggctaatgggtgtctgctgaactatctgcgcgagatgagacacagattccagacacagcagctgctggaaatgtgcaaggatgtgtgtgaggctatggagtacctggagtctaagcagtttctgcaccgggacctggctgctcgcaattgcctggtgaacgatcagggcgtggtgaaggtgagtgacttcggactgtcaaggtatgtgctggatgacgagtacaccagctccgtgggctctaagtttcctgtgagatggtctccacccgaggtgctgatgtatagcaagttctcctctaagagcgatatctgggcctttggcgtgctgatgtgggaaatctacagcctgggcaagatgccttacgagcggttcacaaattccgagacagctgagcacatcgcccagggcctgcgcctgtaccggccacatctggcctctgagaaggtgtacaccatcatgtacagctgttggcacgagaaggccgacgagagacccacattcaagatcctgctgtccaacattctagatgtgatggacgaggagagctga

SEQ ID NO: 46 - DNA, 387 bp, Artificial Sequence
  B29 promoter sequence
aggagggccatcatggccaagttgaccagtgctgtcccagtgctcacagccagggatgtggctggagctgttgagttctggactgacaggttggggttctccagagattttgtggaggatgactttgcaggtgtggtcagagatgatgtcaccctgttcatctcagcagtccaggaccaggtggtgcctgacaacaccctggcttgggtgtgggtgagaggactggatgagctgtatgctgagtggagtgaggtggtctccaccaacttcagggatgccagtggccctgccatgacagagattggagagcagccctgggggagagagtttgccctgagagacccagcaggcaactgtgtgcactttgtggcagaggagcaggactga

(19) United States
(12) Patent Application Publication
(10) Pub. No.: US 2020/0325458 A1
(43) Pub. Date: Oct. 15, 2020

(54) OPTIMIZED LENTIVIRAL VECTOR FOR XLA GENE THERAPY

(71) Applicant: Seattle Children's Hospital (dba Seattle Children's Research Institute), Seattle, WA (US)

(72) Inventors: David J. Rawlings, Seattle, WA (US); Karen Sommer, Seattle, WA (US); Swati Singh, Issaquah, WA (US)

(21) Appl. No.: 16/605,740

(22) PCT Filed: Apr. 19, 2018
(86) PCT No.: PCT/US18/28331

§ 371 Date: Oct. 16, 2019

Related U.S. Application Data
(60) Provisional application No. 62/488,523, filed on Apr. 21, 2017.

(51) Int. Cl. C12N 9/12; C12N 15/86; C12N 7/00; A61K 35/17

(52) U.S. Cl. CPC C12N 9/12; C12Y 207/10002; C12N 2740/15043; C12N 7/00; A61K 35/17; C12N 15/86

(57) ABSTRACT

Described herein are compositions and methods for treating, inhibiting or ameliorating X linked agammaglobulinemia (XLA) in subjects that have been identified or selected as being ones that would benefit from a therapy to treat, inhibit, or ameliorate XLA. Exemplary embodiments include constructs and methods for gene therapy, which restore or increase BTK expression.

### INCORPORATION BY REFERENCE TO ANY PRIORITY APPLICATIONS

This application is the U.S. National Phase of PCT International Application Number PCT/US2018/028331, filed on Apr. 19, 2018, designating the United States of America and published in the English language as WO 2018/195297, which claims priority to U.S. Provisional Application No. 62/488,523, filed on Apr. 21, 2017 which are each expressly incorporated by reference in its entirety.

### REFERENCE TO FEDERAL FUNDING

This invention was made with support under grant number AI084457, awarded by the NIH National Institute of Allergy and Infectious Diseases.

### REFERENCE TO SEQUENCE LISTING

The present application is being filed along with a Sequence Listing in electronic format. The Sequence Listing is provided as a file entitled SCRI148NPSEQLISTING.TXT, created Jun. 22, 2020 which is approximately 141 kb in size. The information provided is the electronic format of the Sequence Listing is incorporated herein by reference in its entirety.

### FIELD OF THE INVENTION

Aspects of the present invention concern compositions and methods for treating, inhibiting or ameliorating X linked agammaglobulinemia (XLA) in subjects that have been identified or selected as being ones that would benefit from a therapy to treat, inhibit, or ameliorate XLA. Exemplary aspects include constructs and methods for gene therapy, which restore or increase Bruton's tyrosine kinase (BTK) expression.

### BACKGROUND OF THE INVENTION

X linked agammaglobulinemia (XLA) is a rare X-linked genetic disorder resulting from mutations in the Bruton's tyrosine kinase (BTK) gene. These mutations contribute to the failure of afflicted individuals to generate mature B cells and the inability of these B cells to respond to B cell antigen receptor engagement, as well as, other cellular signals. Affected males are unable to generate protective antibody responses to pathogen challenge and eventually succumb to viral or bacterial infection. Current therapy has not changed for over 5 decades and consists of immunoglobulin replacement and targeted anti-microbial agents. Despite this therapy, XLA subjects continue to suffer from chronic infections and are at an increased risk for a range of morbid or life-threatening complications. In rare settings, XLA subjects have been treated with stem cell transplantation without conditioning or using reduced intensity conditioning with variable outcomes. The need for more therapies to inhibit, treat, or ameliorate XLA is manifest.

### SUMMARY OF THE INVENTION

Based upon iterative design and testing of candidate promoter, insulator, and enhancer elements and human codon-optimized BTK cDNA constructs, an identified novel lentiviral-based (LV) vector construct that mediates sustained BTK expression in B and myeloid cells derived from (murine or human) hematopoietic stem cells has been manufactured and is described in the alternatives herein. Following ex vivo transduction and transplantation into BTK deficient hosts, these alternatives have been shown to surprisingly sustain BTK expression and rescue B cell development. As shown in one of the exemplary alternatives herein, the constructs set forth herein utilize a truncated ubiquitous chromatin opening element (UCOE) element, a conserved enhancer element derived from intronic regions within the human BTK locus in association with the human BTK proximal promoter to drive expression of a human codon-optimized BTK cDNA.

LV vectors using this construct in mouse gene therapy experiments mediate sustained BTK expression in B and myeloid cells in primary and secondary transplant recipients and rescue B cell development and function without evidence of viral toxicity. Thus, this construct represents a unique LV vector for gene therapy treatment, inhibition, or amelioration of human XLA.

In a first aspect, a polynucleotide for sustained Bruton's tyrosine kinase (BTK) expression, the polynucleotide comprising: a first sequence encoding an ubiquitous chromatin opening element (UCOE), a second sequence encoding a promoter and a third sequence encoding BTK. In some alternatives, the UCOE is 2 kb, 1.5 kb, 1 kb, 0.75 kb, 0.5 kb or 0.25 kb or any number of kilobases in between a range defined by any two afore mentioned values. In some alternatives, the first sequence comprises the nucleic acid sequence set forth in SEQ ID NO: 1, and/or SEQ ID NO: 2. In some alternatives, the promoter is a BTK promoter. In some alternatives, the BTK promoter comprises the nucleic acid sequence set forth in SEQ ID NO: 5. In some alternatives, the third sequence is codon optimized for expression in humans. In some alternatives, the third sequence comprises the sequence set forth in SEQ ID NO: 6 or SEQ ID NO: 7. In some alternatives, the promoter is a B cell specific promoter. In some alternatives, the B cell specific promoter comprises the B cell specific promoter B29. In some alternatives, the B29 promoter sequence comprises a sequence set forth in SEQ ID NO: 46. In some alternatives, the B cell specific promoter is an endogenous promoter. In some alternatives, the polypeptide further comprises one or more enhancer elements. In some alternatives, the one or more enhancer elements comprises at least one DNase Hypersensitive Site (DHS). In some alternatives, the DNase Hypersensitive Site is DNase Hypersensitive Site 1 (DHS1), DNase Hypersensitive Site 2, (DHS2) DNase Hypersensitive Site 3 (DHS3), DNase Hypersensitive Site 4 (DHS4) and/or DNase Hypersensitive Site 5 (DHS5). In some alternatives, the DNase Hypersensitive Site comprises a sequence set forth in SEQ ID NO: 3. In some alternatives, the one or more enhancer elements comprise at least one intronic region. In some alternatives, the at least one intronic region is from a human BTK locus that is in association with a human BTK proximal promoter. In some alternatives, the at least one intronic region is intron 4, intron 5 and/or intron 13 of the human BTK locus that is in association with the human BTK proximal promoter. In some alternatives, the intronic region comprises the sequence set forth in SEQ ID NO: 9 (intron 4), SEQ ID NO: 10 (intron 5) and/or SEQ ID NO: 11 (intron 13). In some alternatives, the one or more enhancer elements comprise the sequence set forth in SEQ ID NO: 4, SEQ ID NO: 14 or SEQ ID NO: 15. In some alternatives, the UCOE is in a reverse orientation or forward orientation. In some alternatives, the UCOE is in a forward orientation. In some alternatives, the one or more enhancer elements comprises the sequences set forth in SEQ ID NO: 16, SEQ ID NO: 17, SEQ ID NO: 18, SEQ ID NO: 19 and/or SEQ ID NO: 20. In some alternatives, the polynucleotide further comprises a gene upstream of a BTK promoter. In some alternatives, the gene upstream of a BTK promoter is a BTK enhancer. In some alternatives, the BTK enhancer comprises the sequence set forth in SEQ ID NO: 21 or SEQ ID NO: 22. In some alternatives, 0.7UCOE comprises a sequence set forth in SEQ ID NO: 2. In some alternatives, the enhancer comprises a sequence set forth in SEQ ID NO: 3 or 4.

In a second aspect a vector for sustaining BTK expression in cells is provided, the vector comprising: a first sequence encoding an ubiquitous chromatin opening element (UCOE), a second sequence encoding a promoter, and a third sequence encoding BTK. In some alternatives, the first sequence comprises the nucleic acid sequence set forth in SEQ ID NO: 1, and/or SEQ ID NO: 2. In some alternatives, the promoter is a BTK promoter. In some alternatives, the promoter comprises the nucleic acid sequence set forth in SEQ ID NO: 5. In some alternatives, the third sequence is codon optimized for expression in humans. In some alternatives, the third sequence comprises the sequence set forth in SEQ ID NO: 6 or SEQ ID NO: 7. In some alternatives, the vector further comprises a B cell specific promoter. In some alternatives, the B cell specific promoter comprises the B cell specific promoter B29. In some alternatives, the B29 promoter sequence comprises a sequence set forth in SEQ ID NO: 46. In some alternatives, the B cell specific promoter is an endogenous promoter. In some alternatives, the vector further comprises one or more enhancer elements. In some alternatives, the one or more enhancer elements comprise at least one intronic region. In some alternatives, the one or more enhancer elements comprises a DNase Hypersensitive Site (DHS). In some alternatives, the DNase Hypersensitive Site is DNase Hypersensitive Site 1 (DHS1), DNase Hypersensitive Site 2, (DHS2) DNase Hypersensitive Site 3 (DHS3), DNase Hypersensitive Site 4 (DHS4) and/or DNase Hypersensitive Site 5 (DHS5). In some alternatives, the DNase Hypersensitive Site comprises a sequence set forth in SEQ ID NO: 3. In some alternatives, the at least one intronic region is from a human BTK locus that is in association with a human BTK proximal promoter. In some alternatives, the at least one intronic region is intron 4 intron 5 and/or intron 13 of the human BTK locus that is in association with the human BTK proximal promoter. In some alternatives, the intronic region comprises the sequence set forth in SEQ ID NO: 9 (intron 4), SEQ ID NO: 10 (intron 5) and/or SEQ ID NO: 11 (intron 13). In some alternatives, the one or more enhancer elements comprise the sequence set forth in SEQ ID NO: 4, SEQ ID NO: 14 or SEQ ID NO: 15. In some alternatives, the UCOE is in a reverse orientation or forward orientation. In some alternatives, the UCOE is in a forward orientation. In some alternatives, the vector is a lentiviral-based vector of a B lineage specific lentiviral vector. In some alternatives, the cells are B cells. In some alternatives, the cells are myeloid cells. In some alternatives, the cells are hematopoietic stem cells. In some alternatives, the cells are CD34+ hematopoietic stem cells. In some alternatives, the one or more enhancer elements comprises the sequences set forth in SEQ ID NO: 16, SEQ ID NO: 17, SEQ ID NO: 18, SEQ ID NO: 19 and/or SEQ ID NO: 20. In some alternatives, the polynucleotide further comprises a gene upstream of a BTK promoter. In some alternatives, the gene upstream of a BTK promoter is a BTK enhancer. In some alternatives, the BTK enhancer comprises the sequence set forth in SEQ ID NO: 21 or SEQ ID NO: 22. In some alternatives, 0.7UCOE comprises a sequence set forth in SEQ ID NO: 2. In some alternatives, the enhancer comprises a sequence set forth in SEQ ID NO: 3 or 4.

In a third aspect, a cell for expression of BTK is provided, wherein the cell comprises a polynucleotide, which comprises a first sequence encoding a UCOE, a second sequence encoding a promoter and a third sequence encoding BTK. In some alternatives, the polynucleotide is in a vector. In some alternatives, the vector is a lentiviral vector. In some alternatives, the cell is a B cell. In some alternatives, the cells are a myeloid cell. In some alternatives, the cell is a hematopoietic stem cell. In some alternatives, the cell is a CD34+ hematopoietic stem cell. In some alternatives, 0.7UCOE comprises a sequence set forth in SEQ ID NO: 2. In some alternatives, the enhancer comprises a sequence set forth in SEQ ID NO: 3 or 4.

In a fourth aspect, methods of promoting or increasing B cell survival, proliferation and/or differentiation in a subject in need thereof are provided, wherein the method comprises administering the cells of any one of the alternatives herein to the subject or cells comprising the polynucleotide of any one of the alternatives herein or the vector of any one of the alternatives herein to the subject in need and, optionally identifying or selecting the subject as one that would benefit from receiving a therapy that would promote B cell survival, proliferation and/or differentiation in advance of administering the cells and/or, optionally, measuring B cell survival, proliferation and/or differentiation in said subject or in a biological sample obtained from said subject after receiving the administration of the cells. In some alternatives, the cells are from the subject and, wherein the cells are genetically modified by introducing the polynucleotide of any one of the alternatives herein or the vector of any one of the alternatives herein into the cells. In some alternatives, the administering is performed by adoptive cell transfer. In some alternatives, the cells are B cells. In some alternatives, the cells are myeloid cells. In some alternatives, the cells are hematopoietic stem cells. In some alternatives, the cells are CD34+ hematopoietic stem cells. In some alternatives, the subject is male. In some alternatives, the subject is suffering from XLA. In some alternatives, the subject is selected to receive immunoglobulin replacement therapy. In some alternatives, the subject is selected to receive targeted anti-microbial agents. In some alternatives, 0.7UCOE comprises a sequence set forth in SEQ ID NO: 2. In some alternatives, the enhancer comprises a sequence set forth in SEQ ID NO: 3 or 4.

In a fifth aspect, methods of treating, inhibiting, or ameliorating X linked agammaglobulinemia (XLA) or disease symptoms associated with XLA in a subject in need thereof are provided, wherein the methods comprise administering the cell of any one of the alternatives herein to the subject or a cell comprising the polynucleotide of any one of the alternatives herein or the vector of any one of the alternatives herein to the subject in need and, optionally identifying or selecting the subject as one that would benefit from receiving a therapy for XLA or disease symptoms associated with XLA and/or, optionally, measuring an improvement in the progression of XLA or an improvement in a disease symptom associated with XLA in said subject. In some alternatives, the cell is from the subject, wherein the cell is genetically modified by introducing the polynucleotide of any one of the alternatives herein or the vector of any one of the alternatives herein into the cell. In some alternatives, the administering is performed by adoptive cell transfer. In some alternatives, the cell is a B cell. In some alternatives, the cells are myeloid cells. In some alternatives, the cells are hematopoietic stem cells. In some alternatives, the cells are CD34+ hematopoietic stem cells. In some alternatives, the subject is male. In some alternatives, the subject is selected to receive immunoglobulin replacement therapy. In some alternatives, the subject is selected to receive targeted anti-microbial agents. In some alternatives, 0.7UCOE comprises a sequence set forth in SEQ ID NO: 2. In some alternatives, the enhancer comprises a sequence set forth in SEQ ID NO: 3 or 4.

### BRIEF DESCRIPTION OF THE DRAWINGS

FIG. 1 shows the optimized gene delivery platform for XLA.

FIG. 2 shows methods for optimizing lentiviral vectors for gene therapy for XLA.

FIG. 3 shows the methods for the preclinical models for the therapy plan.

FIG. 4 shows BTK expression in lymphocyte subsets: BTK expression in bone marrow (BM), spleen (SP) and peritoneal fluid (PF) B cells, monocytes and neutrophils from XLA mice transplanted with lentiviral vector 0.7UCOE or with 0.7UCOE-14,5 expressing human BTK, as measured by flow cytometry and expressed as percent of the control BTK⁺ population.

FIG. 5 shows rescue of B cell development and function. Analysis of BTK expression in B subsets from primary recipients. Splenic B cell subsets (right panel) are represented in order from least to more mature subsets: early B cell development (pre B cell, pro B cell, immature and mature B cell) to late B cell development (transitional 1 (T1), transitional 2 (T2), marginal zone precursor (MZP), marginal zone (MZ) and follicular mature (FM).

FIG. 6 shows both vectors 0.7 UCOE.BTKp. BTK and 0.7UCOE I-4,5 BTKp. BTK rescue B cell development and function. (A) Absolute counts for splenic B cells (Right panel). (B) B cell proliferation in response to IgM stimulation. Naïve B cells were stimulated with soluble IgM. The percentage of BTK+ B cells proliferating at 72 hours is shown as normalized to WT Mock.

FIG. 7 shows responses to immunization with T-dependent antigens and autoantibody production. (A) Cumulative data for the levels of NP-specific IgG in serum from treated mice immunized with NP-CGG in Alum was detected by ELISA and measured relative to an IgG standard. ELISAs were used to detect levels of high-affinity NP-IgG before (−) and after primary (10) immunization, as well as following re-challenge or (20) secondary immunization. (B) Levels of anti-dsDNA IgG and IgG2c in serum from treated mice were measured by ELISA at the end point of the experiment depicted as absorbance readings (OD450). Serum from autoimmune WAS−/− chimeric mice are used as positive control (black triangles).

FIG. 8 shows viral integration number per cell. (A) Average viral integration number per cell in total BM and in splenic CD43− (B cell) vs. CD43+ (non B) cells in primary transplant mice as measured by qPCR. (B) Percent BTK+ B cells divided by average number of viral integrations in CD43− splenic B cell population in primary transplant mice. Data represent mean±SEM from 6 independent experiments, n=9 (WT Mock), 10 (XLA Mock) 16 (0.7UCOE.BTKp), 14 (0.7UCOE.INT4,5 BTKp).

FIG. 9 shows expression profiles of four LV constructs and rescue of B cell development and function in primary recipient mice. (9A) Schematic of the lentiviral constructs with RRL backbone used to express human BTK in murine cells with the BTK promoter (BTKp), a 1.5 kb ubiquitous chromatin opening element (UCOE) or Eμ enhancer, and codon-optimized human BTK cDNA (co). (9B) BTK marking in bone marrow, spleen, peritoneal B and myeloid cells from gene therapy treated mice was determined by flow cytometry and is shown as percent of the population that is BTK+. (Data represent mean±SEM from 10 independent experiments, n=14 (WT Mock), 12 (KO Mock), 5 (BTKp), 24 (1.5 kb.UCOE.BTKp), 18 (1.5 kb.UCOE.BTKp.co), 21 (Eμ.BTKp). (9C-9E) B cells (B220+) in the bone marrow, spleen, and peritoneum were stained for surface markers indicative of B cell subsets and analyzed for counts or percentage of live lymphocytes: (9C) Early B cell development: Pro-B (CD43+, IgM−), Pre−B (CD43−, IgM−, IgD−), Immature (CD43−, IgM+ IgD−), and Mature (CD43−, IgM+, IgD+); (9D) late B cell development: transitional T1 (CD24hi, CD21−), transitional T2 (CD24hi, CD21int), marginal zone/precursor MZ/MZP (CD24hi, CD21hi), and follicular FM (CD24int, CD21int); (9E) peritoneum B cells: B2 (B220hi), B1b (B220lo, CD5−), and B1a (B220lo, CD5+). Data represent mean±SEM from 10 independent experiments with n (BM,SP,PE)=11,13,14 (WT Mock); 10,11,10 (KO Mock); 5,5,5 (BTKp); 20,21,22 (UCOE.BTKP); 14,14,17 (UCOE.BTKp.co); 18,19,20 (Eμ.BTKp) (9F) Total splenocytes from treated mice were stimulated with media alone, anti-murine IgM, LPS or PMA/Ionomycin and proliferation was measured by incorporation of 3H-thymidine, depicted as the counts per minute (cpm) averaged from 3 replicate wells. Data represent mean±SEM from 5 independent experiments, n=10 (WT Mock), 7 (KO Mock), 5 (BTKp), 13 (1.5 kbUCOE.BTKp), 9 (1.5 kbUCOE.BTKp.co), 10 (Eμ.BTKp). (9G-9H) Total serum IgG (9G) and IgM (9H) in serum from treated mice was measured by ELISA. Data represent mean±SEM from 4 independent experiments, n=9 (WT Mock), 10 (KO Mock), 2 (BTKp), 19 (1.5 kb.UCOE.BTKp), 14 (1.5 kbUCOE.BTKp.co), 16 (Eμ.BTKp). (9I) Levels of NP-specific IgG in serum from treated mice immunized with NP-CGG in Alum, measured by ELISA and expressed relative to an IgG standard. ELISAs were used to detect levels of low-affinity (Supp) and high-affinity (i) NP-IgG before (−) and after (+) immunization, as well as following later re-challenge (++). Data represent mean±SEM from 5 independent experiments with n=9 (WT Mock), 10 (KO Mock), 2 (BTKp), 19 (1.5.UCOE.BTKp), 14 (1.5 kb.UCOE.BTKp.co), 16 (Eμ.BTKp). ***P<0.001; **P=0.001-0.01; *P=0.01-0.05, between total B cells or B cell subsets from the indicated experimental group and KO Mock group.

FIG. 10 shows Eμ.BTKp gene therapy treated mice develop broad specificity IgG autoreactive antibodies. Levels of anti-dsDNA IgG (a) in serum from treated mice were measured by ELISA prior to any immunizations and are depicted as absorbance readings (OD450). Data represent mean±SEM from 10 independent experiments, n=18 (WT Mock), 16 (KO Mock), 6 (BTKp), 30 (1.5 kb.UCOE.BTKp), 18 (1.5 kb.UCOE.BTKp.co), 29 (Eμ.BTKp), 2 (WASp control). (b) Anti-dsDNA IgG subclasses IgG2c and IgG3 were measured by ELISA from Eμ.BTKp mice (n=23). (c) Subclasses of anti-dsDNA IgG in sera from 9 Eμ.BTKp treated mice were measured by ELISA. ***P<0.001; **P=0.001-0.01; *P=0.01-0.05, between the indicated experimental groups.

FIG. 11 shows 1.5 kbUCOE.BTKp and 1.5 kbUCOE.BTKp.co lead to sustained BTK expression with lower copy numbers in primary and secondary recipients. Splenic granulocytes (CD11b+, GR1hi) from secondary transplant mice were evaluated for % BTK+ cells by flow cytometry. (a) Graphical analysis of % BTK+ granulocytes in secondary transplant recipients. Data represent mean±SEM from 3 independent experiments with n=3 (WT Mock), 2 (KO Mock), 3 (BTKp), 8 (1.5.UCOE.BTKp), 7 (Eμ.BTKp). (b) Percent BTK+ granulocytes divided by average number of viral integrations in splenocytes from primary transplant mice. (c-d) Average viral integrations per cell in bone marrow (c) and splenocytes (d) from primary and secondary transplant mice measured by qPCR. Data represent mean±SEM from 8 primary and 3 secondary transplant experiments with n (primary and secondary)=5, 3 (BTKp); 18, 8 (UCOE.BTKp); 10, 1 (UCOE.BTKp.co); 18, 7 (Eμ.BTKp). ***P<0.001; **P=0.001-0.01; *P=0.01-0.05, between the indicated experimental groups.

FIG. 12 shows restoration of BTK expression in affected hematopoietic lineages and B cell development in gene therapy mice treated with 0.7 kb UCOE.BTKp.co LV. The proportion of BTK+ cells and numerical reconstitution of B cell subsets was assessed at 16-25 weeks post-transplant. (a) Schematic diagram of a lentiviral construct with RRL backbone expressing codon optimized human BTK with a 0.7 kb ubiquitous chromatin opening element (UCOE). (b-c) Percent of BTK+ cells in bone marrow (b) and splenic (c) lymphocyte subsets (Neutrophils, Monocytes, B cells and T cells) from gene therapy treated groups, determined by flow cytometry. Data represent mean±SD from 7 unique experiments, n=13 (WT Mock), 12 (KO Mock), 36 (0.7 kb.UCOE.BTKp.co). (d) Representative flow plots of BTK expression in splenic Neutrophils (CD11b+ GR1+), Monocytes (CD11b+), B cells (B220+) and T cells (CD4+CD8+), (e) BTK expression in Peritoneal B cells (CD19+) and B cell subsets B1a (CD5− CD43+), B1b (CD5+ CD43+) and B2 (CD5− CD43−). Representative data from 7 unique experiments with SD and n=2 (WT Mock), 2 (KO Mock), 8 (0.7 kb.UCOE.BTKp.co). (f) Rescue of B cell development by Btk expression in the subsets, as measured by flow cytometry. Early B cell development: Pro-B (CD43+, IgM−), Pre−B (CD43−, IgM−, IgD−), Immature (CD43−, IgM+ IgD−), and Mature (CD43−, IgM+, IgD+); and late B cell development: transitional T1 (CD24hi, CD21−), transitional T2 (CD24hi, CD21int), marginal zone/precursor MZ/MZP (CD24hi, CD21hi), and follicular FM (CD24int, CD2 lint). Cumulative data from 7 independent experiments, n=13 (WT Mock), 12 (KO Mock), 36 (0.7 kb.UCOE.BTKp.co). (g-h) Total cell counts of the B cell subsets from in BM (g) and Spleen (h). (i) B cell subsets as percent of total lymphocyte population in peritoneal fluid. Data represent mean±SD from 2 independent experiments, n=4 (WT Mock), 4 (KO Mock), 11 (0.7 kbUCOE.BTK.co) The * represent significant difference between KO Mock and 0.7 kb.UCOE.BTK.co. P value was determined using the One way Anova: Sidak test: ***P<0.001; **P=0.001-0.01; *P=0.01-0.05.

FIG. 13 shows reconstitution of B cell function following 0.7.UCOE.Bkp.co LV gene therapy in primary recipients. Assays of B cell function after 16-25 weeks post bone marrow transplantation. Bars show the mean±SD (a) The percentage of B cells (CD43− splenocyte) that underwent ≥1 cell division 72 hours after incubation with anti-mouse IgM antibodies, LPS or P/I (as measured by CFSE dilution and read out by flow cytometry). Representative data from one independent experiment; n=4 (WT Mock), 3 (KO Mock), and 5 (0.7.UCOE.BTKp.BTK.co). (b) Flow cytometry analysis showing CFSE labeled B cells (CD43− splenocytes) 72 hours post-IgM stimulation, gated on live and B220+ populations (working on the flow plot). (c) Levels of high-affinity NP-specific IgG in serum from treated mice immunized with NP-CGG in Alum, measured by ELISA. The values are measured before (−) and post (+) immunization as well as following re-challenge (++). Data represent 7 independent experiments with n=11 (WT Mock), 10 (KO Mock), 13 (0.7 kb.UCOE.BTKp.co) in primary immunization (+) and n=3 (WT Mock), 3 (KO Mock), 3 (0.7 kb.UCOE.BTKp.co) for re-challenge (++). (d) Total IgG and IgM and (e) anti-dsDNA antibody levels in the serum of immunized primary recipients as determined by ELISA 16-25 weeks post-transplant. Data are from 7 independent experiments; n=10 (WT Mock), 8 (KO Mock) and 26 (0.7.UCOE.BTKp.BTK.co.). Serum from 2 autoimmune WAS chimera with high serum anti-DNA antibodies was run as a positive control (e). P value was determined using the One way Anova: Sidak test: ***P<0.001; **P=0.001-0.01; *P=0.01-0.05.

FIG. 14 shows VCN and BTK expression are maintained after serial passage of gene therapy-treated bone marrow cells into secondary TBK−/− recipients. Percent of BTK+ expressing cells within affected lineages of the BM (a) and spleen (b) of secondary recipients 16 weeks post-transplantation with BM from primary recipients was measured by flow cytometry. Data represents two individual experiments; n=4 (WT Mock), 4 (KO Mock), 19 (0.7 UCOE.BTKp.BTKco). (c) Viral copy number (VCN) determined by qPCR of gDNA extracted from total BM and splenic B (CD43−) and non-B (CD43+) cells of primary recipients. (d) VCN of total BM and splenic cells from secondary recipient mice; each dot represents a single animal. Data are from 7 unique experiments for primary (n=21 0.7.UCOE.BTKp.BTK.co) and secondary recipients (n=11 0.7.UCOE.BTKp.BTK.co) (e) Splenic gDNA from primary and secondary recipients was assessed for methylation. n for primary recipients=2 (BTKp.BTK), 5 (1.5 kb UCOE.BTKp.BTK), 6 (Eu.BTKp.BTK.), 6 (0.7 kb.UCOE.BTKp.co). n for secondary recipients=4 (1.5 kb.UCOE.BTKp.BTK) and 6 (0.7 UCOE.BTKp.co). In all panels the mean±SD value are shown; P values comparing the means of KO Mock to 0.7.UCOE.BTKp.BTK.co. P value was determined using the One way Anova: Sidak test: ***P<0.001; **P=0.001-0.01; *P=0.01-0.05.

FIG. 15 shows 0.7UCOE.BTKp.co leads to sustained BTK expression and lower copy numbers in XLA CD34 cells. XLA and control CD34 cells were transduced with 0.7UCOE.BTKp.co at various multiplicities of infection (MOI) and cultured in vitro for 15 days. (a) Schematic diagram of the lentiviral transduction protocol of healthy and XLA CD34 cells. Cells were stimulated for 48 hr in SCGM media supplemented with 100 ng of human TPO, SCF and Flt3. Cells were transduced with 0.7UCOE.BTKp.co and grown in culture for 15 days. At day 15 cells were analyzed for BTK expression and VCN. (b) Representative flow plots of BTK staining in CD34 cells from an XLA patient 15 days post-transduction at various MOIs. (c) Viability of transduced cells after 24 hr transduction, determined by flow cytometry. (d) Viral copy number (VCN) determined by qPCR of gDNA extracted from total cells collected after 15 days in culture. Data represent mean±SD from 3 unique experiments done in duplicate wells. Each dot represents an individual donor, either healthy or XLA.

FIG. 16 shows the BTK promoter mimics BTK's endogenous expression pattern in mice. (a-b) Bone marrow, spleen, peritoneal wash, peripheral blood and thymus were collected from a wildtype mouse and cells were stained for B, myeloid, T and NK cell markers, intracellular BTK and read on a flow cytometer. Samples from the various tissues are designated by the shape of the symbol, as outlined in the legend. The percent of cells that are BTK+ is shown for each subset (a), as well as the median fluorescence intensity of BTK+ cells (b). (c) Schematic of lentiviral constructs with RRL backbone, Eμ enhancer and human BTK cDNA, under control of either the B29 promoter (top panel) or the endogenous BTK promoter BTKp (bottom panel). (d-e) Bone marrow, spleen and peritoneal cells were collected from Eμ.B29 or Eμ.BTKp gene therapy treated KO mice and stained with markers for B (CD11b−, B220+), myeloid (CD11b+) and T (CD11b−, CD3+) cells, intracellular BTK, and analyzed by flow cytometry. (d) The percent of cells that are BTK+. (e) The median fluorescence intensity of BTK staining in indicated cell populations. Data represent mean±SEM from 2 independent experiments with n=4 (EμB29) and n=5 (EμBTKp). ***P<0.001; **P=0.001-0.01; *P=0.01-0.05, between Eμ.B29 and Eμ.BTKp.

FIG. 17 shows (a) Schematic of lentiviral constructs under control of the BTK promoter with Eμ enhancer and human BTK cDNA, either wild type of codon-optimized, with a T2A-linked GFP. (b) Chicken BTK−/− DT40 cells were transduced with BTK-GFP or coBTK-GFP constructs; histograms show GFP and BTK expression. (c) BTK-GFP and coBTK-GFP transduced cells were stained with an Indo-1 Ester AM fluorescent dye and stimulated with anti-IgM; calcium mobilization was monitored via flow cytometry.

FIG. 18 shows (a) Representative plots of BTK expression by flow cytometry in peripheral blood B cells and myeloid cells from gene therapy-treated KO mice. (b) BTK marking in bone marrow, spleen, peritoneal B and myeloid cells from gene therapy treated mice was determined by flow cytometry and the median fluorescence intensity of the BTK+ cells is shown. (Data represent mean±SEM from 10 independent experiments, n=14 (WT Mock), 12 (KO Mock), 5 (BTKp), 24 (1.5 kb.UCOE.BTKp), 18 (1.5 kb.UCOE.BTKp.co), 21 (Eμ.BTKp). (c) Representative flow cytometry plots from bone marrow of gene therapy treated mice, stained with markers for early B cell development. (d) Graphs depict the percentage of B cells following Hardy fractions of decreasing maturity: Fr I (IgMlo, IgDhi), Fr II (IgMhi, IgDhi), FrIII (IgMhi, IgDlo), each ring represents one mouse and the mean percent of B cells in each fraction is shown inside the graphic. Data represent mean±SEM from 11 independent experiments, n=18 (WT Mock), 14 (KO Mock), 7 (BTKp), 43 (1.5 kb.UCOE.BTKp), 18 (1.5 kb.UCOE.BTKp.co), 23 (Eμ.BTKp). (e-f) Levels of NP-specific IgG in serum from treated mice before (−) and after (+) immunization with NP-CGG in Alum were measured by ELISA and expressed relative to an IgG standard. Low-affinity NP-specific antibodies are shown for all groups (e) and for individual mice (f). Data represent mean±SEM from 5 independent experiments with n=9 (WT Mock), 10 (KO Mock), 2 (BTKp), 19 (1.5.UCOE.BTKp), 14 (1.5 kb.UCOE.BTKp.co), 16 (Eμ.BTKp). (g) Levels of TNP-specific IgM in serum from treated mice immunized with TNP-Ficol, measured by ELISA and expressed relative to an IgM standard. Data represent mean±SEM from 3 independent experiments with n=4 (WT), 6 (WT Mock), 4 (KO), 3 (KO Mock), 14 (UCOE.BTKp). ***P<0.001; **P=0.001-0.01; *P=0.01-0.05, between the indicated experimental group and KO Mock group.

FIG. 19 shows (a) Survival curve of Eμ.BTKp primary transplant mice with high and low anti-dsDNA IgG titers, compared to controls. (b) Correlation of BTK MFI of BTK+ cells and Anti-dsDNA IgG titers as measured by flow cytometry and ELISA, respectively, in Eμ.BTKp primary transplant mice.

FIG. 20 shows sera from WT Mock (4), 1.5 kb.UCOE.BTKp (4) and Eμ.BTKp (8) mice were analyzed by autoantigen array for levels of IgM and IgG reactive to 88 murine antigens. Data from each row was subject to z-transformation and Z-scores are displayed on a colorimetric scale from lowest reactivity (red) to highest (blue).

FIG. 21 shows the development of B cells and the pathway in which the enzyme BTK is involved.

FIG. 22 shows development of the optimal BTK LV gene therapy vector.

FIG. 23 shows LV vectors containing the BTK endogenous promoters. As shown, the 2 kb CBX3-HNRPA2B1 element-UCOE 1.5-eliminates the A2 promoter but retains the CBX3 in reverse orientation.

FIG. 24 shows the development of LV vectors containing the BTK endogenous promoter. As shown, Eμ associated with higher level Btk MFI in B cells.

FIG. 25 shows the results from a FACs assay of LV vectors containing the Btk endogenous promoter.

FIG. 26 shows the expression of BTK in myeloid cells that were obtained from splenocytes of treated mice.

FIG. 27 shows the results of BTK expression in B cells in which the LV expressing the BTK comprises a B cell specific promoter Eμ.B29.

FIG. 28 shows BTK expression in B cells and monocytes from the lentiviral vector comprising the promoter region BTKpro.BTK

FIG. 29 shows BTK expression in B cells and monocytes from the lentiviral vector comprising the promoter region BTKpro.BTK and the ubiquitous chromatin opening element (UCOE).

FIG. 30 shows BTK expression in B cells and monocytes from the lentiviral vector comprising the promoter region Eμ.BTKpro.BTK (BTK promoter) and huBTK.

FIG. 31 shows the expression profile of BTK of several BTK promoters in B cells and myeloid cells of the bone marrow, spleen and peritoneum.

FIG. 32 shows the expression profile of BTK of several BTK promoters in B cells and myeloid cells of the bone marrow, spleen and peritoneum. The vectors used were: WT mock, DKO mock, BTKp, UCOE BTKp, UCOE.BTKp.co and EμBTKp.

FIG. 33 shows B Cell Development Restoration of Mature B Cell Subsets. The black colored regions show that more mature peripheral B cell populations.

FIG. 34 shows B Cell Development Numerical Reconstitution of B Cell Populations (bone marrow, spleen and Peritoneum). As shown, the data summarize findings from up to 40 recipient mice per vector and similar numbers of controls.

FIG. 35 shows B cell proliferation in cells expressing four different types of lentiviral vectors (mock wt, DKO mock, lentiviral vector with UCOE.BTKp and lentiviral vector Eμ.BTKp). The vital dye dilution vs Btk stain showed that UCOE led to very similar results to the WT mice in the experiments.

FIG. 36 shows B cell proliferation in cells with the following vectors: WT, WT mock, DKO, DKO mock, UCOE.BTKp and Eμ.BTKp.

FIG. 37 shows the results of IgM and IgG production in cells with the following vectors: WT, WT mock, DKO, DKO mock, BTKp, UCOE.BTKp and Eμ.BTKp. The bottom panel shows T independent immune responses in cells with the following vectors: WT, WT mock, DKO, DKO mock, UCOE.BTKp, WT-0 and KO-0. It is noted that the Eμ IgG levels actually slightly above WT mock recipients.

FIG. 38 shows T-dependent immune responses in cells with the following vectors: WT, WT mock, DKO, DKO mock, BTKp, UCOE.BTKp, UCOE.BTKp.co, WT-0, Eμ.BTKp and KO-0.

FIG. 39 shows T-dependent immunization: affinity maturation. Affinity maturation for individual UCOE animals and controls led to very similar results.

FIG. 40 shows evidence for auto-antibody production.

FIG. 41 shows evidence for auto-antibody production using a heat map of cells that have the vectors WT mock, UCOE.BTKp and Eμ.BTKp. This was performed for testing the production of IgM and IgG.

FIG. 42 shows association of antibody levels and percent mouse survival with BTK expression.

FIG. 43 shows long-term stem cell marking in neutrophils with BTK expression in secondary recipient mice.

FIG. 44 shows evidence for long-term stem cell marking in VCN in primary and secondary recipients that have the vectors: mock, BTKp, UCOE.BTKp, UCOE.BTKp.co, and Eμ.BTKp.

FIG. 45 shows the impact of LV therapy on long-term survival.

FIG. 46 shows the summary of testing of alternative enhancer elements within BTK promoter LV.

FIG. 47 shows that 0.7UCOE.BTKp and 0.7UCOE.IE.BTKp rescue BTK expression and splenic B cell counts.

FIG. 48 shows that 0.7UCOE.BTKp and 0.7UCOE.IE.BTKp rescue B cell function.

FIG. 49 shows that 0.7UCOE.IE exhibits an improved safety profile over 0.7UCOE.

FIG. 50 shows new BTK constructs with DNase Hypersensitive Sites.

FIG. 51 shows the identification of DNase Hypersensitive sites.

FIG. 52 shows a schematic of the constructs that were evaluated for BTK expression in cells.

FIG. 53 shows in vivo comparison of DHS constructs to 0.7UCOE and 0.7UCOE.IE.

FIG. 54 shows peripheral blood lymphocyte distribution in B cells, T cells, monocytes and neutrophils 15 weeks post-transplant. BTK reconstitution was also shown in lymphocyte subsets in cells with the following vectors: KO unirradiated, KO Mock, 0.7 UCOE, 0.7 UCOE.IE, 0.7 UCOEDHS4, 0.7 UCOE.DHS12, 0.7 UCOE.DHS124, 0.7 UCOE1-5, WT mock and WT unirradiated.

FIG. 55 shows BTK experimental summary in cells with the vectors: KO Mock, 0.7 UCOE, 0.7 UCOE.IE, 0.7 UCOEDHS4, 0.7 UCOE.DHS12, 0.7 UCOE.DHS124, 0.7 UCOE1-5 and WT mock.

FIG. 56 shows BTK expression in cells with vectors with either the UCOE element or DHS4 element.

FIG. 57 shows BTK expression experiments with cells that have the following vectors: 0.7UCOE.BTKp.coBTK, KO Mock, 0.7 UCOE, DHS4, DHS1-5 and WT Mock.

FIG. 58 shows peripheral blood lymphocyte distribution-12 weeks post transplantation with cells with the following vectors: KO Mock, WT Mock, 0.7 UCOE, DHS4 and DHS1-5.

FIG. 59 shows 6 week and 12 week peripheral blood lymphocyte distribution in B cells, monocytes and neutrophils that have the following vectors: KO unirradiated, KO Mock, 0.7 UCOE, DHS4, DHS1-5, WT Mock, and WT unirradiated.

FIG. 60 shows 6 week and 12 week BTK expression in B cells, monocytes and neutrophils that have the following vectors: KO unirradiated, KO Mock, 0.7 UCOE, DHS4, DHS1-5, WT Mock, and WT unirradiated.

FIG. 61 shows that BTK is expressed across cell subsets after a 12 week bleed following cell transplantation.

FIG. 62 shows the experimental set up for the in vitro comparison of original coBTK (codon optimized BTK) vs new coBTK.

FIG. 63 shows in vitro Comparison—TBK Lineage Negative Cells, Volume Matched Virus (day 7 BTK stain).

FIG. 64 shows the percent BTK expression in cells that received different concentrations of the lentivirus vectors for BTK expression. The vectors used were the following: KO Mock, 0.7 UCOE, DHS4, 0.7 UCOE.newcoBTK, 0.7 UCOE.newcoBTK, and WT spleen.

FIG. 65 shows in vivo comparison of original vs new coBTK.

FIG. 66 shows additional techniques for optimizing the UCOE elements to increase or enhance BTK expression.

FIG. 67 shows additional techniques for optimizing the UCOE elements to increase or enhance BTK expression for the 1.5 kb UCOE.

FIG. 68 shows 0.7UCOE.BTKp vs. 0.7UCOEfwd.BTKp: in vitro test with matched volume virus.

FIG. 69 shows an outline for finalizing the clinical BTK LV construct.

FIG. 70 shows an outline for finalizing the clinical BTK LV construct and planned final in vivo testing for a codon optimized BTK.

FIG. 71 is a table showing approved apheresis of XLA patient's stem cells.

FIG. 72 shows a human chimera in NSG.

FIG. 73 shows human lymphocyte reconstitution in B cells of the bone marrow and the spleen.

FIG. 74 shows phenotype of engrafted XLA stem cell (spleen) such as the expression of IgD and IgM.

FIG. 75 shows a phenotype of engrafted XLA stem cell (spleen) such as the expression of CD24 and CD38.

FIG. 76 shows B cell developmental block (BM) indicating that there is an equivalent number of % ProB cells between the XLA group and the healthy group.

FIG. 77 shows B cell developmental block (BM) indicating that patients with XLA have a significantly higher % pre-B cells compared to a healthy control.

FIG. 78 shows B cell developmental block (BM) indicating that patients with XLA have cells that are blocked at the Pre B cell stage. These Pre B cells are able to migrate into the spleen. CD179a/CD179b surrogate light chain is disulfide-linked to membrane-bound Ig mu heavy chain in association with a signal transducer CD79a/CD79b heterodimer to form a B cell receptor-like structure, so-called preB cell receptor (preBCR).

FIG. 79 shows a B cell developmental block (BM). Development of XLA B cells are blocked at Pre B cell stage. Pre B cells are able to migrate to the spleen.

FIG. 80 shows that that B cells function by Ca²⁺ flux. However, the XLA B cells are unable to flux with IgM as compared to the control cells.

FIG. 81 shows the block of the pathway in B cell development, in which XLA causes the B cell development to be blocked at the Pre B cell stage.

FIG. 82 shows an outline of the results and conclusions from the experiments.

FIG. 83 shows a diagram outlining the human lentiviral transduction of stem cells.

FIG. 84 shows a diagram outlining preclinical modeling-human HSC.

FIG. 85 shows results from the Human lentiviral transduction of Stem cells. As shown there is 70% viability with 0.7UCOE.BTKp.BTK compare to DHS4.

FIG. 86 shows BTK expression at D15 with XLA P2. As shown, in a non-selective environment 0.7 UCOE.BTKpBTK leads to higher expression of BTK compare to DHS4.

FIG. 87 shows an outline of the conclusion from the experiments and follow up experiments.

FIG. 88 shows preclinical modeling for Human HSC. For these experiments, CD34+ cells are transduced with either mock vector or the vector: UCOE.BTKp.co.

FIG. 89 shows preclinical modeling-Human HSC. Cells were transduced with the vector UCOE.BTKpro.co-opBTK (UCOE.BTKp.co) GFP.

FIG. 90 shows a diagram of the experimental methods for transduction of the NSG recipient mice.

FIG. 91 shows the results from the transformed recipient mice: % GFP expression, viral copy expression in the cells of the bone marrow and the spleen, as well as, the expression in B cells and the myeloid cells.

FIG. 92 shows the analysis to identify conserved non-coding sequences by comparing mouse and human BTK gene sequences and all the vectors that are tested for expression of BTK. The regions identified and located were within the BTK promoter, upstream from the BTK promoter and proximal to the neighboring gene (BTK enhancer or BTKe), within part of intron 1, introns 4, 5, and 13.

FIG. 93 shows results from truncation of 1.5 kb UCOE to 0.7 kb and identification of potential DNA enhancer elements may improve titer and BTK expression in Lentiviral constructs. (a) Truncation of 1.5 kb Ubiquitous Chromatin Opening Element (UCOE) to 0.7 kb. The UCOE element spans a large, CpG-rich region across the divergently transcribed promoter regions for the housekeeping genes CBX3 and HNRPA2B1, and has traditionally been truncated to a 1.5-2.2kb region by various groups for use in protein expression constructs. The 1.5 kb UCOE used here starts at Exon 1 of CBX3 and spans past CBX3 Alternate Exon 1. Truncation of this region to 0.7 kb eliminates the region downstream of Alt. Ex. 1. (b) DNaseI Hypersensitive sites (DHS) from intronic regions of the BTK gene were identified from the ENCODE database and visualized using the UCSC Genome Browser on Human February 2009 (GRCh37/hg19) Assembly. Five DHS were identified, and were labeled consecutively as DHS1, DHS2, DHS3, DHS4, and DHS5 (blue boxes). ENCODE Genome Segmentation identified a predicted Enhancer element around DHS4 (yellow box). Exons are shown as black boxes. The sequence length is noted beneath each DHS. Various combinations of these DHS sequences were cloned into the 0.7UCOE.BTKp.coBTK construct and tested in vitro (data not shown). (c) An in vitro transduction experiment of murine (TBK) lineage negative cells was performed to compare BTK expression levels in two versions of codon-optimized BTK: coBTK (FIGS. 1-2), and a codon-optimized BTK published by Staal et al. (Leukemia 2010), denoted here as co2BTK. Representative flow plots show BTK expression at 7 days post-transduction. (d) Based on the results of the in vitro testing of the DHS constructs and the coBTK vs co2BTK comparison, four constructs were identified to test in vivo. Shown here are diagrams of the lentiviral constructs with RRL backbone expressing either version of codon optimized human BTK (coBTK or co2BTK) with a 0.7 kb ubiquitous chromatin opening element (0.7UCOE), with or without addition of DHS4 downstream of the 0.7UCOE element.

FIG. 94 shows restoration of BTK expression in affected hematopoietic lineages and B cell development in gene therapy mice treated with 0.7UCOE vectors. The proportion of BTK+ cells and numerical reconstitution of B and myeloid cell subsets was assessed at 19-23 weeks post-transplant. (FIG. 94A) Representative flow plots showing intracellular BTK staining in splenic B cells at endpoint analysis. (b-d) Percent of BTK+ cells in bone marrow (FIG. 94B), spleen (FIG. 94C), and peritoneal (FIG. 94D) lymphocyte subsets from gene therapy treated groups, determined by flow cytometry. Cell subsets are defined as Neutrophils (CD11b+ GR1+), Monocytes (CD11b+), and B cells (B220+). (FIG. 94E-F) Stacked bars show the average counts of B cell subsets in bone marrow (FIG. 94E), spleen (FIG. 94F), and peritoneum (FIG. 94G). Early B cell development (bone marrow): Pro+Pre−B (IgM−, IgD−), Immature (IgM+ IgD−), and Mature (IgM+, IgD+). Late B cell development (spleen): transitional T1 (CD24hi, CD21−), transitional T2 (CD24hi, CD21int), marginal zone/precursor MZ/MZP (CD24hi, CD21hi), and follicular FM (CD24int, CD2 lint). Peritoneal B cell subsets: B1 (IgM+ CD43+), and B2 (CD43−). (FIG. 94H) Rescue of BTK expression in B cell subsets, measured by flow cytometry. (FIG. 94I) BTK+ MFI across B cell development, normalized to WT Mock in each individual experiment. Data represent mean±SD from 4 unique experiments, n=13 (WT Mock), 13 (KO Mock), 11 (0.7UCOE.BTKp.co), 11 (0.7UCOE.BTKp.co2), 16 (0.7UCOE.DHS4.BKTp.co), 11 (0.7UCOE.DHS4.BKTp.co2), and 6 (0.7UCOE.DHS1-5.BTKp.co). P value was determined using the One way Anova: Sidak test: ***P<0.001; **P=0.001-0.01; *P=0.01-0.05.

FIG. 95 shows restoration of B cell function in vivo and in vitro. (FIG. 95A) Mice were immunized with NP-CGG in Alum at 12 weeks post-transplant. Levels of NP-specific IgG in serum from immunized mice was measured by ELISA and expressed relative to an IgG standard. High-affinity NP-IgG was measured from serum prior to (−) and 10 days following primary immunization (1°). One month following primary challenge, mice were re-challenged with NP-CGG in PBS and serum was collected 10 days later (2°). (FIG. 95B-C) Total serum IgG (FIG. 95B) and IgM (FIG. 95C) in serum from treated mice was measured by ELISA and endpoint analysis (21-23 weeks post-transplant). (FIG. 95D-F) At endpoint analysis, B cells were isolated from splenocytes by CD43− magnetic separation, labeled with Cell Trace Violet and stimulated in vitro with IgM, LPS, or a media control. (FIG. 95D) The percentage of BTK+ B cells that underwent ≥1 cell division 72 hours after incubation with anti-mouse IgM antibodies, LPS or media only (read out by flow cytometry). (FIG. 95E) BTK+ MFI of cells after each division (DO-D4), normalized to WT Mock. (FIG. 95F) Representative flow plots showing BTK staining and Cell Trace dilution in B cells 72 hours post-IgM stimulation, gated on live and B220+ BTK+ cells. Data represent mean±SD from 4 unique experiments, n=13 (WT Mock), 13 (KO Mock), 11 (0.7UCOE.BTKp.co), 11 (0.7UCOE.BTKp.co2), 16 (0.7UCOE.DHS4.BKTp.co), 11 (0.7UCOE.DHS4.BKTp.co2), and 6 (0.7UCOE.DHS1-5.BTKp.co). P value was determined using the One way Anova: Sidak test: ***P<0.001; **P=0.001-0.01; *P=0.01-0.05.

FIG. 96 shows vector safety considerations. (FIG. 96A-B) Levels of anti-dsDNA IgG (FIG. 96A) and anti-dsDNA IgG2c (FIG. 96B) in serum from treated mice were measured by ELISA and are depicted as absorbance readings (OD450). Serum from the known autoimmune-prone WAS chimeric mouse model and mice treated with Eu.BTKp were included as positive controls. (FIG. 96C) Genomic DNA was isolated from total bone marrow and spleen at endpoint analysis and the number of viral integrations per cell (VCN) was quantified by qPCR. Data represent mean±SD from 4 unique experiments, n=13 (WT Mock), 13 (KO Mock), 11 (0.7UCOE.BTKp.co), 11 (0.7UCOE.BTKp.co2), 16 (0.7UCOE.DHS4.BKTp.co), 11 (0.7UCOE.DHS4.BKTp.co2), and 6 (0.7UCOE.DHS1-5.BTKp.co). P value was determined using the One way Anova: Sidak test: ***P<0.001; **P=0.001-0.01; *P=0.01-0.05.

FIG. 97 shows a schematic of a transfection protocol.

FIG. 98 shows equivalent bone marrow engraftment of human hematopoietic cells between treatment groups. Human stem cells from XLA patient, either with or without gene therapy treatments, engrafted into the bone marrow equivalently as those from a healthy donor. 98A. Representative flow plots showing various markers of human immune cells including human and mouse CD45 (a marker for hematopoietic cells) CD33 (myeloid cells) and CD19 (B cells), CD4 and CD8 (T cells). Human hematopoietic cells are hCD45+ and mCD45− from total live BM cells; CD33 and CD19 markers were analyzed from hCD45+gate; CD4 and CD8 were analyzed from the CD33-CD19-gate. 98B and 98C. % human CD45 and total number of hCD45 cells engrafted in BM; n=4 for each cohort (XLA3, XLA3+LV 0.7UCOE.BTKp.BTKco2, XLA3+LV 0.7UCOE.DHS4.BTKp.BTKco2, and healthy donor).

FIG. 99 shows Pre−clinical modeling XLA3: Spleen analysis 12 weeks post-transplant.

FIG. 100 shows Pre−clinical modeling XLA3: Spleen analysis 12 weeks post-transplant and results of mouse recipients of XLA3 HSC treated with gene therapy using 0.7UCOE.BTKp BTKco2 or 0.7UCOE.DHS4.BTKp.BTKco2.

FIG. 101 shows representative flow plot for B cell development subsets in spleen. A typical gating strategy for identifying human B cell developmental subsets is shown for each gene therapy cohort (listed at right). Markers used in each panel are shown at bottom (Hist=histogram); predecessor gates are shown at the top of each column if used.

FIG. 102 shows that recipients of LV transduced XLA3 HSC (using 0.7UCOE.BTKp.BTKco2 or 0.7UCOE.DHS4 BTKp.BTKco2) have an increased proportion of splenic immature B cells (including CD19+CD24+CD38+ B cells and CD19+CD24+CD38+IgM+ cells) compared to XLA controls. Graphs summarize the results from flow cytometry shown in FIG. 4, looking at specific subpopulations of immature B cells. A,% of Immature B cells (CD24+ CD38+) in the spleen; B, % of immature B cells that are IgM+; C-D, Total number of immature B cells (CD24+ CD38+) and CD24+hCD38+IgM+ cells in the spleen; E, overlay of CD10 histogram, showing the mean fluorescence intensity MFI shift in CD10 compared to healthy donor. n=4 for each cohort; ***P=0.0004; **P=0.0044; *P=0.4 by one way ANOVA.

FIG. 103 shows that recipients of LV transduced XLA HSC (0.7UCOE.BTKp.BTKco2 or 0.7UCOE.DHS4 BTKp.BTKco2) have increased proportions of mature B cells (CD19⁺CD24^{low}CD38^{low}IgM⁺ and IgD⁺) compared to XLA controls. 104A-C, % of mature B cells (hCD24^{low}hCD38^{low}) and the percent of mature B cells that are IgM⁺ and IgM⁺IgD⁺ in the spleen, respectively; 103D-F, Total number of splenic mature B cells and mature B cells that are IgM⁺ and IgM⁺IgD⁺, respectively; 103G, histogram overlay of CD10, showing similar MFI of CD10 as that of a healthy donor. N=4 for each cohort; ***P=0.0004; **P=0.0044; * P=0.4 by one way ANOVA.

FIG. 104 shows a representative flow plot for B cell development subsets in the bone marrow.

FIG. 105 shows that recipients of LV transduced XLA HSC (using 0.7UCOE BTKp BTKco2 or DHS4 BTKpBTK.co2) exhibit an increased proportion of CD19⁺CD24⁺CD38⁺IgM⁺ immature B cells compared to XLA controls.

FIG. 106 shows that recipients of LV transduced XLA HSC (using LV 0.7UCOE BTKp BTKco2 or LV 0.7UCOE.DHS4 BTKp.BTK.co2) exhibit gene marking of 0.2-2 viral copy number (VCN)/cell in vivo.

FIG. 107 shows that recipients of XLA3 HSC that received gene therapy with LV 0.7UCOE.BTKp.BTKco2 or 0.7UCOE.DHS4.BTK.pBTKco2 produce B cells that secrete IgM in vivo.

FIG. 108 shows that recipients of XLA3 HSC transduced with LV 0.7UCOE.BTKp.BTKco2 or 0.7UCOE.DHS4.BTKp.BTKco2 exhibit a restored capacity for B cells to flux calcium in response to B cell receptor (BCR) engagement.

FIG. 109 shows the B cell differentiation protocol.

FIG. 110 shows a representative flow plot for in vitro B cell class switching.

FIG. 111 shows that recipients of LV transduced XLA HSC (using 0.7UCOE BTKp BTKco2 or DHS4 BTKpBTK.co2) generate B cells that are capable of responding to cytokine and T-cell dependent signals leading to antibody secretion.

### DEFINITIONS

Unless defined otherwise, all technical and scientific terms used herein have the same meaning as commonly understood by one of ordinary skill in the art to which the invention pertains.

As used herein, “a” or “an” may mean one or more than one.

“About” as used herein when referring to a measurable value is meant to encompass variations of ±20% or ±10%, more preferably ±5%, even more preferably ±1%, and still more preferably ±0.1% from the specified value.

“Polynucleotide,” as described herein refers to “nucleic acid” or “nucleic acid molecule,” such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes so-called “peptide nucleic acids,” which comprise naturally-occurring or modified nucleic acid bases attached to a polyamide backbone. Nucleic acids can be either single stranded or double stranded. In some alternatives, a nucleic acid sequence encoding a fusion protein is provided. In some alternatives, the nucleic acid is RNA or DNA.

Coding for” or “encoding” have their plain and ordinary meaning when read in light of the specification, and may include but is not limited to, for example, the property of specific sequences of nucleotides in a polynucleotide, such as a gene, a cDNA, or an mRNA, to serve as templates for synthesis of other macromolecules such as a defined sequence of amino acids. Thus, a gene codes for a protein if transcription and translation of mRNA corresponding to that gene produces the protein in a cell or other biological system.

“Bruton's tyrosine kinase,” (BTK) has its plain and ordinary meaning when read in light of the specification, and may include but is not limited to, for example, an enzyme that in humans is encoded by the BTK gene. BTK is a kinase that plays a crucial role in B-cell development. For example, BTK plays a crucial role in B cell maturation as well as mast cell activation through the high-affinity IgE receptor. Mutations in the BTK gene are implicated in the primary immunodeficiency disease X-linked agammaglobulinemia (Bruton's agammaglobulinemia); sometimes abbreviated to XLA. Patients with XLA have normal pre-B cell populations in their bone marrow but these cells fail to mature and enter the circulation.

“X-linked agammaglobulinemia,” (XLA) as described herein is a genetic disorder that affects the body's ability to fight infection. As the form of agammaglobulinemia that is X-linked, it is much more common in males. In people with XLA, the white blood cell formation process does not generate mature B cells, which manifests as a complete or near-complete lack of proteins called gamma globulins, including antibodies, in their bloodstream. X-linked agammaglobulinemia (XLA) is characterized by recurrent bacterial infections in affected males in the first two years of life. Recurrent otitis is the most common infection prior to diagnosis. Conjunctivitis, sinopulmonary infections, diarrhea, and skin infections are also frequently seen. Approximately 60% of individuals with XLA are recognized as having immunodeficiency when they develop a severe, life-threatening infection such as pneumonia, empyema, meningitis, sepsis, cellulitis, or septic arthritis.

A “promoter” is a region of DNA that initiates transcription of a specific gene. The promoters can be located near the transcription start site of a gene, on the same strand and upstream on the DNA (the 5′region of the sense strand). The promoter can be a conditional, inducible or a constitutive promoter. The promoter can be specific for bacterial, mammalian or insect cell protein expression. In some alternatives, wherein a nucleic acid encoding a fusion protein is provided, the nucleic acid further comprises a promoter sequence. In some alternatives, the promoter is specific for mammalian protein expression. In some alternatives, the promoter is a conditional, inducible or a constitutive promoter.

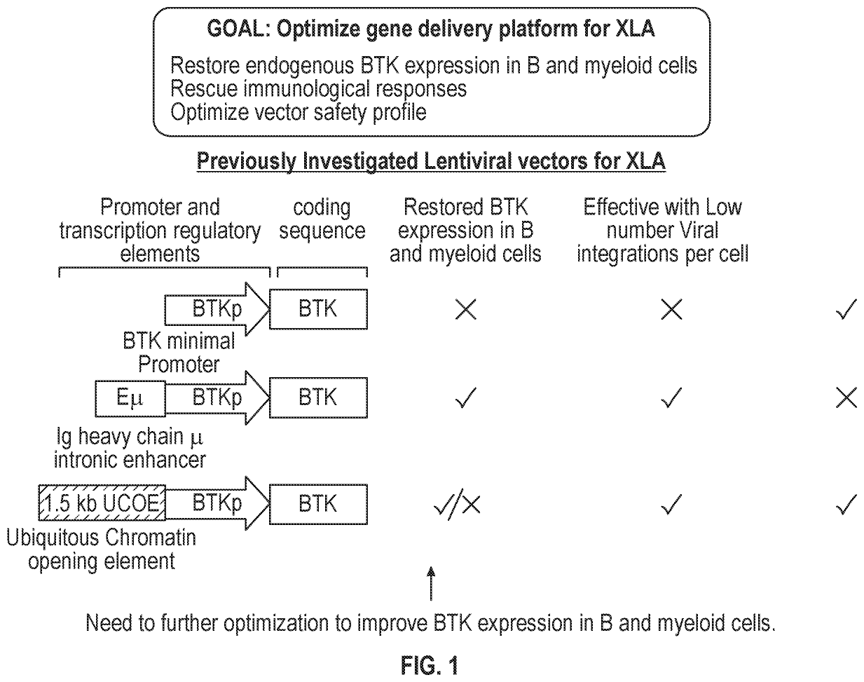

“Ubiquitous chromatin opening elements (UCOE)” as described herein are regulatory elements that are derived from promoter-containing CpG islands of ubiquitously expressed housekeeping genes. It was proposed that regulatory elements from such promoters possess a chromatin-remodeling function allowing the maintenance of chromatin in a permissive configuration resulting in high and consistent expression of genes in their proximity. Although originally relatively large (up to 16 kb), new, smaller, synthetic UCOEs can lead to high expression of the transgene. Ubiquitous chromatin elements and their functions is described in FIG. 1. In some alternatives, 0.7UCOE comprises a sequence set forth in SEQ ID NO: 2. In some alternatives, the enhancer comprises a sequence set forth in SEQ ID NO: 3 or 4.

“Codon optimization” has its plain and ordinary meaning when read in light of the specification, and may include but is not limited to, for example, the design process of altering codons to codons known to increase maximum protein expression efficiency. In some alternatives, codon optimization for expression in human is preferred, wherein codon optimization can be performed by using algorithms that are known to those skilled in the art so as to create synthetic genetic transcripts optimized for high mRNA and protein yield in humans. Programs containing algorithms for codon optimization in humans are readily available. Such programs can include, for example, OptimumGene™ or GeneGPS® algorithms. Additionally human codon optimized sequences can be obtained commercially, for example, from Integrated DNA Technologies.

Optimization can also be performed to reduce the occurrence of secondary structure in a polynucleotide. In some alternatives of the method, optimization of the sequences in the vector can also be performed to reduce the total GC/AT ratio. Strict codon optimization can lead to unwanted secondary structure or an undesirably high GC content that leads to secondary structure. As such, the secondary structures affect transcriptional efficiency. Programs such as GeneOptimizer can be used after codon usage optimization, for secondary structure avoidance and GC content optimization. These additional programs can be used for further optimization and troubleshooting after an initial codon optimization to limit secondary structures that can occur after the first round of optimization. Alternative programs for optimization are readily available. In some alternatives of the method, the vector comprises sequences that are optimized for secondary structure avoidance and/or the sequences are optimized to reduce the total GC/AT ratio and/or the sequences are optimized for expression in humans. In some alternatives herein, the gene encoding BTK is codon optimized. In some alternatives, the codon optimized BTK comprises a sequence set forth in SEQ ID NO: 6 or SEQ ID NO: 7.

“Enhancer elements,” as described herein, are short regions of DNA that can be bound by proteins (activators) to increase the likelihood that transcription of a particular gene will occur. The activators can also be referred to as transcription factors. Enhancers can be either cis-acting, or Trans-acting (acting away from the gene) and can be located up to 1 Mbp (1,000,000 bp) away from the gene and can be upstream or downstream from the start site, and either in the forward or backward direction. The size of an enhancer can be of a size 50, 100, 200, 300, 400, 500, 600, 700, 800, 900, 1000, 1100, 1200, 1300, 1400 or 1500 bp or any number of base pairs in between a range defined by any two aforementioned values.

“Dnase I hypersensitive site” as described herein, is a region of chromatin that is sensitive to cleavage by the DNase I enzyme. In these specific regions of the genome, chromatin has lost its condensed structure, exposing the DNA and making it accessible. This raises the availability of DNA to degradation by enzymes, such as DNase I. These accessible chromatin zones are functionally related to transcriptional activity, since this remodeled state is necessary for the binding of proteins such as transcription factors. As described in the alternatives herein is “Dnase I hypersensitive site 4” (DHS4). DHS4 is an enhancer element that is located at −18 kb from a E-globin promoter and can include binding sites for both erythroid specific and ubiquitous proteins and plays an important role as a regulatory element. In some alternatives herein, the vector for expression of BTK comprises at least one DNase Hypersensitive Site. In some alternatives, the DNase Hypersensitive Site is DNase Hypersensitive Site 1 (DHS1), DNase Hypersensitive Site 2, (DHS2) DNase Hypersensitive Site 3 (DHS3), DNase Hypersensitive Site 4 (DHS4) and/or DNase Hypersensitive Site 5 (DHS5).

“Intron” as described herein, is any nucleotide sequence within a gene that is removed by RNA splicing during maturation of the final RNA product. In some alternatives of the vector herein, the vector comprises at least one intronic region. In some alternatives, the at least one intronic region is from a human BTK locus that is in association with a human BTK proximal promoter.

“Vector,” “Expression vector” or “construct” is a nucleic acid used to introduce heterologous nucleic acids into a cell that has regulatory elements to provide expression of the heterologous nucleic acids in the cell. Vectors include but are not limited to plasmid, minicircles, yeast, and viral genomes. In some alternatives, the vector is a viral vector. In some alternatives, the viral vector is a lentiviral vector.

“B cells” as described herein are a type of white blood cell of the lymphocyte subtype. They are also known as B lymphocytes. B cells can function in the humoral immunity component of the adaptive immune system by secreting antibodies. Additionally, B cells present antigen (they are also classified as professional antigen-presenting cells (APCs) and secrete cytokines. In some alternatives of the cells provided herein, the cells are B cells.

“Myeloid cells” has its plain and ordinary meaning when read in light of the specification, and may include but is not limited to, for example, a granulocyte or monocyte precursor cell in bone marrow or spinal cord, or a resemblance to those found in the bone marrow or spinal cord. The myeloid cell lineage includes circulating monocytic cells in the peripheral blood and the cell populations that they become following maturation, differentiation, and/or activation. These populations include non-terminally differentiated myeloid cells, myeloid derived suppressor cells, and differentiated macrophages. Differentiated macrophages include non-polarized and polarized macrophages, resting and activated macrophages. Without being limiting, the myeloid lineage can also include granulocytic precursors, polymorphonuclear derived suppressor cells, differentiated polymorphonuclear white blood cells, neutrophils, granulocytes, basophils, eosinophils, monocytes, macrophages, microglia, myeloid derived suppressor cells, dendritic cells and erythrocytes. For example, microglia can differentiate from myeloid progenitor cells. In some alternatives of the cells provided herein, the cells are myeloid cells.

“Hematopoietic stem cells” or “HSC” as described herein, are precursor cells that can give rise to myeloid cells such as, for example, macrophages, monocytes, macrophages, neutrophils, basophils, eosinophils, erythrocytes, megakaryocytes/platelets, dendritic cells and lymphoid lineages (such as, for example, T-cells, B-cells, NK-cells). HSCs have a heterogeneous population in which three classes of stem cells exist, which are distinguished by their ratio of lymphoid to myeloid progeny in the blood (L/M). In some alternatives of the cells provided herein, the cells are hematopoietic stem cells. “Subject” or “patient,” have their plain and ordinary meaning when read in light of the specification, and may include but is not limited to, for example, any organism upon which the alternatives described herein may be used or administered, e.g., for experimental, diagnostic, prophylactic, and/or therapeutic purposes. Subjects or patients include, for example, animals. In some alternatives, the subject is mice, rats, rabbits, non-human primates, and humans. In some alternatives, the subject is a cow, sheep, pig, horse, dog, cat, primate or a human. In some alternatives, the subject is a human male.

“Adoptive cellular therapy” or “adoptive cell transfer,” as described herein refers to the transfer of cells, most commonly immune-derived cells, back into the same patient or into a new recipient host with the goal of transferring the immunologic functionality and characteristics into the new host. In some alternatives, adoptive cellular therapy or adoptive cell transfer comprises administering cells for expression of BTK to a subject in need.

### DETAILED DESCRIPTION

BTK is expressed in both B cells and myeloid cells where it also contributes to normal functional responses in both lineages. Failure to express BTK leads to XLA. Conversely, overexpression of activated or wild type BTK can lead to cell transformation and/or developmental blockade (“Early arrest in B cell development in transgenic mice that express the E41K Bruton's tyrosine kinase mutant under the control of the CD19 promoter region” J Immunol. 1999 Jun. 1; 162(11):6526-33; “Correction of B-cell development in Btk-deficient mice using lentiviral vectors with codon-optimized human BTK.” Leukemia. 2010 September; 24(9):1617-30; incorporated by reference in their entireties herein) and dysregulated expression of wild-type BTK can promote autoantibody production and increase the risk for autoimmunity (“Enhanced Expression of Bruton's Tyrosine Kinase in B Cells Drives Systemic Autoimmunity by Disrupting T Cell Homeostasis.” J Immunol. 2016 Jul. 1; 197(1):58-67; incorporated by reference in its entirety herein). Thus, safe and successful clinical gene therapy in XLA requires restoration of BTK expression in each cell lineage that normally expresses the protein as well as tightly regulated expression that does not lead to overexpression in developmental subsets that do not normally express the protein- and where expression might promote altered cell function. To address this challenge, candidate viral vectors in murine XLA animal models and novel human cell models using HSC derived from human subjects with XLA were comprehensively assessed, as described in the alternatives herein. Based upon iterative design and testing of candidate promoter, insulator, and enhancer elements and human codon-optimized BTK cDNA constructs, a novel lentiviral-based (LV) vector construct that mediates sustained BTK expression in B and myeloid cells derived from (murine or human) hematopoietic stem cells following ex vivo transduction and transplantation into BTK deficient hosts has been identified and is shown in the alternatives herein. The unique constructs of the alternatives herein, utilize a truncated UCOE element, a conserved enhancer element derived from intronic regions within the human BTK locus in association with the human BTK proximal promoter to drive expression of a human codon-optimized BTK cDNA. The optimal LV vectors of the one of the exemplary alternatives, is referred to as 0.7 UCOE.DHS4.BTKpro.coBTK. As part of the research described in the alternatives, a series of surprising and unpredicted results leading to this construct choice were identified, as shown: 1) the LV containing the BTK minimal promoter alone were not sufficient to restore B cell development or function as shown in several alternatives; 2) the LV utilizing the Eμ enhancer element (with either a B lineage restricted promoter or the BTK minimal promoter) lead to development of high titer autoantibodies including pathogenic class-switched IgG isotypes indicating that this enhancer created significant safety concerns as shown in several alternatives; 3) silencing of LV vector expression for multiple candidate LV platforms in secondary recipient animals was observed which led to utilization of a UCOE element to resist such silencing; 4) low titers with use of a large UCOE element was observed and therefore led to designing and testing of a novel truncated 0.7 kb element that would lead to improved titer and function and retained resistance to silencing; 5) bioinformatic tools were utilized to identify and test multiple potential candidate enhancer elements derived from the BTK locus and after significant analysis this was used to identify an optimal element, DHS4, that lead to increased BTK expression in vivo without reducing viral titer; 6) alternative human codon-optimized BTK cDNAs were tested and led to an identified construct that best restores BTK expression; 7) HSC was collected from multiple adult subjects with XLA and it was shown that when transplanted into immune deficient NSG mice these stem cells recapitulate the B cell developmental defects seen in XLA subjects and 8) in several alternatives herein, it was shown that XLA HSC can be efficiently transduced using the alternative optimized LV constructs described herein.

X-linked agammaglobulinemia (XLA) is a hereditary X-linked immunodeficiency disorder caused by a mutation in the BTK gene (Bruton's Tyrosine Kinase). This disease affects approximately 1 in 100,000 males. The clinical manifestations can include: lack mature B cells and serum immunoglobulins, susceptibility to lung, sinus, and skin infections with encapsulated bacteria, risks for sudden death due to bacteria sepsis, chronic and systemic infections with enteroviruses, chronic inflammatory bowel disease and increased risk for malignancy such as colon and other types of cancer.

The current treatment options for XLA can include lifelong pooled human immunoglobulin (IVIg or SCIg) every 3-4 weeks. However these treatments are expensive and the current treatments can also lead to risk for infection and sudden death.

The candidates for gene therapy can include those with single gene hematopoietic disorder.

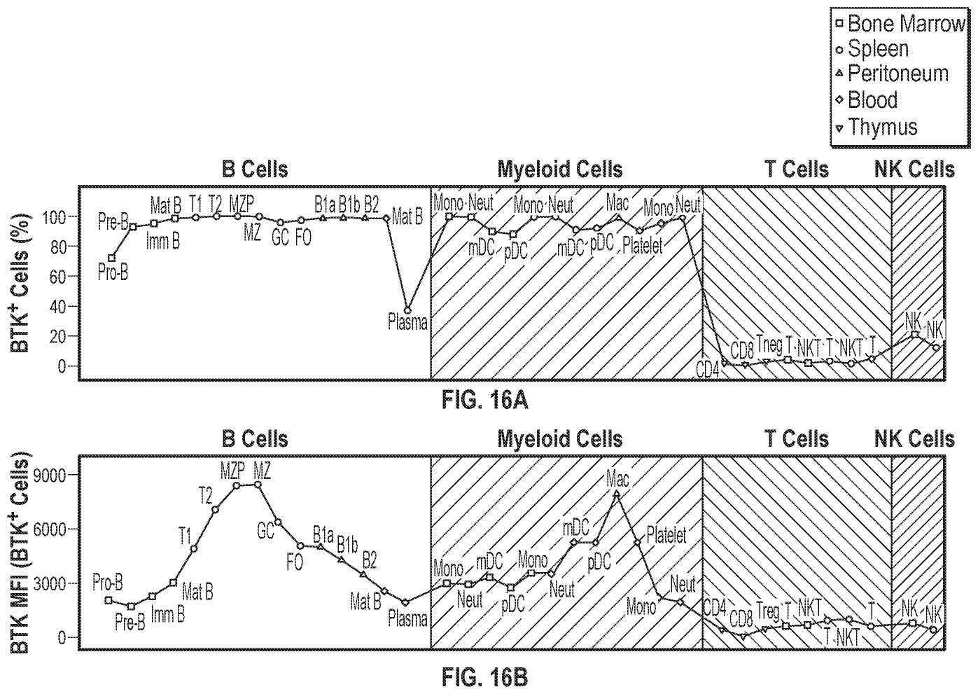

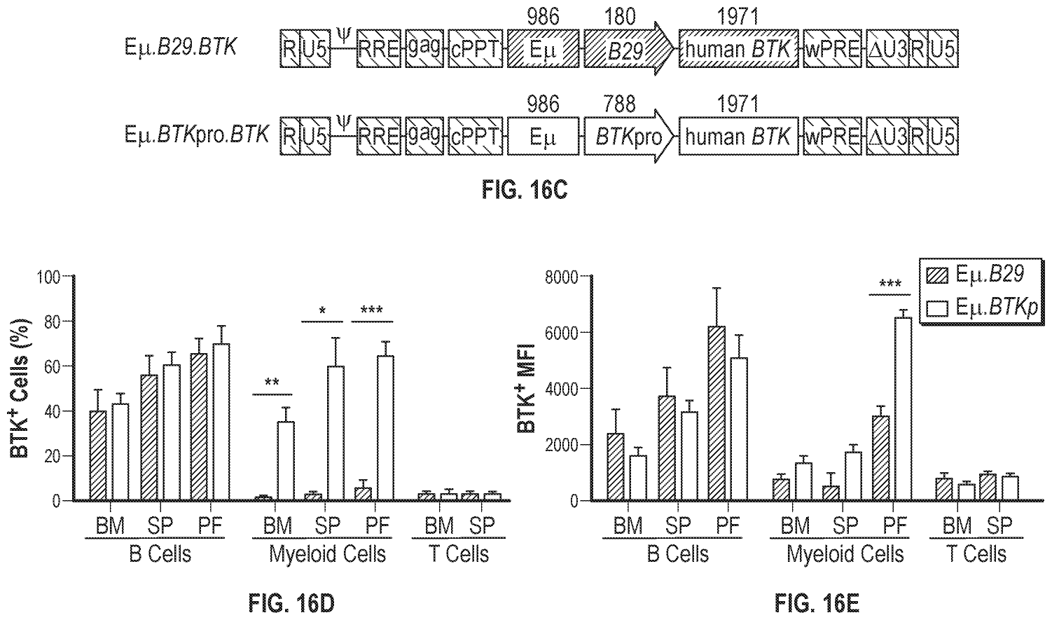

Bruton's Tyrosine Kinase (BTK) is a cytoplasmic non-receptor protein tyrosine kinase whose main role is in B cell receptor signaling pathway. BTK promotes B cell survival, proliferation and differentiation. BTK can maintain sustained calcium signal following BCR (B cell receptor) engagement and promotes NFKB activation. Furthermore, BTK also plays role in cytokine, growth factor, and TLR signaling pathways. A role for BTK is shown in FIG. 21, where BTK leads to the development of the B cells. As shown, XLA leads to a block at the Pre−B cell stage. BTK is expressed in B and myeloid lineages, and not expressed in T cells. The expression profile of endogenous BTK is shown in FIG. 16.

No other investigators have described use of the BTK promoter or any of the additional modifications of the alternatives herein, in association with the BTK in LV vectors. While the BTK promoter and the BTK first intron have been partially evaluated in previous studies of promoter function, no group has described or tested candidate enhancer elements derived from the human BTK locus including the DHS4 element or others (“Analysis of the Bruton's tyrosine kinase gene promoter reveals critical PU.1 and SP1 sites.” Blood. 1996 Feb. 1; 87(3):1036-44; “Cell specific expression of human Bruton's agammaglobulinemia tyrosine kinase gene (Btk) is regulated by Sp1- and Spi-1/PU.1-family members.” Oncogene. 1996 Nov. 7; 13(9):1955-64; “Large-scale comparative sequence analysis of the human and murine Bruton's tyrosine kinase loci reveals conserved regulatory domains.” Genome Res. 1997 April; 7(4):315-29.; “Synergistic activation of the human Btk promoter by transcription factors Sp1/3 and PU.1.” Biochem Biophys Res Commun. 1999 Jun. 7; 259(2):364-9; “Btk expression is controlled by Oct and BOB.1/OBF.1.” Nucleic Acids Res. 2006 Mar. 31; 34(6):1807-15; “Proteasome-dependent autoregulation of Bruton tyrosine kinase (Btk) promoter via NF-kappaB.” Blood. 2008 May 1; 111(9):4617-26.; all references hereby expressly incorporated by reference in their entireties).

An independently derived codon-optimized BTK cDNA has been identified (“Correction of B-cell development in Btk-deficient mice using lentiviral vectors with codon-optimized human BTK.” Leukemia. 2010 September; 24(9):1617-30′ incorporated by reference in its entirety). Other investigators have studied the use of UCOE element alone or in association with a lineage specific or ubiquitous promoter in LV vectors but none have applied this technology to BTK (“Lentiviral vectors containing an enhancer-less ubiquitously acting chromatin opening element (UCOE) provide highly reproducible and stable transgene expression in hematopoietic cells.” Blood. 2007 Sep. 1; 110(5):1448-57. “A ubiquitous chromatin opening element (UCOE) confers resistance to DNA methylation-mediated silencing of lentiviral vectors.” Mol Ther. 2010 September; 18(9):1640-9; “Physiological regulation of transgene expression by a lentiviral vector containing the A2UCOE linked to a myeloid promoter.” Gene Ther. 2012 October; 19(10):1018-29; “Correction of murine Rag2 severe combined immunodeficiency by lentiviral gene therapy using a codon-optimized RAG2 therapeutic transgene.” Mol Ther. 2012 October; 20(10):1968-80; “Promoter and lineage independent anti-silencing activity of the A2 ubiquitous chromatin opening element for optimized human pluripotent stem cell-based gene therapy.” Biomaterials. 2014 February; 35(5):1531-42.; “A ubiquitous chromatin opening element prevents transgene silencing in pluripotent stem cells and their differentiated progeny.” Stem Cells. 2013 March; 31(3):488-99; “Lentiviral MGMT(P140K)-mediated in vivo selection employing a ubiquitous chromatin opening element (A2UCOE) linked to a cellular promoter.” Biomaterials. 2014 August; 35(25): 7204-13; “Detailed comparison of retroviral vectors and promoter configurations for stable and high transgene expression in human induced pluripotent stem cells.” Gene Ther. 2017 Mar. 27; all references hereby expressly incorporated by reference in their entireties herein).

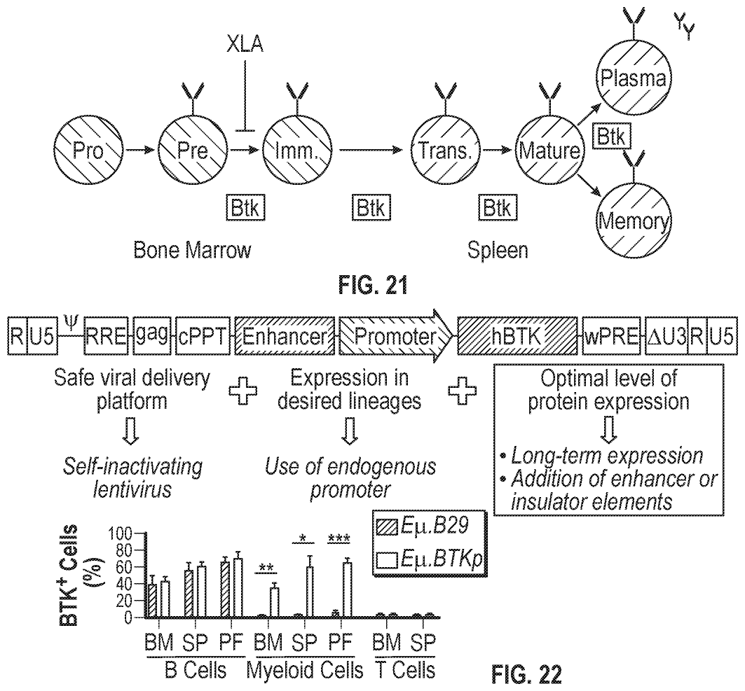

The optimized lentiviral vectors, as described in the alternatives herein, will be used in gene therapy for subjects with X-linked agammaglobulinemia (XLA) designed to lead to long-term curative therapy for this disease. Shown in FIG. 22 is an exemplary alternative in the development of the optimal BTK lentiviral gene therapy vector. This alternative would lead to a safe viral delivery platform and the expression of desired lineages with optimal levels of protein expression.

In some alternatives, the optimized BTK lentiviral vector comprises a ubiquitous chromatin opening element (UCOE). The UCOE can provide stable expression of transgenes regardless of integration site and can also confer resistance to silencing in an adjacent promoter (see FIG. 1).

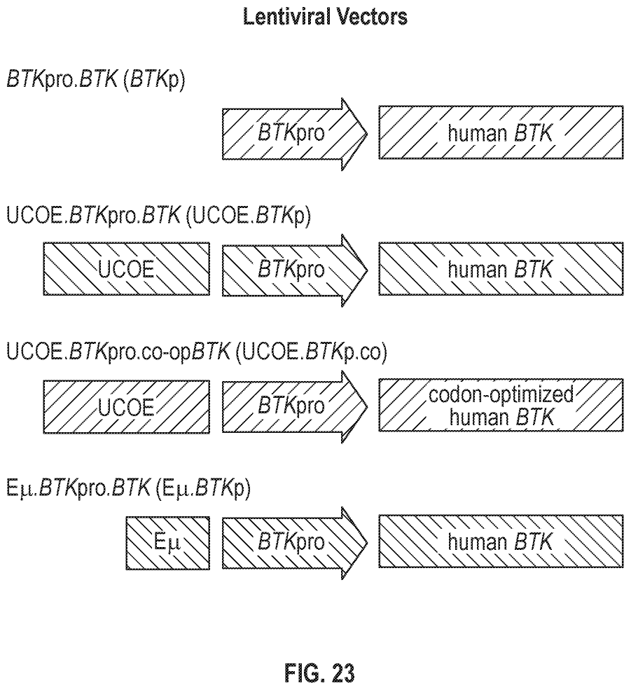

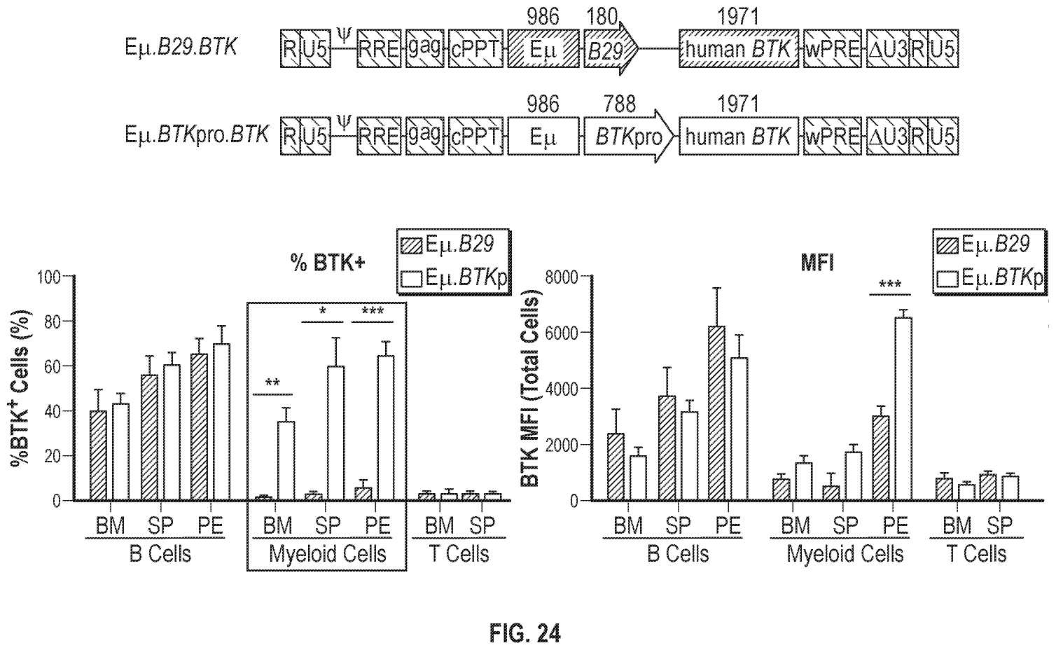

The optimized LV vectors for BTK expression were also shown to increase BTK expression in cells. A few exemplary vectors that were used in several alternatives are shown in FIG. 23. As shown in FIG. 24, the lentiviral vectors comprising EμBTKp promoters also had an increase of BTK in myeloid cells.

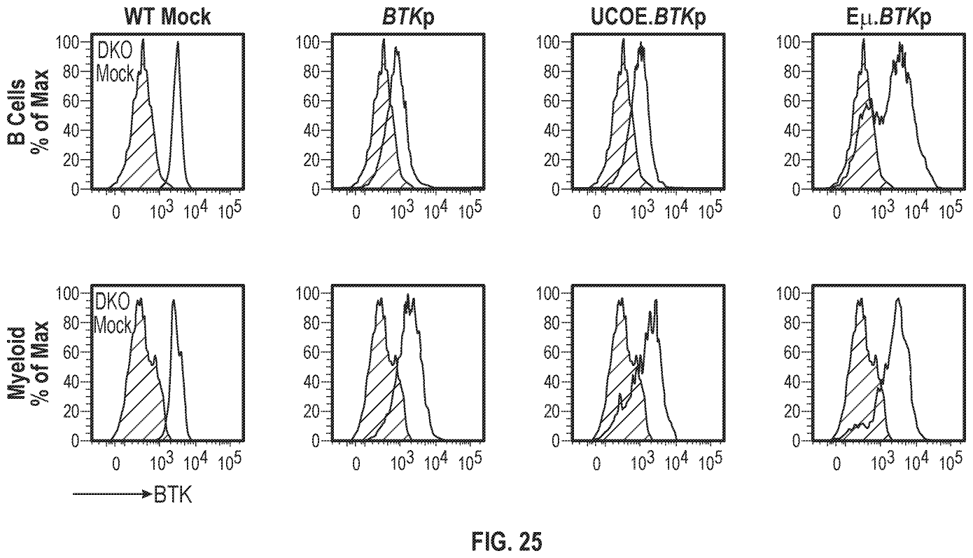

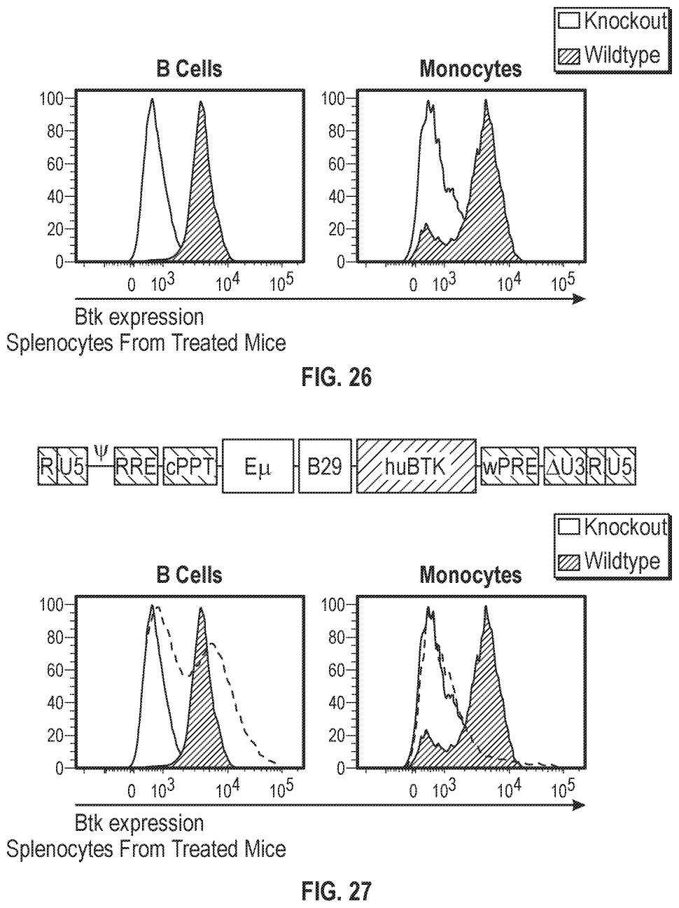

Cells transduced with DKO mock, BTKp, UCOE.BTKp and Eμ.BTKp were tested for BTK expression. As shown in the FACS assay, incorporation of Eμ into the LV vector for BTK expression was also shown to boost BTK expression the most in both B cells and myeloid cells as compared to the BTKp promoter alone and with the UCOE elements. (FIG. 25). The results of the BTK expression in the transduced cells are comparable to the wild type cells that express the BTK naturally as shown in FIG. 26, which shows the comparison between a knock out cell and wild type cell in both B cells and monocytes.

It is contemplated that this LV vector will lead to curative therapy, in particular XLA gene therapy.

In the alternatives described herein, the gene delivery platform was specialized for XLA therapy. This would allow restoration of endogenous BTK expression in B cells and myeloid cells, and rescue of immunological responses in those suffering from XLA. The vector safety profile was further evaluated. Previously investigated lentiviral vectors for XLA included those with promoter and transcription elements such as the BTK minimal promoter, the Ig heavy chain μ. intronic enhancer, as well as the 1.5 kb ubiquitous chromatin opening element. However, as shown in the alternatives herein, there were further modifications needed in order to improve the BTK expression in B and myeloid cells.

### Optimization of the Gene Delivery Platform for XLA

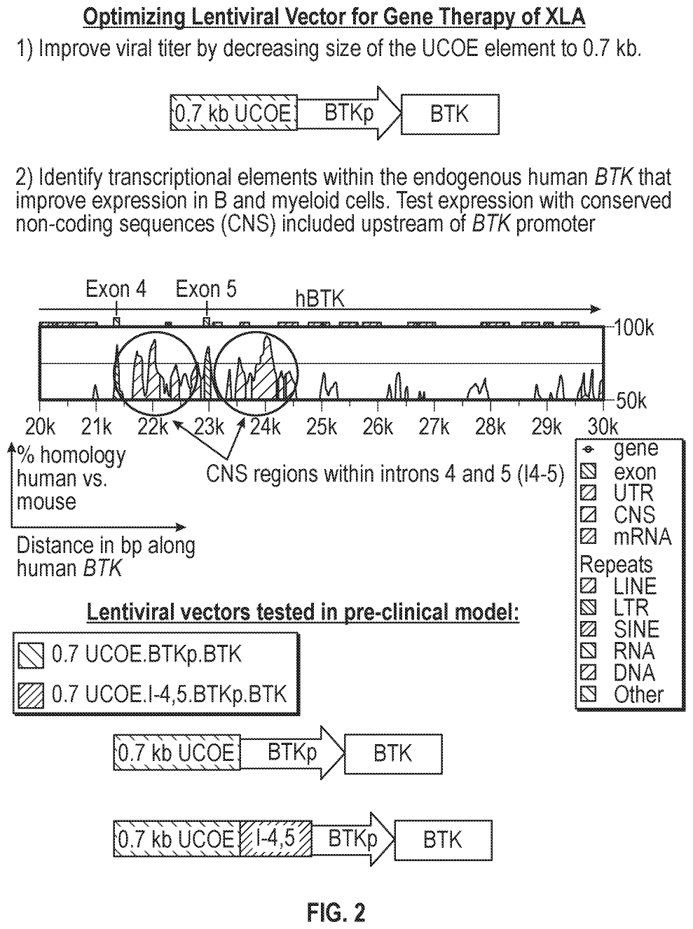

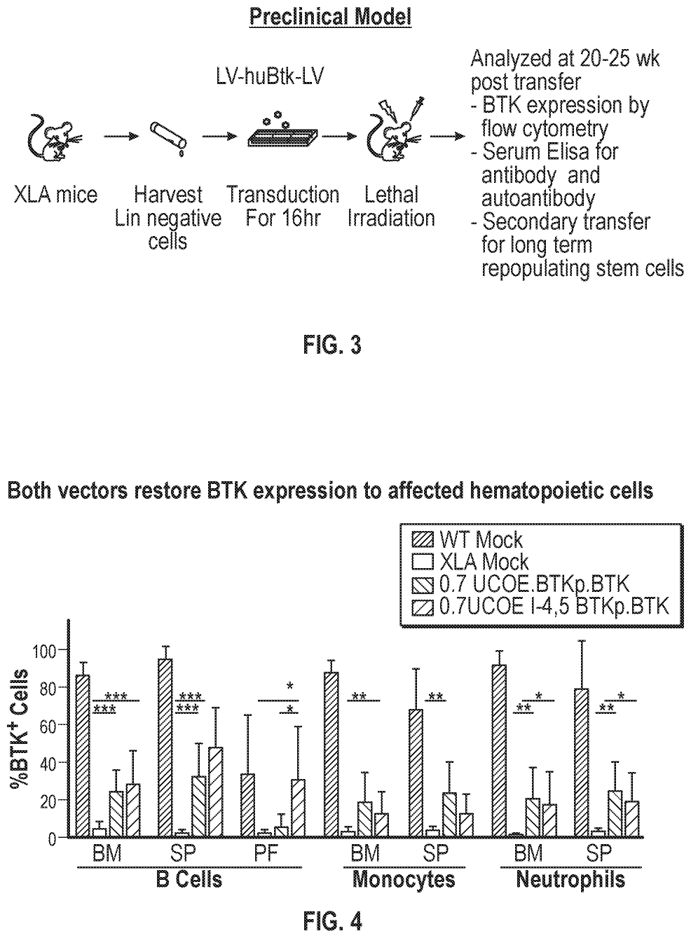

In order to improve the lentiviral vector for gene therapy, several steps were taken, as shown in the alternatives herein: 1) improvement of viral titer by decreasing the size of the UCOE element to 0.7 kb; 2) identifying transcriptional elements within the endogenous human BTK that would improve expression in B and myeloid cells and test the expression with conserved non-coding sequences (CNS) that are included upstream from the BTK promoter (FIG. 1). The lentiviral vectors tested in the pre-clinical models in the alternatives described herein include 0.7 UCOE.BTKp.BTK and 0.7 UCOE.I-4,5.BTKp.BTK. (see FIG. 2). As shown in FIG. 3, blood was taken from XLA mice, in which Lin negative cells were harvested. The cells were then transduced with LC-huBTK-LV. The cells were then administered to the XLA mice, which were analyzed at 20-25 weeks post cell transfer. The BTK expression was analyzed by flow cytometry. A secondary cellular transfer was also performed for long term repopulation of the stem cells (See FIG. 3). A schematic showing the preclinical murine model for XLA gene therapy is shown in FIG. 3.

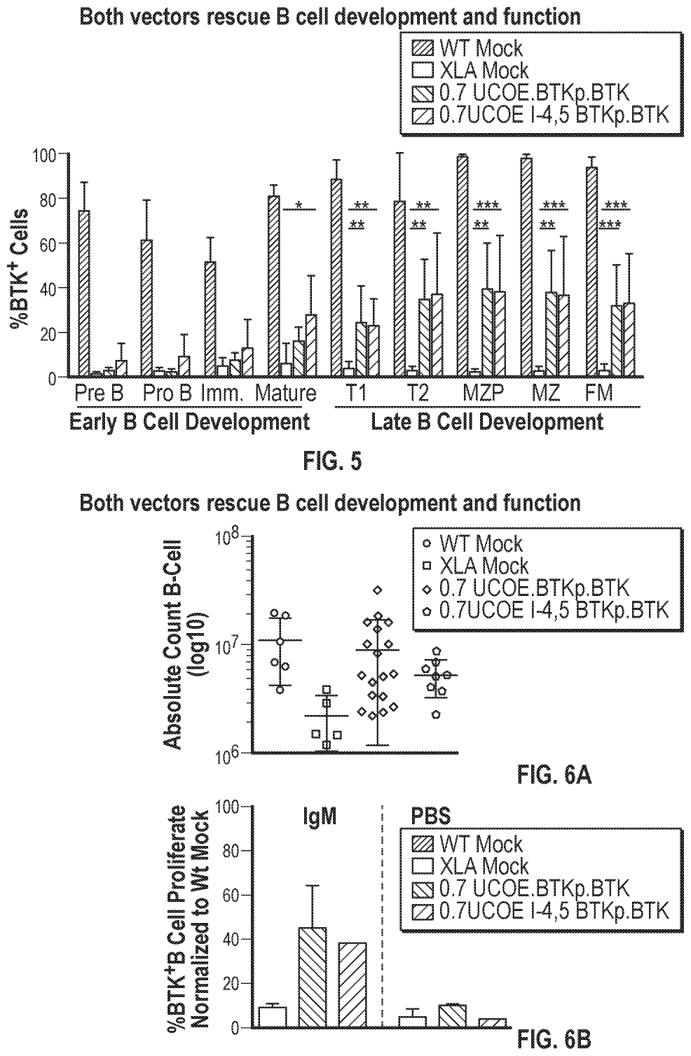

Both vectors (0.7UCOE and 0.7UCOE-I4,5) expressing human BTK, restored BTK expression to affected hematopoietic cells, rescued B cell development and function and restored immune responses (FIGS. 4-7). However it was shown that 0.7UCOE.I-45.BTIpBTK expressed BTK as effectively as 0.7UCOE.BTKp.BTK, but with fewer viral integrations (FIG. 8). Thus several conclusions were drawn.

The LV vector that included conserved BTK regulatory elements (derived from BTK introns 4 and 5) in association with the endogenous BTK improves BTK expression per viral integration. Also 0.7UCOE.I4,5.BTKpBTK LV comprises an efficient candidate for XLA gene therapy.

For expanded pre-clinical studies the following are performed: a) murine modeling to fully evaluate toxicity, safety and efficacy (including use of secondary transplantation and integration site analysis); b) in-vitro immortalization and transactivation assays; c) refine genetic elements within introns 4 and 5 for improving BTK expression in B and myeloid cells; and d) evaluation of 0.7UCOE.I-4,5.BTKp.BTK in healthy control and XLA subject CD34+ stem cells in vitro and in NSG recipient mice.

### Improved Safety and Efficacy of Lentiviral Gene Therapy in a Murine XLA Model Using a UCOE-Insulated BTK Promoter

Experiments were performed in order to optimize the safety and efficacy of a lentiviral gene therapy in mice using a UCOE insulated BTK promoter. For the experiments several vector constructs were used: WT Mock, KO Mock, BTKp, 1.5.UCOE.BTKp, and Eμ.BTKp.

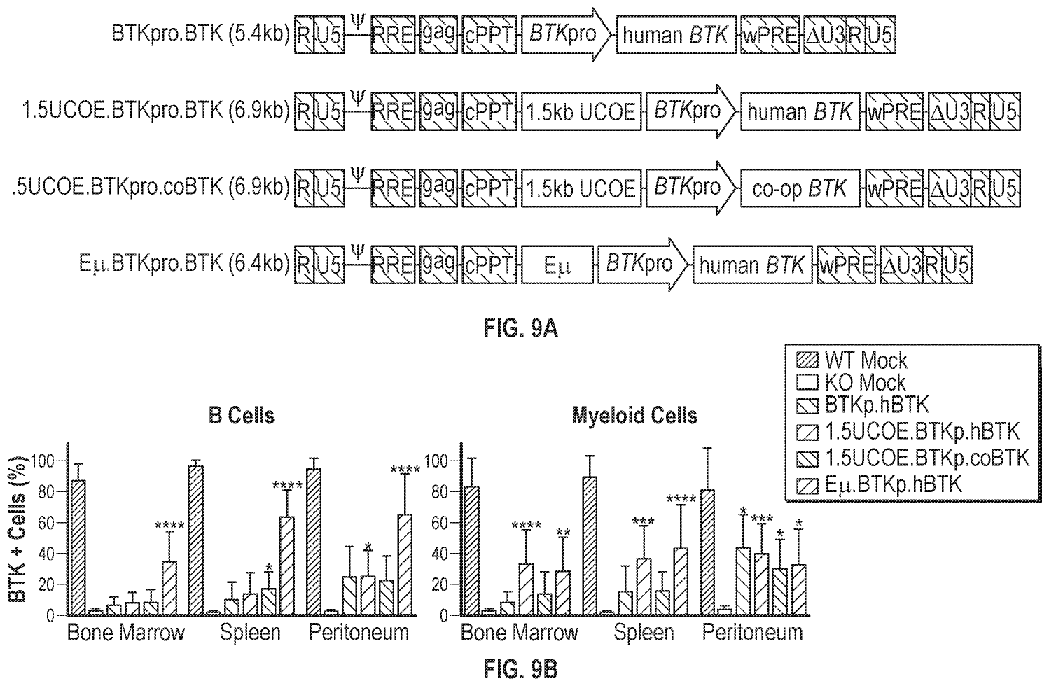

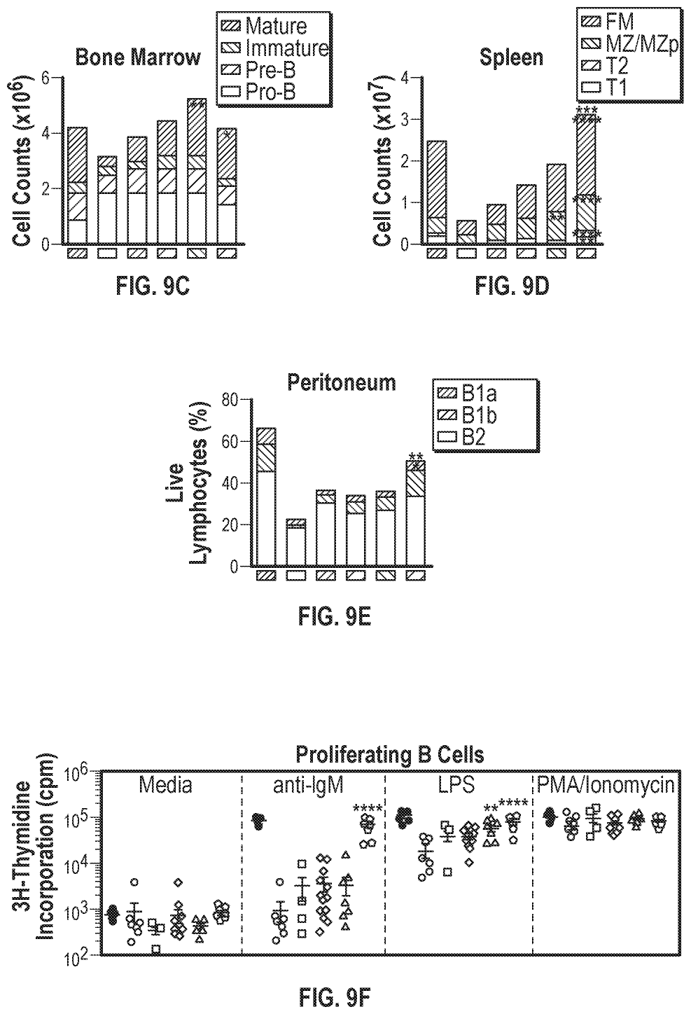

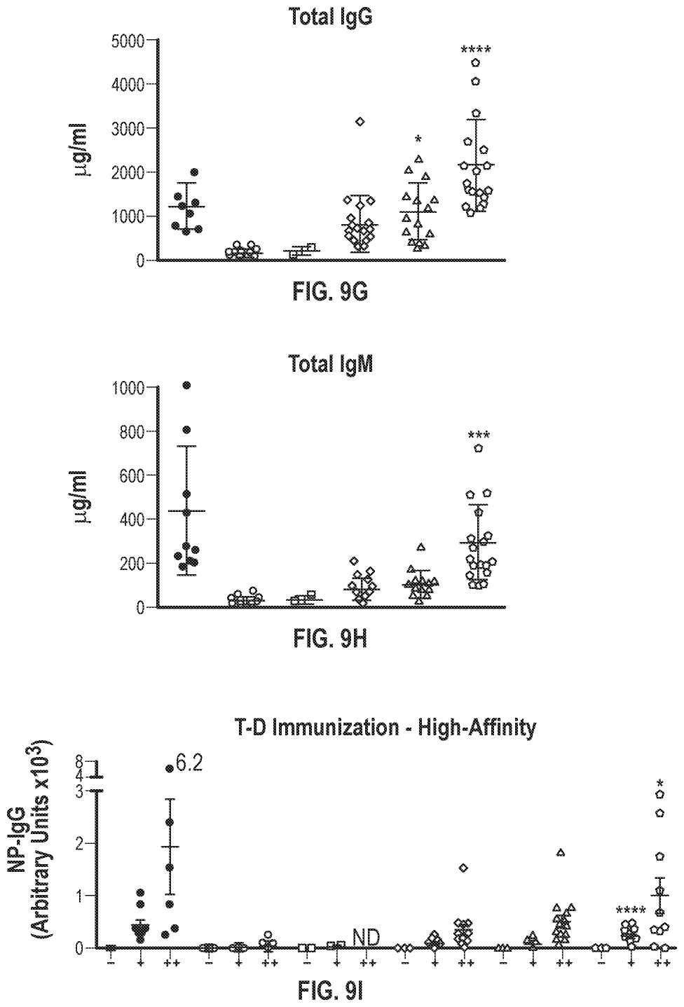

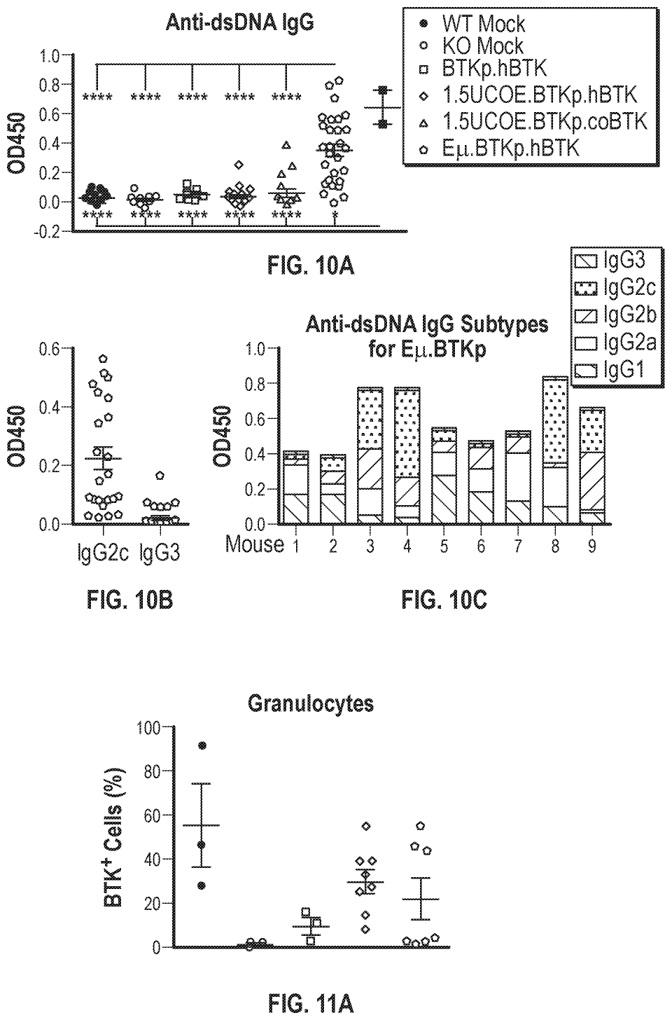

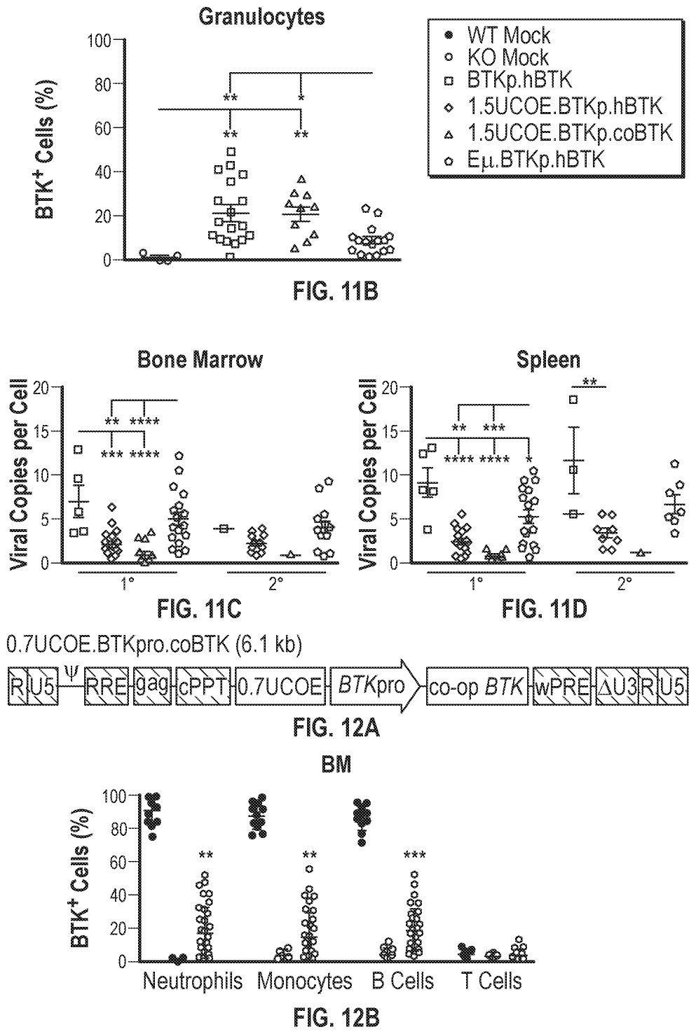

Shown in FIG. 9 are the expression profiles of four LV constructs and rescue of B cell development and function in primary recipient mice. The vectors used were BTKpro.BTK, 1.5UCOE.BTKpro.BTK, 1.5UCOE.BTKpro.coBTK (human codon optimized BTK) and Eμ.BTKpro.BTK. The vector, the Eμ.BTKp.hBTK was shown to increase BTK expression in all cells as shown in FIG. 9. The vector, the Eμ.BTKp.hBTK was also shown to increase IgG expression in all cells as shown in FIG. 10. Increased BTK expression was also seen for the vector Eμ.BTKp.hBTK in granulocytes, bone marrow and spleen B cells as seen in FIG. 11. The 1.5 kbUCOE.BTKp and 1.5 kbUCOE.BTKp.co vectors were shown to lead to sustained BTK expression with lower copy numbers in primary and secondary recipients (FIG. 11).

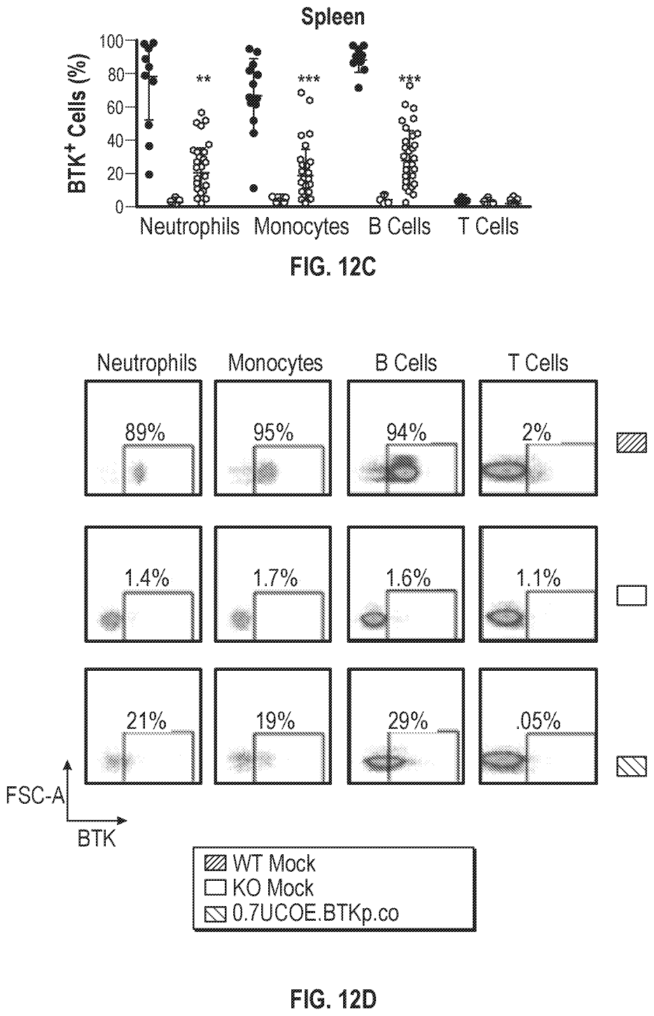

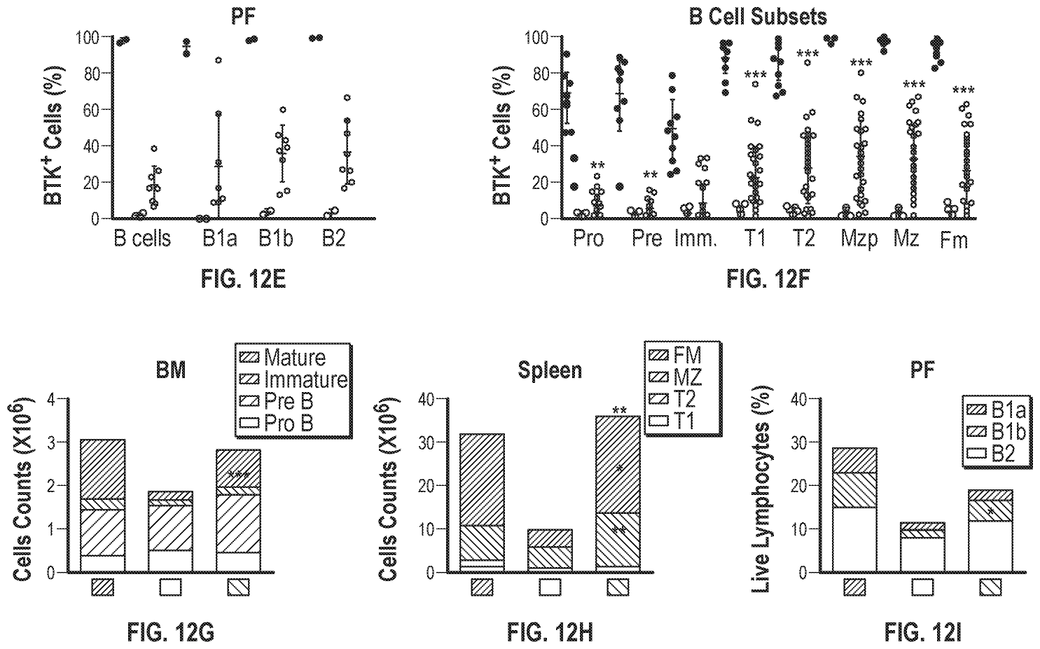

Restoration of BTK expression was shown in affected hematopoietic lineages and B cell development in gene therapy mice treated with 0.7 kb UCOE.BTKpro.coBTK LV (FIG. 12).

Proliferation of B cells, increase of IgM and IgG secretion were also seen in cells following 0.7.UCOE.BTK.co LV gene therapy in primary recipients (FIG. 12).

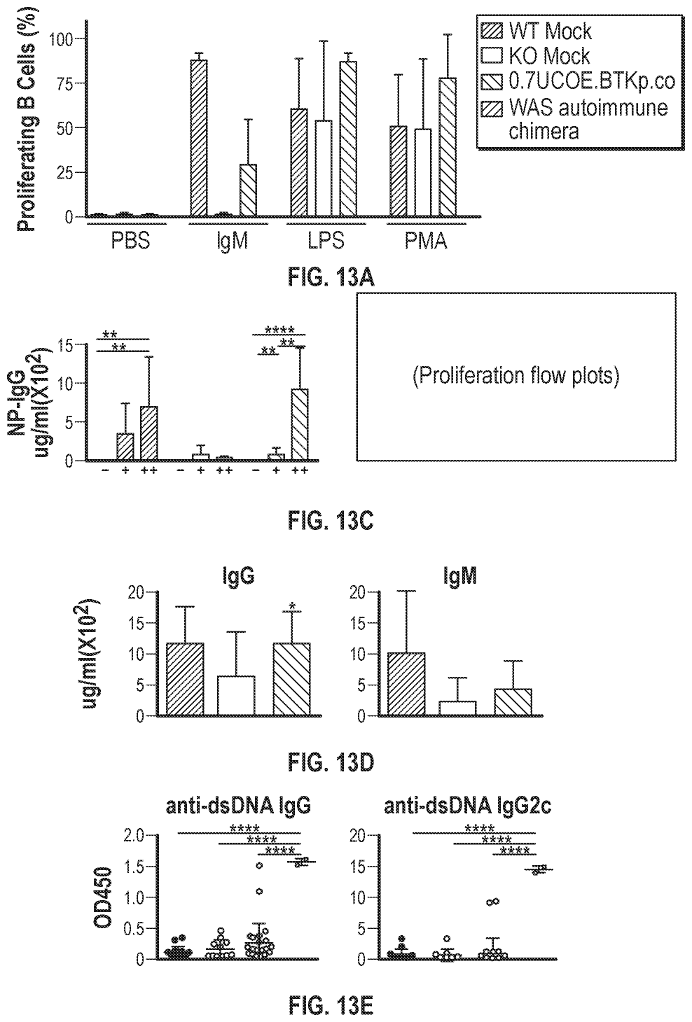

Reconstitution of B cell function was also seen following 0.7.UCOE.Bkp.co LV gene therapy in primary recipients (FIG. 13).

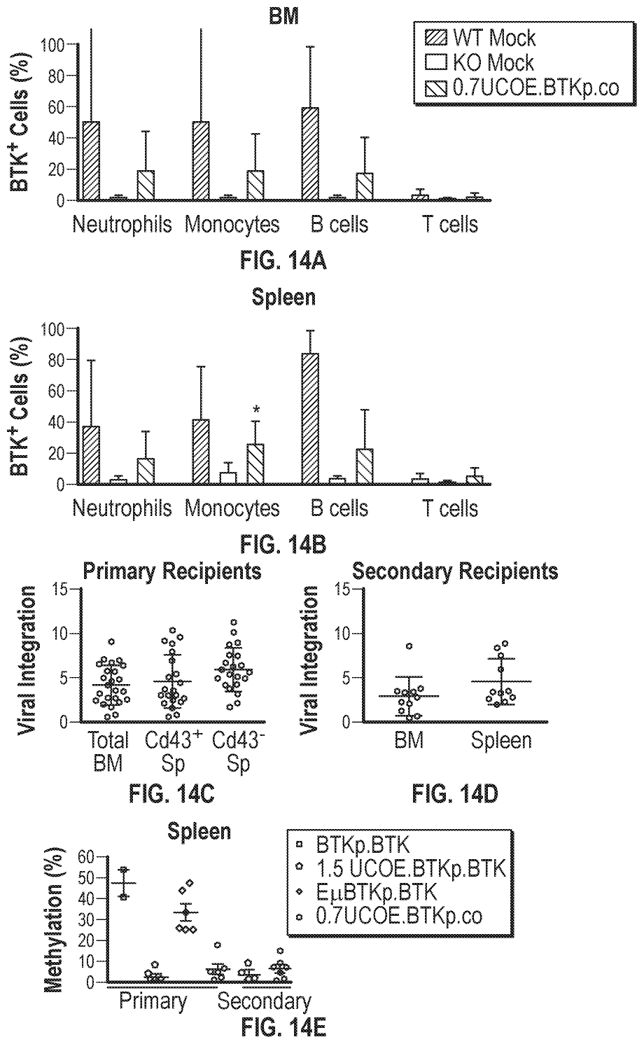

VCN and BTK expression are maintained after serial passage of gene therapy-treated bone marrow cells into secondary TBK^{-/-} recipients (FIG. 14). As shown, cells transduced with the 0.7UCOE.BTKp.co led to expression of BTK in the neutrophils, monocytes, B cells of the bone marrow and the spleen. Methylation of DNA was also measured, which is a modification for suppressing gene transcription.

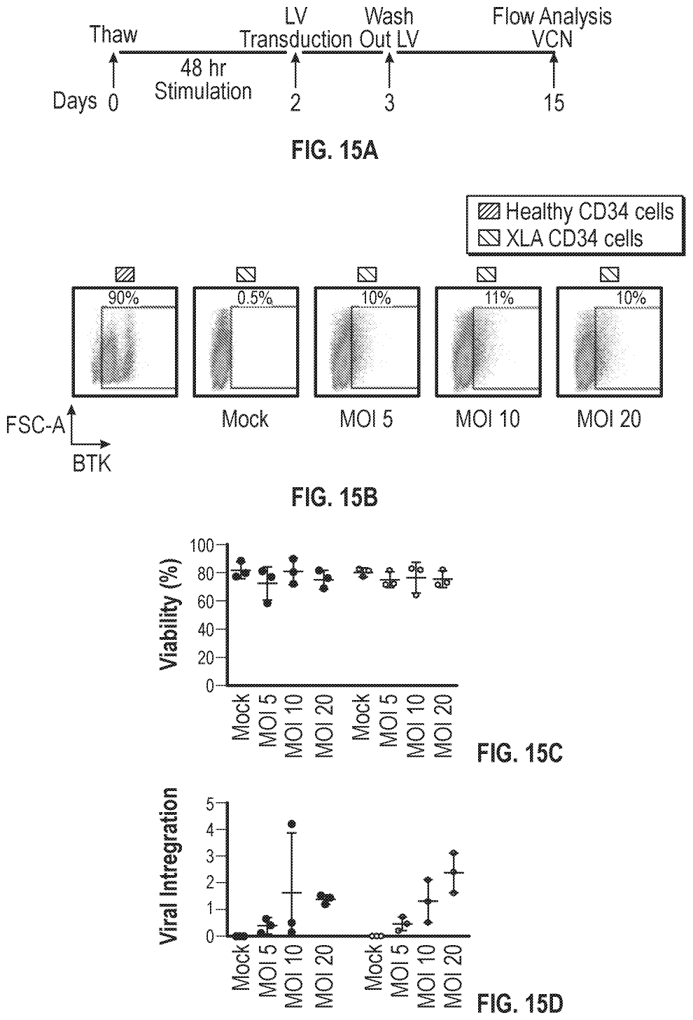

The vector 0.7UCOE.BTKp.co was shown to lead to sustained BTK expression and lower copy numbers in XLA CD34 cells (FIG. 15). XLA and control CD34 cells were transduced with 0.7UCOE.BTKp.co at various multiplicities of infection (MOI) and cultured in vitro for 15 days. As shown, the XLA transduced cells had similar viability as the healthy control cells.

The BTK promoter in the lentiviral vectors were also used to evaluate BTK expression in wild type mice. As shown, the BTK promoter mimics BTK's endogenous expression pattern in mice (FIG. 16).

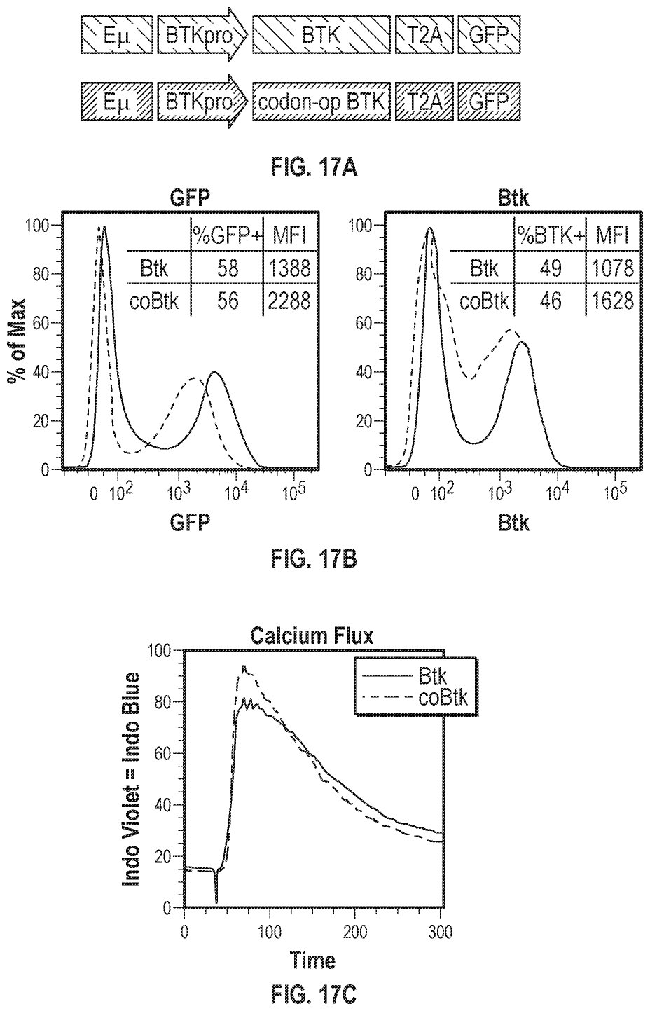

The Eμ promoter was then tested for expression enhancement in the lentiviral vectors. Two vectors were tested, which had the Eμ promoter as shown in FIG. 17 panel a (Eμ enhancer and human BTK cDNA, either wild type or human codon-optimized, with a T2A-linked GFP). Chicken BTK−/− DT40 cells were transduced with BTK-GFP or coBTK-GFP constructs; histograms show GFP and BTK expression (FIG. 17 panel b). BTK-GFP and coBTK-GFP transduced cells were stained with an Indo-1 Ester AM fluorescent dye and stimulated with anti-IgM; calcium mobilization was monitored via flow cytometry. As shown, lentiviral constructs under control of the BTK promoter with enhancer and human BTK cDNA led to controlled expression of the BTK-GFP in the chicken cells.

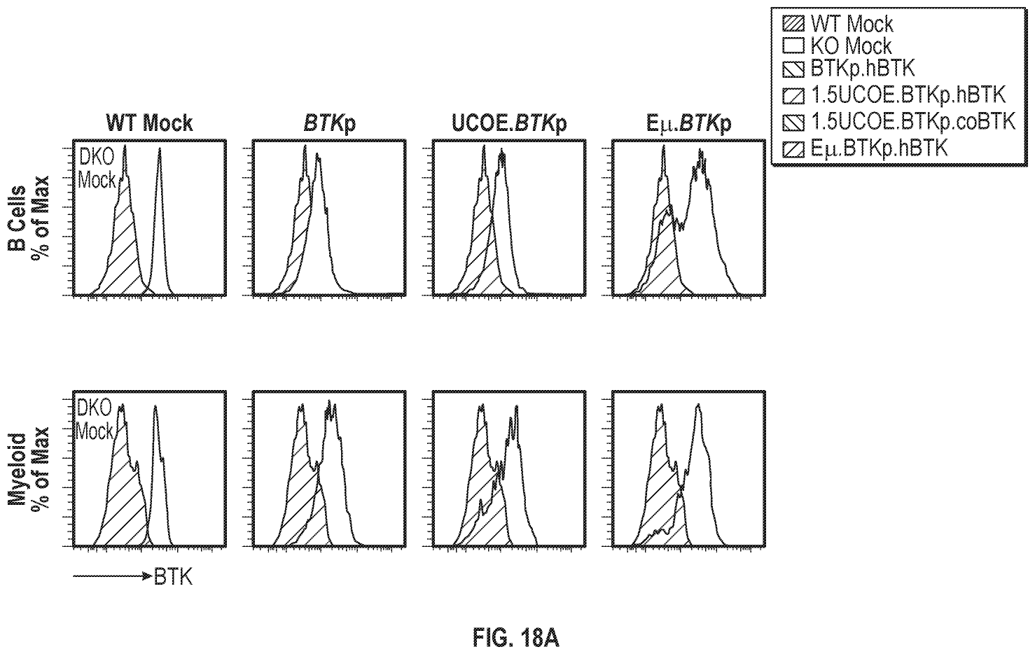

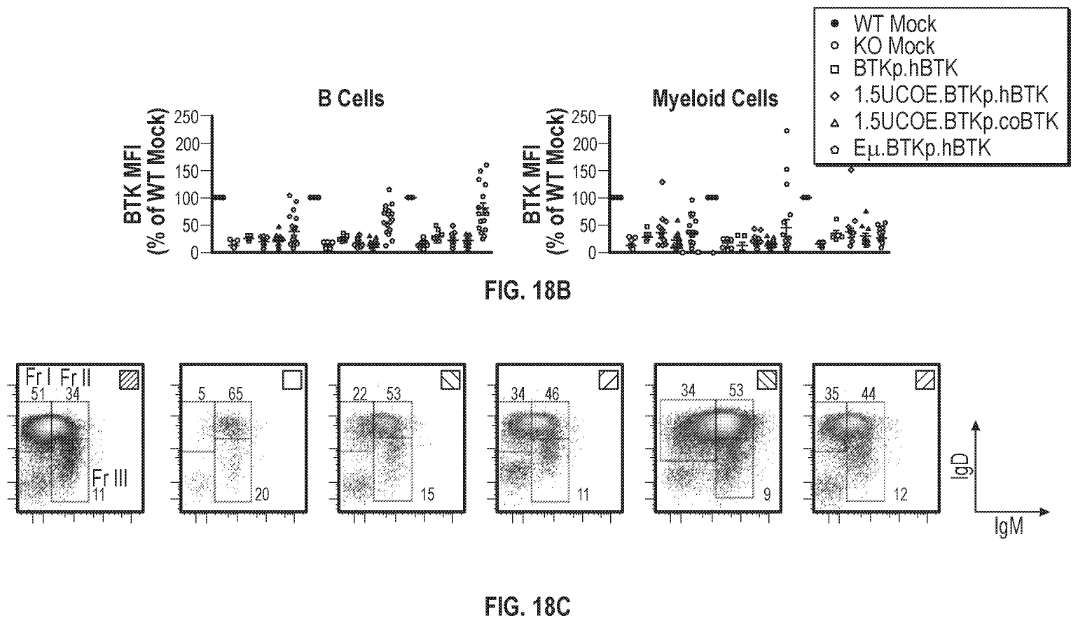

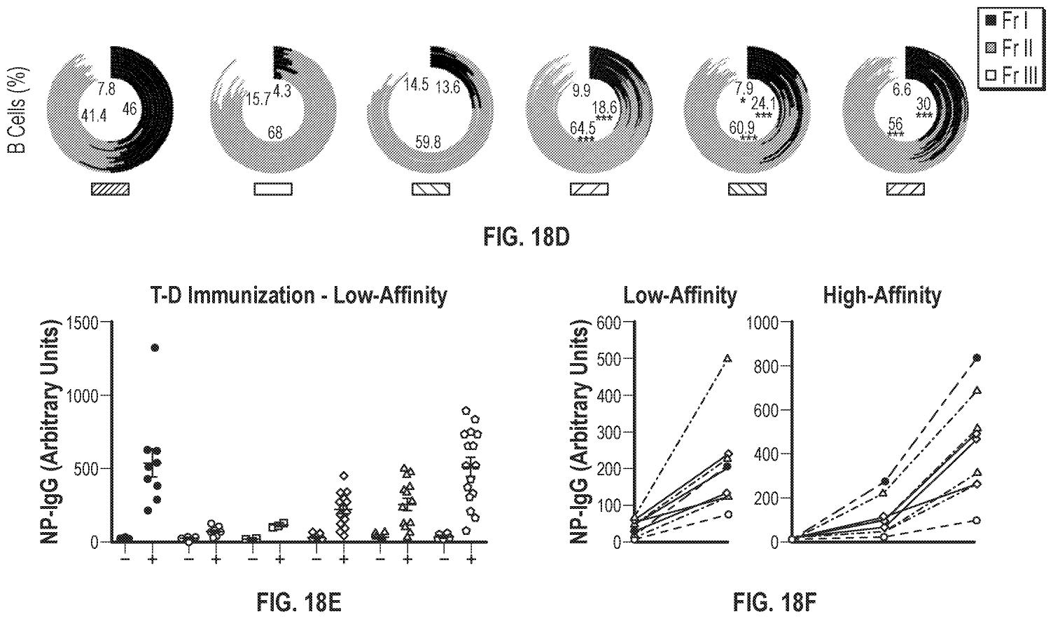

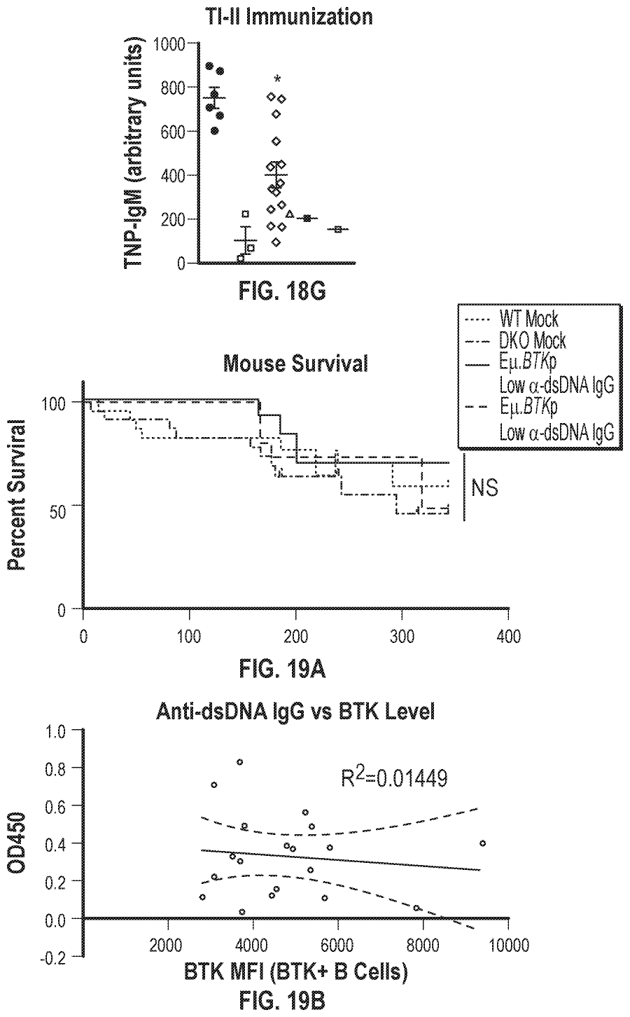

Representative plots of BTK expression after flow cytometry of peripheral blood B cells and myeloid cells from gene therapy-treated KO mice was also performed (FIG. 18). The vectors tested were: WT Mock, KO Mock, BTKp, 1.5 kb.UCOE.BTKp, 1.5 kb.UCOE.BTKp.co, and Eμ.BTKp. Shown in FIG. 18 panel c are the representative flow cytometry plots from bone marrow of gene therapy treated mice, stained with markers for early B cell development. FIG. 18 panel d shows graphs that depict the percentage of B cells following Hardy fractions of decreasing maturity: Fr I (IgMlo, IgDhi), Fr II (IgMhi, IgDhi), FrIII (IgMhi, IgDlo), each ring represents one mouse and the mean percent of B cells in each fraction is shown inside the graphic. Data represent mean±SEM from 11 independent experiments, n=18 (WT Mock), 14 (KO Mock), 7 (BTKp), 43 (1.5 kb.UCOE.BTKp), 18 (1.5 kb.UCOE.BTKp.co), 23 (Eμ.BTKp). As shown, the vector with the enhancer led to the higher expression of BTK in both the B cells and the myeloid cells as well as an increase in IgG secretion.

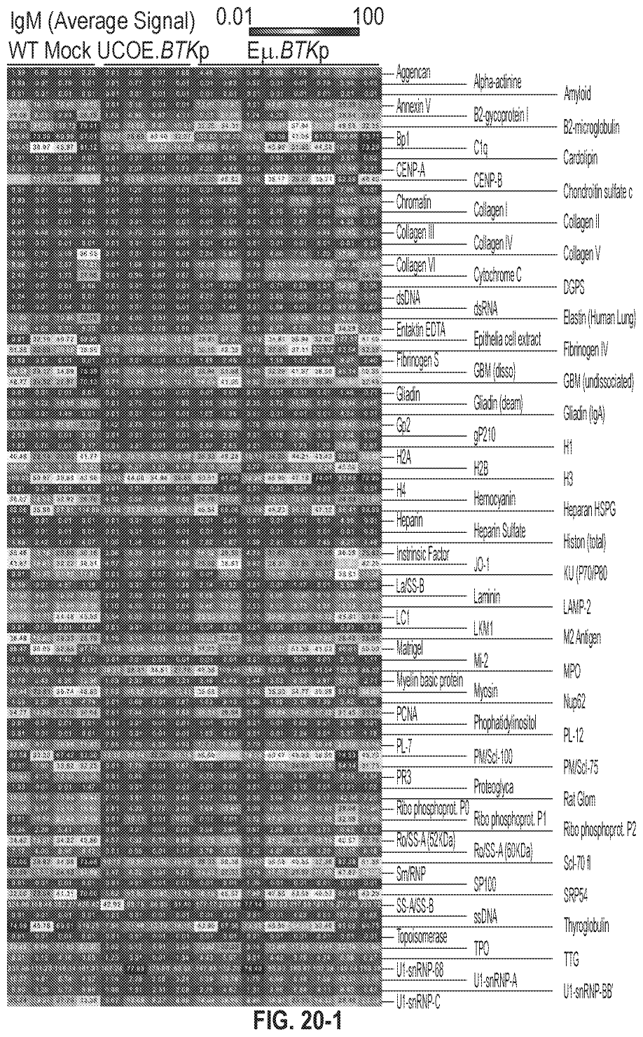

As shown in FIG. 19, Eμ.BTKp primary transplant mice also had increased survival as compared to the control XLA mice treated with cells that were transduced with the mock vectors.

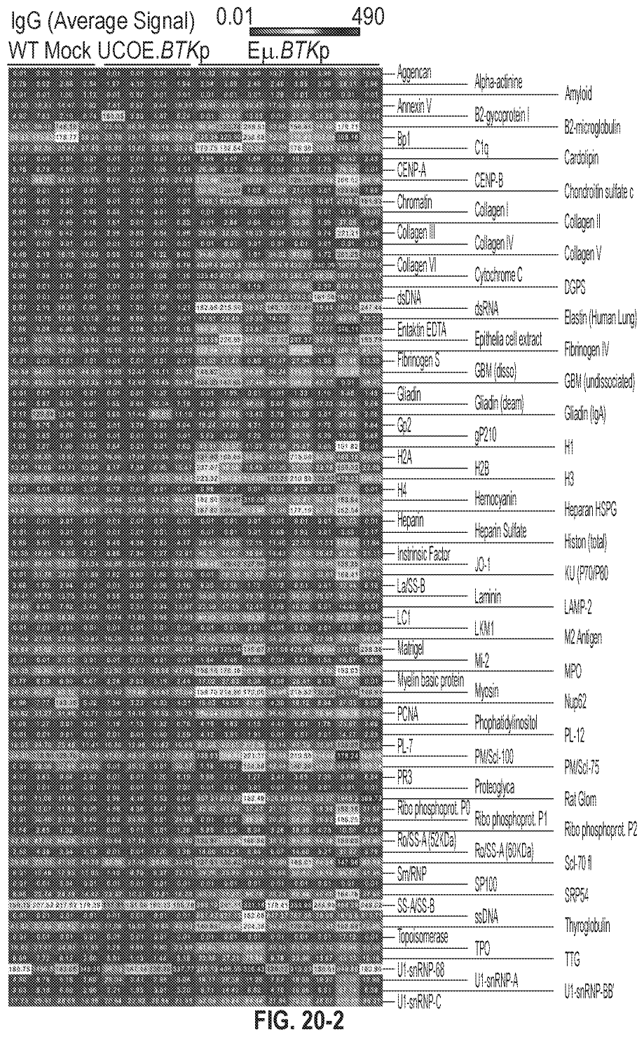

Sera from the mice treated with the cells transduced with the WT Mock, 1.5 kb.UCOE.BTKp and Eμ.BTKp vectors were analyzed by autoantigen array for levels of IgM and IgG reactive to 88 murine antigens. Data from each row was subject to z-transformation and Z-scores are displayed on a colorimetric scale from lowest reactivity (red) to highest (blue). As shown in FIG. 20, the sera with the most reactivity were from the cells transduced with lentiviral vectors that had the Eμ promoter (Eμ.BTKp.)

### Designing Vectors for Increasing BTK Expression in B Cells and Monocytes

B cell specific promoters were also examined for their influence on BTK production. In an exemplary alternative, a lentivector was manufactured, which comprised the Eμ enhancer element and a B cell specific promoter, B29, fused to a gene encoding human BTK (huBTK) (FIG. 27). As shown, the vector led to an increase of BTK expression in B cells; however, the BTK expression in the monocyte was comparable to the knockout cells (FIG. 27).

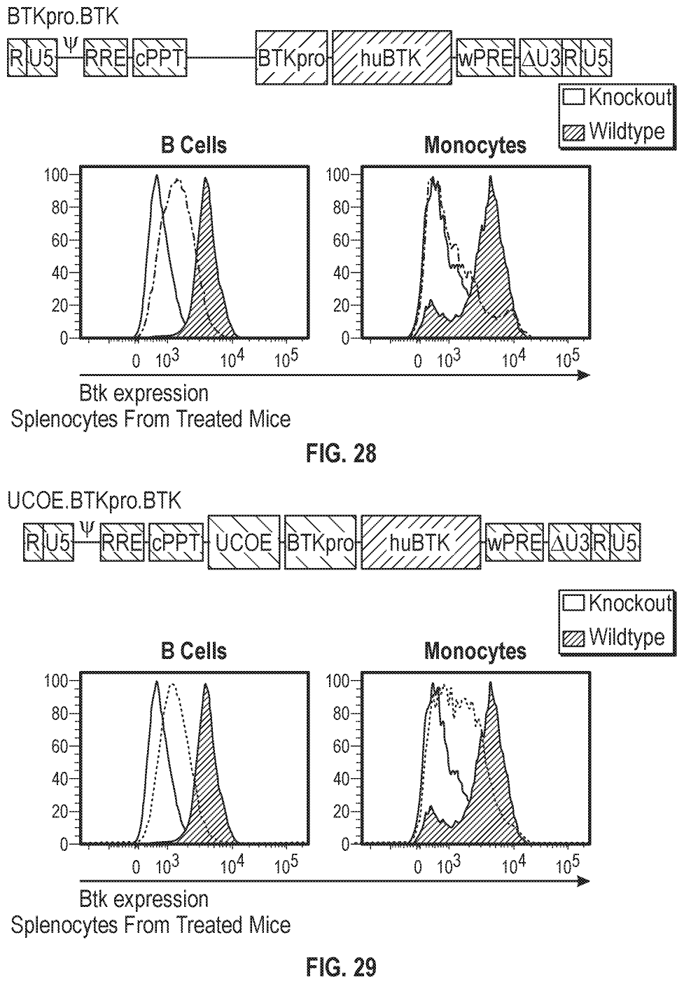

The BTK promoter region was also examined in another alternative, to evaluate the influence of the BTK promoter in a lentiviral vector for BTK expression. As shown in FIG. 28, a lentiviral vector was manufactured, which comprised the BTK promoter (BTKpro) fused to the gene encoding human BTK (huBTK). As shown, the BTK promoter led to increased BTK expression in B cells. However, BTK expression was not comparable to the expression in wild type cells. In the monocytes, the lentiviral vector comprising the BTK promoter region was unable to lead to increased expression of BTK and the expression of BTK was comparable to the knock out cells.

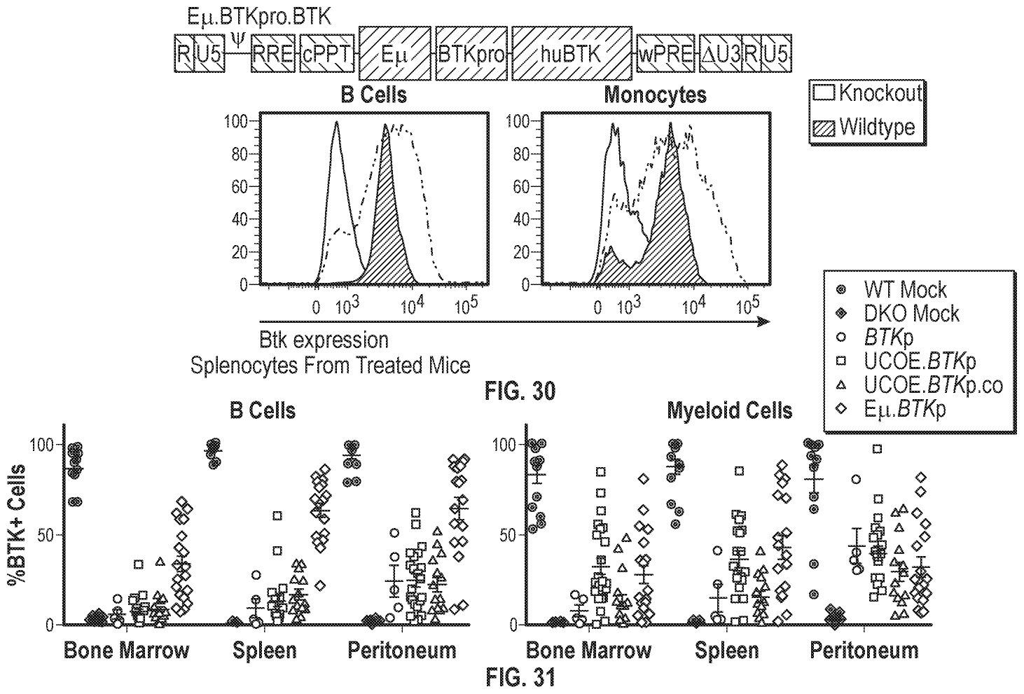

A lentiviral vector was manufactured, which comprised a ubiquitous chromatin opening element and a BTK promoter (BTKpro) fused to a gene encoding human BTK (FIG. 29). As shown, the enhancer element along with the promoter led to increase of BTK expression in both B cells, as well as, monocytes. However, the expression was not comparable to the expression in the wild type cells.

A lentiviral vector was manufactured, which comprised a Eμ enhancer and a BTK promoter (BTKpro) fused to a gene encoding human BTK (FIG. 30). As shown, the enhancer element along with the promoter led to increase of BTK expression in both B cells and monocytes. The results show that the expression BTK in the transduced cells exceeded the amounts of BTK in the wild type cells. The data shows that the Eμ enhancer led to the most BTK expression of the vectors that were tested.

Provided below are exemplary constructs used herein:

1. 0.7UCOE.BTKp.coBTK.

2. 0.7UCOEfwd.BTKp.coBTK

3. 0.7UCOE.DHS4.BTKp.coBTK

4. 0.7UCOEfwd.DHS4.BTKp.coBTK

5. 0.7UCOE.IE.BTKp.coBTK

6. 0.7UCOEfwd.IE.BTKp.coBTK

7. 0.7UCOE.BTKp.co2BTK

8. 0.7UCOEfwd.BTKp.co2BTK

9. 0.7UCOE.DHS4.BTKp.co2BTK

10. 0.7UCOEfwd.DHS4.BTKp.co2BTK

11. 0.7UCOE.IE.BTKp.co2BTK

12. 0.7UCOEfwd.IE.BTKp.co2BTK

Generally, these constructs represent various iterations of 3 different elements (0.7UCOE, Enhancer, or BTK coding sequence, all having the same BTK promoter):

| | | | | | | BTK human | |
|---|---|---|---|---|---|---|---|
| | | | | | PRO- | codon | |
| 0.7UCOE: | | ENHANCER: | | | MOTER | optimization: | |
| 0.7UCOE | 0.7UCOEfwd | none | DHS4 | IE | BTKp | coBTK | co2BTK |

### Human Codon Optimized BTK

Human codon optimization of the gene encoding BTK was performed. Human codon optimization can be performed by using algorithms that are known to those skilled in the art so as to create synthetic genetic transcripts optimized for high mRNA and protein yield in humans. As shown in FIG. 17, two constructs were tested for their ability to lead to increased BTK expression in cells. The lentiviral constructs comprised the element and the BTK promoter region. The genes for expression were BTK and a human codon optimized BTK, both fused to GFP gene transcripts. As shown in FIG. 17, human codon optimized BTK led to an increase in GFP (MFI 2288), as well as, an increase in GFP.

### Expression Profiles of the BTK Lentiviral Vector

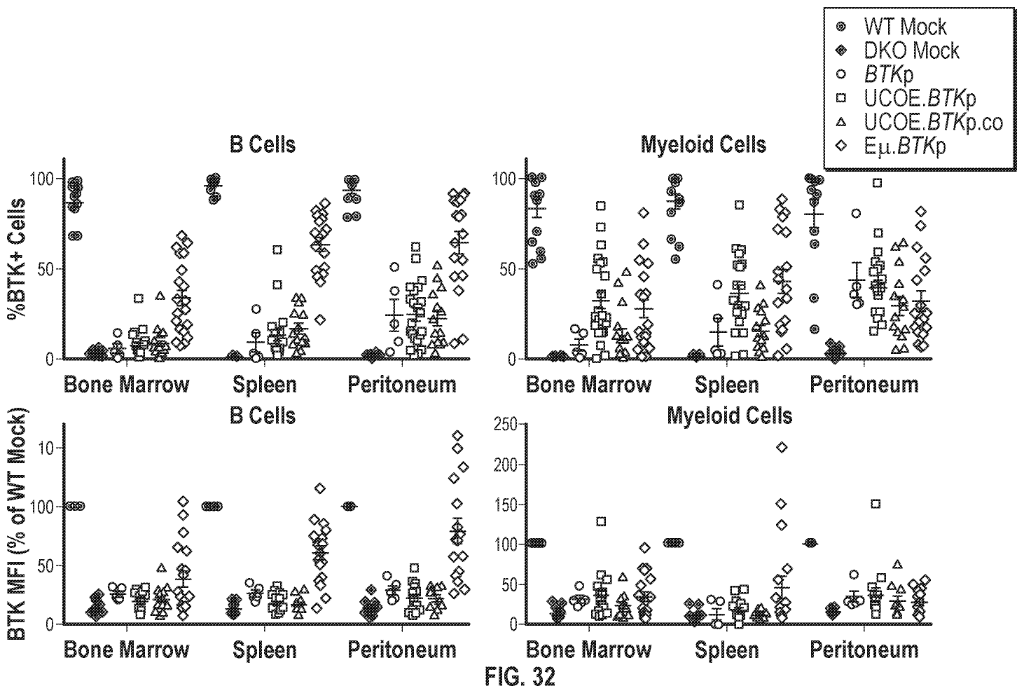

The expression profile of several vectors were examined: WT mock, DKO mock, BTKp (BTK promoter), UCOE. BTKp (ubiquitous chromatin opening element plus BTK promoter), UCOE.BTKp.co (ubiquitous chromatin opening element, BTK promoter plus human codon optimized BTK) and Eμ.BTKp (Eμ element plus BTK promoter). As shown in FIGS. 31 and 32, the lentiviral vector for BTK expression with the Eμ element and BTK promoter led to the highest expression of BTK in bone marrow, spleen and peritoneum from the B cells and the myeloid cells.

### B Cell Development and Restoration of Mature B Cell Subsets

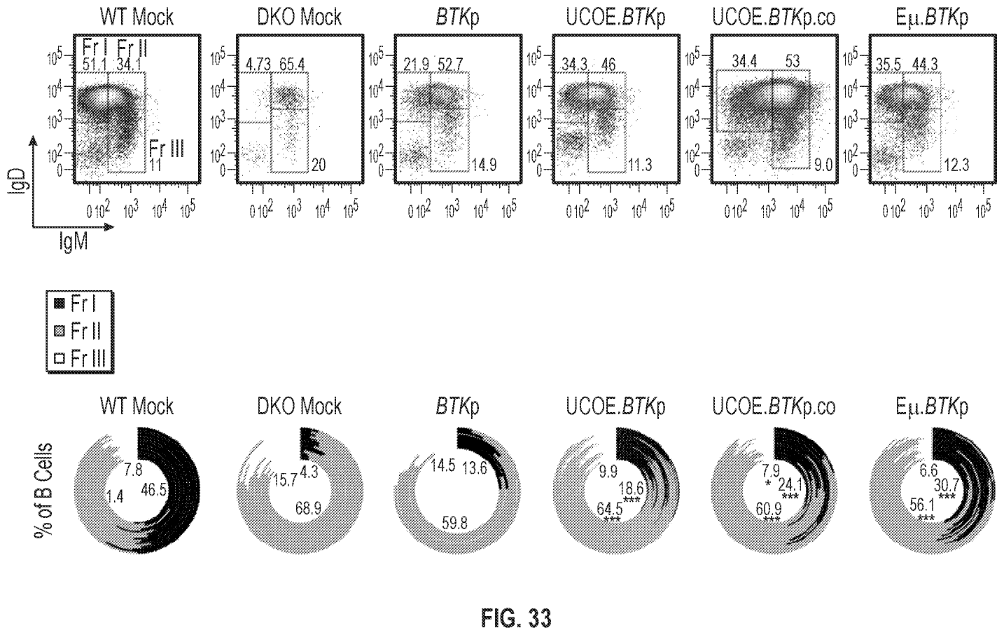

The restoration of mature B cell subsets were also examined in the cells transduced with the vectors: WT mock, DKO mock, BTKp (BTK promoter), UCOE. BTKp (ubiquitous chromatin opening element plus BTK promoter), UCOE.BTKp.co (ubiquitous chromatin opening element, BTK promoter plus codon optimized BTK) and Eμ.BTKp (Eμ element plus BTK promoter). In XLA, development stops at the pre B cell. Accordingly, the cells were examined for further development, in which development of the B cell is dependent on the levels of BTK. As shown, the lentivector comprising the Eμ element plus BTK promoter for BTK expression led to cells that were of the mature peripheral B cell population, indicating that the levels of BTK produced from the transduced cell would lead to restoration of mature B cell subsets in the mice (FIG. 33).

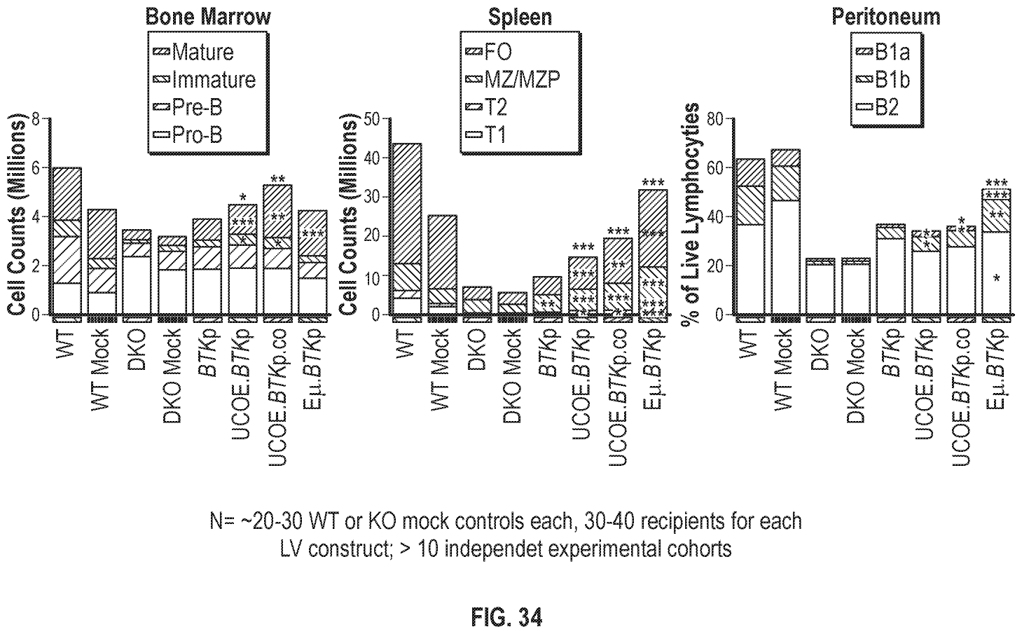

The numerical reconstitution of B cell populations were also examined in BTK deficient mice that were administered cells expressing BTK. The cells were transduced with the following vectors: WT mock, DKO mock, BTKp (BTK promoter), UCOE. BTKp (ubiquitous chromatin opening element plus BTK promoter), UCOE.BTKp.co (ubiquitous chromatin opening element, BTK promoter plus human codon optimized BTK) and Eμ.BTKp (Eμ element plus BTK promoter). Shown in FIG. 34 are the data that summarize the finding from 40 recipient mice that received the transduced cells for BTK expression. Mice that received the cells that were transduced with the BTK expression vector comprising UCOE.BTKp.co, Eμ.BTKp or UCOE.BTKp were able to develop mature B cells in the bone marrow, spleen and peritoneum. Accordingly, aspects of the invention concern a lentiviral vector that comprises ubiquitous chromatin opening element, BTK promoter, and human codon optimized BTK, which can be used in methods to promote mature B cell development in subjects suffering from XLA.

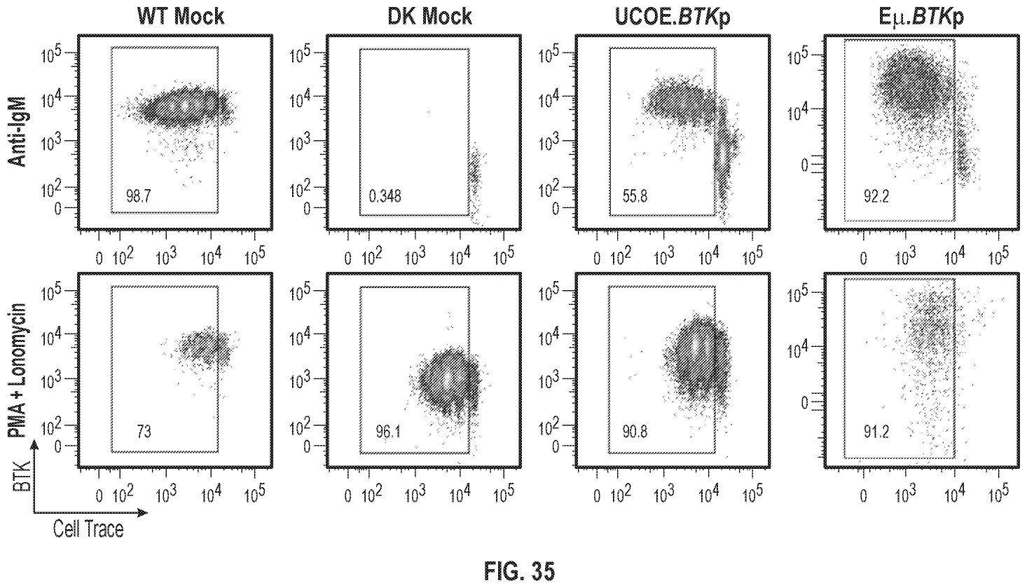

B cell proliferation was also shown in mice that were treated with cells transduced with the lentiviral vectors that comprised the ubiquitous chromatin opening element and BTK promoter (UCOE.BTKp) and a lentiviral vector that comprise the element fused to the BTK promoter region (Eμ.BTKp) (FIG. 35). Controls include the WT mock as well as the DKO mock cells. Fluorescence activated cell sorting was performed on the cells using anti-IgM antibodies. As shown, the cells with expressing BTK from the two lentiviral vectors UCOE.BTKp and Eμ.BTKp had expression of IgM, which is a basic antibody produced by B cells.

The cells were also treated with PMA and lonomycin. PMA is used for activating PKC, while lonomycin, is used to trigger calcium release, which is needed for NFAT signaling (FIG. 35).

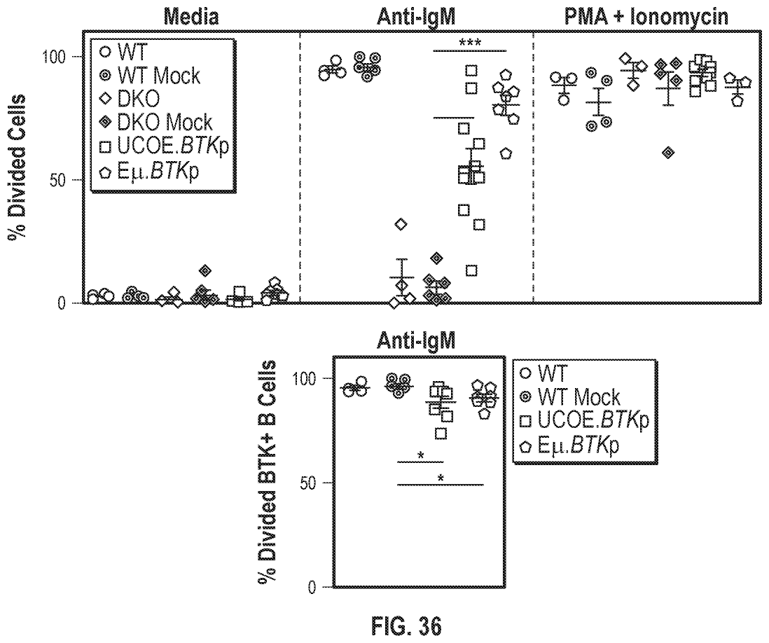

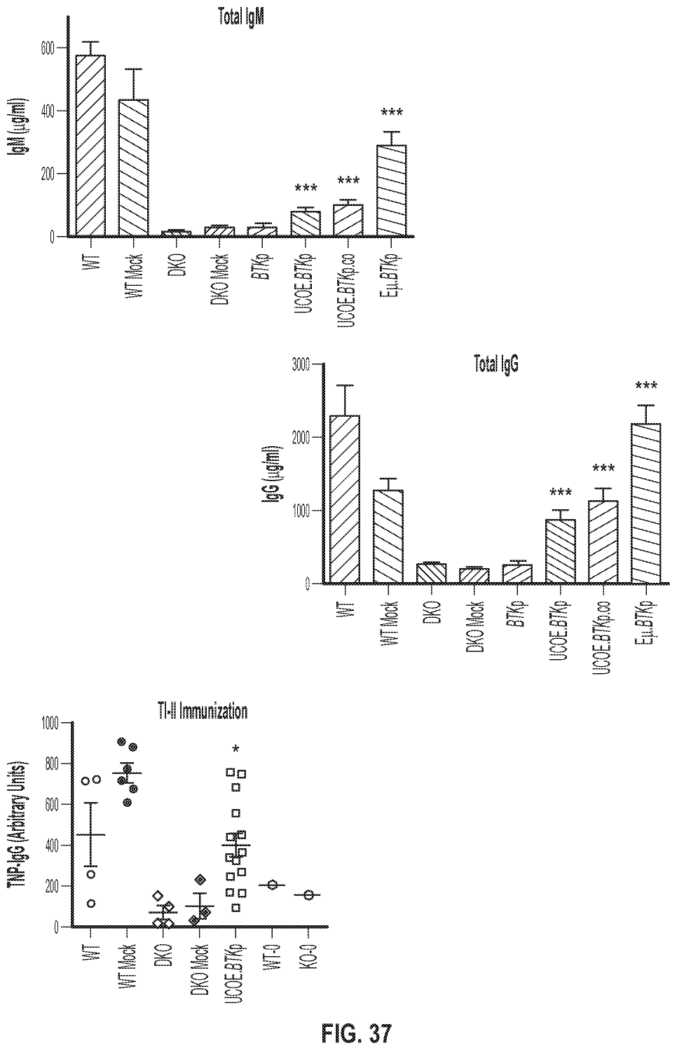

As shown in FIG. 36, the cells that were transduced with the two lentiviral vectors UCOE.BTKp and Eμ.BTKp had an increase in cell division as compared to the wild type, when treated with anti-IgM and PMA/Ionomycin. Additionally, these cells were also shown to have an increase in total IgM and IgG secretion as compared to the vectors that had only the BTK promoter and the BTK promoter with the UCOE element (FIG. 37).

### T Cell Dependent Immune Responses

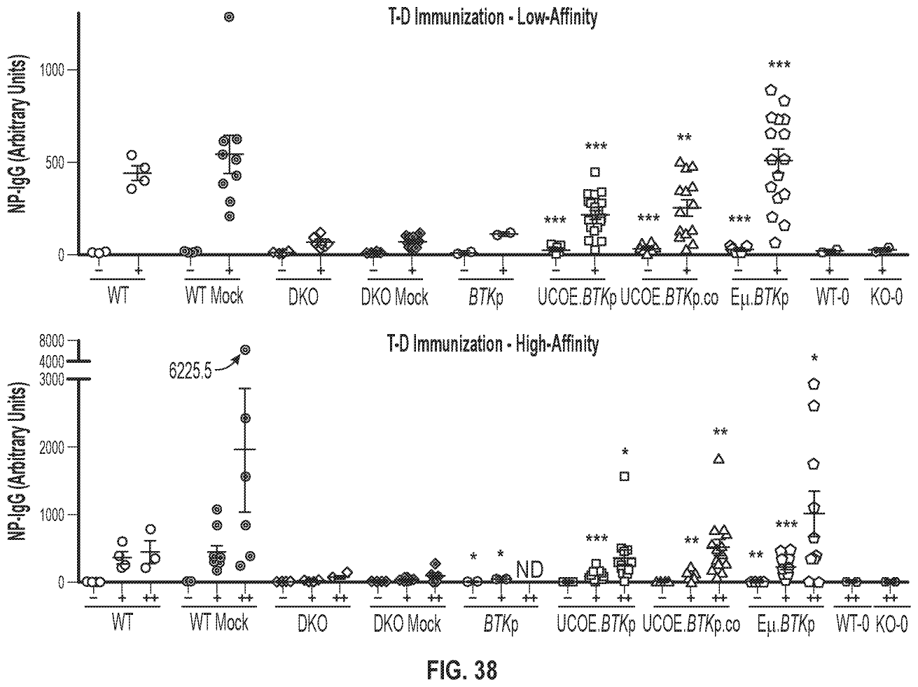

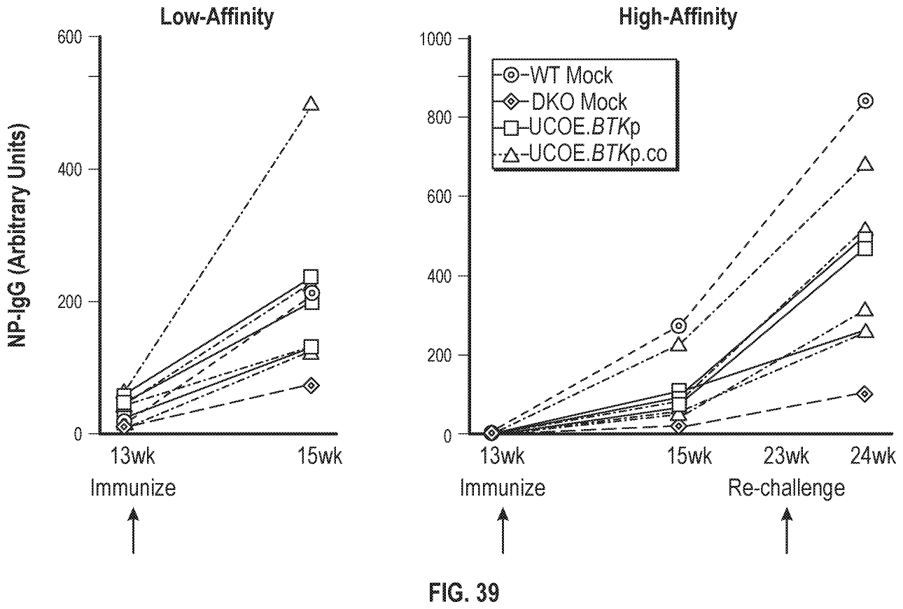

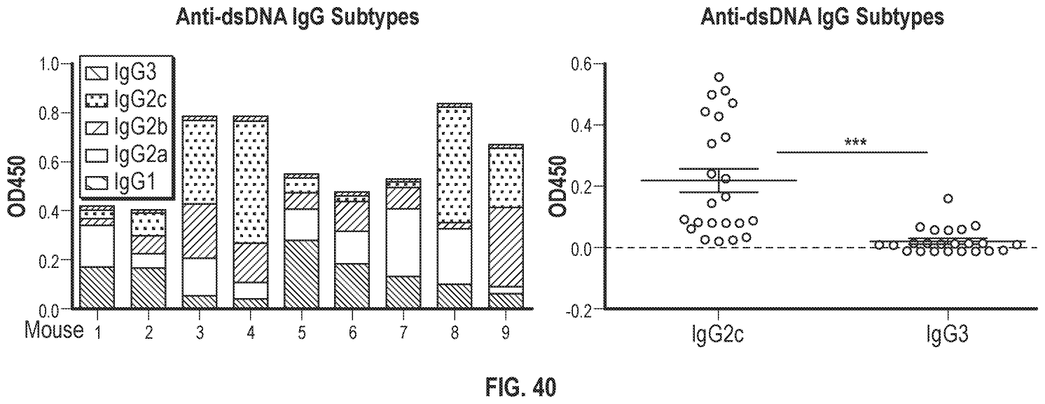

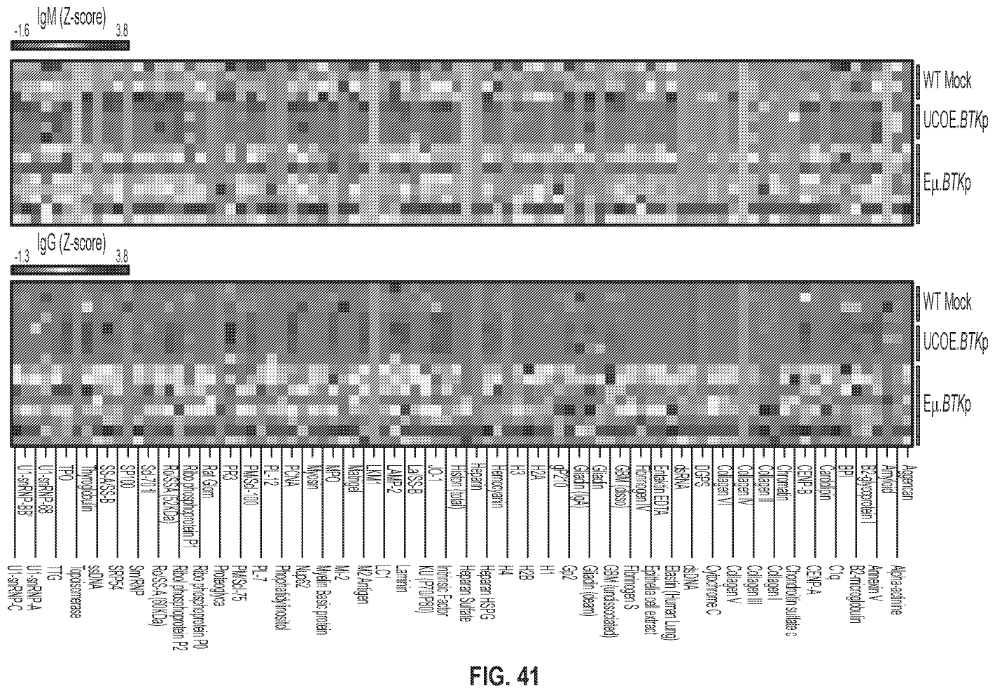

The transduced cells were administered to mice and further tested for T cell dependent immune responses. The cells were transduced with the following lentiviral vectors: WT mock, DKO, DKO mock, BTKp, UCOE.BTKp, and UCOE.BTKp.co (human codon optimized BTK), Eμ.BTKp.co (human codon optimized BTK), and Eμ.BTKp. T cell immune responses were then evaluated. As shown, the cells transduced with Eμ.BTKp lentiviral vectors led to increased IgG and IgM expression (FIG. 38-41).

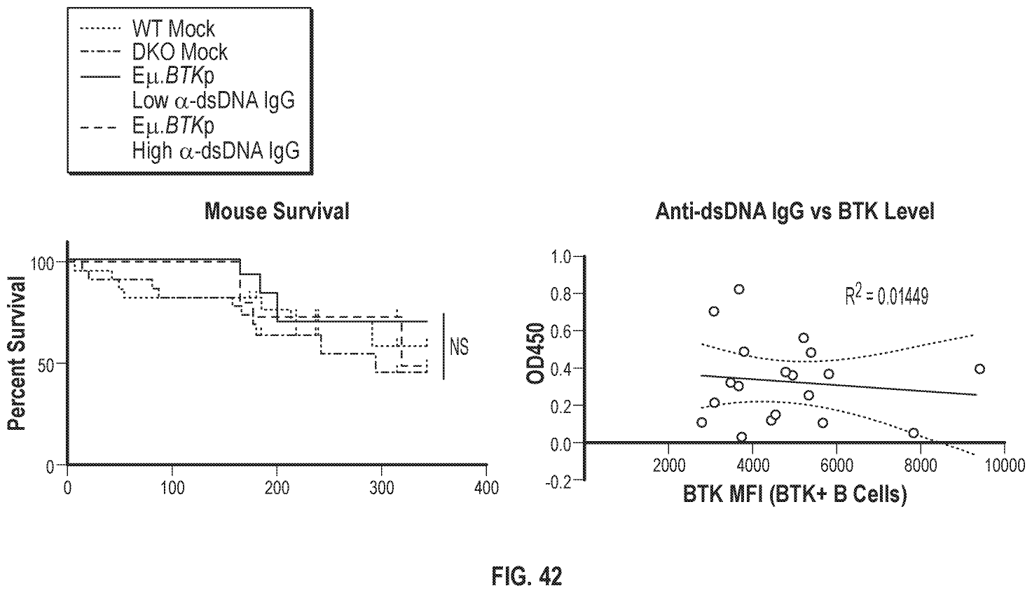

The antibody levels were also associated with BTK expression, as well as, survival of the mice. As shown in FIG. 42, mice that were administered cells transduced with Eμ.BTKp lentiviral vectors led to increased mouse survival, as compared to the mice that were administered the DKO mock cells.

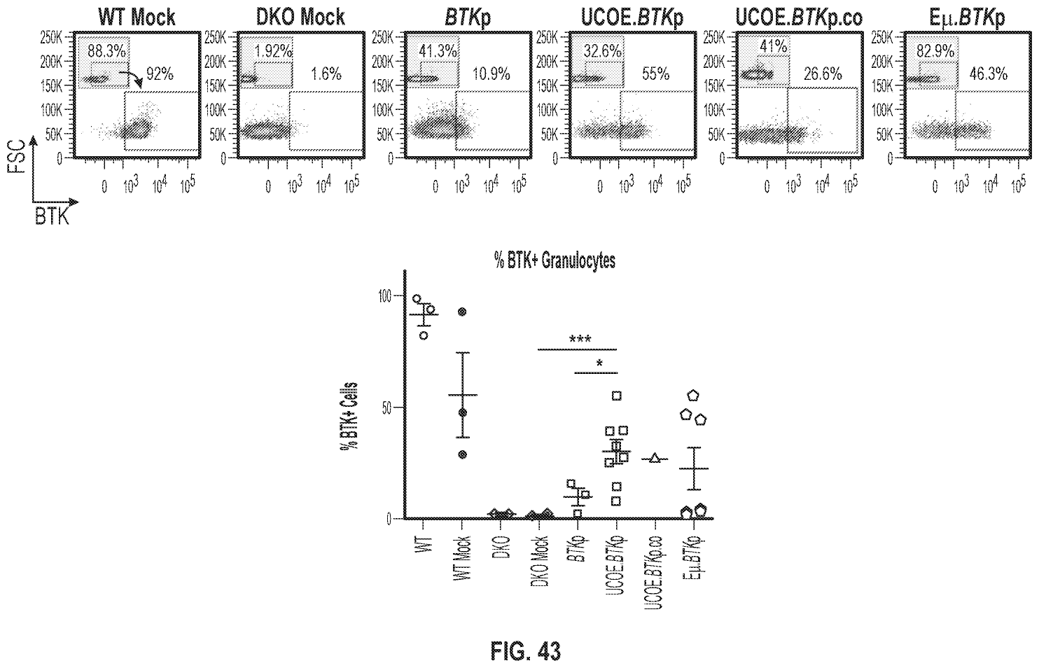

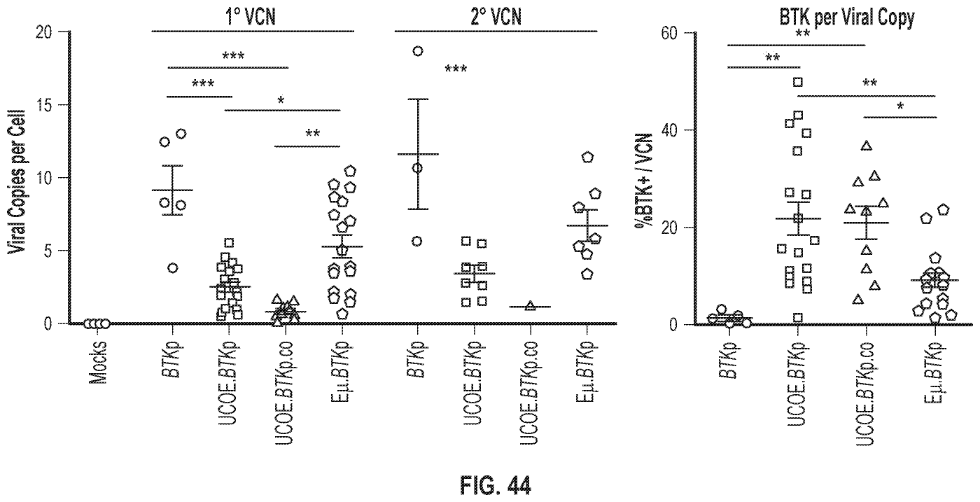

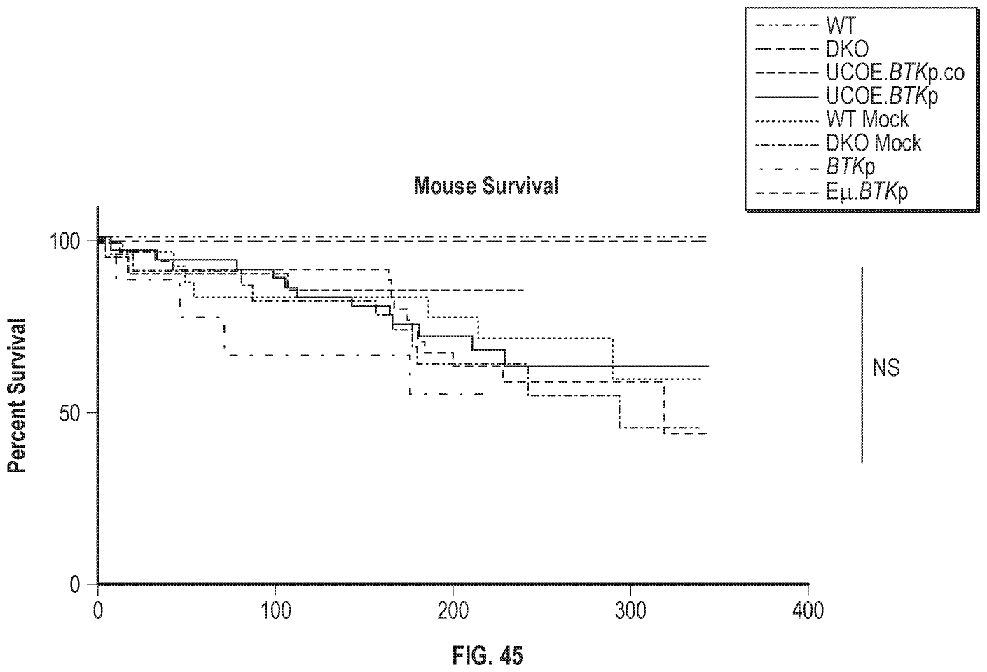

BTK expression was also evaluated in neutrophils and secondary recipient mice. As shown in FIG. 43, administered cells transduced with Eμ.BTKp lentiviral vectors led to increased BTK expression in secondary recipient mice. However, there was increased viral copy number in secondary recipients in mice that were administered the viral vector BTKp (FIG. 44). Evaluation of the lentiviral therapy on long term survival provided evidence that the vectors UCOE.BTKp.co, UCOE.BTKp and Eμ.BTKp led to increased mouse survival.

Overall the BTK promoter studies demonstrated that the BTK promoters in the lentiviral vectors exhibit significant BTK expression in B and myeloid cells. UCOE.BTKpro and Eμ.BTKpro rescue B cell development, absolute B cell numbers, B cell proliferation, and immune responses. Myeloid expression is also rescued with UCOE.BTKpro and Eμ.BTKpro. Unexpectedly, Eμ-containing vectors lead to high-titer autoantibody production, thus, making them potentially unsafe for clinical use. UCOE-Btkp-Btk vectors exhibit functional rescue at much lower viral copy number compared with non-UCOE vectors. UCOE-Btkp-Btk vectors exhibit sustained marking in both murine and human HSC. Thus, the UCOE.BTKp-coBTK lentiviral vector represents an improved and unique clinical vector platform for additional modification.

### Alternative Enhancer Elements

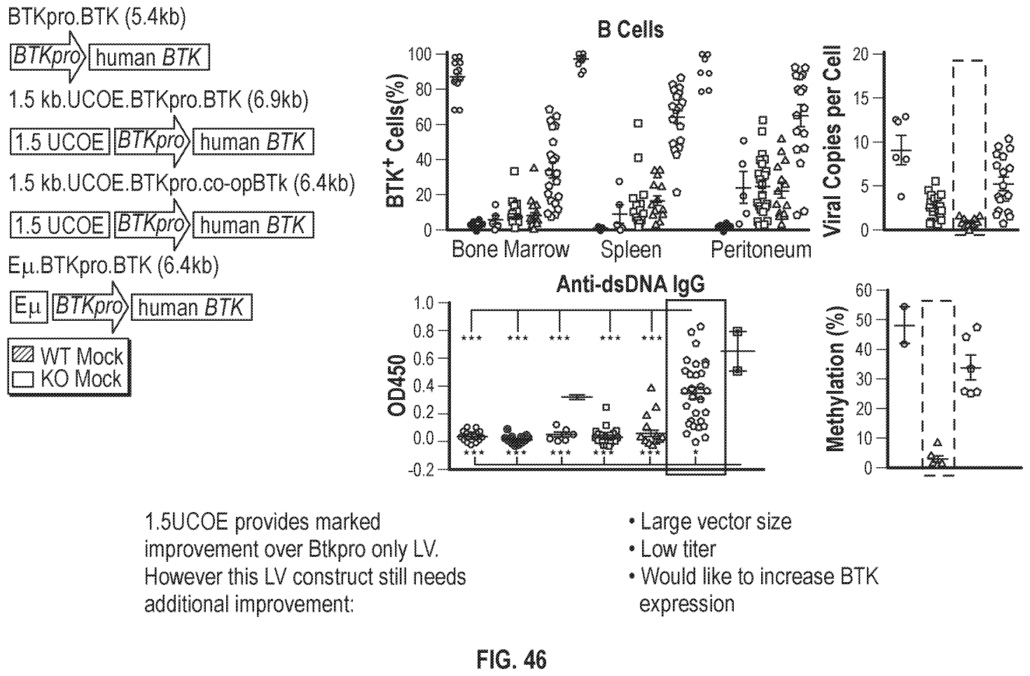

Alternative enhancer elements within the BTK promoter were evaluated for their abilities to improve BTK expression in cells. The lentiviral constructs used are shown in FIG. 46. As shown, the 1.5 kb UCOE gave improvement over the BTK promoter in B cells. However, additional improvement was sought for decreasing the large vector size and the low viral titers (FIG. 46).

The 1.5 kb UCOE was truncated to 0.7UCOE to create the lentiviral vector 0.7UCOE.BTKp (FIG. 2). Potential human BTK enhancer elements were also added to create 0.7UCOE.IE.BTKp as shown in FIG. 2. The intronic regions included intron 4 and 5 from a human BTK locus that is in association with a human BTK proximal promoter.

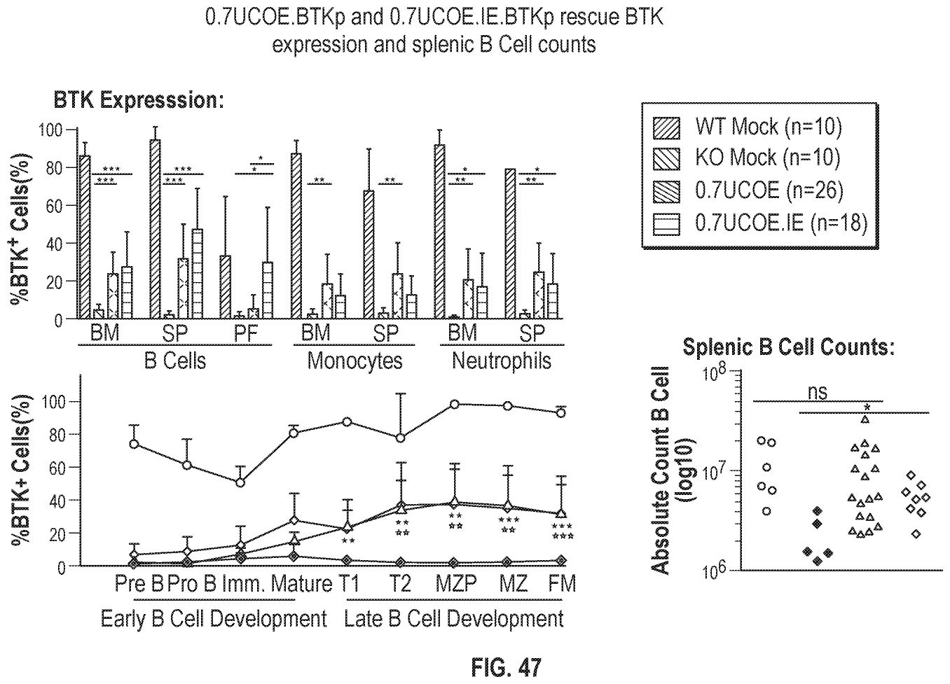

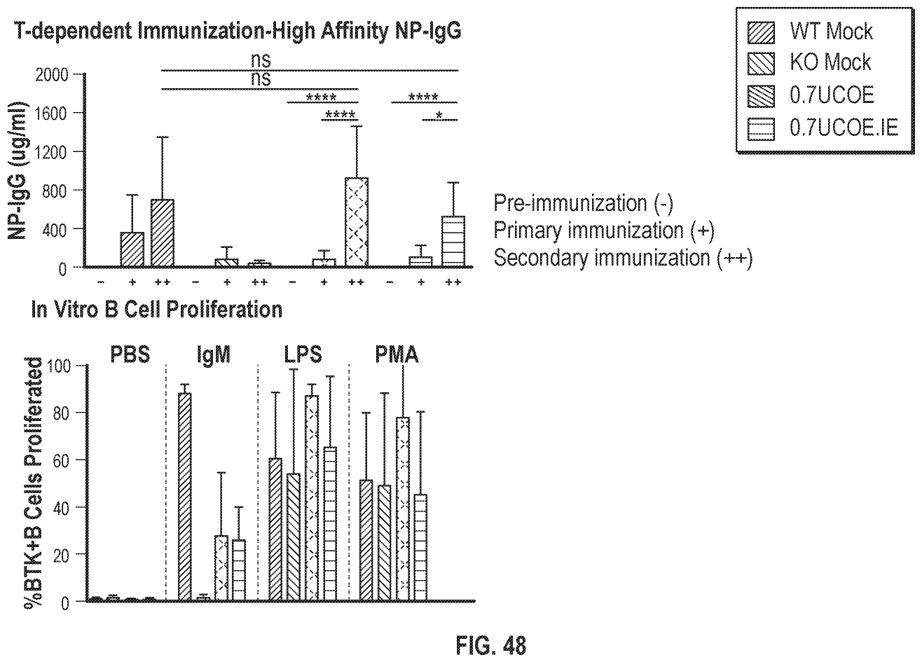

The 0.7UCOE.BTKp and 0.7UCOE.IE.BTKp were transduced into cells and the methods outlined in FIG. 3 were used for administering the cells into the mouse. As shown in FIG. 47, the cells transduced with 0.7UCOE.BTKp and 0.7UCOE.IE.BTKp rescue BTK expression and splenic B cell counts. The cells were also shown to rescue B cell function (FIG. 48).

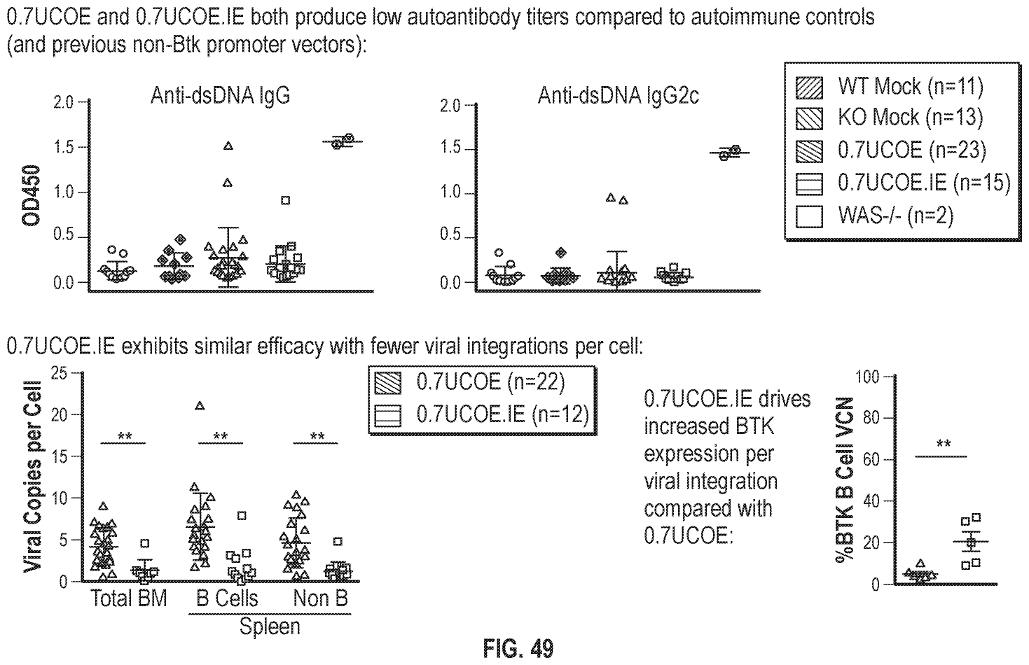

The cells transduced with 0.7UCOE.IE exhibited an improved safety profile over 0.7UCOE, as well (see FIG. 49). As shown, 0.7UCOE and 0.7UCOE.IE both produce low autoantibody titers compared to autoimmune controls (and previous non-Btk promoter vectors, while 0.7UCOE.IE exhibits similar efficacy with few viral integrations per cell.

### Testing of BTK Constructs Comprising DNase Hypersensitive Sites

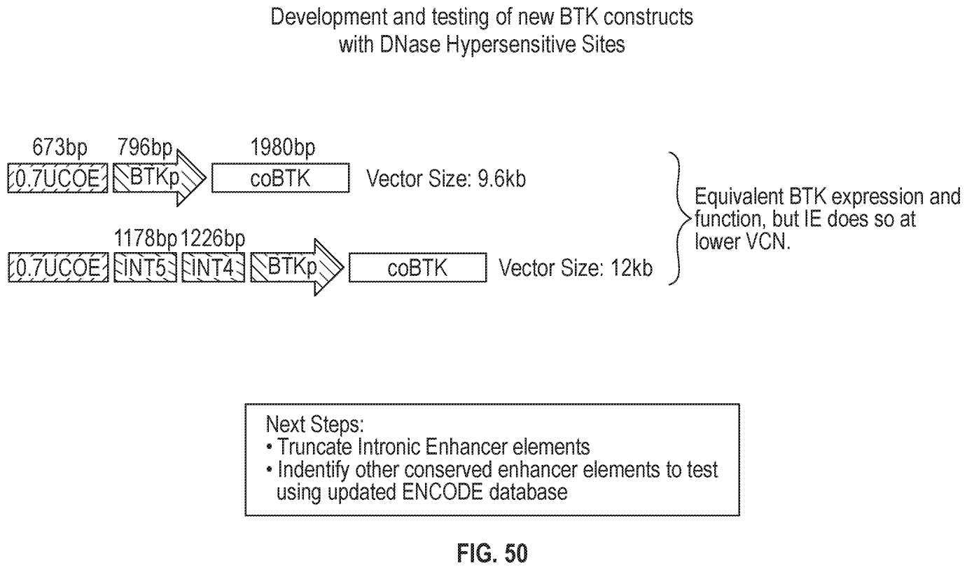

Lentiviral constructs for expression of BTK were designed with DNase Hypersensitive sites (FIG. 50). The vectors were the following: 0.7UCOE.BTKp.coBTK (0.7 UCOE enhancer, BTK promoter, and human codon optimized BTK) and 0.7UCOE.IE4,5.BTKp.coBTK (0.7UCOE element, intron 4 and 5 of the human BTK locus that is in association with the human BTK proximal promoter, BTK promoter and the human codon optimized BTK).

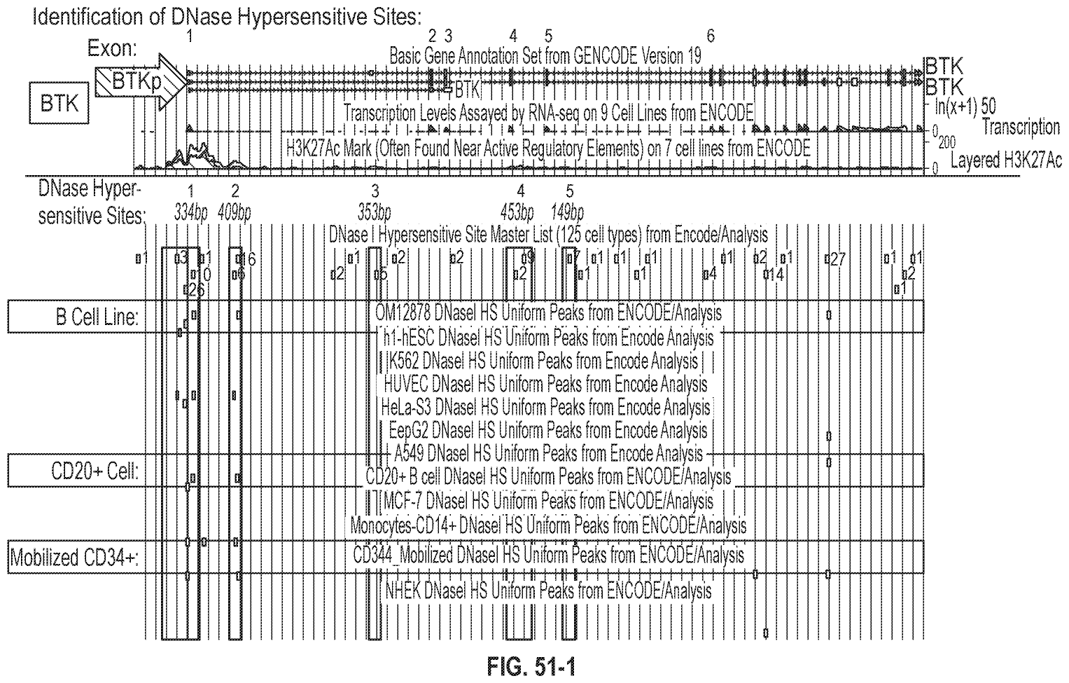

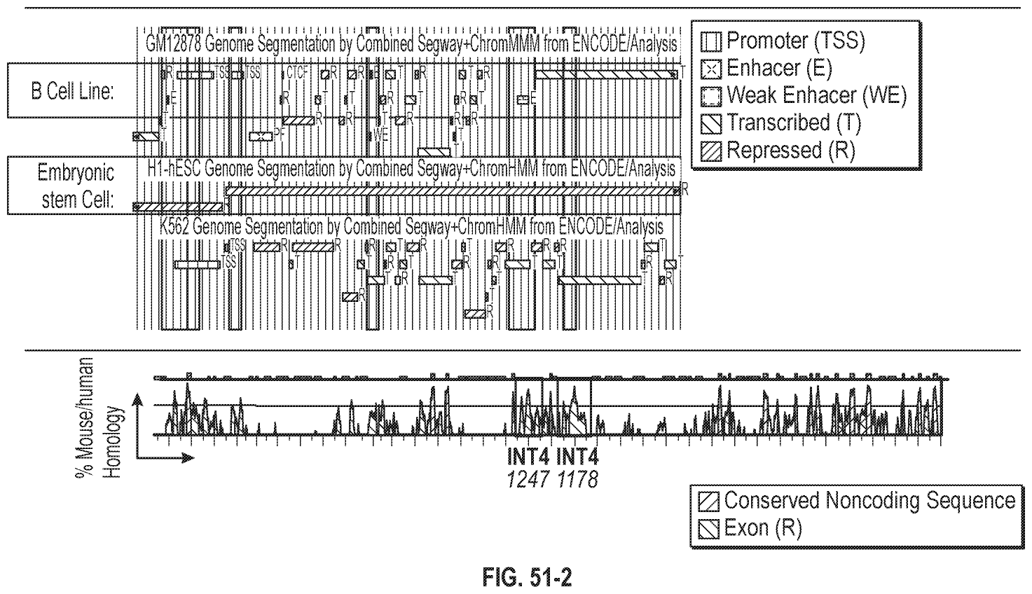

The DNAse hypersensitivity sites were identified as shown in FIG. 51.

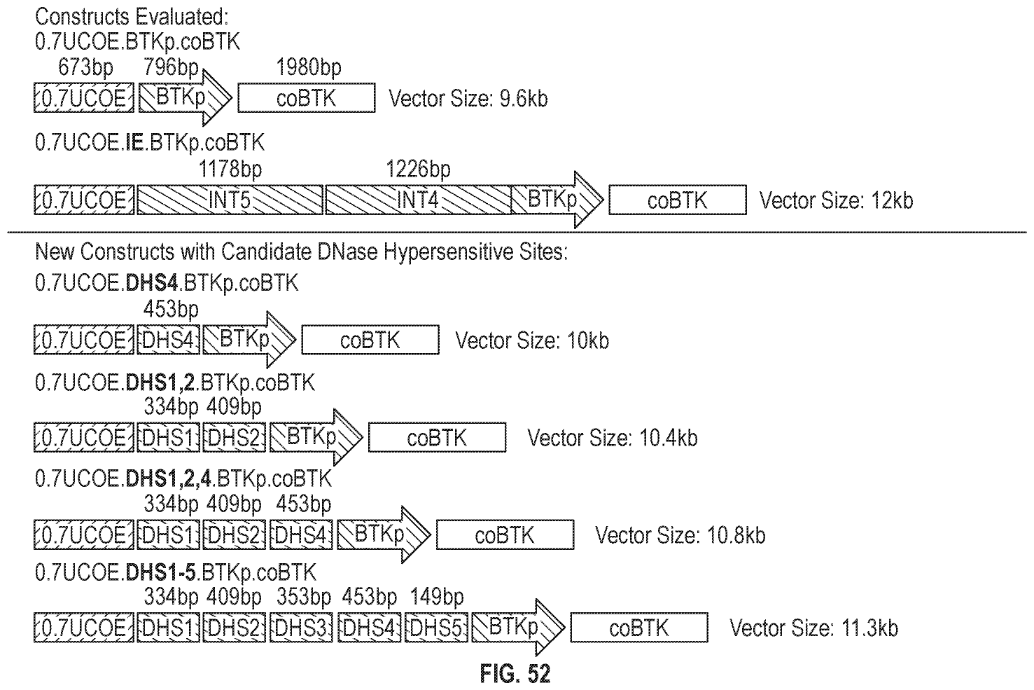

Base on the identification of the introns and the DNAse hypersensitivity sites, constructs with candidate introns and DNAse hypersensitive sites were constructed (FIG. 52). Constructs were the following: 0.7UCOE.BTKp.coBTK, 0.7UCOE.IE.BTKp.coBTK, 0.7UCOE.DHS4.BTKp.coBTK, 0.7UCOE.DHS1,2.BTKp.coBTK, 0.7UCOE.DHS1,2,4.BTKp.coBTK, and 0.7UCOE.DHS1-5.BTKp.coBTK.

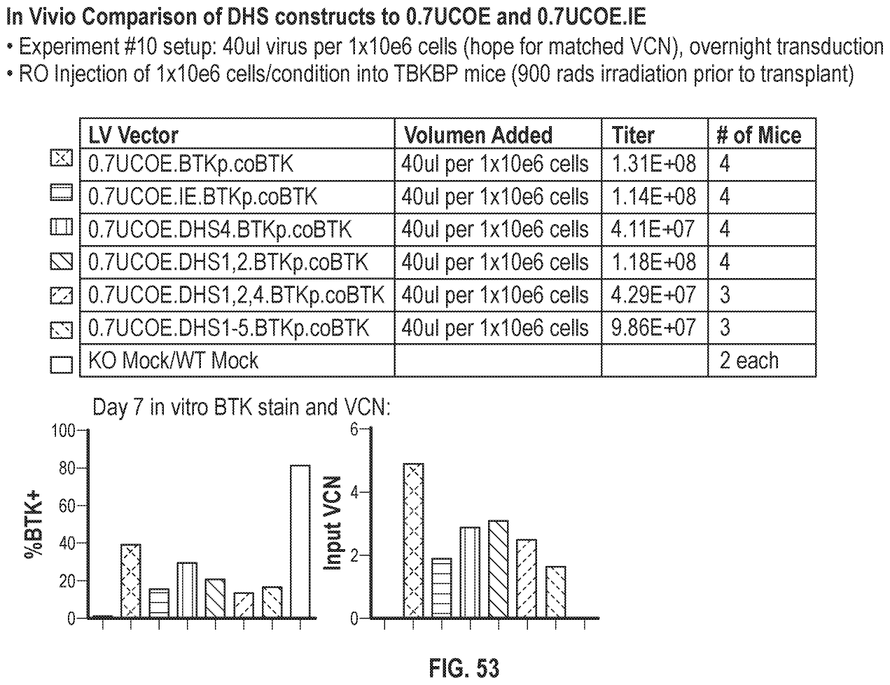

In vivo comparisons of the DHS constructs to the 0.7UCOE and 0.7UCOE.IE vectors were performed. In an experimental set up, 40 ul virus per 1×10⁶ cells (optimized for matched viral count numbers (VCN) for an overnight transduction. This was followed by RO injection of 1×10⁶ cells/condition into TBKBP mice (900 rads irradiation prior to transplant). As shown, the cells that were transduced with the 0.7UCOE.BTKp.coBTK led to higher expression of BTK with higher VCN (FIG. 53).

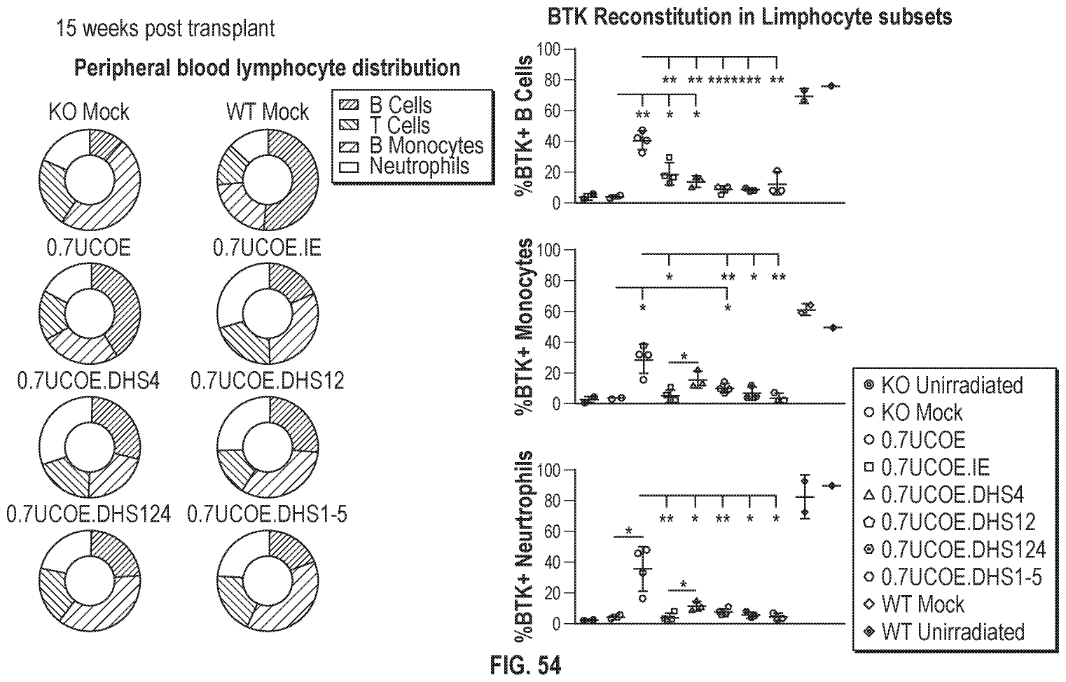

After 15 weeks post-transplant, B cell development was evaluated and a peripheral blood lymphocyte distribution was examined. As shown in FIG. 54, the cells transduced with the vectors led to an increase of B cells over the KO mock cells with 0.7UCOE lentivector showing an increase in B cells. However, higher levels of BTK reconstitution in lymphocyte subsets was seen with cells transduced with the lentiviral vector comprising the 0.7UCOE elements.

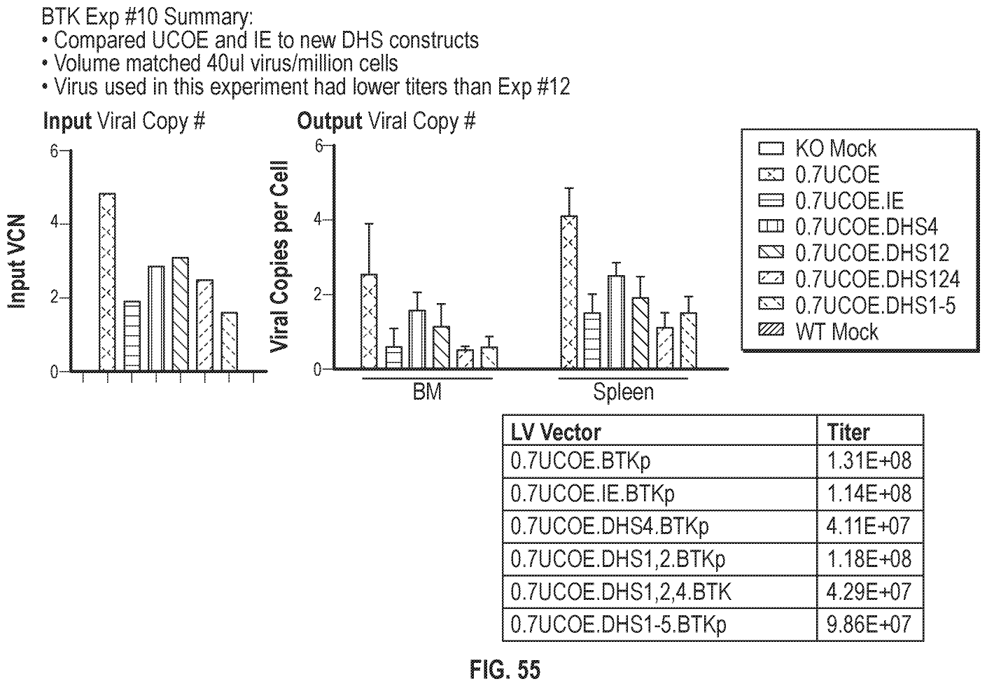

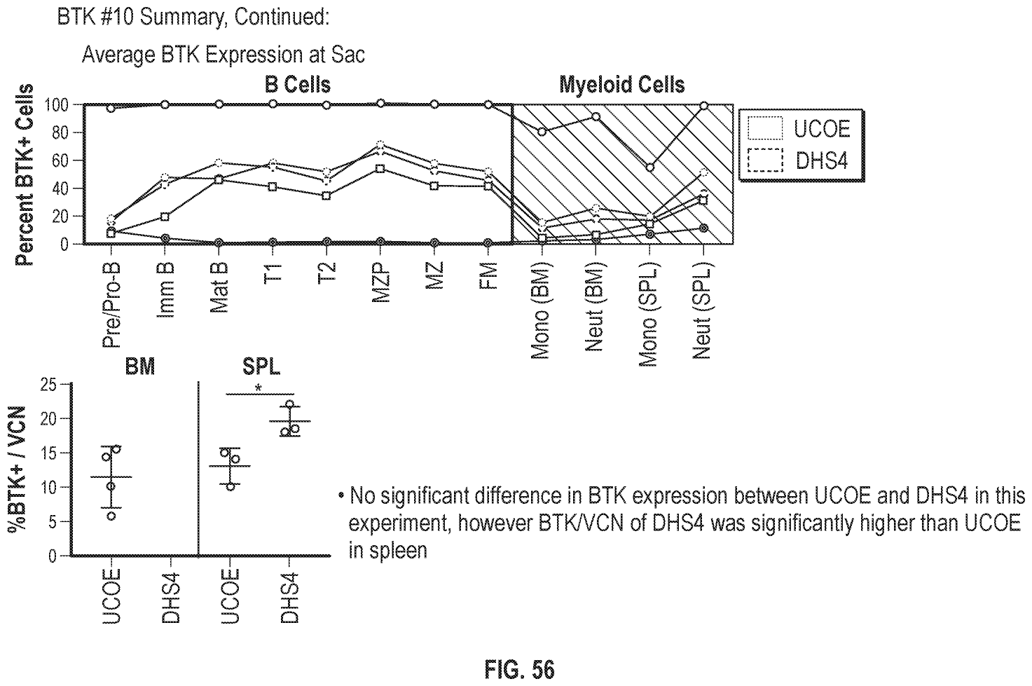

Experiments were performed to compare the lentiviral constructs, which had the UCOE and intronic elements to the new DHS constructs. The volumes were matched for 40 uL virus/million cells. As shown in FIG. 55, the input VCN was increased in the cells transduced with the lentiviral vector comprising the 0.7UCOE element, as compared to the vectors comprising the DHS elements. There was also no significant difference in BTK expression between UCOE and DHS4 in these experiments; however the BTK/VCN of DHS4 was significantly higher than UCOE in spleen (FIG. 56).

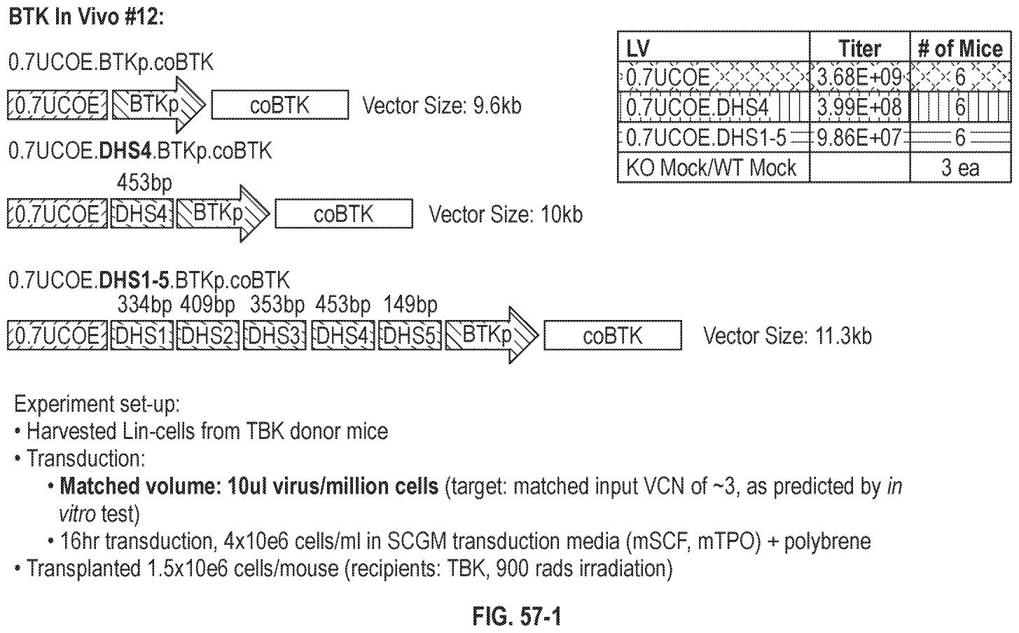

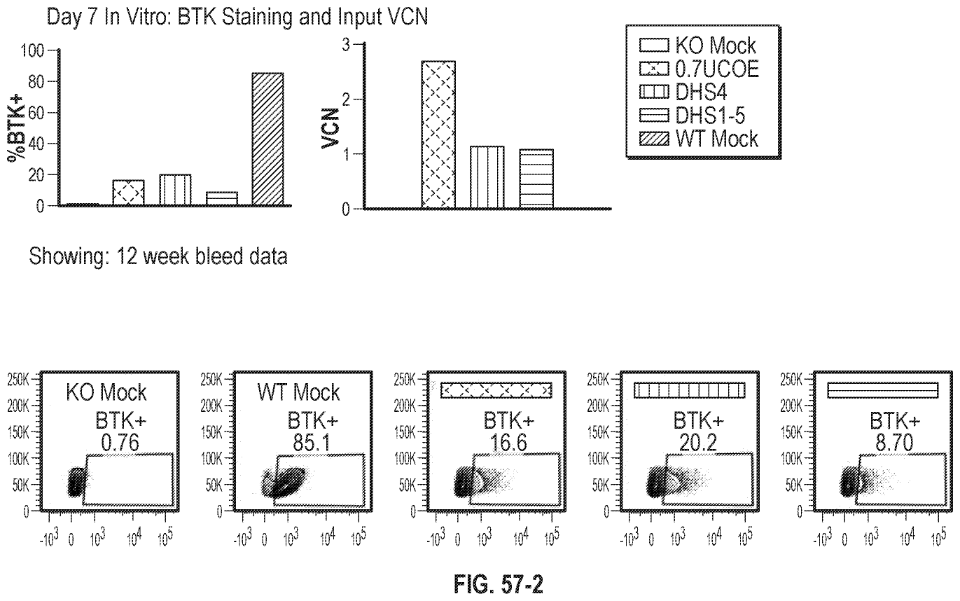

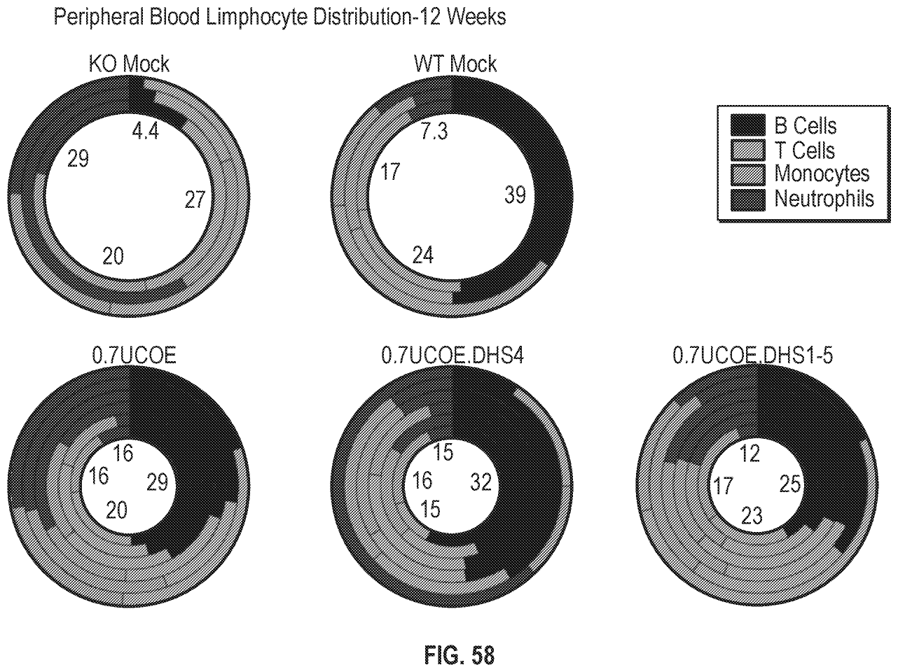

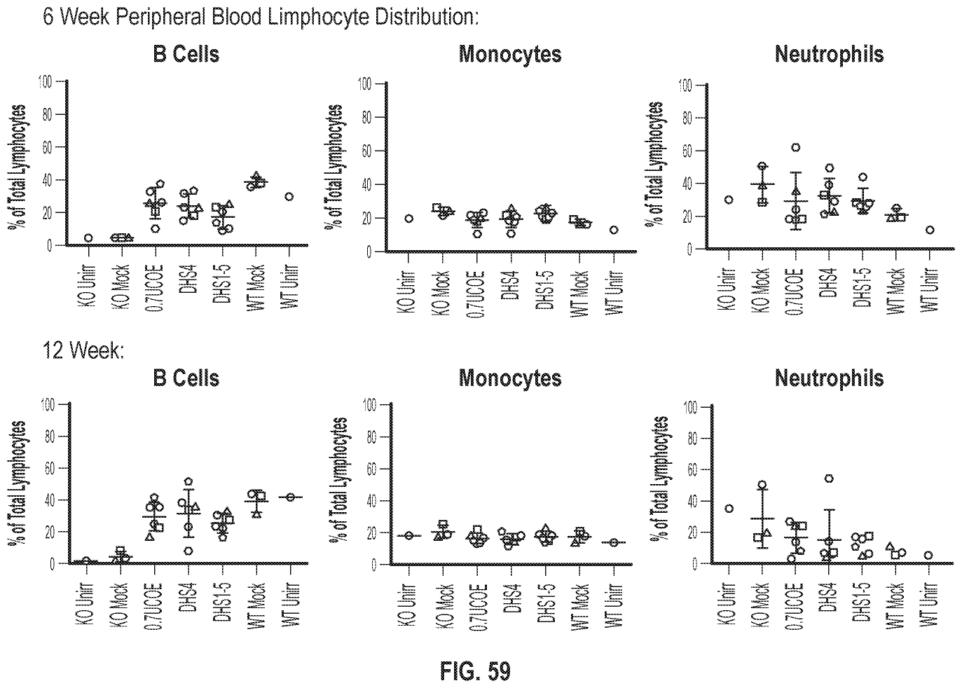

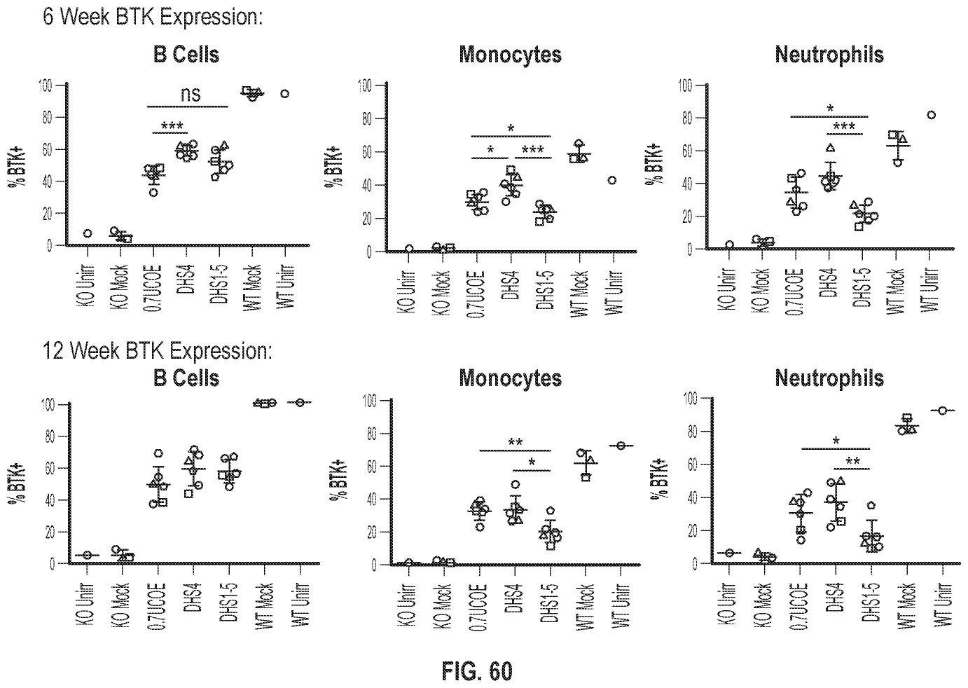

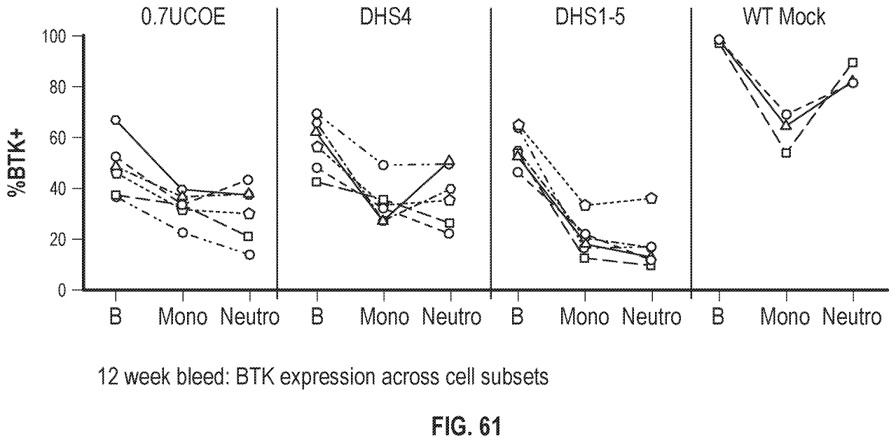

In another alternative, BTK expression in vivo was examined using the following vectors: 0.7UCOE.BTKp.coBTK, 0.7UCOE.DHS4.BTKp.coBTK and 0.7UCOE.DHS1-5.BTKp.coBTK. For the experiment, Lin-cells were harvested from TBK donor mice. For the transduction: a matched volume was achieved by 10 ul virus/million cells (target: matched input VCN of ˜3, as predicted by in vitro test) followed by a 16 hour transduction, 4×10e⁶ cells/ml in SCGM transduction media (mSCF, mTPO)+ polybrene. The cells were then transplanted in which 1.5×10e⁶ cells were administered per mouse (recipients: TBK, 900 rads irradiation) (FIG. 57). As shown in FIG. 57, cells transduced with the 0.6UCOE construct had a higher VCN; however the levels of BTK expression were similar to the lentiviral construct comprising the DHS4 element. Additionally, analysis of the peripheral blood lymphocyte distribution at 12 weeks showed that cell transduced with the lentiviral vectors 0.7UCOE, 0.7UCOE.DHS4 and 0.7UCOE.DHS1-4 had an increase in B cells indicating that the lentiviral vectors allowed expression of BTK thus leading to mature B cell development in the mice (FIG. 58). The 6 week peripheral blood lymphocyte distribution is shown in FIG. 59. As shown, the cells expressing BTK from 0.7UCOE, 0.7UCOE.DHS4 and 0.7UCOE.DHS1-4 had an increase in B cell production (FIG. 59). Additionally, BTK expression was shown in the B cells, monocytes and neutrophils at the evaluation at 6 weeks and 12 weeks in mice that were administered the cells comprising the 0.7UCOE, 0.7UCOE.DHS4 and 0.7UCOE.DHS1-4 lentiviral vectors (FIG. 60). At the 12 week bleed experiments, BTK was shown to be expressed across all the subsets (FIG. 61).

### Testing Alternative Human Codon Optimized BTK Constructs

Experiments were performed to examine the effect of human codon optimized BTK constructs on cellular expression of BTK. Human codon optimized BTK is described in Ng et al. (“Correction of B cell development in Btk-deficient mice using lentiviral vectors with codon-optimized human BTK.” Leukemia. 2010 September; 24(9):1617-30; incorporated by reference in its entirety).

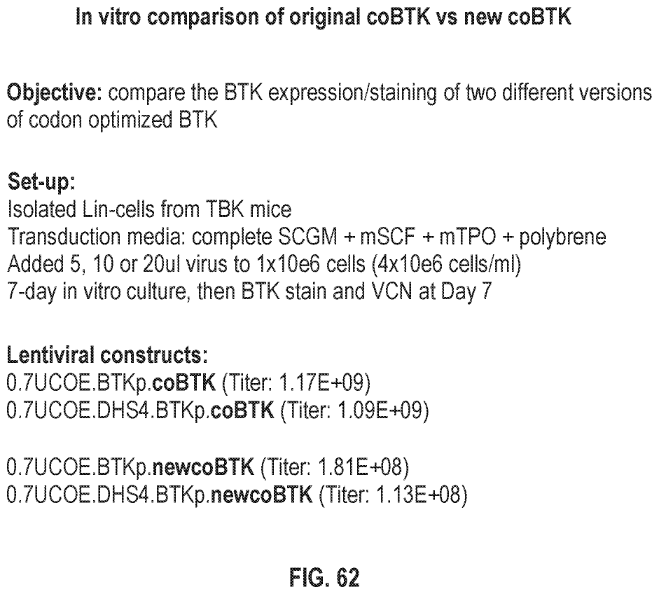

The objective of the following experiments was to compare the BTK expression/staining of two different versions of human codon optimized BTK. The set-up included: Isolated Lin-cells from TBK mice, Transduction media: complete SCGM+mSCF+mTPO+polybrene, addition of 5, 10, or 20 ul virus to 1×106 cells (4×106 cells/ml), 7-day in vitro culture, then BTK stain and VCN at Day 7. The lentiviral constructs were: 0.7UCOE.BTKp.coBTK (Titer: 1.17E⁺⁰⁹), 0.7UCOE.DHS4.BTKp.coBTK (Titer: 1.09E⁺⁰⁹), 0.7UCOE.BTKp.newcoBTK (Titer: 1.81E⁺⁰⁸), and 0.7UCOE.DHS4.BTKp.newcoBTK (Titer: 1.13E⁺⁰⁸) (FIG. 62).

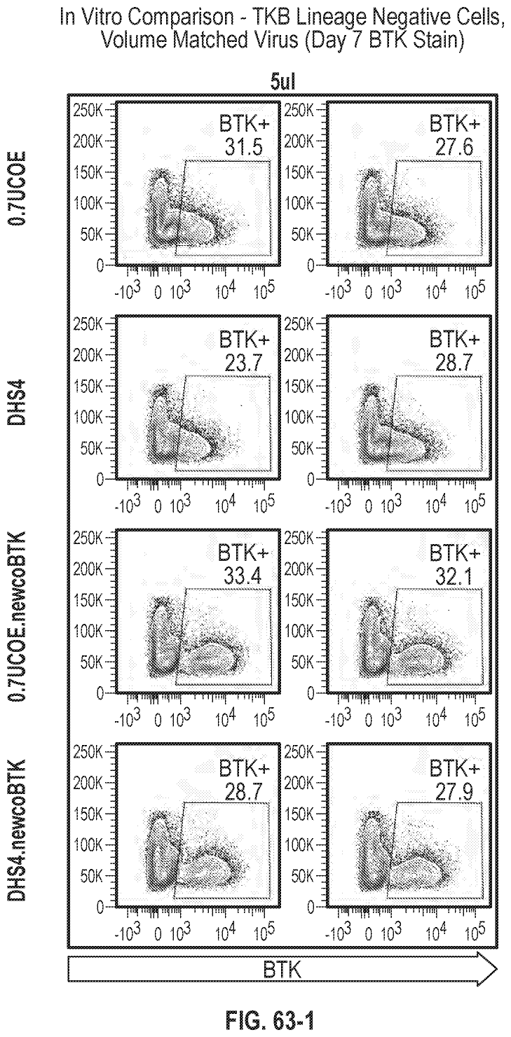

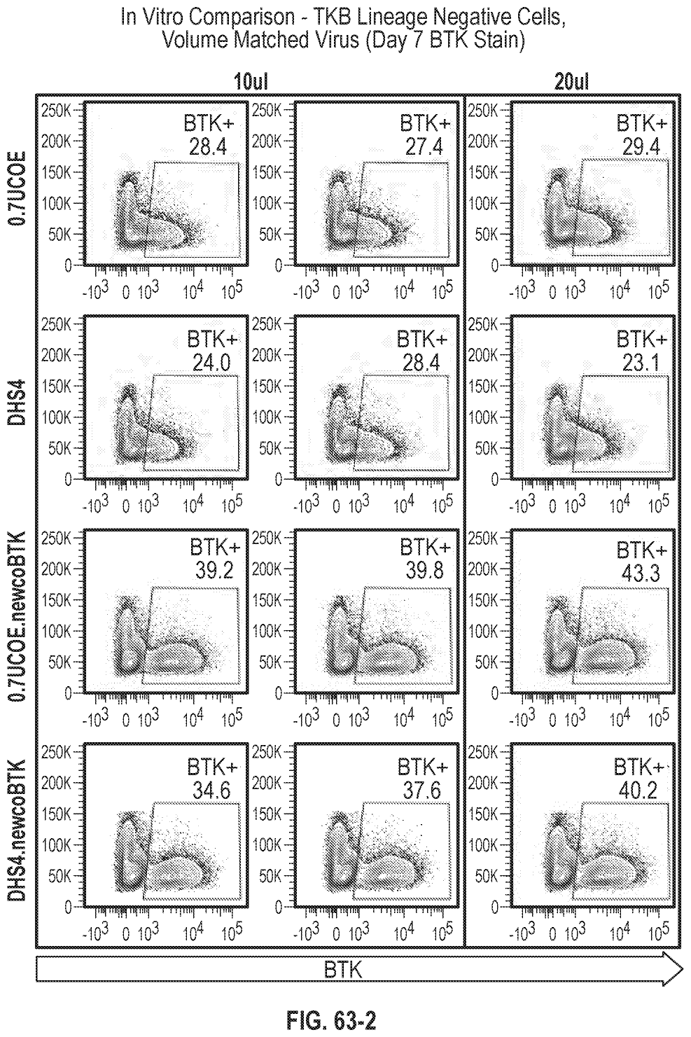

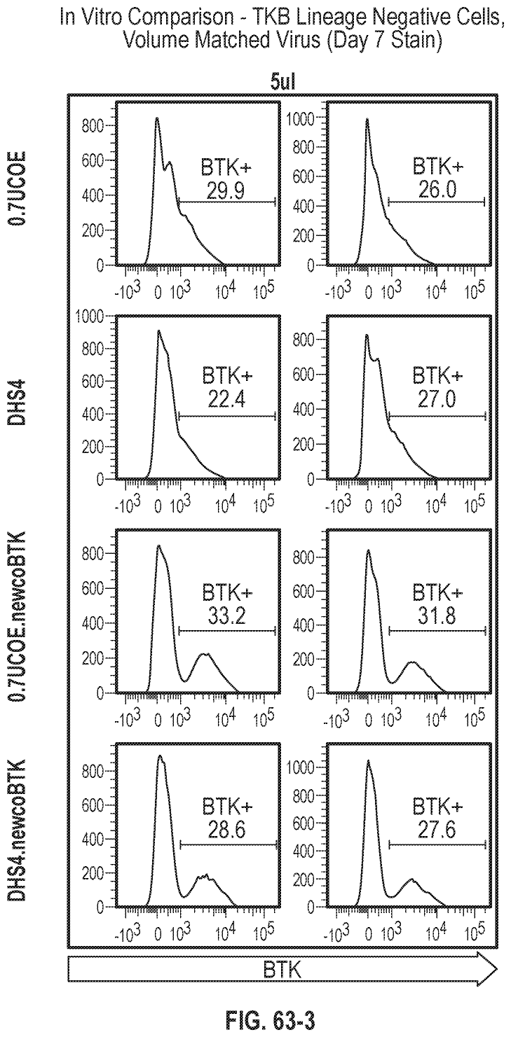

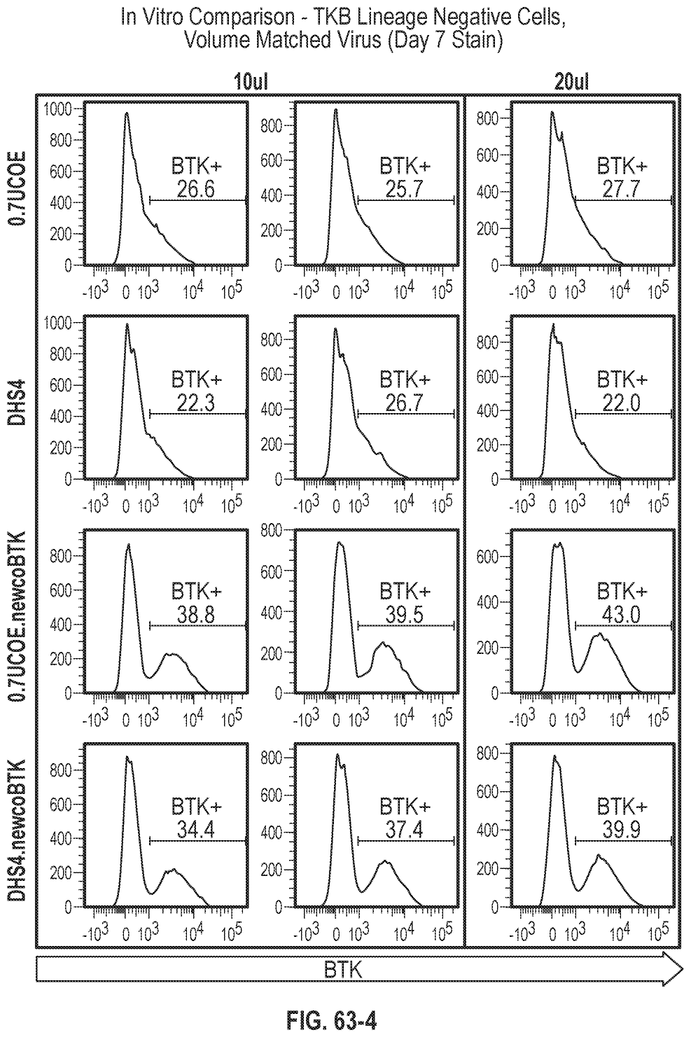

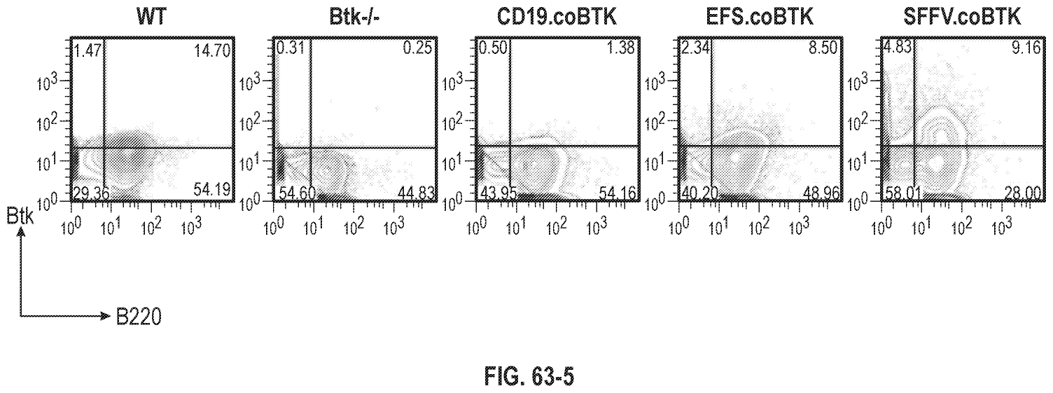

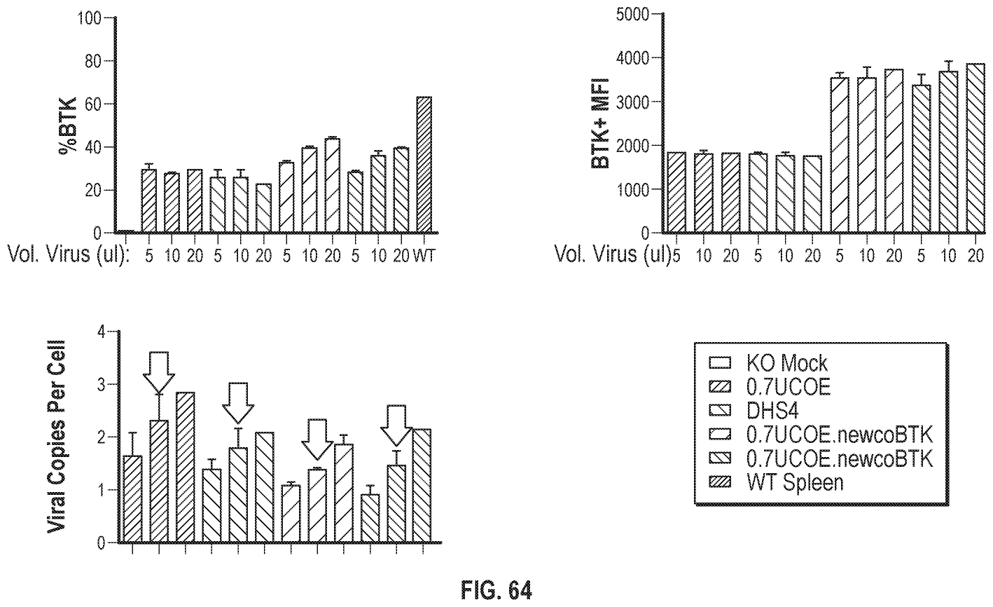

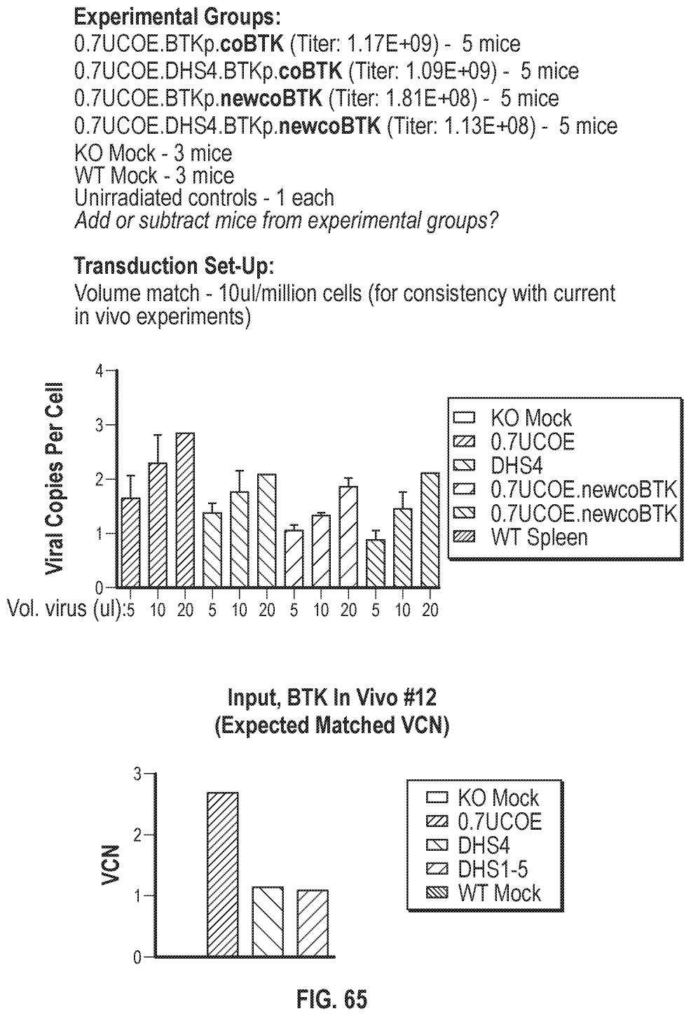

As shown in FIG. 63, BTK expression was increased in the cells transduced with the 0.7COE.newcoBTK and the DHS4.newcoBTK at 5, 10 and 20 uL of virus additions. Increased MFI was also shown for the cells that were transduced with 0.7COE.newcoBTK and the DHS4.newcoBTK at 5, 10 and 20 uL of virus additions (FIG. 64). The optimal viral copy number/cell was shown in cells that were transduced with 10 uL additions of both 0.7COE.newcoBTK and the DHS4.newcoBTK lentiviral vectors (FIG. 64).

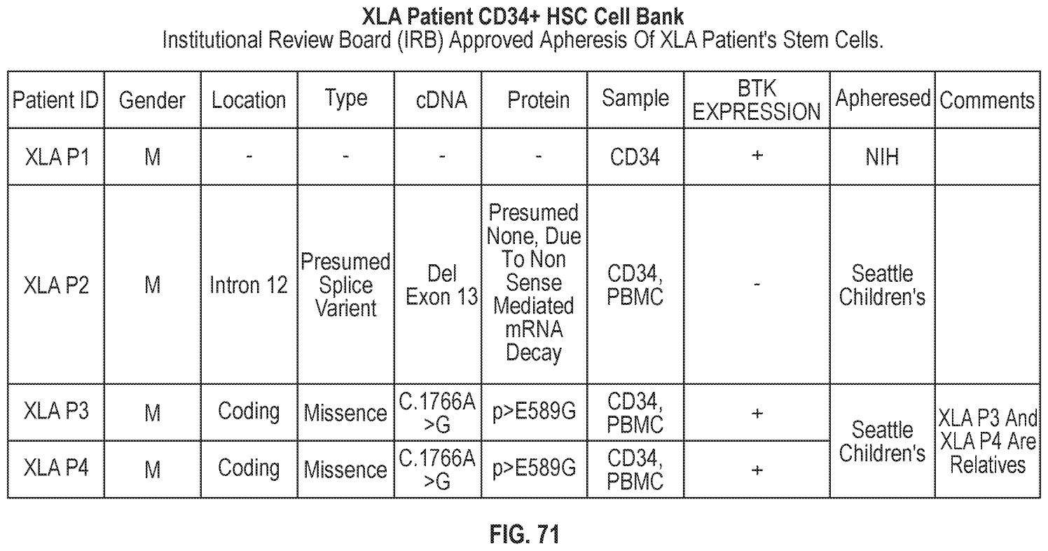

In vivo comparison of original vs new human codon optimized BTK is also contemplated. The experimental groups include: 0.7UCOE.BTKp.coBTK (Titer: 1.17E⁺⁰⁹) (5 mice), 0.7UCOE.DHS4.BTKp.coBTK (Titer: 1.09E⁺⁰⁹) (5 mice), 0.7UCOE.BTKp.newcoBTK (Titer: 1.81E+⁺⁰⁸) (5 mice), 0.7UCOE.DHS4.BTKp.newcoBTK (Titer: 1.13E⁺⁰⁸) (5 mice), KO Mock (3 mice), WT Mock (5 mice) and Unirradiated controls (1 mouse). Transduction set-up includes: Volume match with 10 ul/million cells (for consistency with current in vivo experiments) (FIG. 71).

Testing Alternative Orientations with the UCOE Elements in the Lentiviral Vector

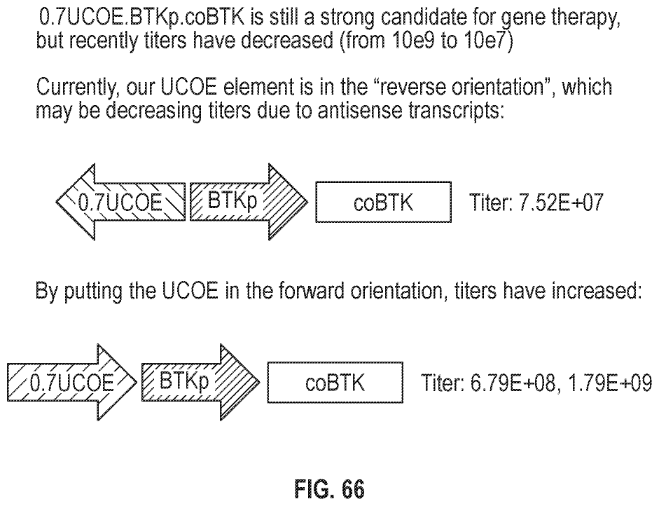

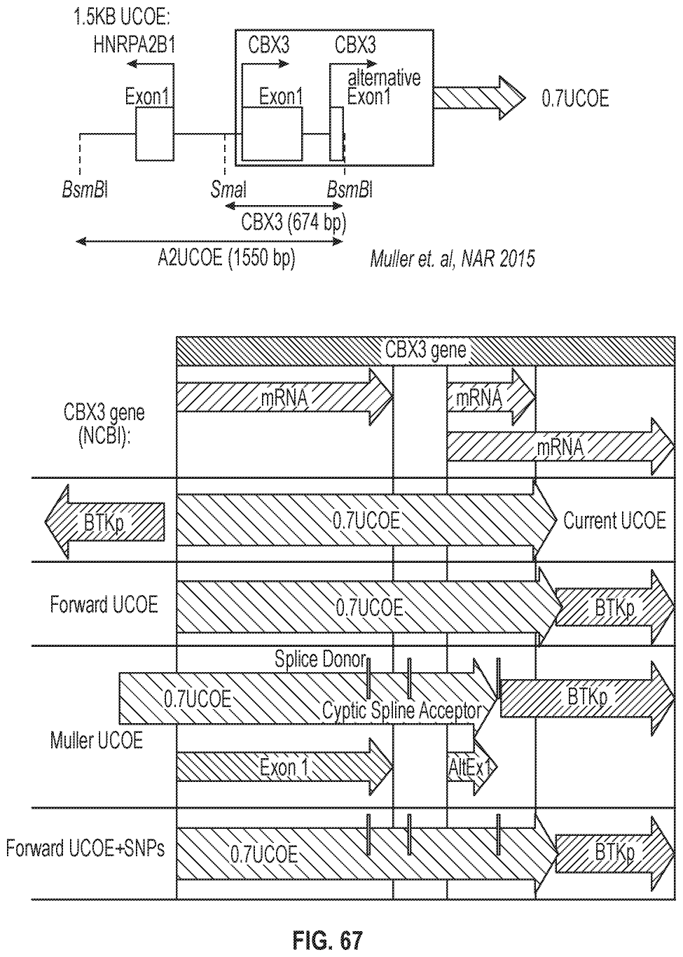

Experiments were performed to examine the effect of UCOE element orientation on cellular expression of BTK (FIG. 66). Constructs were manufactured as shown in FIG. 67, to evaluate whether the orientation of the UCOE would affect the expression of BTK.

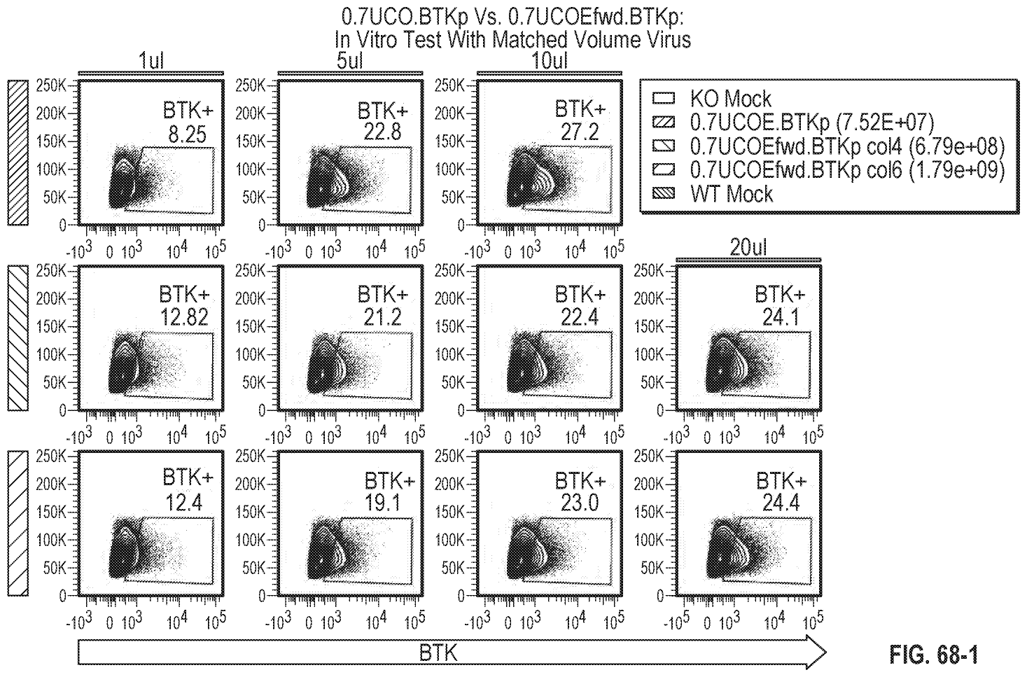

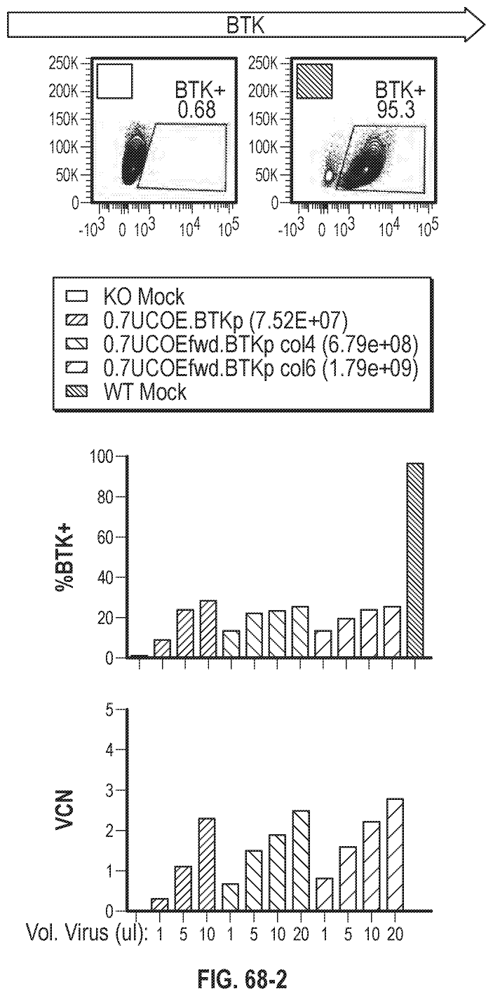

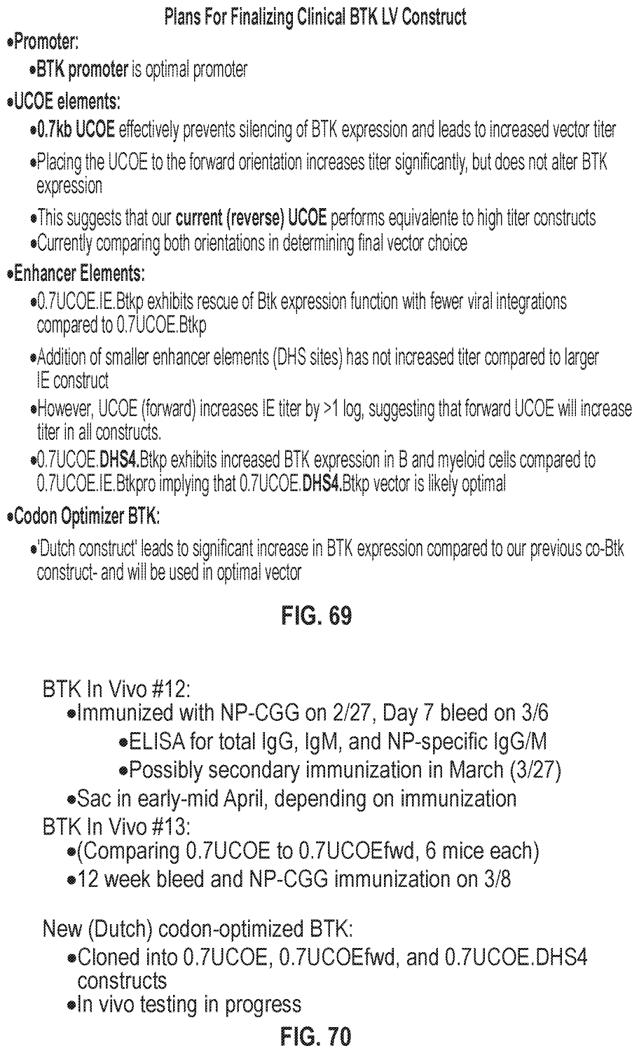

For the experiments, 4 lentiviral vectors were tested: KO mock, 0.7UCOE.BTKp (7.52E⁺⁰⁷), 0.7UCOEfwd.BTKp co14 (6.79E⁺⁰⁸), 0.7UCOEfwd.BTKp co16 (1.79E⁺⁰⁹), and WT mock. As shown in FIG. 68, the levels of BTK between the lentiviral vectors 0.7UCOE.BTKp (7.52E⁺⁰⁷), 0.7UCOEfwd.BTKp co14 (6.79E⁺⁰⁸), 0.7UCOEfwd.BTKp co16 (1.79E⁺⁰⁹) were similar, however VCN was increased at increased volume of virus.

As shown from the experiments, the BTK promoter is a robust promoter. In regards to the UCOE elements, 0.7 kb UCOE effectively prevents silencing of BTK expression and leads to increased vector titer. Furthermore, placing the UCOE in the forward orientation increases titer significantly, but does not alter BTK expression. This indicates that the reverse UCOE orientation performs equivalent to high titer constructs. In regards to enhancer elements, it was shown that 0.7UCOE.IE.Btkp exhibits rescue of BTK expression function with fewer viral integrations compared to 0.7UCOE.Btkp and the addition of smaller enhancer elements (DHS sites) has not increased titer compared to larger IE construct.

UCOE in the forward orientation increases IE titer by >1 log indicating that the forward orientation of the UCOE will increase titer in all constructs.

For example, it was shown that 0.7UCOE.DHS4.Btkp exhibits increased BTK expression in B and myeloid cells as compared to 0.7UCOE.IE.Btkpro indicating that 0.7UCOE.DHS4.Btkp vector is a particularly robust vector.

In regards to the human codon optimized BTK, the new construct leads to a significant increase in BTK expression as compared to the previous co-Btk construct.

### Testing IgG, IgM and NP Specific Ig/M Human HSC Studies-Control and XLA Subjects:

The rescue of B cell development and function of XLA cells in vivo were examined using a lentiviral vector. The experiments were performed to evaluate whether it would be feasible to recapitulate XLA patient's B cell phenotype in NSG mice. Also contemplated was whether the methods would rescue the B cell development in vivo by transducing XLA stem cells with the clinical LV vector. Shown in FIG. 71 FIG. 71 is a table of XLA patients selected to receive therapy.

The XLA B cell phenotype in the periphery include a markedly reduced percentage of B cells, higher percentage of transition/immature B cells (CD38+ CD24+ CD10 high), and a lack of mature B cells (CD38− CD24− CD10^{low}).

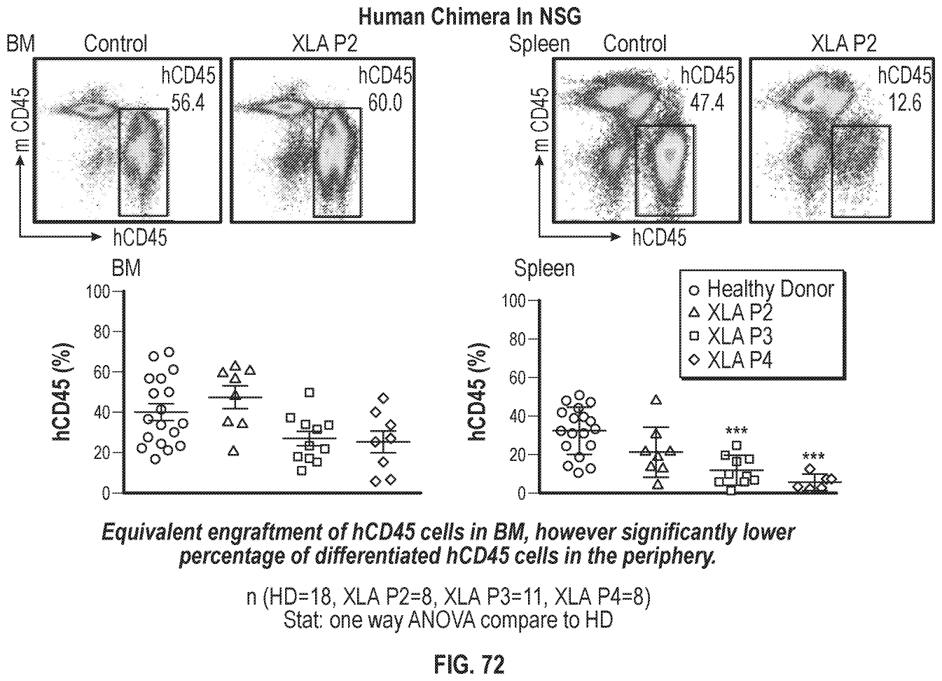

The human chimera in NSG was shown in patient XLA P2, P3 and P4 in FIG. 72. As shown, there was an equivalent engraftment of hCD45 cells in bone marrow, however, a significantly lower percentage of differentiated hCD45 cells were in the periphery.

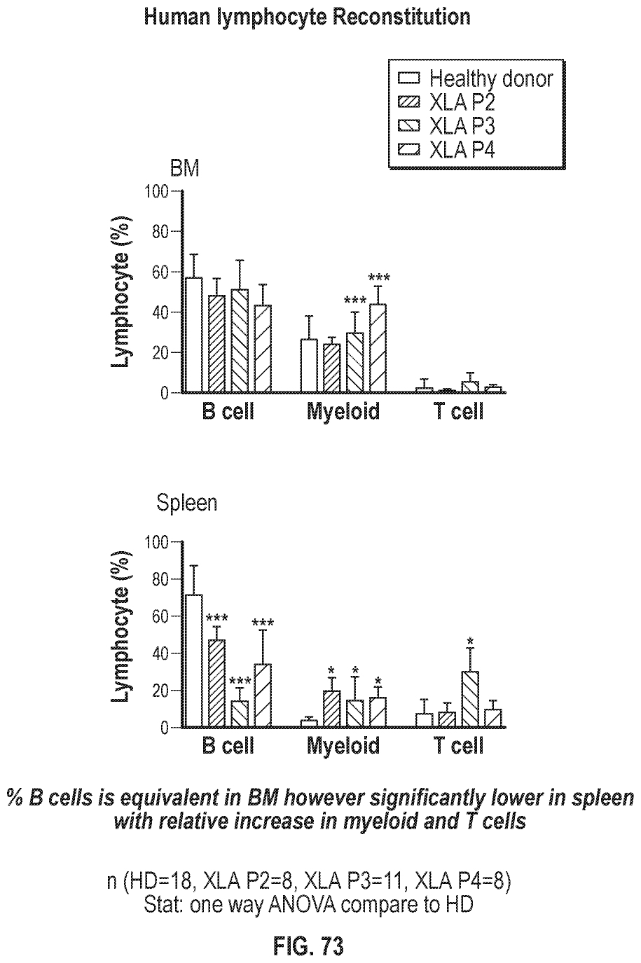

For the human lymphocyte reconstitution, the percentage of B cells was equivalent in bone marrow, however, a significantly lower percentage in spleen with a relative increase in myeloid and T cells was seen in all XLA patients P2, P3 and P4 (FIG. 73).

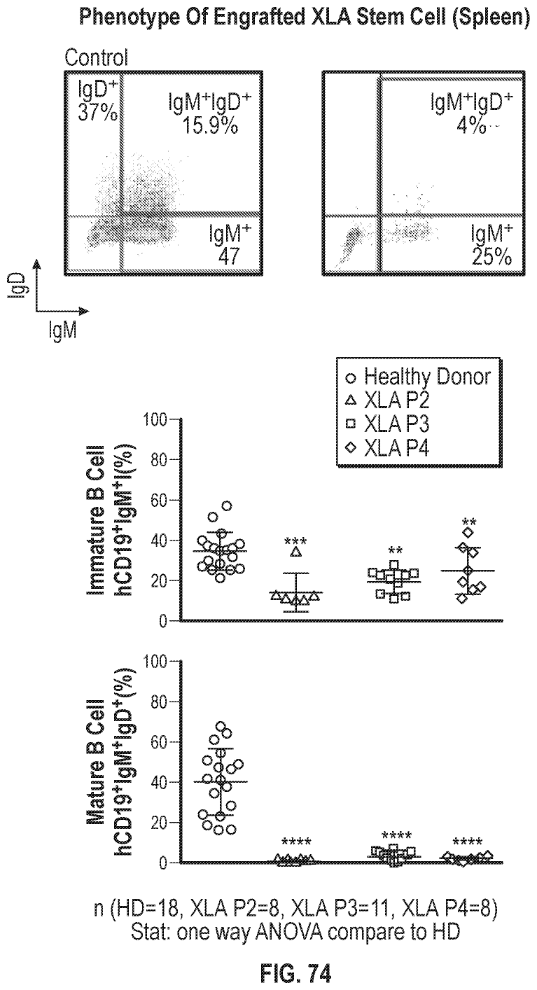

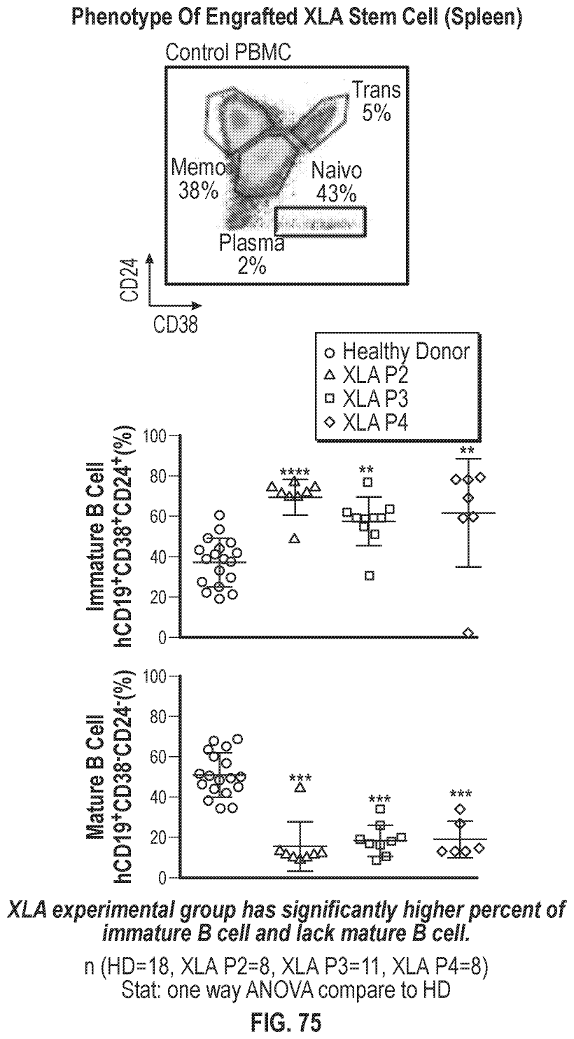

The phenotype of the engrafted XLA stem cell (spleen) is shown in FIG. 74 for patients XLA P2, P3 and P4. As shown, the patients exhibited low levels of mature B cells; however, the patients had similar levels of immature B cells (FIG. 75).

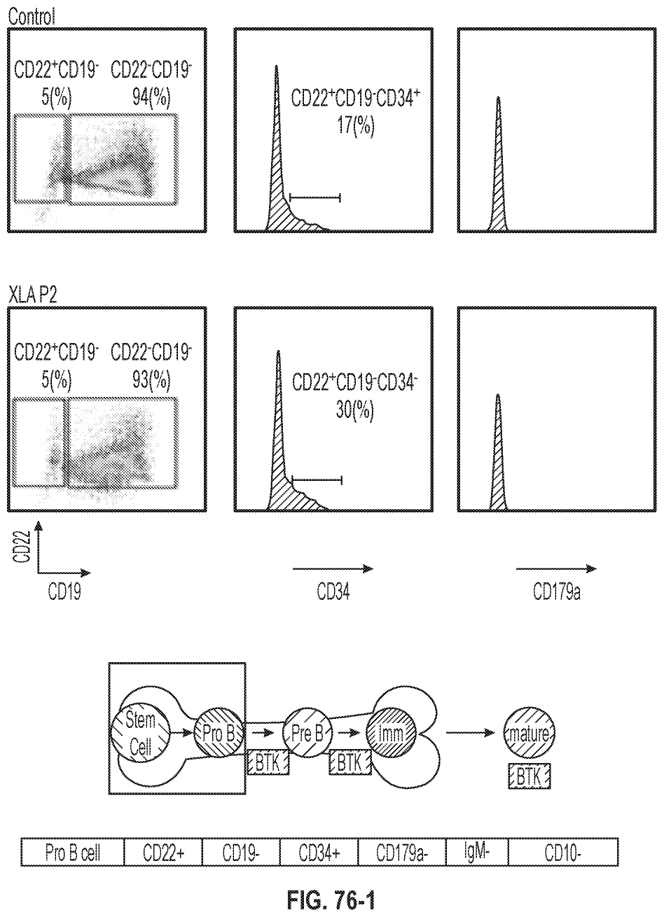

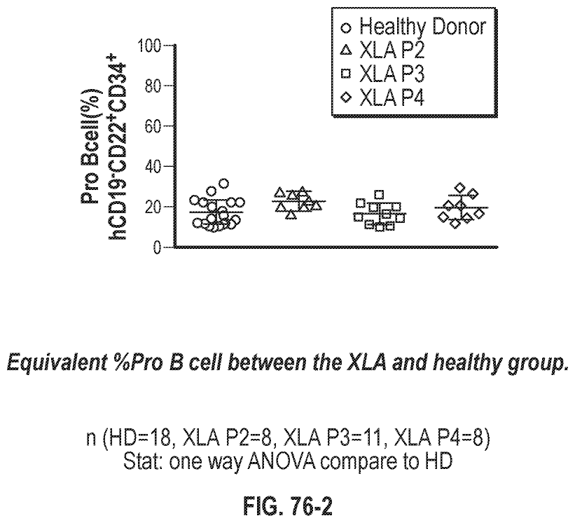

The phenotype of the engrafted XLA stem cell (spleen) is shown in FIG. 76 for patients XLA P2, P3 and P4. As shown, the patients exhibited equivalent % Pro B cell between the XLA and healthy group.

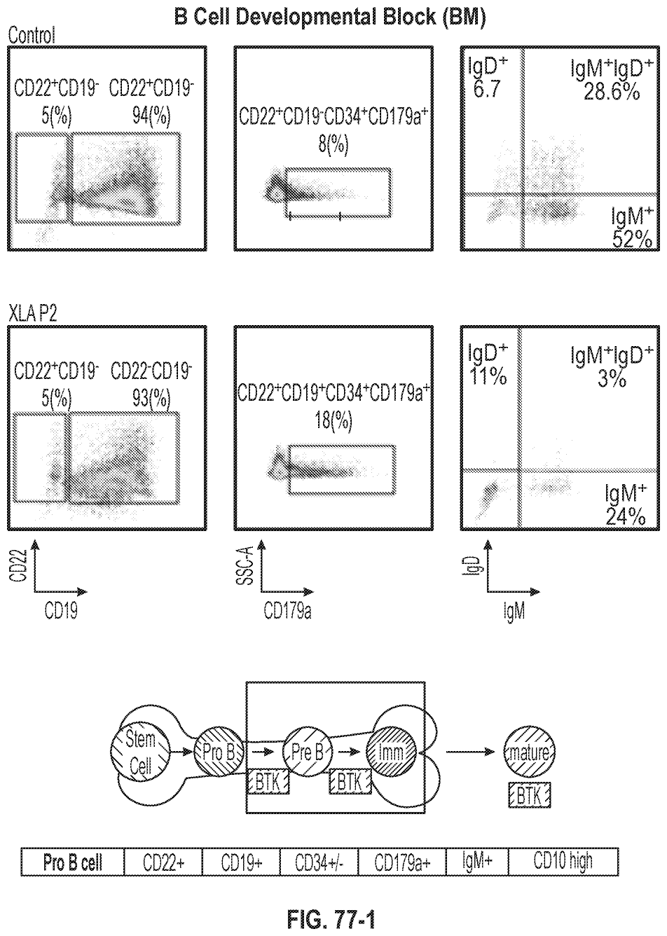

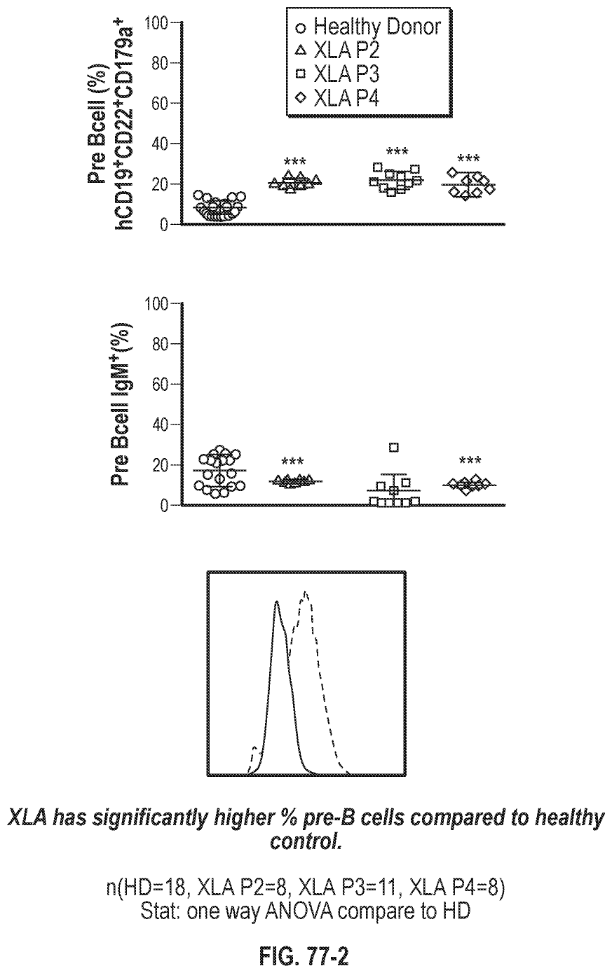

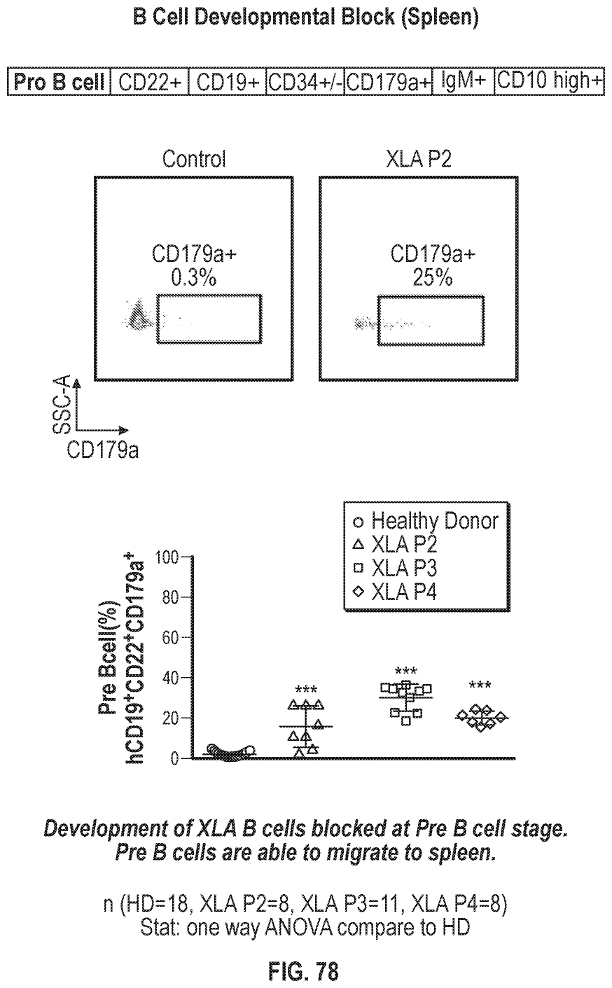

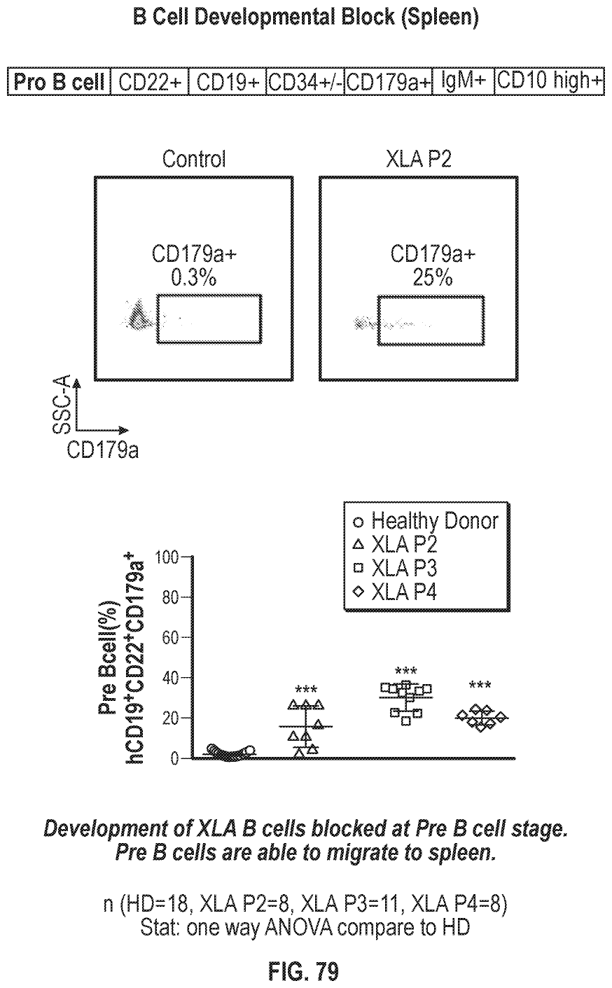

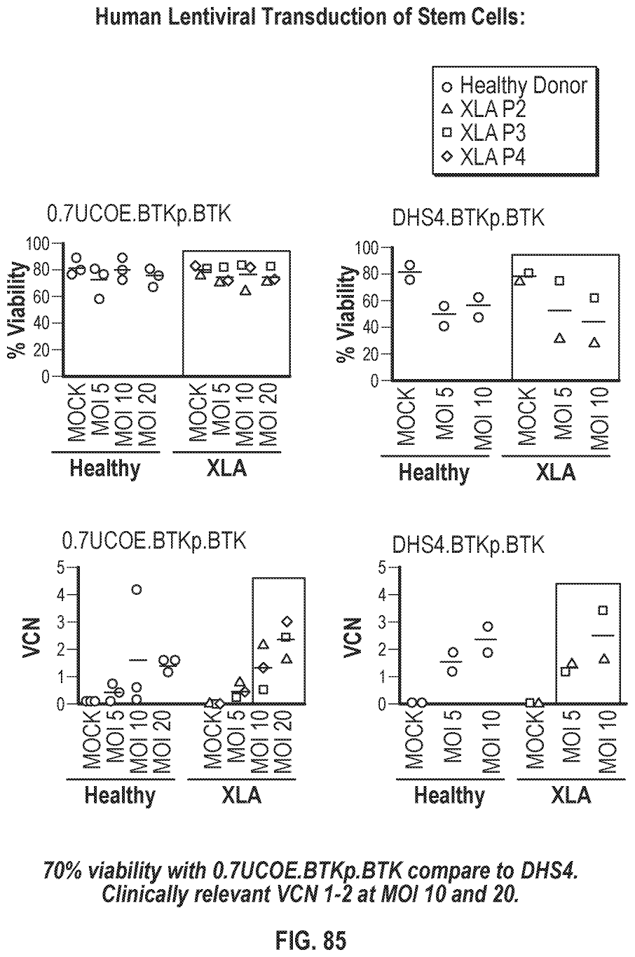

The B cell developmental block was examined for all XLA patients. As shown in FIG. 77, BTK allows development of Pro B cell to a mature B cell. The cells were analyzed for hCD19+, CD22+ and CD179a+. As shown, the XLA patients had a significantly higher percentage of pre-B cells as compared to the healthy control. These XLA B cells are thus blocked at the Pre B cell stage in which they are then able to migrate to the spleen (FIGS. 78 and 85).

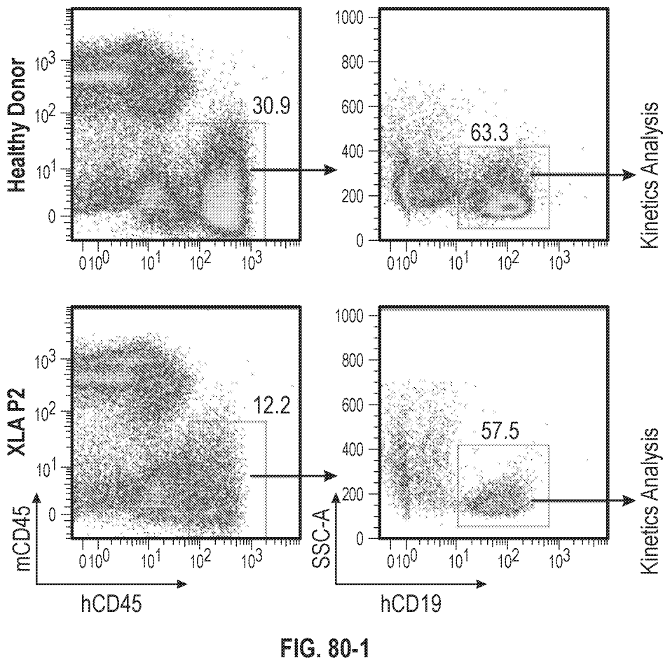

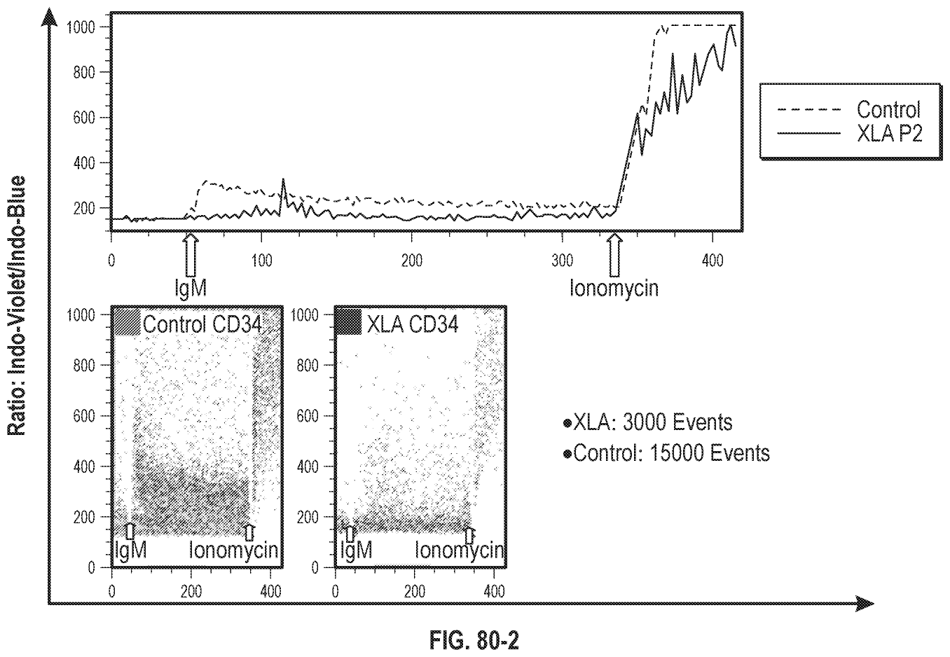

The B cell function by Ca²⁺ was also examined in the XLA cells (FIG. 80. As shown, the XLA B cells were not able to flux with IgM as compared to the control.

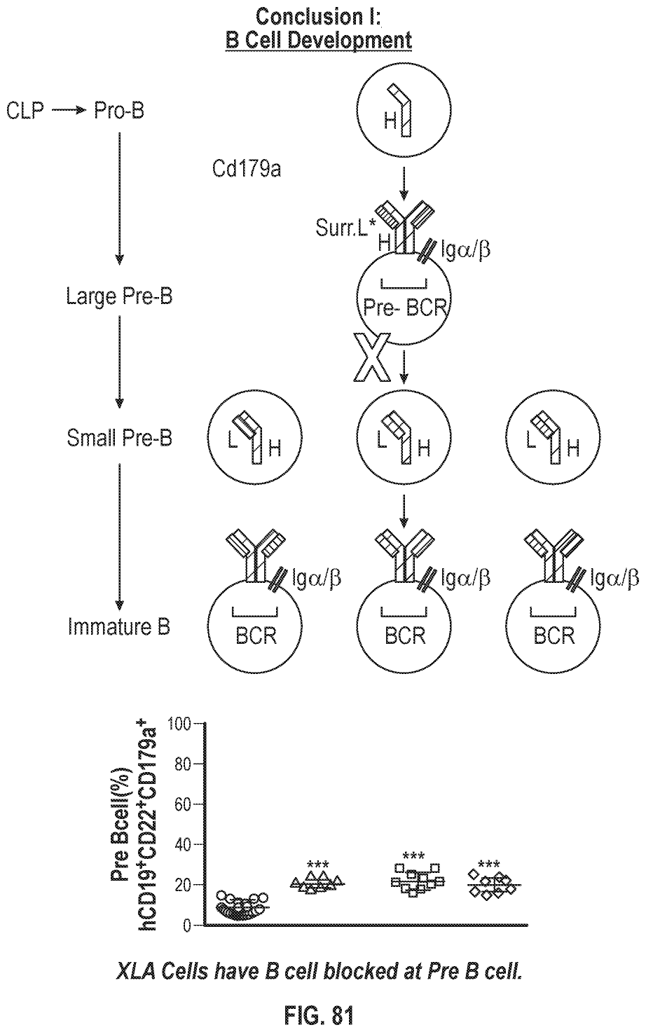

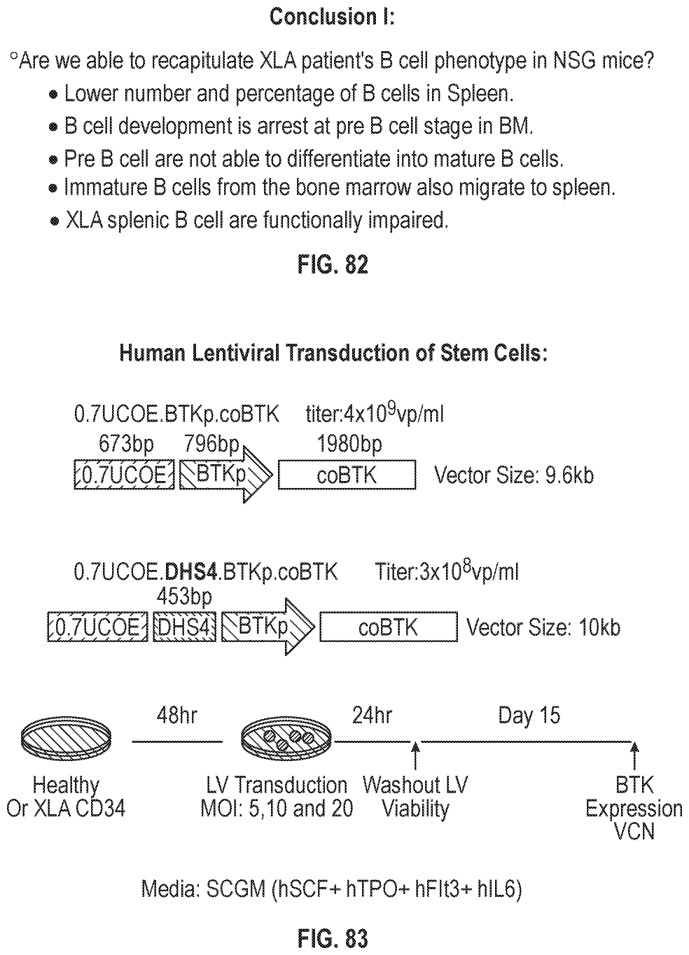

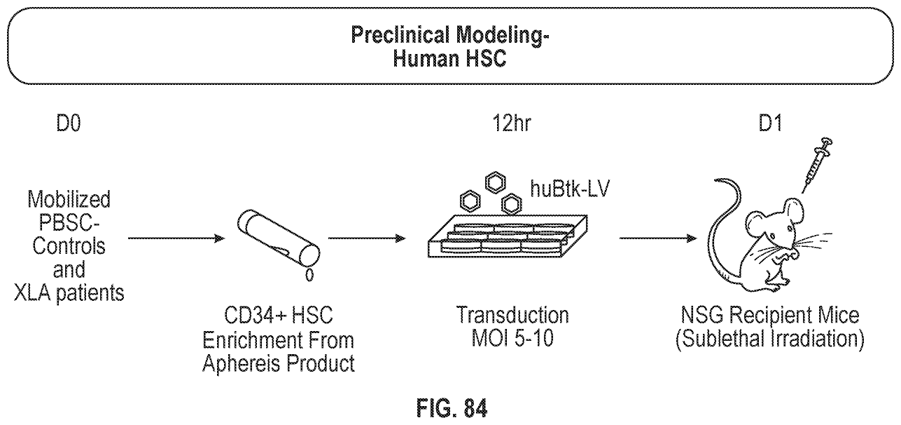

From the evaluation of the XLA B cells, it was shown the XLA cells have B cell development blocked at the Pre B cell stage (FIG. 81).

As shown, XLA patients have a lower number and percentage of B cells in spleen. Additionally, the B cell development is arrested at the pre-B cell stage in the bone marrow. The next step was to recapitulate the XLA patient's B cell phenotype in NSG mice to examine the effects of the BTK expressing lentiviral vector system.

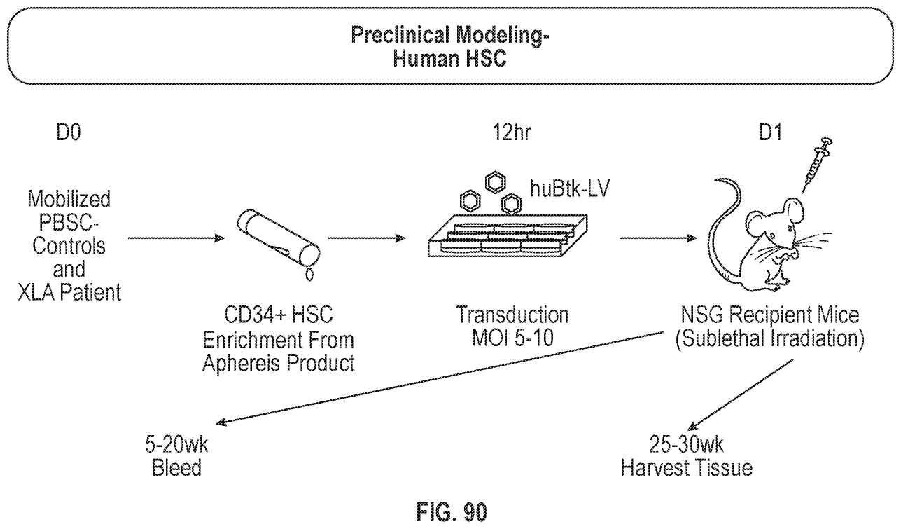

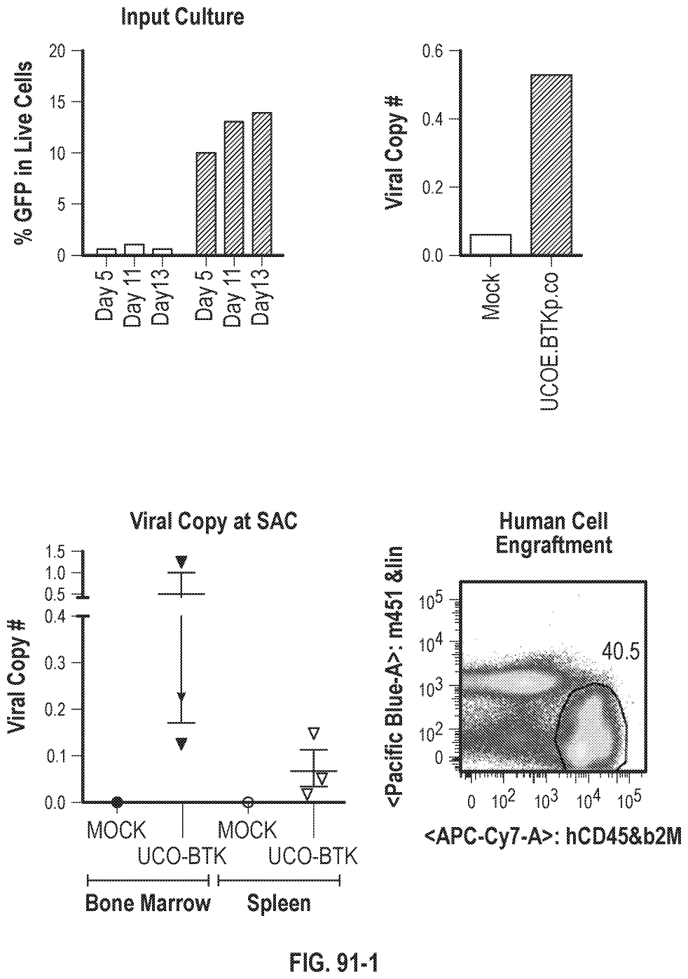

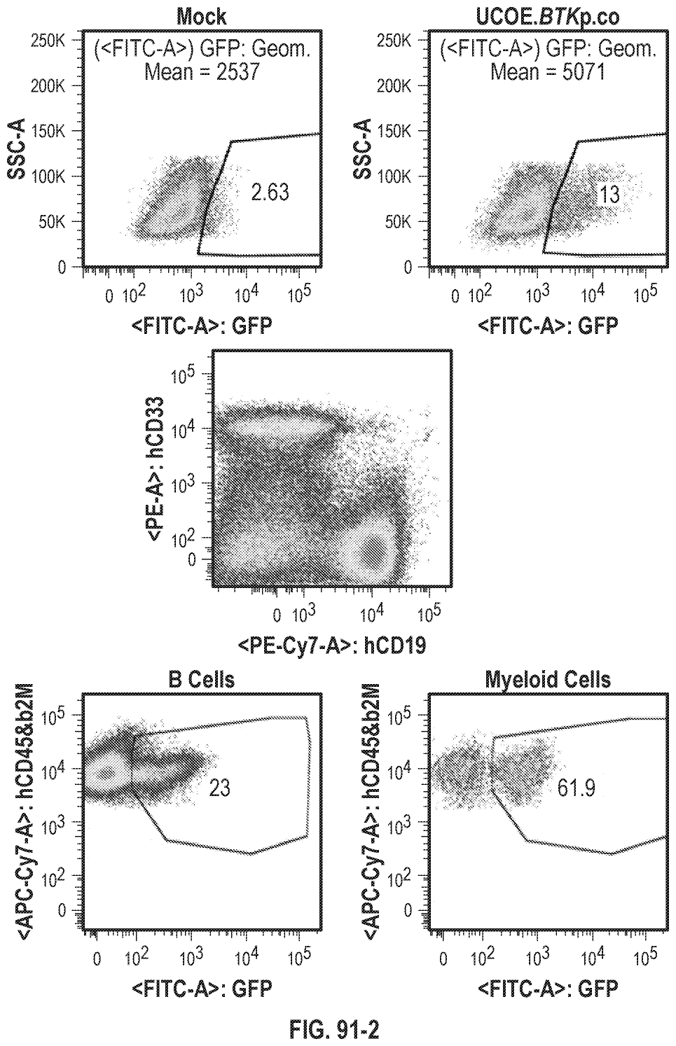

Human lentiviral transduction of stem cells was performed with the following vectors: 0.7 UCOE.BTKp.coBTK and 0.7UCOE.DHS4.BTKp.coBTK according to the methods shown in FIGS. 83 and 90.

The human lentiviral transduction of the stem cells led to 70% viability with the 0.7UCOEBTKp.BTK, as compared to the vector with the DNAse hypersensitivity site 4 region (FIG. 85).

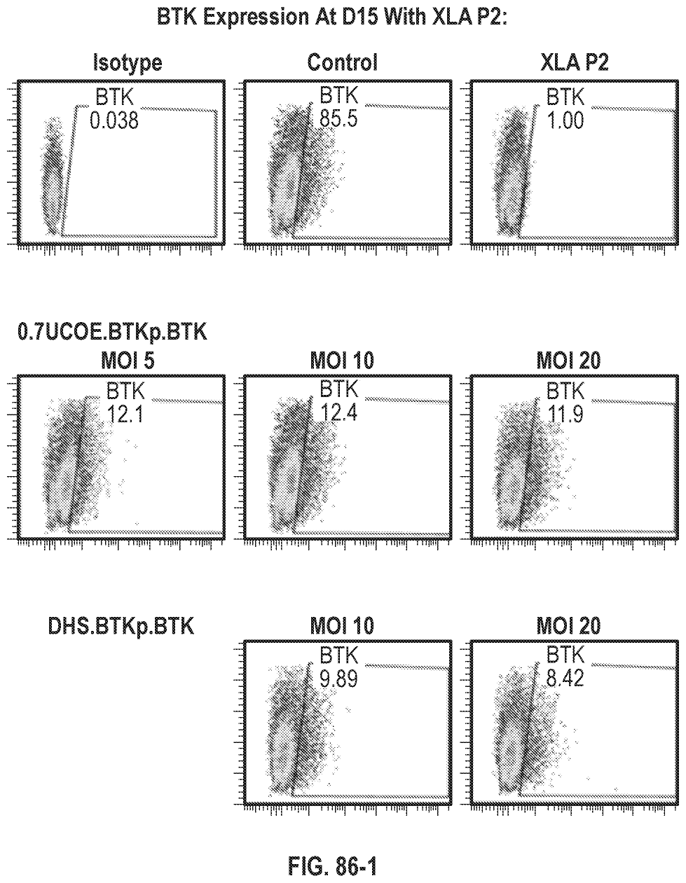

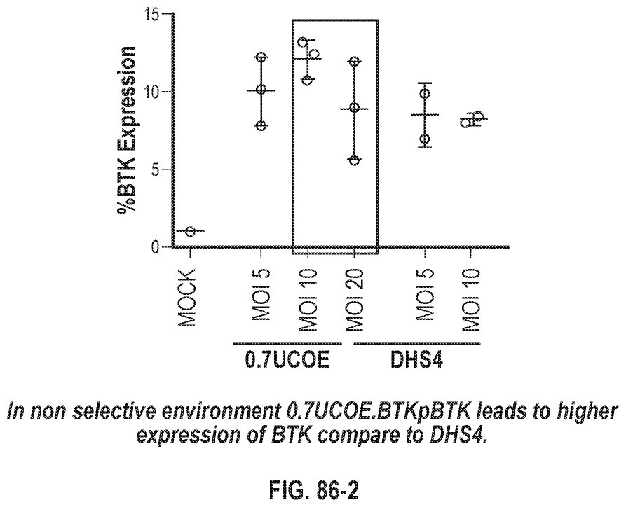

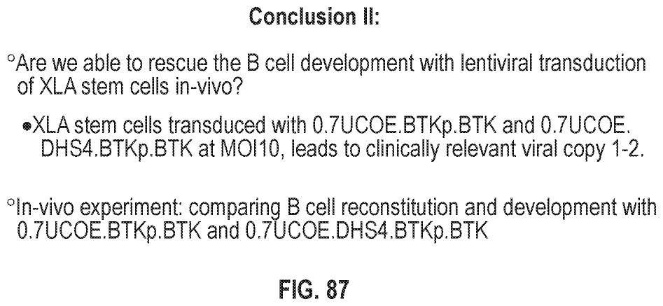

BTK expression was also seen in the cells derived from patient XLA P2 transduced with the 0.7UCOE.BTK.pBTK lentiviral vector. As shown in a non-selective environment, 0.7UCOE.BTKpBK leads to a higher expression of BTK, as compared to the lentiviral vector comprising the DNAse hypersensitivity site 4 region (FIG. 86).

In view of the above experimentation, it was concluded that the XLA stem cells transduced with 0.7UCOE.BTKp.BTK and 0.7UCOE. DHS4.BTKp.BTK at MOI10, leads to clinically relevant viral copy 1-2. As such, it is contemplated that lentiviral transduction of XLA stem cells in vivo will lead to rescue of B cell development.

### Preclinical Modeling of Human HSC

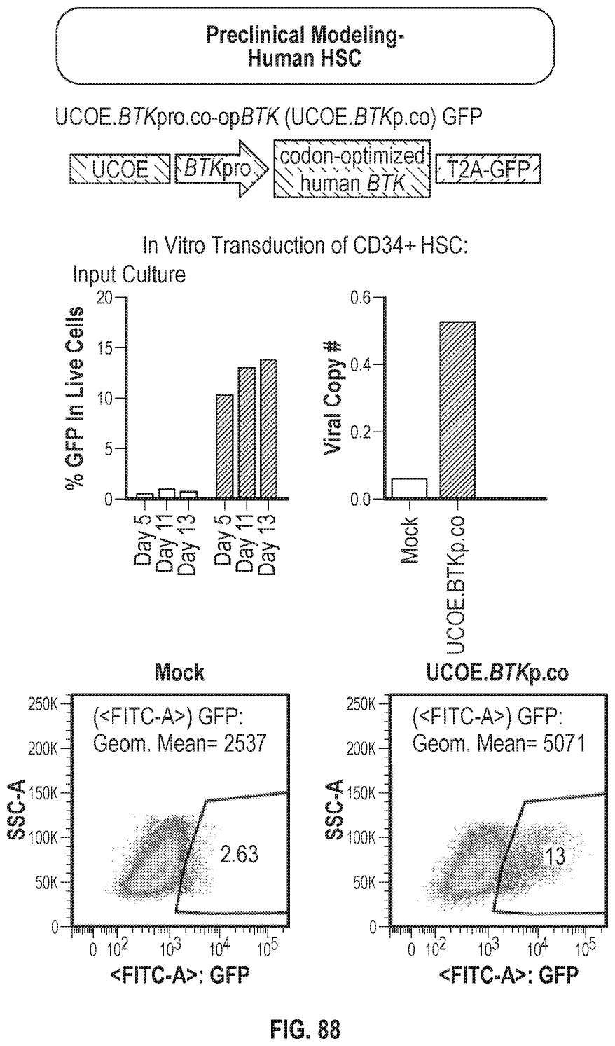

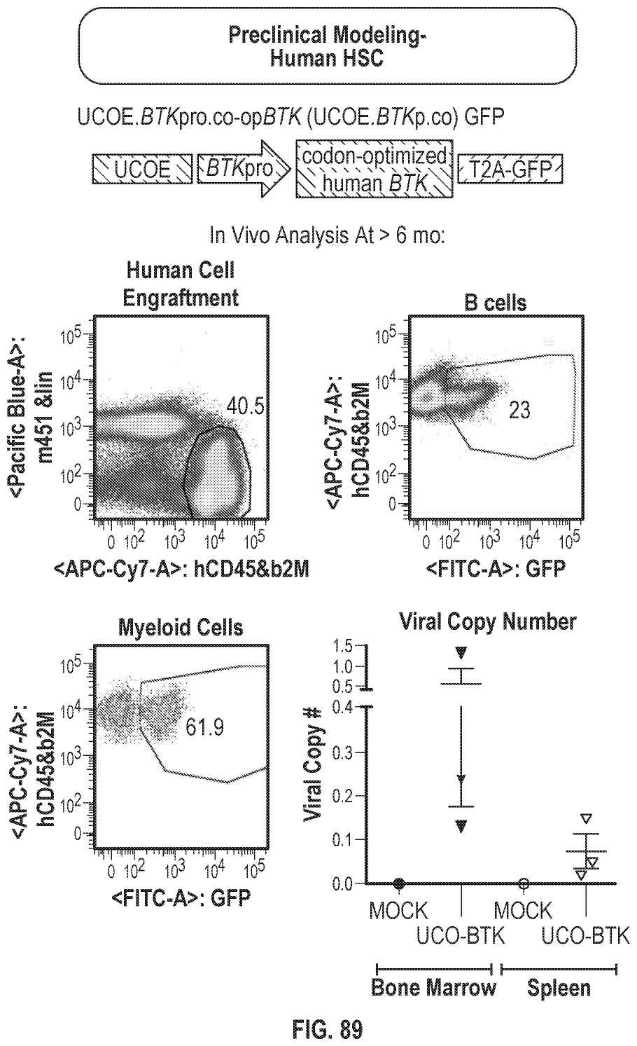

The preclinical modeling of human HSC was examined using a lentiviral vector expressing human codon optimized BTK fused to a GFP protein, which was self-cleavable with a T2A linker (See FIG. 88). As shown, the test construct comprises the UCOE element, a BTK promoter, a human codon optimized BTK gene, as well as, the T2A-GFP marker protein. In vitro transduction of the CD34+ HSC indicated an increase of GFP in the cells transduced with the lentivector (FIG. 88). Cells were then administered to NSG recipient mice as shown in FIG. 90. In vivo analysis after 6 months showed that there was an increase in B cells, as well as, myeloid cells (FIGS. 89 and 97).

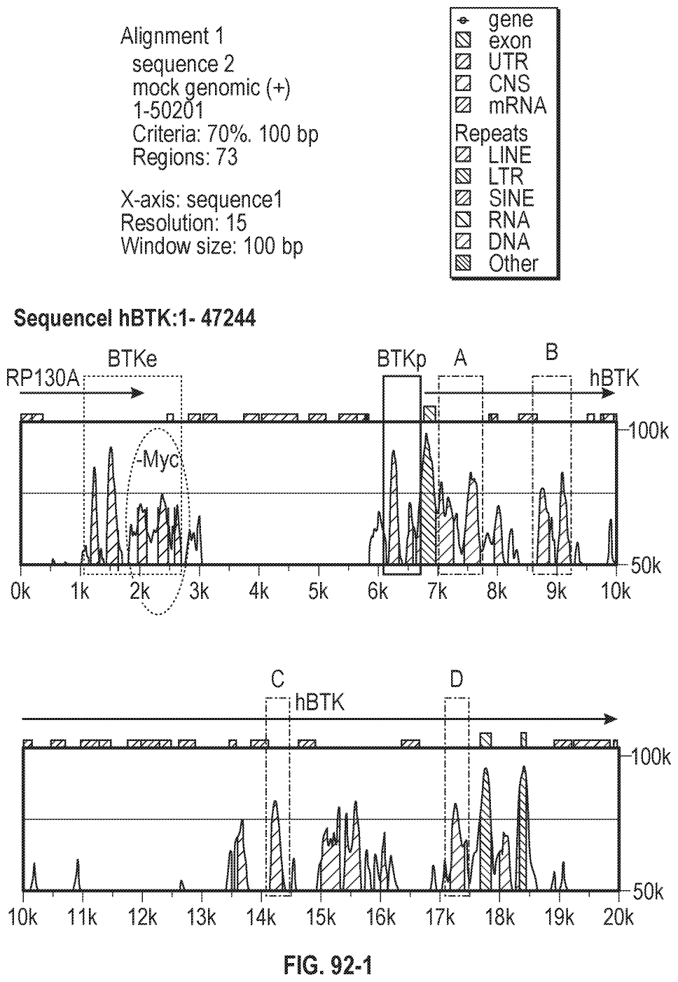

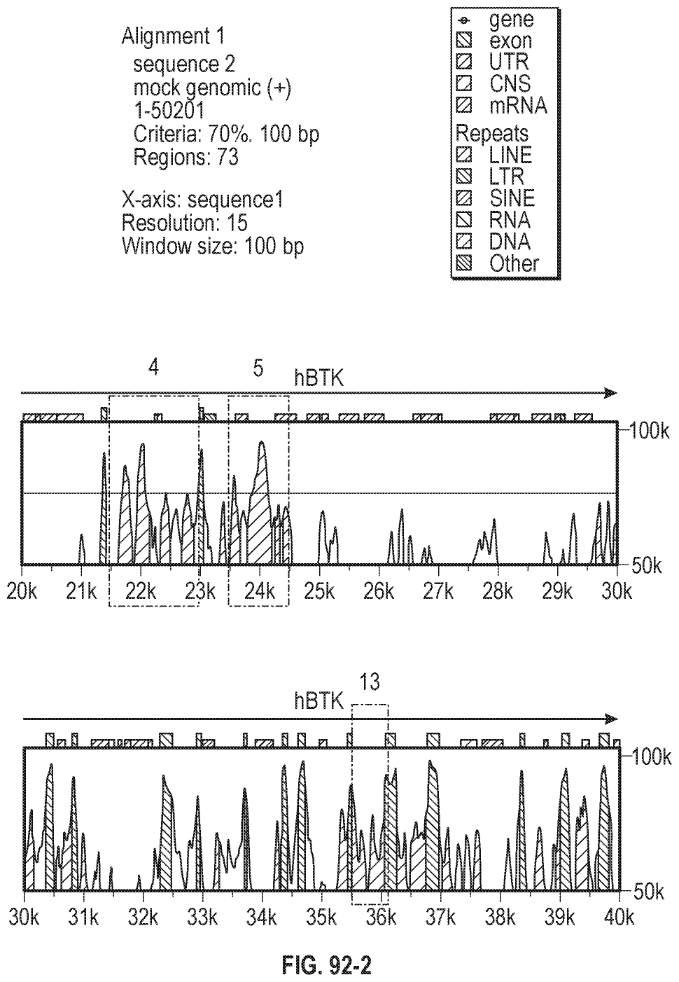

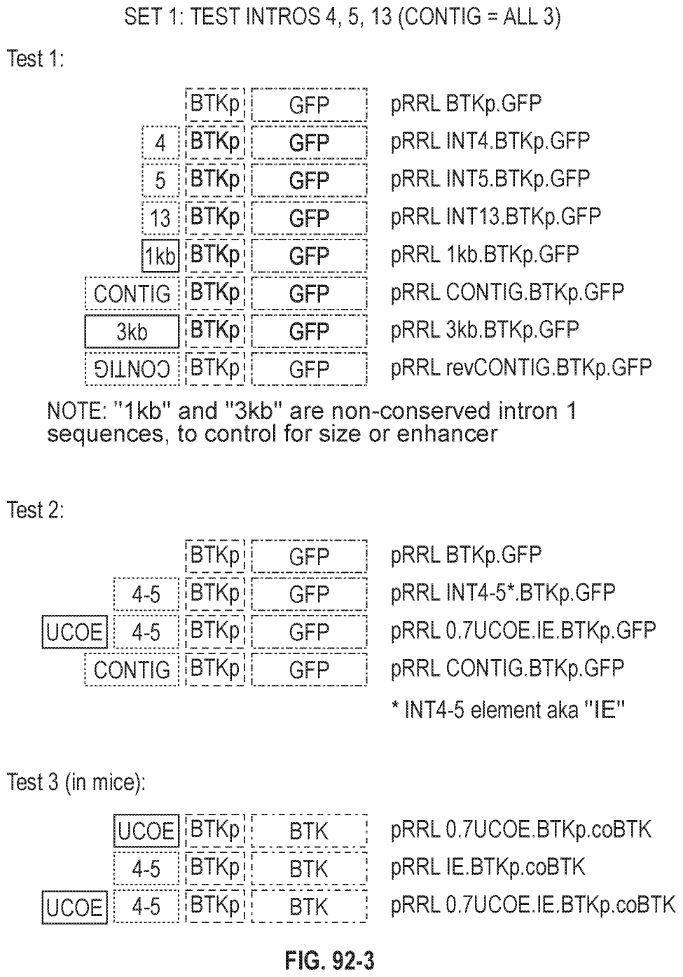

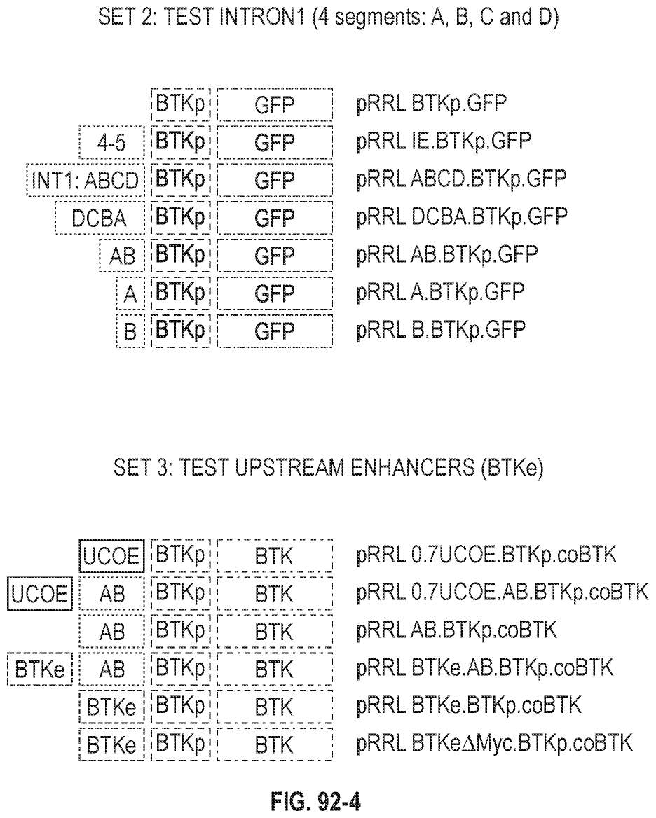

Further tests are contemplated with constructs as shown in FIG. 92. A bioinformatics approach was undertaken utilizing the ENCODE genome-wide database information on regulatory markers in specific cell lineages. The flow of the discovery was the use of conserved non-coding sequence information to identify candidate enhancers

In effort to improve tissue specific BTK expression, conserved non-coding sequences were identified by comparing human vs. mouse non-coding sequences and identifying areas that are highly conserved. Conserved non-coding sequences were cloned in front of BTKp and see if they improve the expression in conjunction with the BTK promoter (BTKp). The first pass read-out used GFP reporter to track expression-improved expression would increase the GFP signal or “MFI”. Introns 4 and 5 were identified as improving MFI, as well as a contig of introns 4, 5, and 13. GFP was encoded by the sequence set forth in SEQ ID NO: 8. Sequences that are shown in Set 1 of FIG. 92 are in SEQ ID NO's 23-30. The tested sequences that encoded the GFP are set forth in SEQ ID NO's: 31, 32, and 36-40.

The contig containing introns 4, 5 and 13 vs. a contig with introns 4 and 5 only were next tested again in vitro with GFP. The Intron 4,5 together had better MFI.

Intron 4 and 5 contig were tested in vivo in mouse gene therapy model, calling it intronic enhancer 4, 5 or IE4-5. These studies used the enhancer/promoter elements to drive expression of a codon-optimized human BTK coding sequence, and showed that inclusion of IE4-5 improved expression of BTK per viral copy number.

The polypeptides that encode the sequences for the BTK for expression are set forth in SEQ ID NO's 33-35, and 41-45.

Refining Candidate Enhancers Using Information from Encode Database

To more narrowly define DNA elements mediating enhancer activities in IE4-5, the ENCODE database was used to find DNase hypersensitive sites located in these introns, and one was found in each intron that were both in B cells and in myeloid cells, but not always present in non-relevant tissues. These were called DHS 4 and 5, and were 725 bp and 1077 bp less than the sizes of the previous intronic regions that were included. There were also DNase hypersensitive sites in B and myeloid cells identified within intron 1 (DHS1, 2, and 3) that were also for testing, due to some evidence that intron 1 may improve MFI in vitro. DHS 3 and 4 were identified as having properties of transcriptional enhancers in B cells by the ENCODE segmentation analysis; DHS5 was identified as a CTCF binding site/candidate insulator.

Various combinations of DHS sites 1 through 5 were also tested.

As shown in FIG. 92, “1 kb” and “3 kb” are non-conserved intron 1 sequences to control for the size of the enhancers.

### Truncation of 1.5 kb UCOE to 0.7 kb and Identification of Potential DNA Enhancer Elements May Improve Titer and BTK Expression in Lentiviral Constructs.

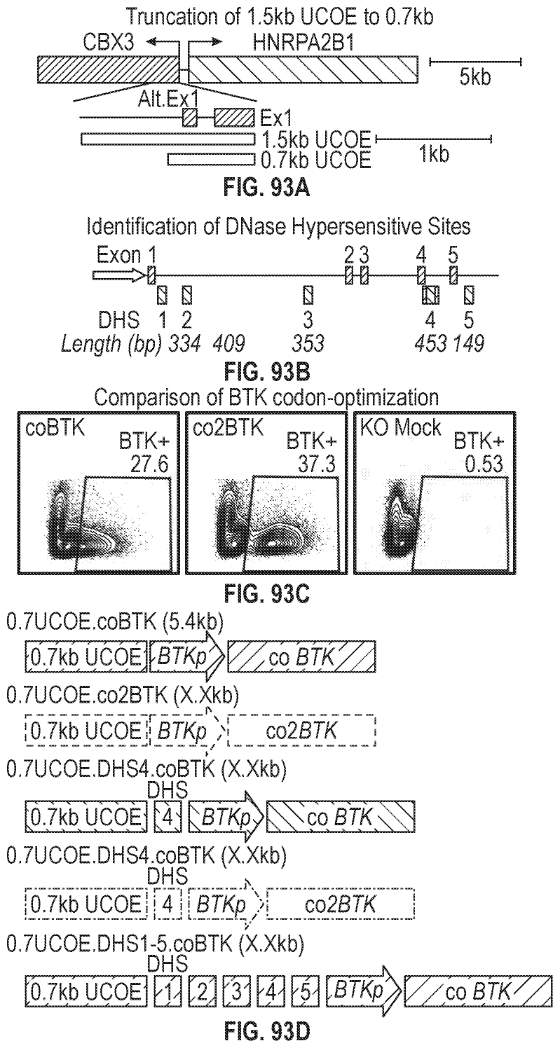

Shown in FIG. 93A, are the results from truncation of 1.5 kb Ubiquitous

Chromatin Opening Element (UCOE) to 0.7 kb. The UCOE element spans a large, CpG-rich region across the divergently transcribed promoter regions for the housekeeping genes CBX3 and HNRPA2B1, and has traditionally been truncated to a 1.5-2.2kb region by various groups for use in protein expression constructs. The 1.5 kb UCOE used here starts at Exon 1 of CBX3 and spans past CBX3 Alternate Exon 1. Truncation of this region to 0.7 kb eliminates the region downstream of Alt. Ex. 1. (93B) DNaseI Hypersensitive sites (DHS) from intronic regions of the BTK gene were identified from the ENCODE database and visualized using the UCSC Genome Browser on Human Feb. 2009 (GRCh37/hg19) Assembly. Five DHS were identified, and were labeled consecutively as DHS1, DHS2, DHS3, DHS4, and DHS5 (blue boxes). ENCODE Genome Segmentation identified a predicted Enhancer element around DHS4 (yellow box). Exons are shown as black boxes. The sequence length is noted beneath each DHS. Various combinations of these DHS sequences were cloned into the 0.7UCOE.BTKp.coBTK construct and tested in vitro as follows (data not shown). Murine Btk^{-/-}Tec^{-/-} bone marrow cells were transduced with LV containing the various expression cassettes shown below. Mean fluorescence intensity of transgene expression was then compared by flow cytometry. (93C) An in vitro transduction experiment of murine (TBK) lineage negative cells was performed to compare BTK expression levels in two versions of codon-optimized BTK: coBTK (FIGS. 1-2), and a codon-optimized BTK published by Staal et al. (Leukemia 2010), denoted here as co2BTK. Representative flow plots show BTK expression at 7 days post-transduction. (93D) Based on the results of our in vitro testing of the DHS constructs and the coBTK vs co2BTK comparison, we identified four constructs to test in vivo. Shown here are diagrams of the lentiviral constructs with RRL backbone expressing either version of codon optimized human BTK (coBTK or co2BTK) with a 0.7 kb ubiquitous chromatin opening element (0.7UCOE), with or without addition of DHS4 downstream of the 0.7UCOE element.

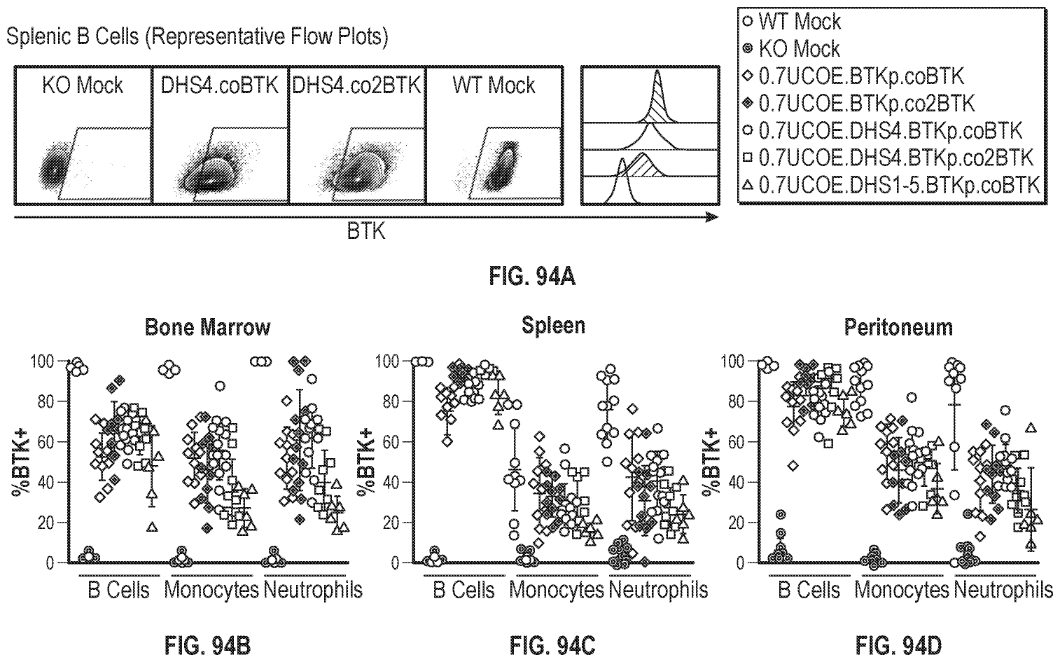

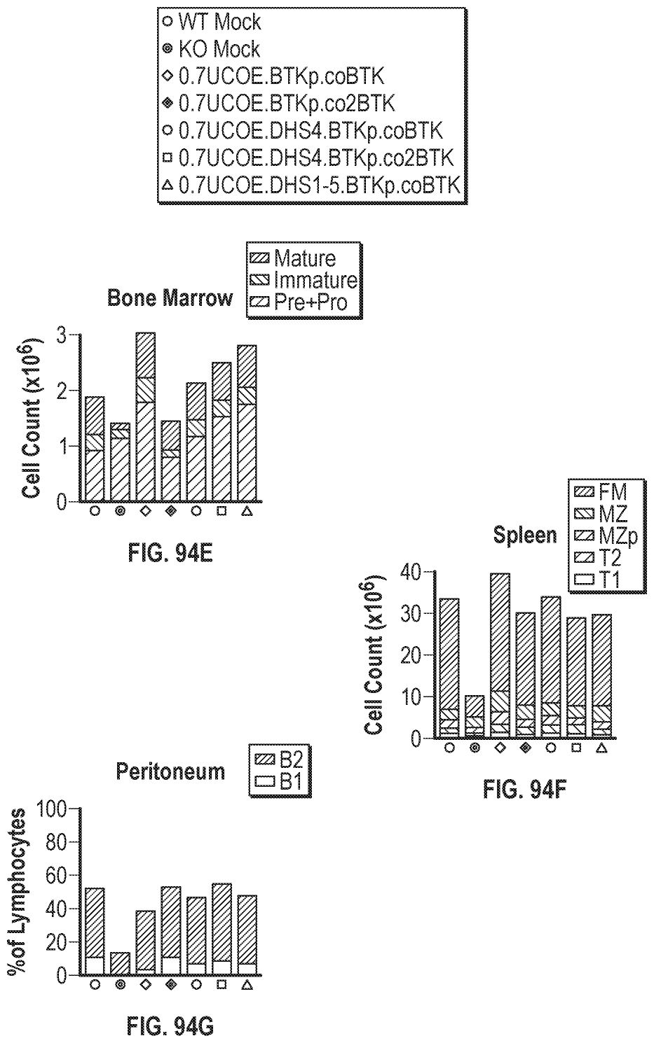

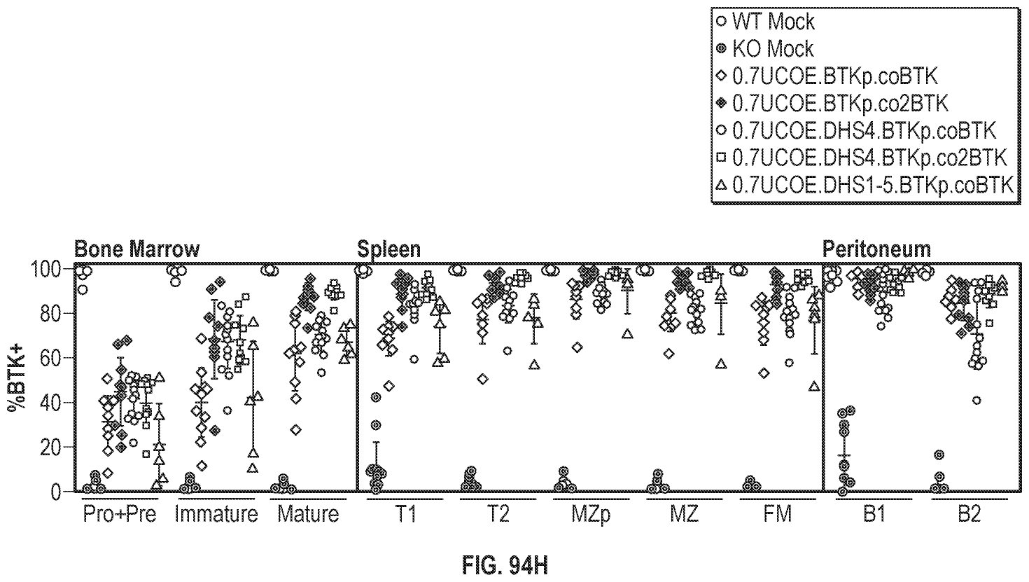

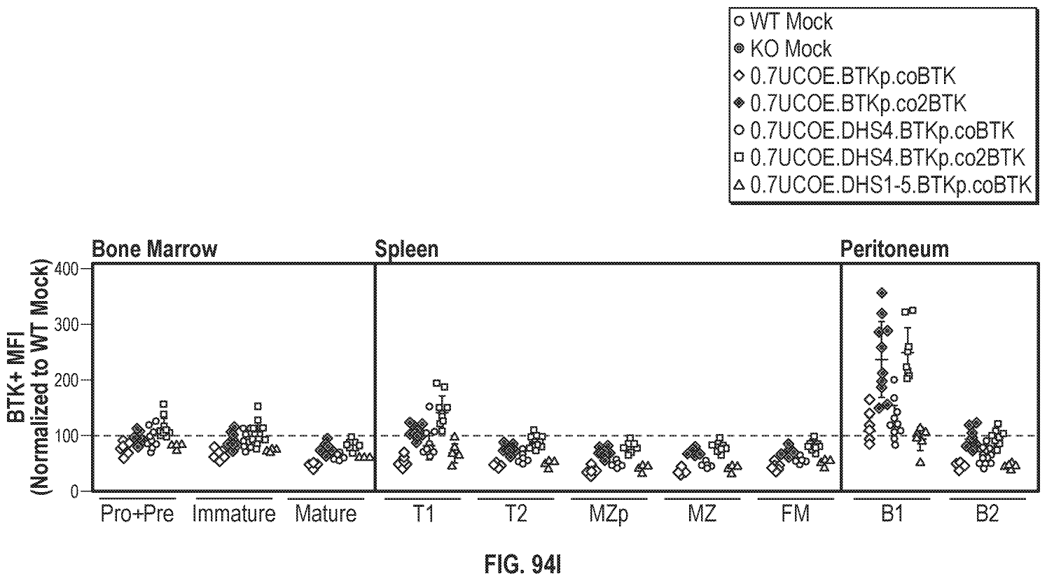

Shown in FIG. 94: are the results from restoration of BTK expression in affected hematopoietic lineages and B cell development in gene therapy mice treated with 0.7UCOE vectors. The proportion of BTK+ cells and numerical reconstitution of B and myeloid cell subsets was assessed at 19-23 weeks post-transplant. (FIG. 94A) Representative flow plots showing intracellular BTK staining in splenic B cells at endpoint analysis. (FIG. 94B-D) Percent of BTK+ cells in bone marrow (FIG. 94B), spleen (FIG. 94C), and peritoneal (FIG. 94D) lymphocyte subsets from gene therapy treated groups, determined by flow cytometry. Cell subsets are defined as Neutrophils (CD11b+ GR1+), Monocytes (CD11b+), and B cells (B220+). (FIG. 94E-F) Stacked bars show the average counts of B cell subsets in bone marrow (FIG. 94E), spleen (FIG. 94F), and peritoneum (FIG. 94G. Early B cell development (bone marrow): Pro+Pre−B (IgM−, IgD−), Immature (IgM+ IgD−), and Mature (IgM+, IgD+). Late B cell development (spleen): transitional T1 (CD24hi, CD21−), transitional T2 (CD24hi, CD21int), marginal zone/precursor MZ/MZP (CD24hi, CD21hi), and follicular FM (CD24int, CD21int). Peritoneal B cell subsets: B1 (IgM+ CD43+), and B2 (CD43−). (FIG. 94H) Rescue of BTK expression in B cell subsets, measured by flow cytometry. (FIG. 94I) BTK+ MFI across B cell development, normalized to WT Mock in each individual experiment. Data represent mean±SD from 4 unique experiments, n=13 (WT Mock), 13 (KO Mock), 11 (0.7UCOE.BTKp.co), 11 (0.7UCOE.BTKp.co2), 16 (0.7UCOE.DHS4.BKTp.co), 11 (0.7UCOE.DHS4.BKTp.co2), and 6 (0.7UCOE.DHS1-5.BTKp.co). P value was determined using the One way Anova: Sidak test: ***P<0.001; **P=0.001-0.01; *P=0.01-0.05.

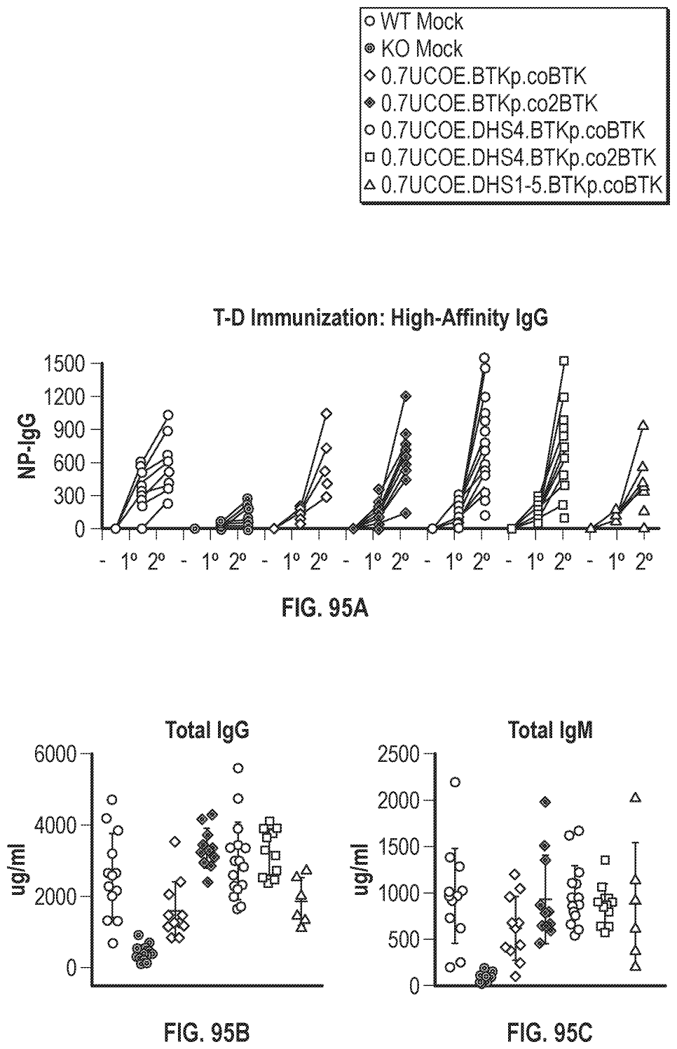

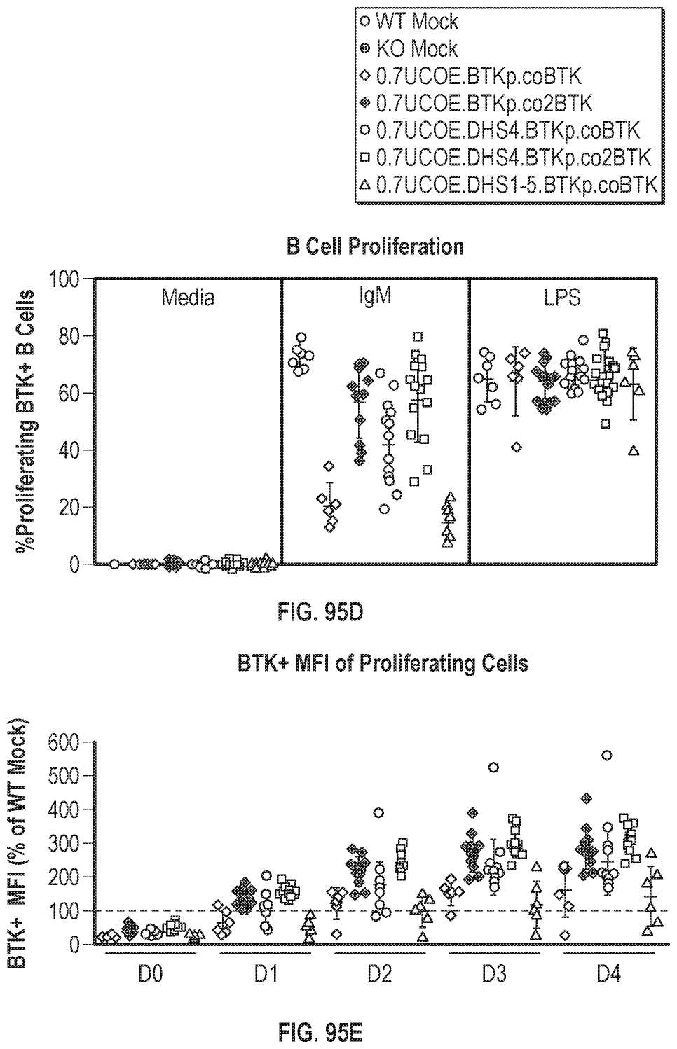

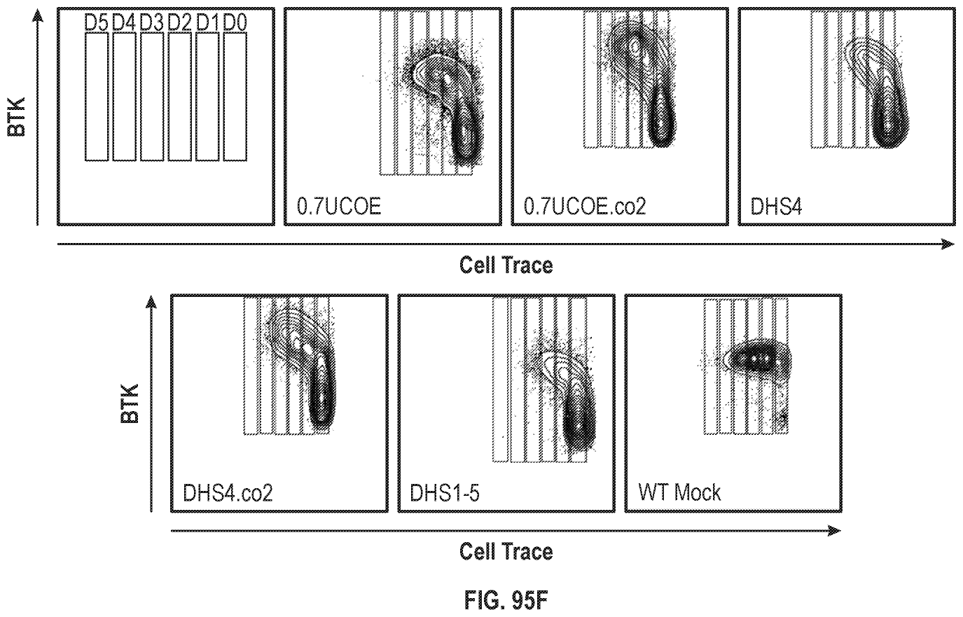

FIG. 95 shows the restoration of B cell function in vivo and in vitro. (FIG. 95A) Mice were immunized with NP-CGG in Alum at 12 weeks post-transplant. Levels of NP-specific IgG in serum from immunized mice was measured by ELISA and expressed relative to an IgG standard. High-affinity NP-IgG was measured from serum prior to (−) and 10 days following primary immunization (1°). One month following primary challenge, mice were re-challenged with NP-CGG in PBS and serum was collected 10 days later (2°). (FIG. 95B-C) Total serum IgG (FIG. 95B) and IgM (FIG. 95C) in serum from treated mice was measured by ELISA and endpoint analysis (21-23 weeks post-transplant). (FIG. 95D-F) At endpoint analysis, B cells were isolated from splenocytes by CD43− magnetic separation, labeled with Cell Trace Violet and stimulated in vitro with IgM, LPS, or a media control. (FIG. 95D) The percentage of BTK+ B cells that underwent ≥1 cell division 72 hours after incubation with anti-mouse IgM antibodies, LPS or media only (read out by flow cytometry). (FIG. 95E) BTK+ MFI of cells after each division (DO-D4), normalized to WT Mock. (FIG. 95F) Representative flow plots showing BTK staining and Cell Trace dilution in B cells 72 hours post-IgM stimulation, gated on live and B220+ BTK+ cells. Data represent mean±SD from 4 unique experiments, n=13 (WT Mock), 13 (KO Mock), 11 (0.7UCOE.BTKp.co), 11 (0.7UCOE.BTKp.co2), 16 (0.7UCOE.DHS4.BKTp.co), 11 (0.7UCOE.DHS4.BKTp.co2), and 6 (0.7UCOE.DHS1-5.BTKp.co). P value was determined using the One way Anova: Sidak test: ***P<0.001; **P=0.001-0.01; *P=0.01-0.05.

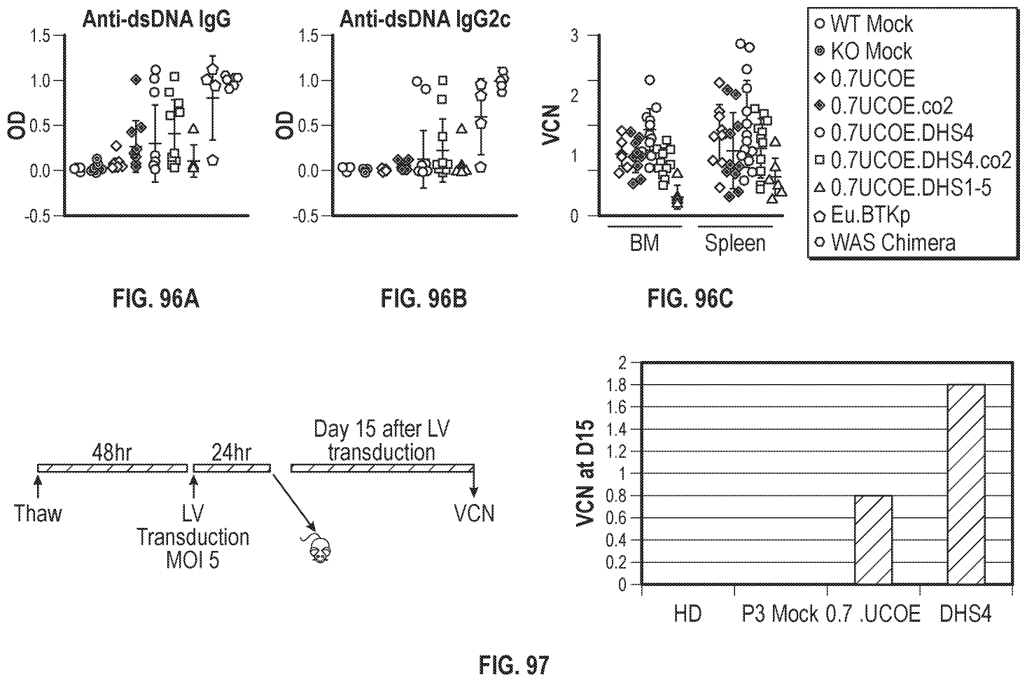

FIG. 96 shows some vector safety considerations. Levels of anti-dsDNA IgG (FIG. 96A) and anti-dsDNA IgG2c (FIG. 96B) in serum from treated mice were measured by ELISA and are depicted as absorbance readings (OD450). Serum from the known autoimmune-prone WAS chimeric mouse model and mice treated with Eu.BTKp were included as positive controls. (FIG. 96C) Genomic DNA was isolated from total bone marrow and spleen at endpoint analysis and the number of viral integrations per cell (VCN) was quantified by qPCR. Data represent mean±SD from 4 unique experiments, n=13 (WT Mock), 13 (KO Mock), 11 (0.7UCOE.BTKp.co), 11 (0.7UCOE.BTKp.co2), 16 (0.7UCOE.DHS4.BKTp.co), 11 (0.7UCOE.DHS4.BKTp.co2), and 6 (0.7UCOE.DHS1-5.BTKp.co). P value was determined using the One way Anova: Sidak test: ***P<0.001; **P=0.001-0.01; *P=0.01-0.05.

Pre−Clinical Modeling LV Vectors in Human HSC from XLA Patients

The aim of the following experiments was to determine if gene therapy vectors can rescue the development of B cells from XLA HSCs when transplanted into immunodeficient NSG mice. CD34 cell source: XLA Patient 3 (missense mutation) and healthy donor #15. The transduction protocol comprises one hit of LV after a 48 hour prestimulation in SCGM (TPO, FLT3 and SCF at 100 ng/ml). The MOI used: 5. The lentivirus used was 0.7 UCOE. BTKp.BTK.co2 (titer: 7×10⁸) and DHS4.co2 (titer: 1×10⁹)

Experimental mice: 4 mice=Healthy donor #15 (HD)
3 mice=XLA P3 mock treated (XLAP3)
5 mice=0.7 UCOE.BTKp.BTK.co2 (0.7UCOE)
4 mice=0.7 UCOE.DHS4.BTKp.BTK.co2 (DHS4)

Analyzed at: 12 week post transfer (FIG. 97).

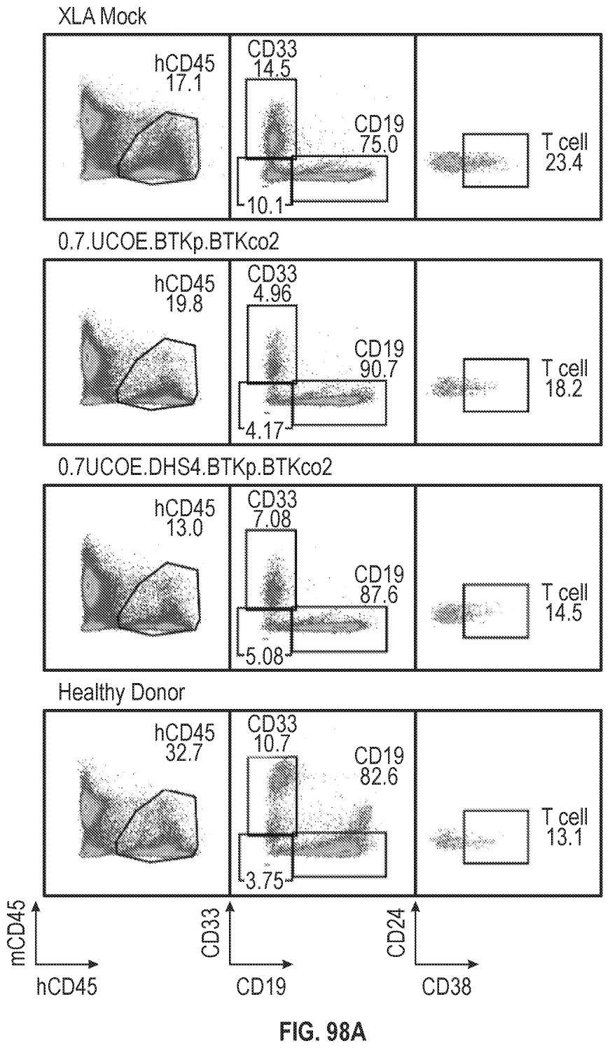

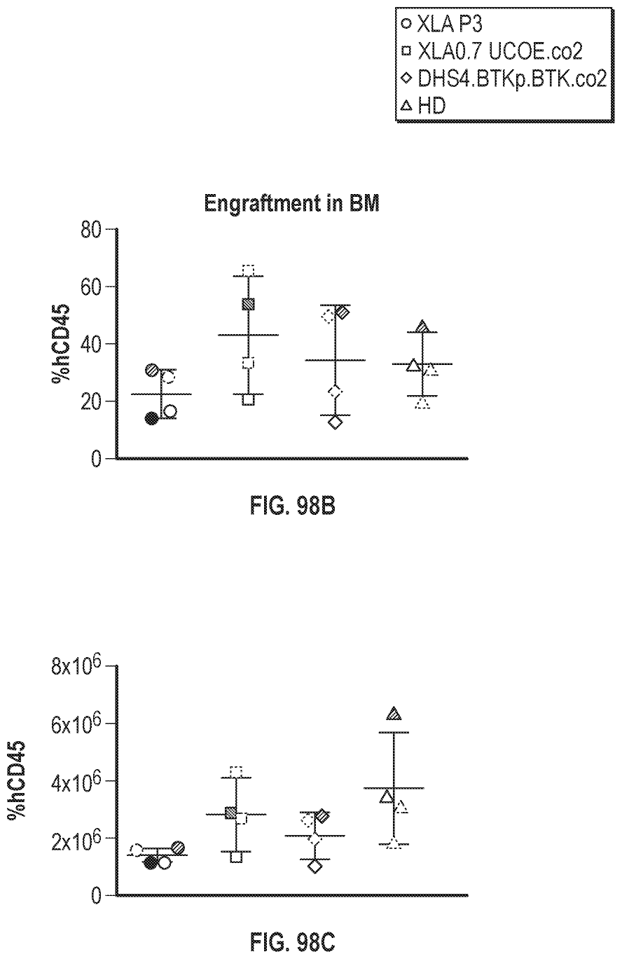

Shown in FIG. 98 are the results from an equivalent bone marrow engraftment of human hematopoietic cells between treatment groups. Human stem cells from XLA patient, either with or without gene therapy treatments, engrafted into the bone marrow equivalently as those from a healthy donor. Representative flow plots showing various markers of human immune cells including human and mouse CD45 (a marker for hematopoietic cells) CD33 (myeloid cells) and CD19 (B cells), CD4 and CD8 (T cells). Human hematopoietic cells are hCD45+ and mCD45− from total live BM cells; CD33 and CD19 markers were analyzed from hCD45+gate; CD4 and CD8 were analyzed from the CD33-CD19− gate. FIGS. 98B and 98C. % human CD45 and total number of hCD45 cells engrafted in BM; n=4 for each cohort (XLA3, XLA3+LV 0.7UCOE.BTKp.BTKco2, XLA3+LV 0.7UCOE.DHS4.BTKp.BTKco2, and healthy donor).

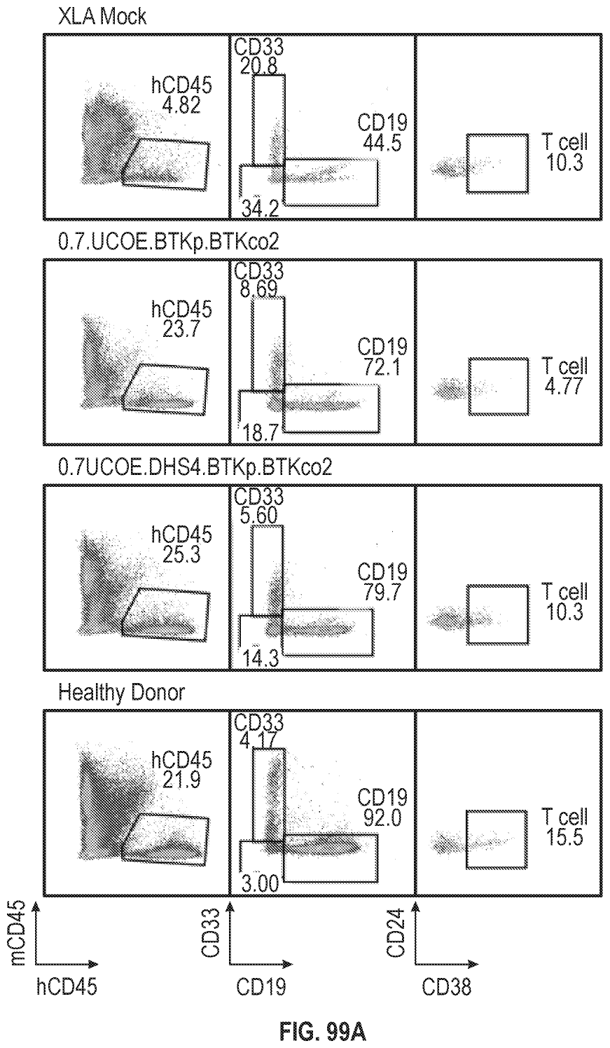

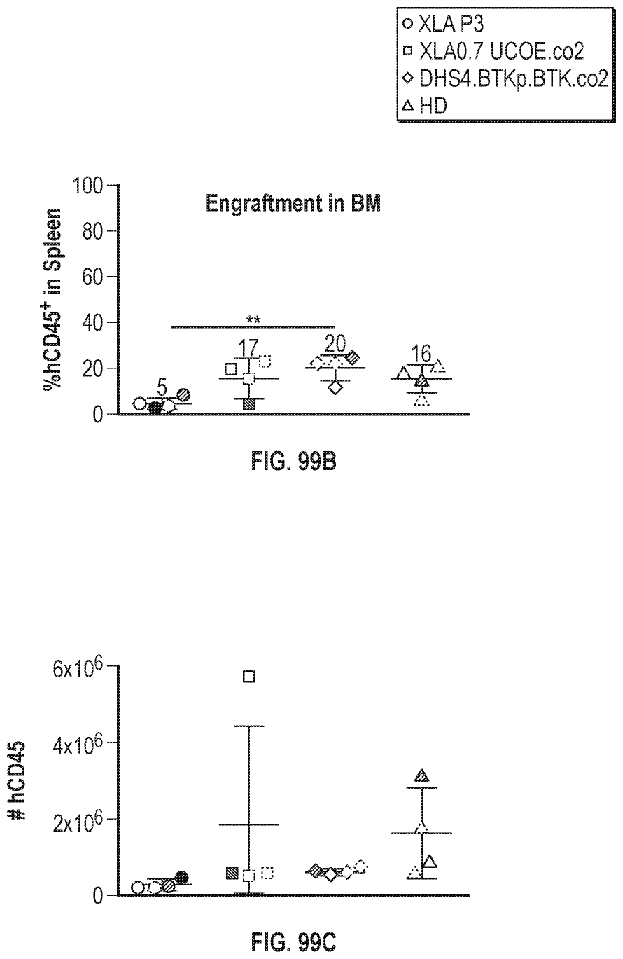

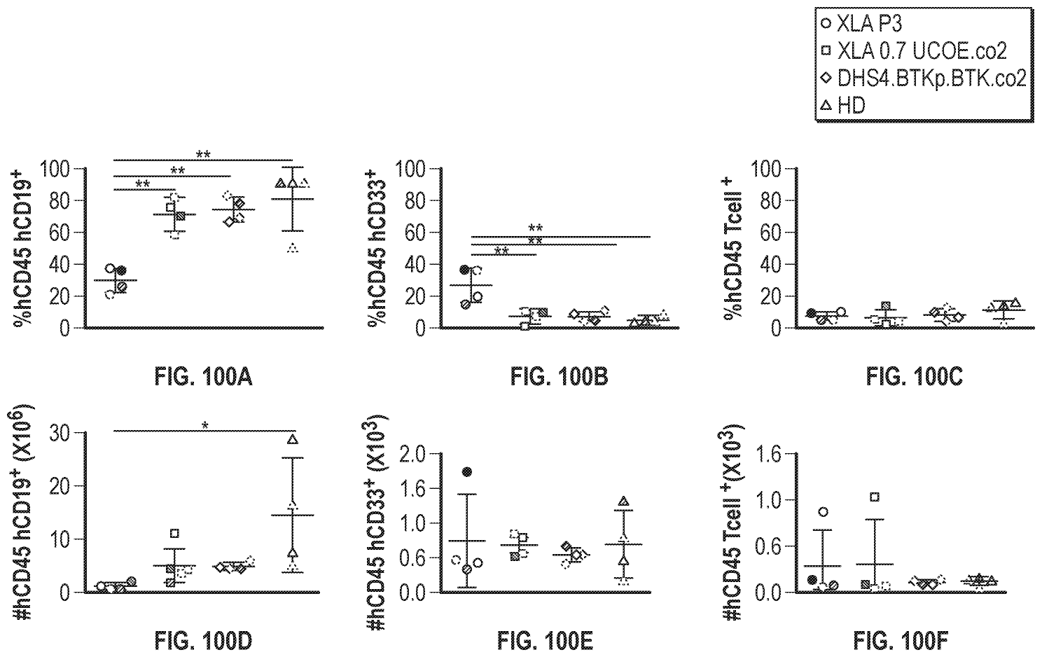

Shown in FIG. 99, are the results of mouse recipients of XLA3 HSC given gene therapy using 0.7UCOE.BTKp BTKco2 or 0.7UCOE.DHS4.BTKp.BTKco2 showing significantly increased numbers of splenic human hematopoietic cells. FIG. 99A. Representative flow plots showing markers of human immune cells including human and mouse CD45 (a marker for hematopoietic cells) CD33 (myeloid cells) and CD19 (B cells), CD4 and CD8 (T cells). Human hematopoietic cells are both hCD45+ and mCD45−; CD33 and CD19 markers were analyzed from the hCD45+gate; CD4 and CD8 were analyzed from the CD33-CD19− gate. FIGS. 99B and 99C: % and total number of splenic cells that were hCD45+. n=4 for each cohort; ** P=0.004 by one way ANOVA. Shown in FIG. 100, are the results of recipients of LV transduced XLA HSC (using 0.7UCOE BTKp BTKco2 or DHS4 BTKpBTK.co2), which exhibit an increased proportion of splenic B cells (CD19+cells) relative to non-treated XLA patient cells Here the results from flow cytometry shown in FIG. 2 are summarized, looking at specific categories of human immune cells: A-C, % of human CD45+ cells in the spleen that are B cells (CD19+), myeloid cells (CD33+) and T cells (CD4+ or CD8+); D-F, Total number of human CD45+ cells in the spleen that are B cells (CD19+), myeloid cells (CD33+) and T cells (CD4+ or CD8+); n=4 for each cohort. **P=0.0044 by one way ANOVA.

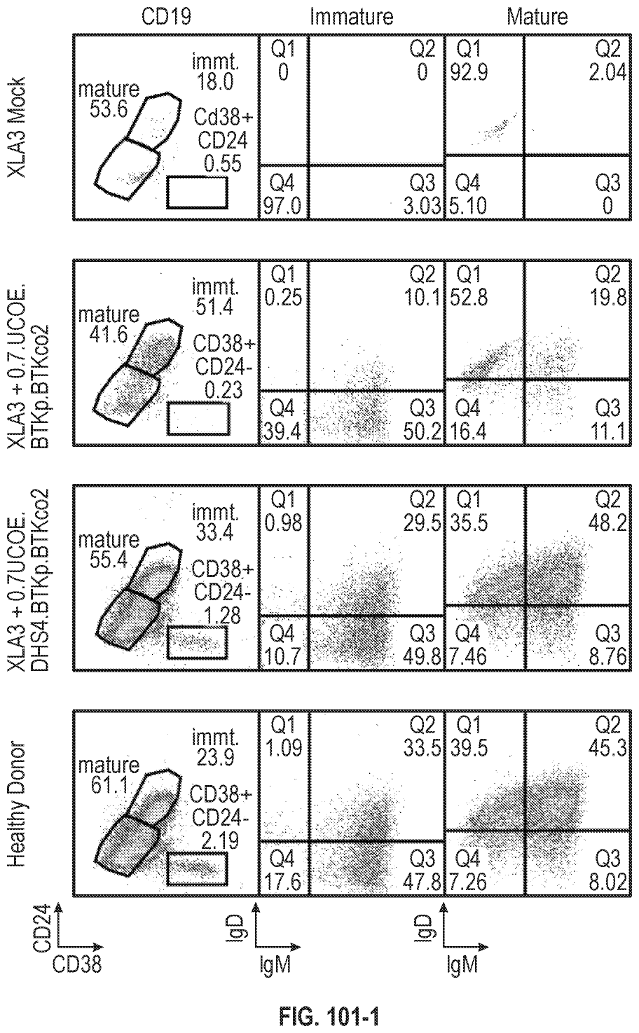

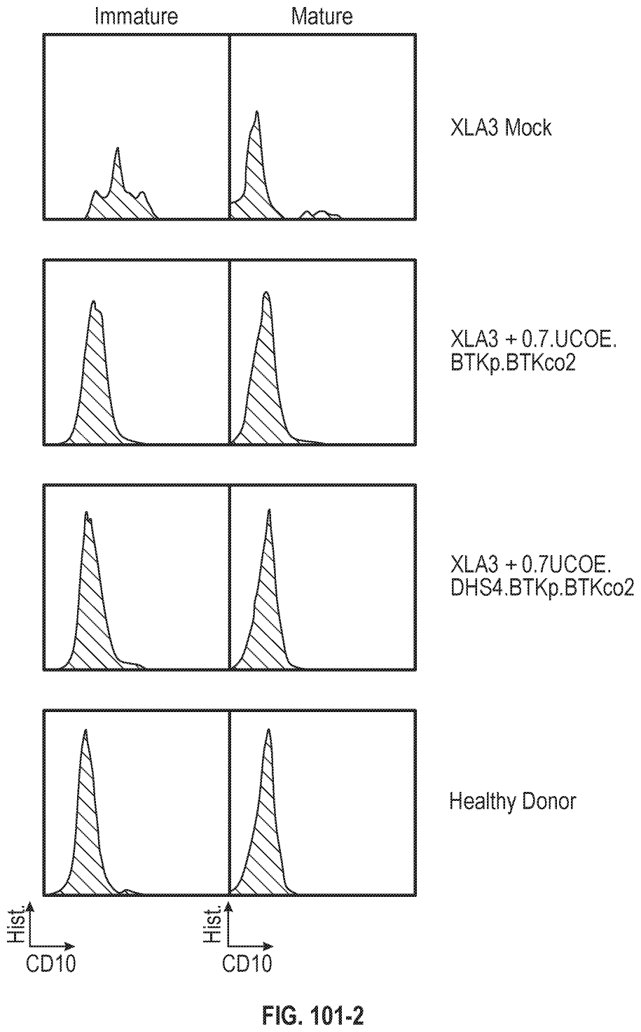

Shown in FIG. 101, is a representative flow plot for B cell development subsets in spleen. A typical gating strategy for identifying human B cell developmental subsets is shown for each gene therapy cohort (listed at right). Markers used in each panel are shown at bottom (Hist=histogram); predecessor gates are shown at the top of each column if used. Human CD24 and hCD38 cells were gated from human CD19+cells. Immature B cells (hCD24+hCD38+) are IgM+ IgD−CD10high; mature B cells (CD24low CD38low) are IgM+IgD+ CD10 low.

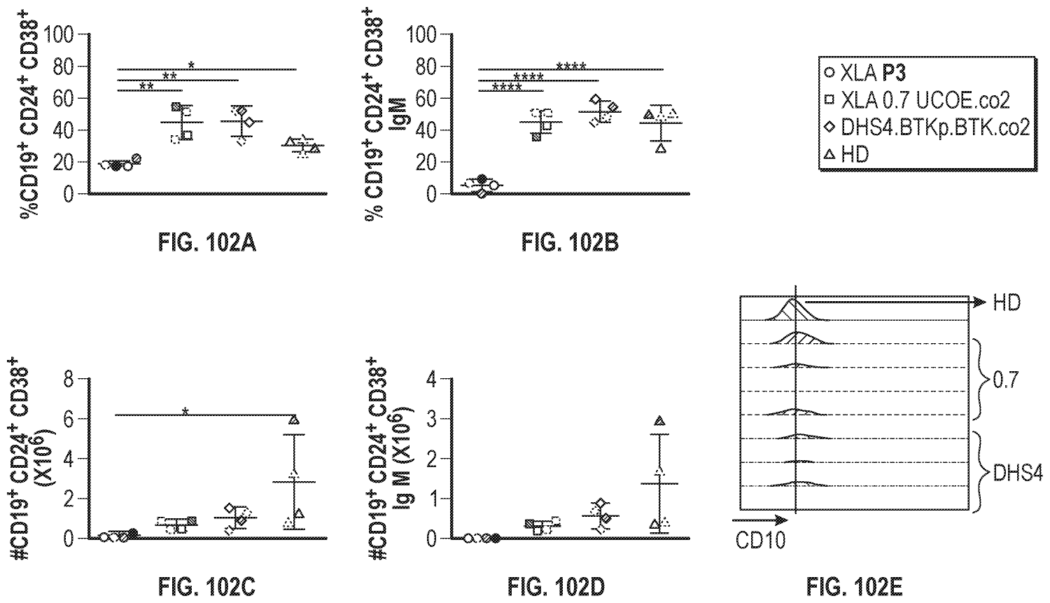

Shown in FIG. 102 are the results of recipients of LV transduced XLA3 HSC (using 0.7UCOE.BTKp.BTKco2 or 0.7UCOE.DHS4 BTKp.BTKco2). As shown, the recipients have an increased proportion of splenic immature B cells (including CD19+CD24+CD38+ B cells and CD19+CD24+CD38+IgM+ cells) compared to XLA controls. Graphs summarize the results from flow cytometry shown in FIG. 4, looking at specific subpopulations of immature B cells. A,% of Immature B cells (CD24+ CD38+) in the spleen; B, % of immature B cells that are IgM+; C-D, Total number of immature B cells (CD24+ CD38+) and CD24+hCD38+IgM+cells in the spleen; E, overlay of CD10 histogram, showing the mean fluorescence intensity MFI shift in CD10 compared to healthy donor. n=4 for each cohort; ***P=0.0004; **P=0.0044; *P=0.4 by one way ANOVA.

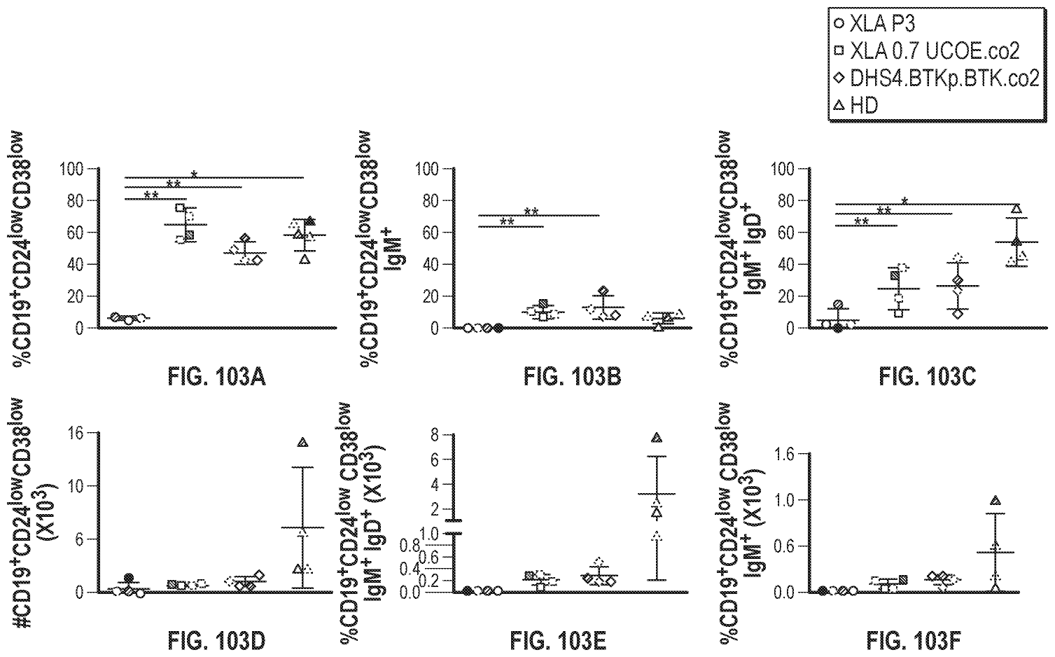

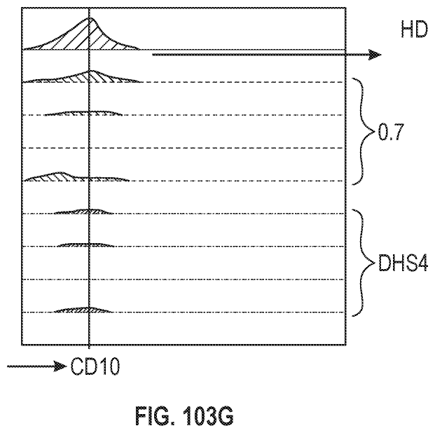

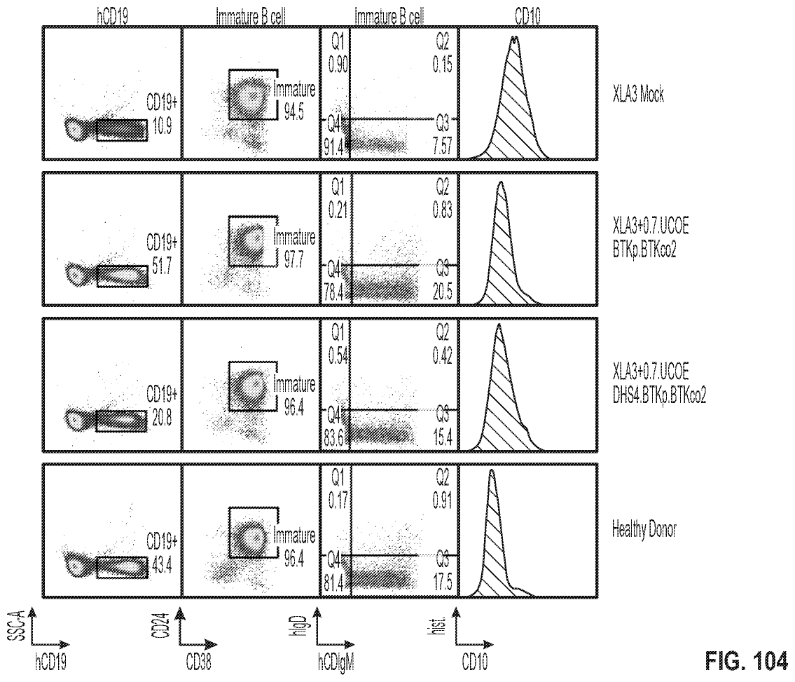

As shown in FIG. 103, recipients of LV transduced XLA HSC (0.7UCOE.BTKp.BTKco2 or 0.7UCOE.DHS4 BTKp.BTKco2) have increased proportions of mature B cells (CD19⁺CD24^{low}CD38^{low}IgM⁺ and IgD⁺) compared to XLA controls. FIGS. 104A-C, % of mature B cells (hCD24^{low}hCD38^{low}) and the percent of mature B cells that are IgM⁺ and IgM⁺IgD⁺ in the spleen, respectively; FIGS. 103D-F, Total number of splenic mature B cells and mature B cells that are IgM⁺ and IgM⁺IgD⁺, respectively; FIG. 103G, histogram overlay of CD10, showing similar MFI of CD10 as that of a healthy donor. N=4 for each cohort; ***P=0.0004; **P=0.0044; * P=0.4 by one way ANOVA.

Shown in FIG. 104 is a representative flow plot for B cell development subsets in the bone marrow. Flow plots and gating strategy for identifying human B cell developmental subsets in the bone marrow is shown for each gene therapy cohort (listed at right). Markers used in each panel are shown at bottom (Hist=histogram); predecessor gates are shown at the top of each column if used. Human CD24 and hCD38 cells were gated from human CD19+ cells. Immature B cells in the bone marrow (hCD24+hCD38+) are IgM+ IgD−CD10high.

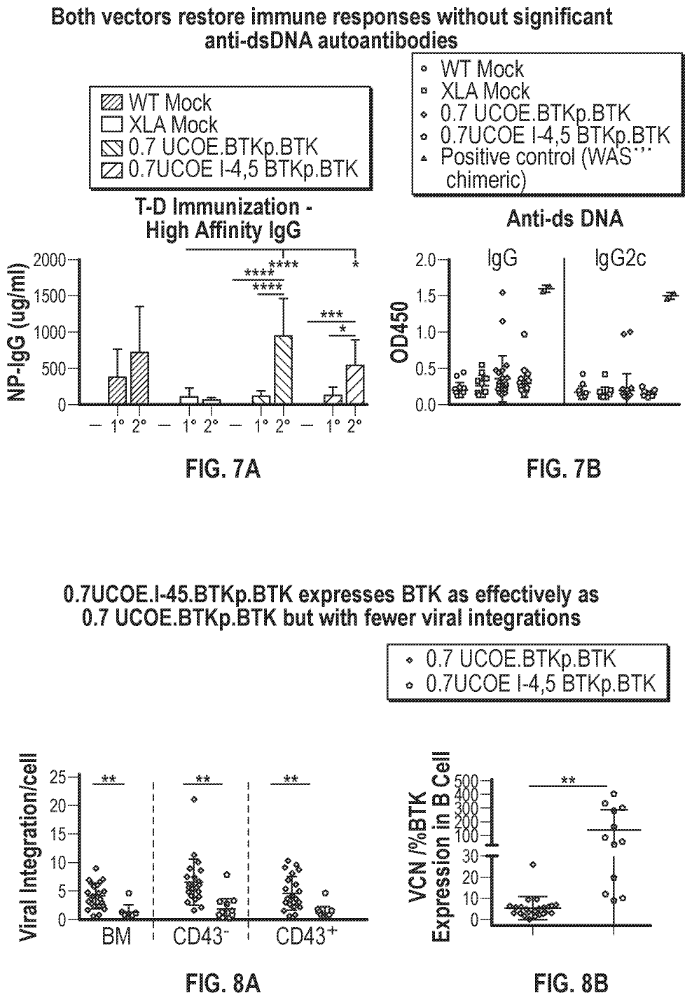

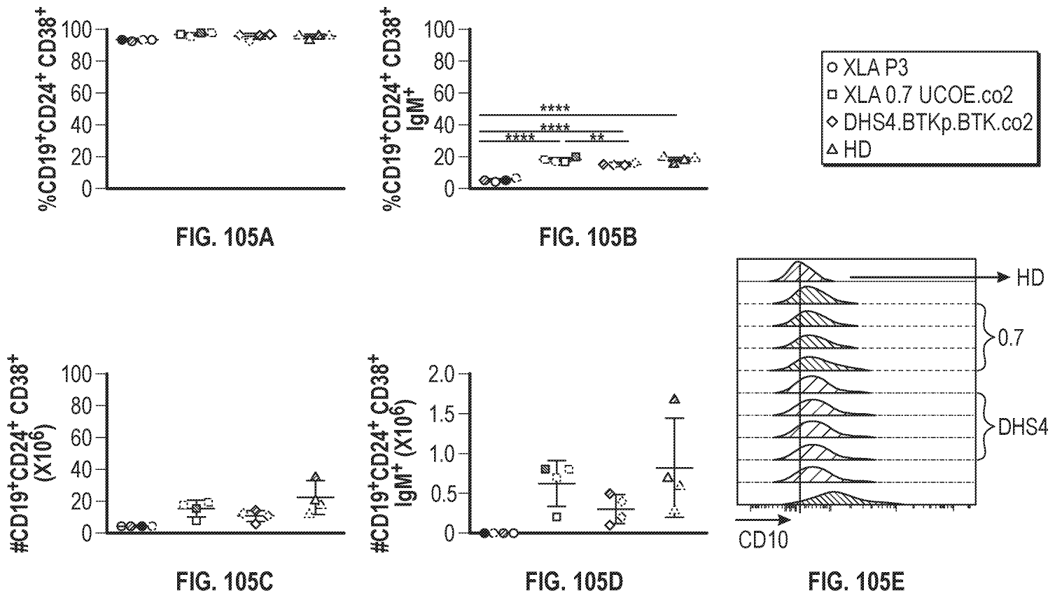

As shown in FIG. 105, recipients of LV transduced XLA HSC (using 0.7UCOE BTKp BTKco2 or DHS4 BTKpBTK.co2) exhibit an increased proportion of CD19⁺CD24⁺CD38⁺IgM⁺ immature B cells compared to XLA controls. Graphs summarize flow analysis based on gating strategy shown in FIG. 7. Each dot represents individual mice A-D, Percent and number of immature B cells (CD24⁺CD38⁺) and immature B cells that are IgM⁺, respectively; E, Overlay of CD10 histogram showing the MFI shift in CD10 compared to healthy donor. Blue color represents individual mice treated with LV 0.7.UCOE.BTKp.BTKco2, and orange color individual mice treated with LV 0,7UCOE.DHS4.UCOE.BTKp.BTKco2. n=4 for each cohort. Statistical significance were determined by one way ANOVA; ***P=0.0004; **P=0.0044; * P=0.4.

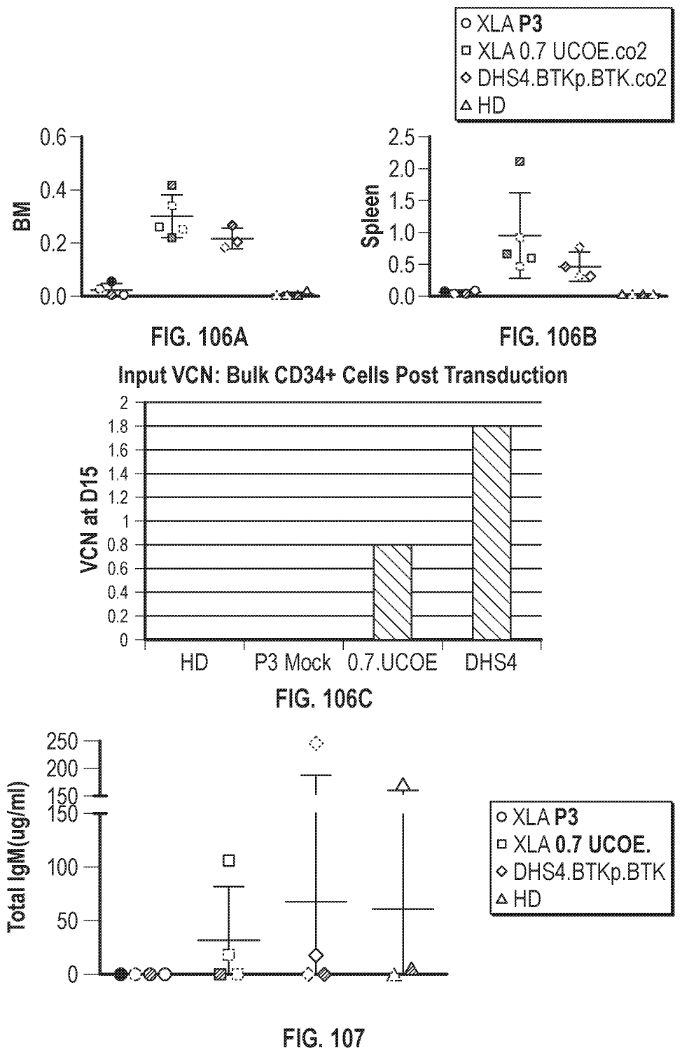

As shown in FIG. 106, recipients of LV transduced XLA HSC (using LV 0.7UCOE BTKp BTKco2 or LV 0.7UCOE.DHS4 BTKp.BTK.co2) exhibit gene marking of 0.2-2 viral copy number (VCN)/cell in vivo. The number of viral integrations per cell were calculated in BM (A) and Spleen (B), as well as the original CD34 cells prior to transplantation (C) as determined by quantitative PCR. No significant difference between 0.7UCOE.Co2.BTKp.BTK and DHS4.BTKp.BTKCo2. Each dot represents individual mice (N=4 for each in A and B; N=1 in C)

As shown in FIG. 107, recipients of XLA3 HSC that received gene therapy with LV 0.7UCOE.BTKp.BTKco2 or 0.7UCOE.DHS4.BTK.pBTKco2 produce B cells that secrete IgM in vivo. Total IgM levels were quantified by ELISA from serum obtained 12 weeks post-transplant. The value of IgM (μg/mL) were determined using a human IgM standard. Each dot represents the result from an individual mouse; N=4 for each cohort.

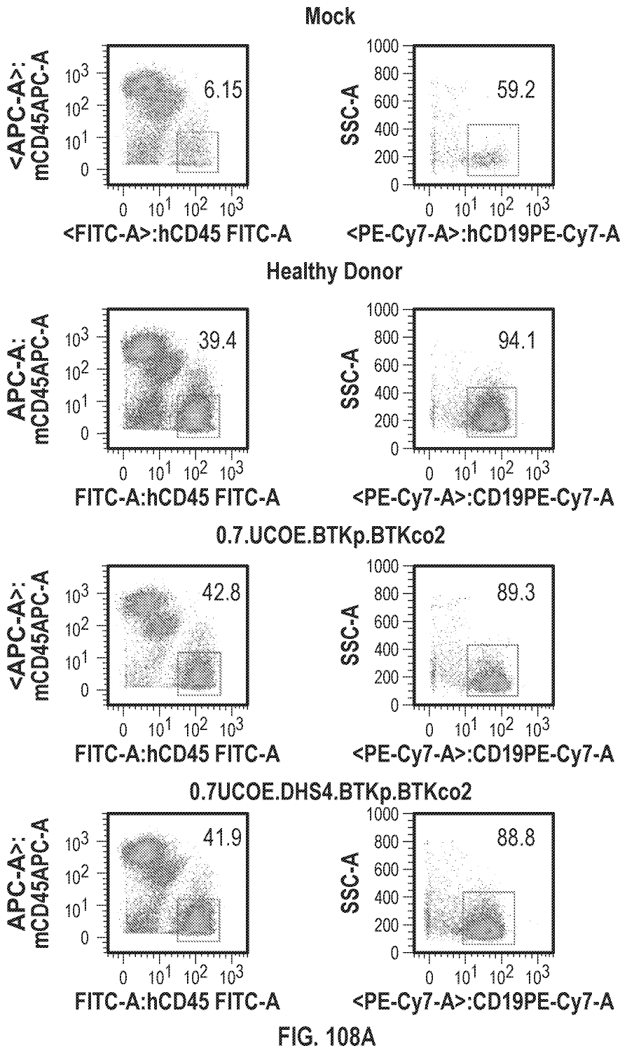

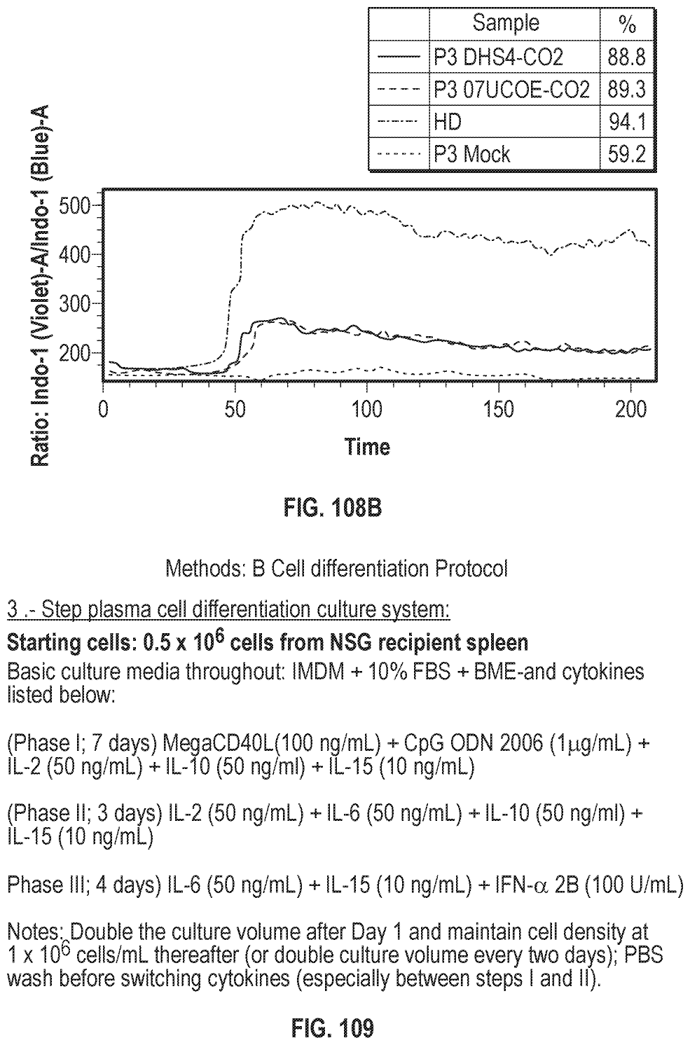

As shown in FIG. 108, recipients of XLA3 HSC transduced with LV 0.7UCOE.BTKp.BTKco2 or 0.7UCOE.DHS4.BTKp.BTKco2 exhibit a restored capacity for B cells to flux calcium in response to B cell receptor (BCR) engagement. Pooled 5×10⁶ total splenic cells from mice within each cohort were analyzed for their ability to flux calcium in response to BCR signaling, an important event downstream of BTK activation. FIG. 108A, provides a representative flow plots showing the gating strategy for human CD19+ cells evaluated for the calcium flux. FIG. 108B provides a kinetic analysis of intracellular calcium levels by flow cytometry after BCR engagement with human IgM antibodies.

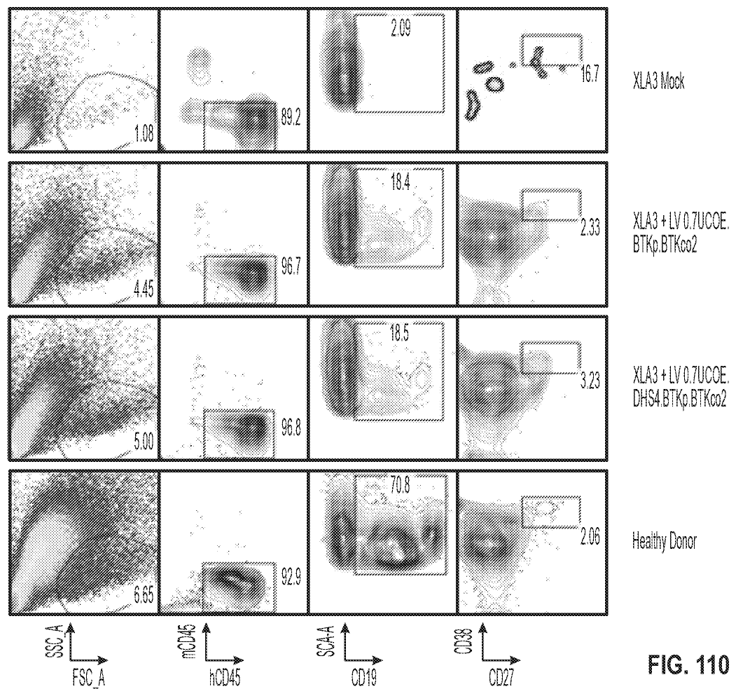

Shown in FIG. 110, is a representative flow plot for in vitro B cell class switching. Splenic cells from recipient mice were cultured in B cell differentiation protocol (previous slide) then stained for markers allowing the identification of plasma B cells. Media supernatant was also collected for determining human IgM and IgG levels by ELISA.

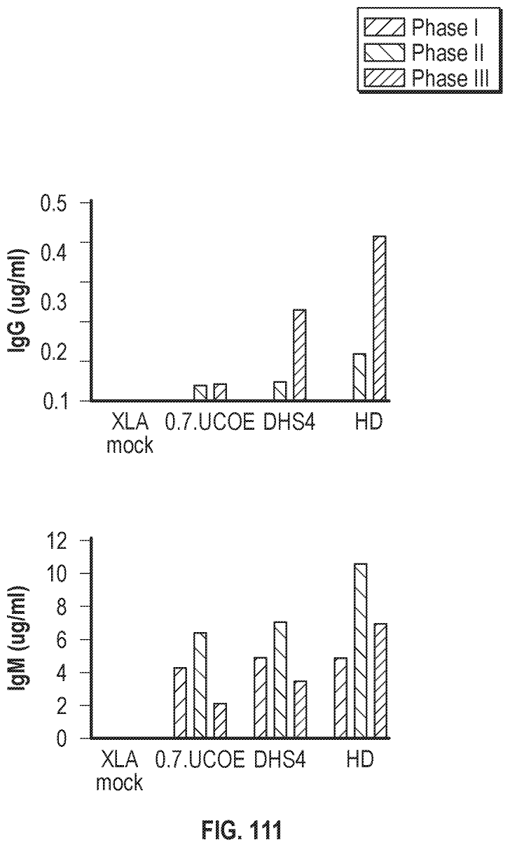

As shown in FIG. 111, recipients of LV transduced XLA HSC (using 0.7UCOE BTKp BTKco2 or DHS4 BTKpBTK.co2) generate B cells that are capable of responding to cytokine and T-cell dependent signals leading to antibody secretion. 0.5×10⁶ total splenic cells (pooled from mice in each cohort) were cultured in IMDM+10% FBS+2-mercaptoethanol media. In Phase I cells were cultured for 7 days with MegaCD40L (100 ng/ml)+CpG ODN 2006 (1 μg/ml)+IL-2 (50 ng/ml)+IL-10 (50 ng/ml)+IL-15 (10 ng/ml). After phase I cells were washed with PBS 2× and cultured for 3 days in PhaseII media (IMDM+10% FBS+BME supplemented with IL-2 (50 ng/ml)+IL-6 (50 ng/ml)+IL-10 (50 ng/ml)+IL-15 (long/ml), then switched to Phase III media (IMDM+10% FBS+BME supplemented with IL-6 (50 ng/ml)+IL-15 (long/ml)+IFN-α 2B (100 U/ml) for 4 days. At this time point media supernatants were collected and the levels of human IgG (A) and IgM (B) were determined by ELISA using a human IgM and IgG standard.

### Additional Alternatives

As described herein, a polynucleotide for sustained Bruton's tyrosine kinase (BTK) expression is provided. The polynucleotide can comprise a first sequence encoding a ubiquitous chromatin opening element (UCOE), a second sequence encoding a promoter and a third sequence encoding BTK. In some alternatives, the UCOE is 2kb, 1.5 kb, 1 kb, 0.75 kb, 0.5 kb or 0.25 kb or any number of kilobases in between a range defined by any two afore mentioned values. In some alternatives, the first sequence comprises the nucleic acid sequence set forth in SEQ ID NO: 1, and/or SEQ ID NO: 2. In some alternatives, the promoter is a BTK promoter. In some alternatives, the BTK promoter comprises the nucleic acid sequence set forth in SEQ ID NO: 5. In some alternatives, the third sequence is codon optimized for expression in humans. In some alternatives, the third sequence comprises the sequence set forth in SEQ ID NO: 6 or SEQ ID NO: 7. In some alternatives, the promoter is a B cell specific promoter. In some alternatives, the B cell specific promoter comprises the B cell specific promoter, B29. In some alternatives, the B29 promoter sequence comprises a sequence set forth in SEQ ID NO: 46. In some alternatives, the B cell specific promoter is an endogenous promoter. In some alternatives, the polypeptide further comprises one or more enhancer elements. In some alternatives, the one or more enhancer elements comprise at least one DNase Hypersensitive Site (DHS). In some alternatives, the DNase Hypersensitive Site is DNase Hypersensitive Site 1 (DHS1), DNase Hypersensitive Site 2, (DHS2) DNase Hypersensitive Site 3 (DHS3), DNase Hypersensitive Site 4 (DHS4) and/or DNase Hypersensitive Site 5 (DHS5). In some alternatives, the DNase Hypersensitive Site comprises a sequence set forth in SEQ ID NO: 3. In some alternatives, the one or more enhancer elements comprise at least one intronic region. In some alternatives, the at least one intronic region is from a human BTK locus that is in association with a human BTK proximal promoter. In some alternatives, the at least one intronic region is intron 4, intron 5 and/or intron 13 of the human BTK locus that is in association with the human BTK proximal promoter. In some alternatives, the intronic region comprises the sequence set forth in SEQ ID NO: 9 (intron 4), SEQ ID NO: 10 (intron 5) and/or SEQ ID NO: 11 (intron 13). In some alternatives, the one or more enhancer elements comprise the sequence set forth in SEQ ID NO: 4, SEQ ID NO: 14 or SEQ ID NO: 15. In some alternatives, the UCOE is in a reverse orientation or forward orientation. In some alternatives, the UCOE is in a forward orientation. In some alternatives, the one or more enhancer elements comprises the sequences set forth in SEQ ID NO: 16, SEQ ID NO: 17, SEQ ID NO: 18, SEQ ID NO: 19 and/or SEQ ID NO: 20. In some alternatives, the polynucleotide further comprises a gene upstream of a BTK promoter. In some alternatives, the gene upstream of a BTK promoter is a BTK enhancer. In some alternatives, the BTK enhancer comprises the sequence set forth in SEQ ID NO: 21 or SEQ ID NO: 22. In some alternatives, the polypeptide comprises a sequence set forth in SEQ ID NO's: 33, 34, 35, 41, 42, 43, 44 or 45. In some alternatives, 0.7UCOE comprises a sequence set forth in SEQ ID NO: 1. In some alternatives, 0.7UCOE comprises a sequence set forth in SEQ ID NO: 2. In some alternatives, the enhancer comprises a sequence set forth in SEQ ID NO: 3 or 4.

A vector for sustaining BTK expression in cells is also provided. The vector can comprise a first sequence encoding an ubiquitous chromatin opening element (UCOE), a second sequence encoding a promoter, and a third sequence encoding BTK. In some alternatives, the first sequence comprises the nucleic acid sequence set forth in SEQ ID NO: 1, and/or SEQ ID NO: 2. In some alternatives, the promoter is a BTK promoter. In some alternatives, the promoter comprises the nucleic acid sequence set forth in SEQ ID NO: 5. In some alternatives, the third sequence is codon optimized for expression in humans. In some alternatives, the third sequence comprises the sequence set forth in SEQ ID NO: 6 or SEQ ID NO: 7. In some alternatives, the vector further comprises a B cell specific promoter. In some alternatives, the B cell specific promoter comprises the B cell specific promoter, B29. In some alternatives, the B29 promoter sequence comprises a sequence set forth in SEQ ID NO: 46. In some alternatives, the B cell specific promoter is an endogenous promoter. In some alternatives, the vector further comprises one or more enhancer elements. In some alternatives, the one or more enhancer elements comprise at least one intronic region. In some alternatives, the one or more enhancer elements comprise a DNase Hypersensitive Site (DHS). In some alternatives, the DNase Hypersensitive Site is DNase Hypersensitive Site 1 (DHS1), DNase Hypersensitive Site 2, (DHS2) DNase Hypersensitive Site 3 (DHS3), DNase Hypersensitive Site 4 (DHS4) and/or DNase Hypersensitive Site 5 (DHS5). In some alternatives, the DNase Hypersensitive Site comprises a sequence set forth in SEQ ID NO: 3. In some alternatives, the at least one intronic region is from a human BTK locus that is in association with a human BTK proximal promoter. In some alternatives, the at least one intronic region is intron 4 intron 5 and/or intron 13 of the human BTK locus that is in association with the human BTK proximal promoter. In some alternatives, the intronic region comprises the sequence set forth in SEQ ID NO: 9 (intron 4), SEQ ID NO: 10 (intron 5) and/or SEQ ID NO: 11 (intron 13). In some alternatives, the one or more enhancer elements comprise the sequence set forth in SEQ ID NO: 4, SEQ ID NO: 14 or SEQ ID NO: 15. In some alternatives, the UCOE is in a reverse orientation or forward orientation. In some alternatives, the UCOE is in a forward orientation. In some alternatives, the vector is a lentiviral-based vector of a B lineage specific lentiviral vector. In some alternatives, the cells are B cells. In some alternatives, the cells are myeloid cells. In some alternatives, the cells are hematopoietic stem cells. In some alternatives, the cells are CD34+ hematopoietic stem cells. In some alternatives, the one or more enhancer elements comprise the sequences set forth in SEQ ID NO: 16, SEQ ID NO: 17, SEQ ID NO: 18, SEQ ID NO: 19 and/or SEQ ID NO: 20. In some alternatives, the polynucleotide further comprises a gene upstream of a BTK promoter. In some alternatives, the gene upstream of a BTK promoter is a BTK enhancer. In some alternatives, the BTK enhancer comprises the sequence set forth in SEQ ID NO: 21 or SEQ ID NO: 22. In some alternatives, the polypeptide or vector comprises a sequence set forth in SEQ ID NO's: 33, 34, 35, 41, 42, 43, 44 or 45. In some alternatives, 0.7UCOE comprises a sequence set forth in SEQ ID NO: 2. In some alternatives, the enhancer comprises a sequence set forth in SEQ ID NO: 3 or 4.

In some alternatives, a cell for expression of BTK is provided, the cell comprising: a polynucleotide, which comprises a first sequence encoding an ubiquitous chromatin opening element (UCOE), a second sequence encoding a promoter and a third sequence encoding BTK. In some alternatives, the polynucleotide is in a vector. The vector can comprise a first sequence encoding a ubiquitous chromatin opening element (UCOE), a second sequence encoding a promoter, and a third sequence encoding BTK. In some alternatives, the first sequence comprises the nucleic acid sequence set forth in SEQ ID NO: 1, and/or SEQ ID NO: 2. In some alternatives, the promoter is a BTK promoter. In some alternatives, the promoter comprises the nucleic acid sequence set forth in SEQ ID NO: 5 In some alternatives, the third sequence is codon optimized for expression in humans. In some alternatives, the third sequence comprises the sequence set forth in SEQ ID NO: 6 or SEQ ID NO: 7. In some alternatives, the vector further comprises a B cell specific promoter. In some alternatives, the B cell specific promoter comprises the B cell specific promoter, B29. In some alternatives, the B29 promoter sequence comprises a sequence set forth in SEQ ID NO: 46. In some alternatives, the B cell specific promoter is an endogenous promoter. In some alternatives, the vector further comprises one or more enhancer elements. In some alternatives, the one or more enhancer elements comprise at least one intronic region. In some alternatives, the one or more enhancer elements comprises a DNase Hypersensitive Site (DHS). In some alternatives, the DNase Hypersensitive Site is DNase Hypersensitive Site 1 (DHS1), DNase Hypersensitive Site 2, (DHS2) DNase Hypersensitive Site 3 (DHS3), DNase Hypersensitive Site 4 (DHS4) and/or DNase Hypersensitive Site 5 (DHS5). In some alternatives, the DNase Hypersensitive Site comprises a sequence set forth in SEQ ID NO: 3. In some alternatives, the at least one intronic region is from a human BTK locus that is in association with a human BTK proximal promoter. In some alternatives, the at least one intronic region is intron 4 intron 5 and/or intron 13 of the human BTK locus that is in association with the human BTK proximal promoter. In some alternatives, the intronic region comprises the sequence set forth in SEQ ID NO: 9 (intron 4), SEQ ID NO: 10 (intron 5) and/or SEQ ID NO: 11 (intron 13). In some alternatives, the one or more enhancer elements comprise the sequence set forth in SEQ ID NO: 4 (SEQ ID NO: 4: intron 4-5), SEQ ID NO: 14 or SEQ ID NO: 15. In some alternatives, the UCOE is in a reverse orientation or forward orientation. In some alternatives, the UCOE is in a forward orientation. In some alternatives, the vector is a lentiviral-based vector of a B lineage specific lentiviral vector. In some alternatives, the cells are B cells. In some alternatives, the cells are myeloid cells. In some alternatives, the cells are hematopoietic stem cells. In some alternatives, the cells are CD34+ hematopoietic stem cells. In some alternatives, the one or more enhancer elements comprises the sequences set forth in SEQ ID NO: 16, SEQ ID NO: 17, SEQ ID NO: 18, SEQ ID NO: 19 and/or SEQ ID NO: 20. In some alternatives, the polynucleotide further comprises a gene upstream of a BTK promoter. In some alternatives, the gene upstream of a BTK promoter is a BTK enhancer. In some alternatives, the BTK enhancer comprises the sequence set forth in SEQ ID NO: 21 or SEQ ID NO: 22. In some alternatives, the vector is a lentiviral vector. In some alternatives, the cell is a B cell. In some alternatives, the cells are myeloid cells. In some alternatives, the cell is a hematopoietic stem cell. In some alternatives, the cell is a CD34+ hematopoietic stem cell. In some alternatives, 0.7UCOE comprises a sequence set forth in SEQ ID NO: 2. In some alternatives, the enhancer comprises a sequence set forth in SEQ ID NO: 3 or 4.

In some alternatives, a method of promoting B cell survival, proliferation and/or differentiation in a subject in need thereof is provided, the method comprising administering the cell of any one of the alternatives herein to the subject or a cell comprising the polynucleotide of any one of the alternatives herein or the vector of any one of the alternatives herein to the subject in need and, optionally identifying the subject as one that would benefit from receiving a therapy that would promote B cell survival, proliferation and/or differentiation in advance of administering the cell and/or, optionally, measuring B cell survival, proliferation and/or differentiation in said subject or in a biological sample obtained from said subject. The vector can comprise a first sequence encoding a ubiquitous chromatin opening element (UCOE), a second sequence encoding a promoter, and a third sequence encoding BTK. In some alternatives, the first sequence comprises the nucleic acid sequence set forth in SEQ ID NO: 1, and/or SEQ ID NO: 2. In some alternatives, the promoter is a BTK promoter. In some alternatives, the promoter comprises the nucleic acid sequence set forth in SEQ ID NO: 5. In some alternatives, the third sequence is codon optimized for expression in humans. In some alternatives, the third sequence comprises the sequence set forth in SEQ ID NO: 6 or SEQ ID NO: 7. In some alternatives, the vector further comprises a B cell specific promoter. In some alternatives, the B cell specific promoter comprises the B cell specific promoter, B29. In some alternatives, the B29 promoter sequence comprises a sequence set forth in SEQ ID NO: 46. In some alternatives, the B cell specific promoter is an endogenous promoter. In some alternatives, the vector further comprises one or more enhancer elements. In some alternatives, the one or more enhancer elements comprise at least one intronic region. In some alternatives, the one or more enhancer elements comprise a DNase Hypersensitive Site (DHS). In some alternatives, the DNase Hypersensitive Site is DNase Hypersensitive Site 1 (DHS1), DNase Hypersensitive Site 2, (DHS2) DNase Hypersensitive Site 3 (DHS3), DNase Hypersensitive Site 4 (DHS4) and/or DNase Hypersensitive Site 5 (DHS5). In some alternatives, the DNase Hypersensitive Site comprises a sequence set forth in SEQ ID NO: 3. In some alternatives, the at least one intronic region is from a human BTK locus that is in association with a human BTK proximal promoter. In some alternatives, the at least one intronic region is intron 4 intron 5 and/or intron 13 of the human BTK locus that is in association with the human BTK proximal promoter. In some alternatives, the intronic region comprises the sequence set forth in SEQ ID NO: 9 (intron 4), SEQ ID NO: 10 (intron 5) and/or SEQ ID NO: 11 (intron 13). In some alternatives, the one or more enhancer elements comprise the sequence set forth in SEQ ID NO: 4 (SEQ ID NO: 4: intron 4-5), SEQ ID NO: 14 or SEQ ID NO: 15. In some alternatives, the UCOE is in a reverse orientation or forward orientation. In some alternatives, the UCOE is in a forward orientation. In some alternatives, the vector is a lentiviral-based vector of a B lineage specific lentiviral vector. In some alternatives, the cells are B cells. In some alternatives, the cells are myeloid cells. In some alternatives, the cells are hematopoietic stem cells. In some alternatives, the cells are CD34+ hematopoietic stem cells. In some alternatives, the one or more enhancer elements comprises the sequences set forth in SEQ ID NO: 16, SEQ ID NO: 17, SEQ ID NO: 18, SEQ ID NO: 19 and/or SEQ ID NO: 20. In some alternatives, the polynucleotide further comprises a gene upstream of a BTK promoter. In some alternatives, the gene upstream of a BTK promoter is a BTK enhancer. In some alternatives, the BTK enhancer comprises the sequence set forth in SEQ ID NO: 21 or SEQ ID NO: 22. In some alternatives, the cell is from the subject and, wherein the cell is genetically modified by introducing the polynucleotide or the vector of any one of alternatives described above into the cell. In some alternatives, the administering is performed by adoptive cell transfer. In some alternatives, the cell is a B cell. In some alternatives, the cells are myeloid cells. In some alternatives, the cell is a hematopoietic stem cell. In some alternatives, the cell is a CD34+ hematopoietic stem cell. In some alternatives, the subject is male. In some alternatives, the subject is suffering from X linked agammaglobulinemia (XLA). In some alternatives, the subject is selected to receive immunoglobulin replacement therapy. In some alternatives, the subject is selected to receive targeted anti-microbial agents. In some alternatives, the polypeptide or vector comprises a sequence set forth in SEQ ID NO's: 33, 34, 35, 41, 42, 43, 44 or 45. In some alternatives, the polypeptide or vector comprises a sequence set forth in SEQ ID NO's: 33, 34, 35, 41, 42, 43, 44 or 45. In some alternatives, 0.7UCOE comprises a sequence set forth in SEQ ID NO: 2. In some alternatives, the enhancer comprises a sequence set forth in SEQ ID NO: 3 or 4.

In some alternatives, a method of treating, inhibiting, or ameliorating X linked agammaglobulinemia (XLA) or disease symptoms associated with XLA in a subject in need thereof is provided, the method comprising: administering the cell of any one of the alternatives herein to the subject or a cell comprising the polynucleotide of any one of the alternatives herein or the vector of any one of the alternatives herein to the subject in need and, optionally identifying the subject as one that would benefit from receiving a therapy for XLA or disease symptoms associated with XLA and/or, optionally, measuring an improvement in the progression of XLA or an improvement in a disease symptom associated with XLA in said subject. The vector can comprise a first sequence encoding a ubiquitous chromatin opening element (UCOE), a second sequence encoding a promoter, and a third sequence encoding BTK. In some alternatives, the first sequence comprises the nucleic acid sequence set forth in SEQ ID NO: 1 and/or SEQ ID NO: 2. In some alternatives, the promoter is a BTK promoter. In some alternatives, the promoter comprises the nucleic acid sequence set forth in SEQ ID NO: 5. In some alternatives, the third sequence is codon optimized for expression in humans. In some alternatives, the third sequence comprises the sequence set forth in SEQ ID NO: 6 or SEQ ID NO: 7. In some alternatives, the vector further comprises a B cell specific promoter. In some alternatives, the B cell specific promoter comprises the B cell specific promoter, B29. In some alternatives, the B29 promoter sequence comprises a sequence set forth in SEQ ID NO: 46. In some alternatives, the B cell specific promoter is an endogenous promoter. In some alternatives, the vector further comprises one or more enhancer elements. In some alternatives, the one or more enhancer elements comprise at least one intronic region. In some alternatives, the one or more enhancer elements comprise a DNase Hypersensitive Site (DHS). In some alternatives, the DNase Hypersensitive Site is DNase Hypersensitive Site 1 (DHS1), DNase Hypersensitive Site 2, (DHS2) DNase Hypersensitive Site 3 (DHS3), DNase Hypersensitive Site 4 (DHS4) and/or DNase Hypersensitive Site 5 (DHS5). In some alternatives, the DNase Hypersensitive Site comprises a sequence set forth in SEQ ID NO: 3. In some alternatives, the at least one intronic region is from a human BTK locus that is in association with a human BTK proximal promoter. In some alternatives, the at least one intronic region is intron 4 intron 5 and/or intron 13 of the human BTK locus that is in association with the human BTK proximal promoter. In some alternatives, the intronic region comprises the sequence set forth in SEQ ID NO: 9 (intron 4), SEQ ID NO: 10 (intron 5) and/or SEQ ID NO: 11 (intron 13). In some alternatives, the one or more enhancer elements comprise the sequence set forth in SEQ ID NO: 4 (SEQ ID NO: 4: intron 4-5), SEQ ID NO: 14 or SEQ ID NO: 15. In some alternatives, the UCOE is in a reverse orientation or forward orientation. In some alternatives, the UCOE is in a forward orientation. In some alternatives, the vector is a lentiviral-based vector of a B lineage specific lentiviral vector. In some alternatives, the cells are B cells. In some alternatives, the cells are myeloid cells. In some alternatives, the cells are hematopoietic stem cells. In some alternatives, the cells are CD34+ hematopoietic stem cells. In some alternatives, the one or more enhancer elements comprises the sequences set forth in SEQ ID NO: 16, SEQ ID NO: 17, SEQ ID NO: 18, SEQ ID NO: 19 and/or SEQ ID NO: 20. In some alternatives, the polynucleotide further comprises a gene upstream of a BTK promoter. In some alternatives, the gene upstream of a BTK promoter is a BTK enhancer. In some alternatives, the BTK enhancer comprises the sequence set forth in SEQ ID NO: 21 or SEQ ID NO: 22. In some alternatives, the vector is a lentiviral-based vector of a B lineage specific lentiviral vector. In some alternatives, the cells are B cells. In some alternatives, the cells are myeloid cells. In some alternatives, the cells are hematopoietic stem cells. In some alternatives, the cells are CD34+ hematopoietic stem cells. In some alternatives, the cell is from the subject, wherein the cell is genetically modified by introducing the polynucleotide of any one of the alternatives herein or the vector of any one of the alternatives herein into the cell. In some alternatives, the administering is performed by adoptive cell transfer. In some alternatives, the cell is a B cell. In some alternatives, the cells are myeloid cells. In some alternatives, the cell is a hematopoietic stem cell. In some alternatives, the cell is a CD34+ hematopoietic stem cell. In some alternatives, the subject is male. In some alternatives, the subject is selected to receive immunoglobulin replacement therapy. In some alternatives, the subject is selected to receive targeted anti-microbial agents. In some alternatives, the polypeptide or vector comprises a sequence set forth in SEQ ID NO's: 33, 34, 35, 41, 42, 43, 44 or 45. In some alternatives, 0.7UCOE comprises a sequence set forth in SEQ ID NO: 2. In some alternatives, the enhancer comprises a sequence set forth in SEQ ID NO: 3 or 4.

### Sequences

Listed below are sequences used in the alternatives herein:

| | | | | | PRO- | BTK codon | |
|---|---|---|---|---|---|---|---|
| 0.7UCOE: | | ENHANCER: | | | MOTER | optimization: | |
| 0.7UCOE | 0.7UCOEfwd | one | HS4 | IE4-5 | BTKP | coBTK | co2BTK |

>0.7UCOE (SEQ ID NO: 1)
(SEQ ID #1)
Cgcgtgtggcatctgaagcaccaccagcgagcgagagctagagagaagga
aagccaccgacttcaccgcctccgagctgctccgggtcgcgggtctgcag
cgtctccggccctccgcgcctacagctcaagccacatccgaagggggagg
gagccgggagctgcgcgcggggccgctggggggaggggtggcaccgccca
cgccgggcggccacgaagggcggggcagcgggcgcgcgcccggcgggggg
aggggccgcgcgccgcgcccgctgggaattggggccctagggggagggcg
gaggcgccgacgaccgcggcacttaccgttcgcggcgtggcgcccggtgg
tccccaaggggagggaagggggaggcggggcgaggacagtgaccggagtc
tcctcagcggtggcttttctgcttggcagcctcagcggctggcgccaaaa
ccggactccgcccacttcctcgcccctgcggtgcgagggtgtggaatcct
ccagacgctgggggagggggagttgggagataaaaactagtacccattgg
gaccactttcagcagcgaactctcctgtacaccaggggtcagttccacag
acgcgggccaggggtgggtcattgcggcgtgaacaataatttgactagaa
gttgattcgggtgttt
>0.7UCOEfwd (SEQ ID NO: 2)
(SEQ ID NO: 2)
Cgcaaacacccgaatcaacttctagtcaaattattgttcacgccgcaatg
acccacccctggcccgcgtctgtggaactgacccctggtgtacaggagag
ttcgctgctgaaagtggtcccaaaggggtactagtttttaagctcccaac
tccccctcccccagcgtctggaggattccacaccctcgcaccgcaggggc
gaggaagtgggcggagtccggttttggcgccagccgctgaggctgccaag
cagaaaagccaccgctgaggagactccggtcactgtcctcgccccgcctc
ccccttccctccccttggggaccaccgggcgccacgccgcgaacggtaag
tgccgcggtcgtcggcgcctccgccctccccctagggccccaattcccag
cgggcgcggcgcgcggcccctccccccgccgggcgcgcgcccgctgcccc
gcccttcgtggccgcccggcgtgggcggtgccacccctccccccagcggc
cccgcgcgcagctcccggctccctcccccttcggatgtggcttgagctgt
aggcgcggagggccggagacgctgcagacccgcgacccggagcagctcgg
aggcggtgaagtcggtggctttccttctctctagctctcgctcgctggtg
gtgcttcagatgccacac
>DHS4
(SEQ ID NO: 3)
aattctatcatagtgtgtcttgtctatgataactgcattgagaaagatgc
tctgcttgttgagtgagcatttcacttccttctggttctgactatctgtc
taatagtggtcatgtgggttgaaaagatagaaaaggggagtagtattagg
aagttcagtatgaggaagacttattagacttatgcataaacctaaattct
gttgtaatctggaagagctgaagtgccacatatgcatctgtttaggagag
caagaactacaaatttggtcttcagtttggcttgcttacatcctgagaac
tctgtaggccacatgtcgtgaatatagcagcctctgcaacagtgaaagcc
agaaaaggaagtggaaagtctcaggggagggggctttctgtcatggattt
atgagcacagcaagactaacaagcaaaaagaaaaatgtaaaaggatcttg
ttcgt
>1E4-5
(SEQ ID NO: 4)
acatctttgagcttcagtttcctcatctgtaaataggggaataatacata
cttcttaaggctactgcaaagatcaaataagtaatacatttgaagcactt
gggacagagcctgctacatagtaagtgctcattaagtgttagttatcatt
gttgttgtttttaggccaaggttgttgtgaaaattaaatgagataatata
taaaaggtatttagctcaatatctggcacatagcaataattgaatagatg
atccttcatcttcgttcctcctgttccctttcagtttgaaagacttggct
aatataattttgaccaaccaaacttgcattcaagggagtgtacaaggctg
gtatagccagccagtgagtatcagaatctaaatgtttattaagacaaagg
gctgtcatgcaataacccaaccataccattatcagtctgccatccttcct
gtttctctaggcagcctttcctgatgtcaactcaaccagttaatctctca
gtcacttgacatgtggctatatatacacacaaatatgtgtgcatgcatcc
tgtgctgcaagcatttacagtcaagtttatctgaacacactgtatggttg
atgtgaaatgctgaaactgttcaagtttaggtcctcacaaagcaaggaat
atgaaatatttccttgggaaatatttatccacaacaaagagatgtacagt
gattcgtatacagtgatttacagttttccatgtgcttttacatgtattat
tacttcatttgatccttacaacaaccccagaggtagatgtggcatgaatt
accattattctcctttgagaagaagaaactgagcatcaaagaagcttgtt
ggccttcttgccagaaatcacccagtttgtaaatggtaaaagagggcttg
aaaccaggttctctgactctgacttcaagcactctcatacatcatctatt
taattttttggagctaggtattttatacttaggattctaaatattgcata
accattgaatgccacaccacccttgtattcagtgcaaaaaatgggacttt
tcttaataaatagagaaatggaggtgcctaaaattacaaaattgcactag
agagatagtgatagaactgggaaactcttagtctaatattttatctttta
ttcatatgatggaatactaagctcaatggcagaatcttcagtcagcaatg
gtgttcaggttatgtgaactatctgaaggattcctgaactatcatatcta
ggaatgtagcgtttaaaagctcttagaatttttcatactcttaggtcctc
ctgacttgtgcttcaattcatgcataaacttattttataaggtctccgtc
tgcccttgctggagataacatttttgtttatccaacaaagggtattttat
cttattattaaattctgactttgtatagaagagaaatgaagtgataatct
atataaattaagtatgattagtacatatgggttattcacttggataatat
ggagtaaaattttaattcatggctaattacctccacctccactacctagt
ggcctcccctcaccaatattagccaaaataaatcaaatttggaactacaa
acctacttcaaaaagggtaaggtatataataagcaatatcatcaagtcaa
atagtatttttttaaccatgtacaaggcatcatgctaggtgttacgaaga
tgcatgaaatatataagatgtggttccaaccctcacagagtttatagaca
tcacataataaattctgaagtcaaatataaattaatttaaaattatctgc
tgttcagcattgttcactagtgcagccaaacaatgtcatcttgttgaaag
gcattggtagtaaaaactgtgtctgaaatacccctattcaaaggtttcgt
aaatttgatatagtcagggacacgaacaagatccttttacatttttattt
tgatgttagtatgctgtgctcataaatccatgacagaaagccccctcccc
tgagactttccacttccttttctggctttcactgttgcagaggctgctat
attcacgacatgtggcctacagagttctcaggatgtaagcaagccaaact
gaagaccaaatttgtagttcttgctctcctaaacagatgcatatgtggca
cttcagctcttccagattacaacagaatttaggtttatgcataagtctaa
taagtcttcctcatactgaacttcctaatactactccccttttctatctt
ttcaacccacatgaccactattagacagatagtcagaaccagaaggaagt
gaaatgctcactcaacaagcagagcatattctcaatgcagttatcataga
caagacacactatgatagaattggtgctgctatgtattattttga
>BTKp
(SEQ ID NO: 5)
Tgcatttcctaggagaatccctgggggaatcattgcagttggagcataat
gtagggggcccctgagaaaacctccaggcttcaagtgacatacctagtct
gctttaccggtttacaggactcaagagaaaggtggacattgagagttaat
ccctgaggccaaatcttaaatggagaaagtcaacatccacagaaaatggg
gaagggcacaagtatttctgtgggcttatattccgacatttttatctgta
ggggaaaaatgctttcttagaaaatgactcagcacggggaagtcttgtct
ctacctctgtcttgttttgtcctttggggtcccttcactatcaagttcaa
ctgtgtgtccctgagactcctctgccccggaggacaggagactcgaaaaa
cgctcttcctggccagtctctttgctctgtgtctgccagcccccagcatc
tctcctctttcctgtaagcccctctccctgtgctgactgtcttcatagta
ctttaggtatgttgtccctttacctctgggaggatagcttgatgacctgt
ctgctcaggccagccccatctagagtctcagtggccccagtcatgttgag
aaaggttctttcaaagatagactcaagatagtagtgtcagaggtcccaag
caaatgaagggcggggacagttgagggggtggaatagggacggcagcagg
gaaccagatagcatgctgctgagaagaaaaaaagacattggtttaggtca
ggaagcaaaaaaagggaactgagtggctgtgaaagggtggggtttgctca
gactgtccttcctctctggactgtaagaattagtctc
>coBTK
(SEQ ID NO: 6)
atggccgctgtgatcctggagagcattttcctgaagaggtcccagcagaa
aaagaaaacctctcccctgaactttaagaaaagactgttcctgctgacag
tgcacaagctgtcttactatgagtacgactttgagcggggccgccgagga
tcaaaaaaggggagcatcgatgtggagaagattacatgcgtggagaccgt
ggtccctgaaaagaatccaccccctgagaggcagatcccaagacggggcg
aggagtcctctgagatggagcagattagtatcattgagcgcttcccctat
ccttttcaggtggtgtacgacgagggaccactgtatgtgttctcacccac
agaggagctgagaaagaggtggattcaccagctgaagaacgtgattagat
acaatagcgatctggtgcagaagtatcacccttgtttttggatcgacggg
cagtacctgtgctgttcccagacagctaagaacgctatgggatgccagat
tctggaaaatcggaacggatctctgaaaccagggagttcacaccgcaaga
ccaaaaagcccctgcctccaacacccgaggaggatcagatcctgaaaaag
cctctgccacccgagcctgctgcagccccagtcagcacttccgaactgaa
aaaggtggtggctctgtatgactacatgcccatgaatgctaacgatctgc
agctgagaaagggcgacgagtatttcattctggaagagtctaatctgcct
tggtggagggccagagataagaacggacaggaggggtacatcccatctaa
ttatgtgaccgaggctgaggactctattgagatgtacgagtggtatagca
agcacatgacacggtcccaggctgagcagctgctgaagcaggagggcaaa
gagggagggtttatcgtgcgcgattctagtaaggccggcaaatacactgt
gtcagtgttcgctaagagcaccggagacccccagggcgtgatcagacact
atgtggtgtgttccacacctcagtctcagtactatctggctgagaagcac
ctgtttagtacaatcccagagctgattaactaccaccagcacaattctgc
cggcctgatcagcaggctgaagtatcccgtctcccagcagaacaaaaatg
ctccttctaccgctggactggggtacggcagttgggagattgatccaaag
gacctgacattcctgaaggagctgggaactgggcagtttggcgtggtgaa
gtatggaaaatggagagggcagtacgatgtggccatcaagatgatcaagg
agggctcaatgagcgaggacgagttcatcgaggaggctaaggtcatgatg
aacctgtcccacgagaaactggtgcagctgtatggagtgtgcaccaagca
gcggcccatttttatcattacagagtacatggctaatgggtgtctgctga
actatctgcgcgagatgagacacagattccagacacagcagctgctggaa
atgtgcaaggatgtgtgtgaggctatggagtacctggagtctaagcagtt
tctgcaccgggacctggctgctcgcaattgcctggtgaacgatcagggcg
tggtgaaggtgagtgacttcggactgtcaaggtatgtgctggatgacgag
tacaccagctccgtgggctctaagtttcctgtgagatggtctccacccga
ggtgctgatgtatagcaagttctcctctaagagcgatatctgggcctttg
gcgtgctgatgtgggaaatctacagcctgggcaagatgccttacgagcgg
ttcacaaattccgagacagctgagcacatcgcccagggcctgcgcctgta
ccggccacatctggcctctgagaaggtgtacaccatcatgtacagctgtt
ggcacgagaaggccgacgagagacccacattcaagatcctgctgtccaac
attctagatgtgatggacgaggagagctga
>co2BTK
(SEQ ID NO: 7)
atggccgccgtgatcctggaaagcatcttcctgaagcggagccagcagaa
gaagaaaaccagccccctgaacttcaagaagcggctgttcctgctgaccg
tgcacaagctgtcctactacgagtacgacttcgagcggggcagacggggc
agcaagaagggcagcatcgacgtcgagaagatcacctgcgtggagaccgt
ggtgcccgagaagaacccccctcccgagcggcagatccccagacggggcg
aggaaagcagcgagatggaacagatcagcatcatcgagcggttcccttac
ccattccaagtggtgtacgacgagggccccctgtacgtgttcagccccac
cgaggaactgcggaagcggtggattcaccagctgaagaacgtgatccggt
acaacagcgacctggtgcagaagtaccacccctgcttttggatcgacggc
cagtacctgtgctgcagccagaccgccaagaacgctatgggctgccagat
tctggaaaaccggaacggcagcctgaagcccggcagcagccacagaaaga
ccaagaagcccctgccccccacccccgaagaggaccagatcctgaagaag
cctctgcctcccgagcccgccgctgcacctgtgagcaccagcgagctgaa
gaaagtggtggccctgtacgactacatgcccatgaacgccaacgacctgc
agctgcggaagggcgacgagtacttcatcctggaagaaagcaacctgccc
tggtggcgggccagggacaagaacggccaggaaggctacatccccagcaa
ctacgtgaccgaggccgaggactccatcgagatgtacgagtggtacagca
agcacatgaccagaagccaggccgaacagctgctgaagcaggaaggcaaa
gagggcggcttcatcgtccgggacagcagcaaggccggcaagtacaccgt
gagcgtgttcgccaagagcaccggcgacccccagggcgtgatccggcact
acgtggtgtgcagcaccccccagagccagtactacctggccgagaagcac
ctgttcagcaccatccccgagctgatcaactatcaccagcacaacagcgc
tggactgatttctcggctgaagtaccccgtgtcccagcagaacaaaaacg
cccccagcacagccggcctgggctacggcagctgggagatcgaccccaag
gacctgaccttcctgaaagagctgggcaccggccagttcggcgtggtgaa
gtacggcaagtggaggggccagtacgacgtggccatcaagatgatcaagg
aaggcagcatgagcgaggacgagttcatcgaggaagccaaagtgatgatg
aacctgagccacgagaagctggtgcagctgtacggcgtgtgcaccaagca
gcggcccatcttcatcatcaccgagtacatggccaacggctgcctgctga
actacctgcgggagatgcggcacaggttccagacacagcagctgctcgaa
atgtgcaaggacgtgtgcgaggctatggaatacctggaatccaagcagtt
cctgcaccgggacctggccgccagaaactgcctggtgaacgaccaggggg
tggtgaaggtgtccgacttcggcctgagcagatacgtgctggacgacgag
tacaccagcagcgtgggcagcaagttccccgtgcggtggagcccccctga
ggtgctgatgtacagcaagttcagcagcaagagcgacatctgggccttcg
gcgtgctgatgtgggagatctacagcctgggcaagatgccctacgagcgg
ttcaccaacagcgagaccgccgagcacatcgcccagggcctgcggctgta
caggccccacctggccagcgagaaggtgtacaccatcatgtacagctgct
ggcacgagaaggccgacgagaggcccaccttcaagatcctgctgtccaac
atcctggacgtgatggacgaggaaagctga

Sequence elements relative to FIG. 92 (if not already shown above)-these are constructs that were tested as part of the process of identifying the above sequences as our top candidates for clinical gene therapy vectors:

>GFP
(SEQ ID NO: 8)
atggtgagcaagggcgaggagctgttcaccggggtggtgcccatcctggt
cgagctggacggcgacgtaaacggccacaagttcagcgtgtccggcgagg
gcgagggcgatgccacctacggcaagctgaccctgaagttcatctgcacc
accggcaagctgcccgtgccctggcccaccctcgtgaccaccctgaccta
cggcgtgcagtgcttcagccgctaccccgaccacatgaagcagcacgact
tcttcaagtccgccatgcccgaaggctacgtccaggagcgcaccatcttc
ttcaaggacgacggcaactacaagacccgcgccgaggtgaagttcgaggg
cgacaccctggtgaaccgcatcgagctgaagggcatcgacttcaaggagg
acggcaacatcctggggcacaagctggagtacaactacaacagccacaac
gtctatatcatggccgacaagcagaagaacggcatcaaggtgaacttcaa
gatccgccacaacatcgaggacggcagcgtgcagctcgccgaccactacc
agcagaacacccccatcggcgacggccccgtgctgctgcccgacaaccac
tacctgagcacccagtccgccctgagcaaagaccccaacgagaagcgcga
tcacatggtcctgctggagttcgtgaccgccgccgggatcactctcggca
tggacgagctgtacaagtaa
>Intron 4
(SEQ ID NO: 9)
ctcaaaaagaatacatagcagcaccaattctatcatagtgtgtatgtcta
tgataactgcattgagaaagatgctctgcttgttgagtgagcatttcact
tccttctggttctgactatctgtctaatagtggtcatgtgggttgaaaag
atagaaaaggggagtagtattaggaagttcagtatgaggaagacttatta
gacttatgcataaacctaaattctgttgtaatctggaagagctgaagtgc
cacatatgcatctgtttaggagagcaagaactacaaatttggtatcagtt
tggcttgcttacatcctgagaactctgtaggccacatgtcgtgaatatag
cagcctctgcaacagtgaaagccagaaaaggaagtggaaagtctcagggg
agggggctttctgtcatggatttatgagcacagcaagactaacaagcaaa
aagaaaaatgtaaaaggatcttgttcgtgtccctgactatatcaaattta
cgaaacctttgaaagaggggtatttcagacacagtttttactaccaatgc
attcaacaagatgacattgtttggctgcactagtgaacaatgctgaacag
cagataattttaaattaatttatatttgacttcagaatttattatgtgat
gtctataaactctgtgagggttggaaccacatcttatatatttcatgcat
cttcgtaacacctagcatgatgccttgtacatggttaaaaaaatactatt
tgacttgatgatattgatattatataccttaccattttgaagtaggtttg
tagttccaaatttgatttattttggctaatattggtgaggggaggccact
aggtagtggaggtggaggtaattagccatgaattaaaattttactccata
ttatccaagtgaataacccatatgtactaatcaagacttaatttatatag
attatcacttcatttctatctatacaaagtcagaatttaataataagata
aaataccattgttggataaacaaaaatgttatctccagcaagggcagacg
gagaccttataaaataagtttatgcatgaattgaagcacaagtcaggagg
acctaagagtatgaaaaattctaagagcttttaaacgctacattcctaga
tatgaagagttcaggaatccttcagatagttcacataacctgaacaccat
tgctgactgaagattctgcc
>Intron 5
(SEQ ID NO: 10)
Ccatcatatgaataaaagataaaatattagactaagagtttcccagttct
atcactatctctctagtgcaattttgtaattttaggcacctccatttctc
tatttattaagaaaagtcccattttttgcactgaatacaagggtggtgtg
gcattcaatggttatgcaatatttagaatcctaagtataaaatacctagc
tccaaaaaattaaatagatgatgtatgagagtgatgaagtcagagtcaga
gaacctggtttcaagccctcttttaccatttacaaactgggtgatttctg
gcaagaaggccaacaagcttctttgatgctcagtttcttcttctcaaagg
agaataatggtaattcatgccacatctacctctggggttgttgtaaggat
caaatgaagtaataatacatgtaaaagcacatggaaaactgtaaatcact
gtatacgaaagcactgtacatctattgttgtggataaatatttcccaagg
aaatatttcatattccttgattgtgaggacctaaacttgaacagtttcag
catttcacatcaaccatacagtgtgttcagataaacttgactgtaaatgc
ttgcagcacaggatgcatgcacacatatttgtgtgtatatatagccacat
gtcaagtgactgagagattaactggttgagttgacatcaggaaaggctgc
ctagagaaacaggaaggatggcagactgataatggtatggttgggttatt
gcatgacagccattgtataataaacatttagattctgatactcactggct
ggctataccagccttgtacactccatgaatgcaagtttggttggtcaaaa
ttatattagccaagtctttcaaactgaaagggaacaggaggaacgaagat
gaaggatcatctattcaattattgctatgtgccagatattgagctaaata
ccttttatatattatctcatttaattttcacaacaaccttggcctaaaaa
caacaacaatgataactaacacttaatgagcacttactatgtagcaggct
ctgtcccaagtgcttcaaatgtattacttatttgatctttgcagtagcct
taagaagtatgtattattcccctatttacagatgaggaaactgaagctca
aagatgt
>Intron 13
(SEQ ID NO: 11)
ggtagggtggactggccagttgcaaaaactacctttgctggccttgcctt
agggagtgtccttgaggtacactgttctgcagcagctgcctcaaggacgc
tcaagacagatccaagcaaaagttattcactgattttcttcctctagtgg
ctacgactgggactgcaaaaacatagattcataaagggctttgtcgttgt
cttgggtctttttgtcttttatttttaattgtgggaaaattttcagtact
atccctgagttcattaactaccatcactaacataatcataaagggatttg
gggaggttgcttagtctatcttcttgccttatggccaccttgaacctaaa
attcccagattcctctaaccaatgaatcccgtttctgagattgacttaag
caaagacagattagtacttctaaaaatttcccttttactagttttcctat
ttctaccccagtagggatttttgtctattgtaagaattatacattcatga
ccccaaagaatcacaccaagacttta
>1kb
>CONTIG
(SEQ ID NO: 14)
tcaaaaagaatacatagcagcaccaattctatcatagtgtgtatgtctat
gataactgcattgagaaagatgctctgcttgttgagtgagcatttcactt
ccttctggttctgactatctgtctaatagtggtcatgtgggttgaaaaga
tagaaaaggggagtagtattaggaagttcagtatgaggaagacttattag
acttatgcataaacctaaattctgttgtaatctggaagagctgaagtgcc
acatatgcatctgtttaggagagcaagaactacaaatttggtatcagttt
ggcttgcttacatcctgagaactctgtaggccacatgtcgtgaatatagc
agcctctgcaacagtgaaagccagaaaaggaagtggaaagtctcagggga
gggggctttctgtcatggatttatgagcacagcaagactaacaagcaaaa
agaaaaatgtaaaaggatcttgttcgtgtccctgactatatcaaatttac
gaaacctttgaaagaggggtatttcagacacagtttttactaccaatgcc
tttcaacaagatgacattgtttggctgcactagtgaacaatgctgaacag
cagataattttaaattaatttatatttgacttcagaatttattatgtgat
gtctataaactctgtgagggttggaaccacatcttatatatttcatgcat
cttcgtaacacctagcatgatgccttgtacatggttaaaaaaatactatt
tgacttgatgatattgatattatataccttaccattttgaagtaggtttg
tagttccaaatttgatttattttggctaatattggtgaggggaggccact
aggtagtggaggtggaggtaattagccatgaattaaaattttactccata
ttatccaagtgaataacccatatgtactaatcaagacttaatttatatag
attatcacttcatttctatctatacaaagtcagaatttaataataagata
aaataccattgttggataaacaaaaatgttatctccagcaagggcagacg
gagaccttataaaataagtttatgcatgaattgaagcacaagtcaggagg
acctaagagtatgaaaaattctaagagcttttaaacgctacattcctaga
tatgaagagttcaggaatccttcagatagttcacataacctgaacaccat
tgctgactgaagattctgccattgagatagtattccatcatatgaataaa
agataaaatattagactaagagtttcccagttctatcactatctctctag
tgcaattttgtaattttaggcacctccatttctctatttattaagaaaag
tcccattttttgcactgaatacaagggtggtgtggcattcaatggttatg
caatatttagaatcctaagtataaaatacctagctccaaaaaattaaata
gatgatgtatgagagtgcttgaagtcagagtcagagaacctggtttcaag
ccctcttttaccatttacaaactgggtgatttctggcaagaaggccaaca
agcttctttgatgctcagtttcttcttctcaaaggagaataatggtaatt
catgccacatctacctctggggttgttgtaaggatcaaatgaagtaataa
tacatgtaaaagcacatggaaaactgtaaatcactgtatacgaaagcact
gtacatctattgttgtggataaatatttcccaaggaaatatttcatattc
cttgctttgtgaggacctaaacttgaacagtttcagcatttcacatcaac
catacagtgtgttcagataaacttgactgtaaatgcttgcagcacaggat
gcatgcacacatatttgtgtgtatatatagccacatgtcaagtgactgag
agattaactggttgagttgacatcaggaaaggctgcctagagaaacagga
aggatggcagactgataatggtatggttgggttattgcatgacagccctt
tgtcttaataaacatttagattctgatactcactggctggctataccagc
cttgtacactcccttgaatgcaagtttggttggtcaaaattatattagcc
aagtctttcaaactgaaagggaacaggaggaacgaagatgaaggatcatc
tattcaattattgctatgtgccagatattgagctaaataccttttatata
ttatctcatttaattttcacaacaaccttggcctaaaaacaacaacaatg
ataactaacacttaatgagcacttactatgtagcaggctctgtcccaagt
gcttcaaatgtattacttatttgatctttgcagtagccttaagaagtatg
tattattcccctatttacagatgaggaaactgaagctcaaagatgttatc
cttatttctctggggtagggtggactggccagttgcaaaaactacctttg
ctggccttgccttagggagtgtccttgaggtacactgttctgcagcagct
gcctcaaggacgctcaagacagatccaagcaaaagttattcactgatttt
cttcctctagtggctacgactgggactgcaaaaacatagattcataaagg
gattgtcgttgtatgggtattttgtatttatttttaattgtgggaaaatt
ttcagtactatccctgagttcattaactaccatcactaacataatcataa
agggatttggggaggttgcttagtctatcttcttgccttatggccacctt
gaacctaaaattcccagattcctctaaccaatgaatcccgtttctgagat
tgacttaagcaaagacagattagtacttctaaaaatttcccttttactag
ttttcctatttctaccccagtagggatttttgtctattgtaagaattata
cattcatgaccccaaagaatcacaccaagacttta
>revCONTIG
(SEQ ID NO: 15)
taacaataaagtatggtgtgattctttggggtcatgaatgtataattctt
acaatagacaaaaatccctactggggtagaaataggaaaactagtaaaag
ggaaatttttagaagtactaatctgtattgataagtcaatctcagaaacg
ggattcattggttagaggaatctgggaattttaggttcaaggtggccata
aggcaagaagatagactaagcaacctccccaaatccctttatgattatgt
tagtgatggtagttaatgaactcagggatagtactgaaaattttcccaca
attaaaaataaaagacaaaaagacccaagacaacgacaaagccctttatg
aatctatgtttttgcagtcccagtcgtagccactagaggaagaaaatcag
tgaataacttttgcttggatctgtcttgagcgtccttgaggcagctgctg
cagaacagtgtacctcaaggacactccctaaggcaaggccagcaaaggta
gtttttgcaactggccagtccaccctaccccagagaaataaggataacat
ctttgagcttcagtttcctcatctgtaaataggggaataatacatacttc
ttaaggctactgcaaagatcaaataagtaatacatttgaagcacttggga
cagagcctgctacatagtaagtgctcattaagtgttagttatcattgttg
ttgtttttaggccaaggttgttgtgaaaattaaatgagataatatataaa
aggtatttagctcaatatctggcacatagcaataattgaatagatgatcc
ttcatcttcgttcctcctgttccctttcagtttgaaagacttggctaata
taattttgaccaaccaaacttgcattcaagggagtgtacaaggctggtat
agccagccagtgagtatcagaatctaaatgtttattaagacaaagggctg
tcatgcaataacccaaccataccattatcagtctgccatccttcctgttt
ctctaggcagcctttcctgatgtcaactcaaccagttaatctctcagtca
cttgacatgtggctatatatacacacaaatatgtgtgcatgcatcctgtg
ctgcaagcatttacagtcaagtttatctgaacacactgtatggttgatgt
gaaatgctgaaactgttcaagtttaggtcctcacaaagcaaggaatatga
aatatttccttgggaaatatttatccacaacaaagagatgtacagtgatt
cgtatacagtgatttacagttttccatgtgcttttacatgtattattact
tcatttgatccttacaacaaccccagaggtagatgtggcatgaattacca
ttattctcctttgagaagaagaaactgagcatcaaagaagcttgttggcc
ttcttgccagaaatcacccagtttgtaaatggtaaaagagggcttgaaac
caggttctctgactctgacttcaagcactctcatacatcatctatttaat
tttttggagctaggtattttatacttaggattctaaatattgcataacca
ttgaatgccacaccacccttgtattcagtgcaaaaaatgggacttttctt
aataaatagagaaatggaggtgcctaaaattacaaaattgcactagagag
atagtgatagaactgggaaactcttagtctaatattttatcttttattca
tatgatggaatactaagctcaatggcagaatcttcagtcagcaatggtgt
tcaggttatgtgaactatctgaaggattcctgaactatcatatctaggaa
tgtagcgtttaaaagctcttagaatttttcatactcttaggtcctcctga
cttgtgcttcaattcatgcataaacttattttataaggtctccgtctgcc
cttgctggagataacatttttgtttatccaacaaagggtattttatctta
ttattaaattctgactttgtatagaagagaaatgaagtgataatctatat
aaattaagtcttgattagtacatatgggttattcacttggataatatgga
gtaaaattttaattcatggctaattacctccacctccactacctagtggc
ctcccctcaccaatattagccaaaataaatcaaatttggaactacaaacc
tacttcaaaaagggtaaggtatataataagcaatatcatcaagtcaaata
gtatttttttaaccatgtacaaggcatcatgctaggtgttacgaagatgc
atgaaatatataagatgtggttccaaccctcacagagtttatagacatca
cataataaattctgaagtcaaatataaattaatttaaaattatctgctgt
tcagcattgttcactagtgcagccaaacaatgtcatcttgttgaaaggca
ttggtagtaaaaactgtgtctgaaatacccctattcaaaggtttcgtaaa
tttgatatagtcagggacacgaacaagatccttttacatttttctttttg
cttgttagtcttgctgtgctcataaatccatgacagaaagccccctcccc
tgagactttccacttccttttctggctttcactgttgcagaggctgctat
attcacgacatgtggcctacagagttctcaggatgtaagcaagccaaact
gaagaccaaatttgtagttcttgctctcctaaacagatgcatatgtggca
cttcagctcttccagattacaacagaatttaggtttatgcataagtctaa
taagtcttcctcatactgaacttcctaatactactcccatttctatcttt
tcaacccacatgaccactattagacagatagtcagaaccagaaggaagtg
aaatgctcactcaacaagcagagcatctttctcaatgcagttatcataga
caagacacactatgatagaattggtgctgctatgtattctttttga
>ABCD
(SEQ ID NO: 16)
ctggggtatggcaggggctgggcagcagcagcaatgtaccttgcttggga
cccctaaaaaccagagagacagcatggctggtgccatttatcagctagtg
gaggaggctgacggagggtgggagtgtcatcagcacaaggccctggcagt
cccttctggtgattagagaggccgaaagggtcctttccgacaagggctga
gggtgggcggaacaggaagagaaaaatgtgacatgaggtgaccatccgaa
caggtagcaaatgttagaaaggggtacctctggcaaacttagtggaaaag
taatattgcagggagcagtcagataaaaacaagcccttctgtcaaatagt
gcttgaagactcaatagggatacatgggtcaatgaagcctttagaaaaag
aaatactaagaggcagattctctgagaacatggtaaaagctcacgctcca
cgttatgaagttgacattgtgagctagggaaaggcctggctaggccaggg
tgtaggctacctgccttgagctgtaccaggccaaatgtcgccagggtcag
agctggcttattaaaggactgtgtggaagctgtgccaacctcgtggtaac
aatgggtaaaagactgggccaggagaaagcagcctctgcctcagcccaga
cagtgcggccaaccatgaggttgtggcaaaggtttctcctcttaccattg
ccctccatgtgcatggcttgcttttctcttgtcttcattatttctccttt
cctttcctcggatccacgcgtgaattctttgtaaactccttatggtgcga
actaatgtaactttccatccagttatgggggattggtgcaattttaaatt
atcactatgatttgctatttccatttgagcaaatttcctatagagtttcc
tttcagtggactagacccatatcaggaagtgacttaggtataaagggaag
atacagctttcgaaaaccaaagtttgggcgttctccaaagagttatcaga
tacccccttctacacccacaatgatctgattgctgagatctgattgctaa
ctactgaaaataaggaagaactagaattttcagtgacacagtgctcagca
agaagctagaaaagaggccttgacatatttgactccaaagctacttggtt
atgcatgaagccatctggggaggggaaggaggagggagaactcctctgag
gaccctgaaacaattgggccacgtgtgactttcagtttctatggagattc
atgtgcagtggctgagggcaatctgagagcattggaaacccagaagcttt
aaacgcgtaggaaagacagggaatggccagaatcttcctagccaattgag
tagtgattcaaggagaaatcaagagaaaacactacttatggatattttgg
ctaagtagtcatttgaagtacagttgactggttattttattttaaatcat
atctcatagactatcccaatcataggctggctgtgtagttttctgaattt
tgctctggtattcttcttatttttttttttatttttattttttatttttt
ttttgagactccaggctggagtgcagtgtcacgatcttggctcactgcaa
cctccgcctcccgagttcaagcgattctcctgcctcagcctcctgagttg
ctgggactacaggcgcctgtcaccacgcccggctaattttttgtatttta
atagagacgggatttcaccatgttggccaggctggtctctaactcctgac
ctcaagtgatccgccagcctcagcctcccaaagtgctgggattacgggca
tgagccactgcgcctggacttattattataatagtattttatcttatgag
cgaagataagagcccaagatggtttagtttactgattctgcaagtgctat
ttctattaattccttggcatactgcagtttgtatgatggctgcactatgt
taataagatcgtattctgaattctgttgctccatagggagctgggaggct
gcaaaaggtggccctgtaaaaatctttgcatttataatttaataattaca
gaccccagtgggacaatgtttgaaaaattatattcaccgtctaggaaatt
gggaactgaaagtccaatatctgcctcagtggagttctggcacctgcatt
atcccttctgggtatatcaagatcaacagctgcacagatacttttgcttt
tcacagattctacacatatcatataaaggtgaatagtgtaaagctacctc
tacaccttaccaagcacacagg
>DCBA
(SEQ ID NO: 17)
cctgtgtgatggtaaggtgtagaggtagattacactattcacctttatat
gatatgtgtagaatctgtgaaaagcaaaagtatctgtgcagctgttgatc
ttgatatacccagaagggataatgcaggtgccagaactccactgaggcag
atattggactttcagttcccaatttcctagacggtgaatataatttttca
aacattgtcccactggggtctgtaattattaaattataaatgcaaagatt
tttacagggccaccttttgcagcctcccagctccctatggagcaacagaa
ttcagaaagacgaagcttattaacaagagtgcagccatcatacaaactgc
agtatgccaaggaattaatagaaatagcacttgcagaatcagtaaactaa
accatcttgggctcttatcttcgctcataagataaaatactattaagaat
aataagtccaggcgcagtggctcatgcccgtaatcccagcactttgggag
gctgaggctggcggatcacttgaggtcaggagttagagaccagcctggcc
aacatggtgaaatcccgtctctattaaaatacaaaaaattagccgggcgt
ggtgacaggcgcctgtagtcccagcaactcaggaggctgaggcaggagaa
tcgcttgaactcgggaggcggaggttgcagtgagccaagatcgtgacact
gcactccagcctggagtctcaaaaaaaaaataaaaaataaaaataaaaaa
aaaaaagaagaagaataccagagcaaaattcagaaaactacacagccagc
ctatgattgggaagagtctatgagatatgatttaaaataaaataaccagt
caactgtacttcaaatgactacttagccaaaatatccaagaagtagtgtt
ttctcttgatttctccttgaaagcactactcaattggctaggaagattct
ggccattccctgtctttcctacgcgtttaaagatctgggtttccaatgct
ctcagattgccctcagccactgcacatgaatctccatagaaactgaaagt
cacacgtggcccaattgtttcagggtcctcagaggagttctccctcctcc
ttcccctccccagatggcttcatgcataaccaagtagctttggagtcaaa
tatgtcaaggcctatttctagatcttgctgagcactgtgtcactgaaaat
tctagttcttccttattttcagtagttagcaatcagatctcagcaatcag
atcattgtgggtgtagaagggggtatctgataactctttggagaacgccc
aaactttggttttcgaaagctgtatcttccctttatacctaagtcacttc
ctgatatgggtctagtccactgaaaggaaactctataggaaatttgctca
aatggaaatagcaaatcatagtgataatttaaaattgcaccaatccccca
taactggatggaaagttacattagttcgcaccataaggagtttacaaaga
attcacgcgtggatccgaggaaaggaaaggagaaataatgaagacaagag
aaaagcaagccatgcacatggagggcaatggtaagaggagaaacctttgc
cacaacctcaagggttggccgcactgtctgggctgaggcagaggctgatt
ctcctggcccagtatttacccattgttaccacgaggttggcacagcttcc
acacagtcctttaataagccagctctgaccctggcgacatttggcctggt
acagctcaaggcaggtagcctacaccctggcctagccaggcctttcccta
gctcacaaaggtcaacttcataacgtggagcgtgagcttttaccatgttc
tcagagaatctgcctcttagtatttctttttctaaaggcttcattgaccc
atgtatccctattgagtcttcaagcactatttgacagaagggcttgtttt
tatctgactgctccctgcaatattacttttccactaagtttgccagaggt
acccctttctaacatttgctacctgttcggatggtcacctcatgtcacat
ttttctcttcctgttccgcccaccctcagcccttgtcggaaaggaccctt
tcggcctctctaatcaccagaagggactgccagggccttgtgctgatgac
actcccaccctccgtcagcctcctccactagctgataaatggcaccagcc
atgctgtctctctggtttttaggggtcccaagcaaggtacattgctgctg
ctgcccagcccctgccataccccag
>AB
(SEQ ID NO: 18)
ctggggtatggcaggggctgggcagcagcagcaatgtaccttgcttggga
cccctaaaaaccagagagacagcatggctggtgccatttatcagctagtg
gaggaggctgacggagggtgggagtgtcatcagcacaaggccctggcagt
cccttctggtgattagagaggccgaaagggtcctttccgacaagggctga
gggtgggcggaacaggaagagaaaaatgtgacatgaggtgaccatccgaa
caggtagcaaatgttagaaaggggtacctctggcaaacttagtggaaaag
taatattgcagggagcagtcagataaaaacaagcccttctgtcaaatagt
gcttgaagactcaatagggatacatgggtcaatgaagcctttagaaaaag
aaatactaagaggcagattctctgagaacatggtaaaagctcacgctcca
cgttatgaagttgacattgtgagctagggaaaggcctggctaggccaggg
tgtaggctacctgccttgagctgtaccaggccaaatgtcgccagggtcag
agctggcttattaaaggactgtgtggaagctgtgccaacctcgtggtaac
aatgggtaaaagactgggccaggagaaagcagcctctgcctcagcccaga
cagtgcggccaaccatgaggttgtggcaaaggtttctcctcttaccattg
ccctccatgtgcatggcttgcttttctcttgtcttcattatttctccttt
cctttcctcggatccacgcgtgaattctttgtaaactccttatggtgcga
actaatgtaactttccatccagttatgggggattggtgcaattttaaatt
atcactatgatttgctatttccatttgagcaaatttcctatagagtttcc
tttcagtggactagacccatatcaggaagtgacttaggtataaagggaag
atacagctttcgaaaaccaaagtttgggcgttctccaaagagttatcaga
tacccccttctacacccacaatgatctgattgctgagatctgattgctaa
ctactgaaaataaggaagaactagaattttcagtgacacagtgctcagca
agaagctagaaaagaggccttgacatatttgactccaaagctacttggtt
atgcatgaagccatctggggaggggaaggaggagggagaactcctctgag
gaccctgaaacaattgggccacgtgtgactttcagtttctatggagattc
atgtgcagtggctgagggcaatctgagagcattggaaacccagaagcttt
aa
>A
(SEQ ID NO: 19)
ctggggtatggcaggggctgggcagcagcagcaatgtaccttgcttggga
cccctaaaaaccagagagacagcatggctggtgccatttatcagctagtg
gaggaggctgacggagggtgggagtgtcatcagcacaaggccctggcagt
cccttctggtgattagagaggccgaaagggtcctttccgacaagggctga
gggtgggcggaacaggaagagaaaaatgtgacatgaggtgaccatccgaa
caggtagcaaatgttagaaaggggtacctctggcaaacttagtggaaaag
taatattgcagggagcagtcagataaaaacaagcccttctgtcaaatagt
gcttgaagactcaatagggatacatgggtcaatgaagcctttagaaaaag
aaatactaagaggcagattctctgagaacatggtaaaagctcacgctcca
cgttatgaagttgacattgtgagctagggaaaggcctggctaggccaggg
tgtaggctacctgccttgagctgtaccaggccaaatgtcgccagggtcag
agctggcttattaaaggactgtgtggaagctgtgccaacctcgtggtaac
aatgggtaaaagactgggccaggagaaagcagcctctgcctcagcccaga
cagtgcggccaacccttgaggttgtggcaaaggtttctcctcttaccatt
gccctccatgtgcatggcttgcttttctcttgtcttcattatttctcctt
tcctttcctc
>B
(SEQ ID NO: 20)
gaattattgtaaactccttatggtgcgaactaatgtaactttccatccag
ttatgggggattggtgcaattttaaattatcactatgatttgctatttcc
atttgagcaaatttcctatagagtttcattcagtggactagacccatatc
aggaagtgacttaggtataaagggaagatacagctttcgaaaaccaaagt
ttgggcgttctccaaagagttatcagatacccccttctacacccacaatg
atctgattgctgagatctgattgctaactactgaaaataaggaagaacta
gaattttcagtgacacagtgctcagcaagaagctagaaaagaggccttga
catatttgactccaaagctacttggttatgcatgaagccatctggggagg
ggaaggaggagggagaactcctctgaggaccctgaaacaattgggccacg
tgtgactttcagtttctatggagattcatgtgcagtggctgagggcaatc
tgagagcattggaaacccagaagctttaa
>BTKe
(SEQ ID NO: 21)
tccatcacctactagatatatcagtgcagtgaaaacttcgctaaactaac
gctatacctatatcatgaagtgtgtggactagagacaagtgcatatcctt
acggcaattaactgggaaacgtcaaatagtaactaccactcacctttttc
cggaaaatcggcttagtttgcccaccatagccactctgcttcctgtcata
acgccgctttcctgggaaaacgaattggtatttgttataaaatactgaag
atcagcaagtaagtcttacaggttttatcttaatttcgcagcagaaatat
taacgctcaagccaggcgtggagggagagagacccggactcgtatgttat
tctacaacacaaatgtcacattaacaccaaattatgcggaatccatctta
ccctgggcgtacagagaatccttgcccttcttgtactgtgtcactttatg
gggttggtgcttgccacacttcttacagaaagtccggcgggttttaggga
cgttaacctagtaaagaaacagttcagaacgtgcaatgttatttgaccac
aatggcacaacgccctaccttacccagctaaagctgaggcactccaggag
gactcctcattacttgctacctctgactacagggtgggccagccccatgt
gcttcaagcagagcttcctccctccgtcgagccccaaagagggaagagac
ctcattaactccacccccggctaactctacctattgaacccatcacttca
attcctggccccgtagcccggtccctttagggttgatcccggcaagattg
ggttgctctgatatatcgagtccacacaggagcctggacccatcccggca
tagcacgggcgacgaagggggggaaagattaagctggatgttactcggcc
cccaccagcaagtcctaccatgcttgcgtgagcgctatcggcgcggaaag
aaagaaaccgcgaggcaaacggaagtatataggaggttcccgatcgcact
tcctcatgggagtcggtaggagcaatcatagagtgtaaggctcagcgcag
cgccctcgggcggctgagaggactcagttcggagccgcgggcgggagctt
aaggaaggactccgcctaaagggtggtccactcaccccgacttcctcccg
ccccgcagattcaacgtttcgtcactttatctatttggtggactctgcta
cgtagtggcgttcagtgaagggagcagtgtttttcccagatcctctggcc
tccccgtccccgagggaagccaggactagggtcgaatgaaggggtcctcc
acctccacgttccattcctgttccacctcaaggtcactgggaacaccttt
cgcagcaaactgctaattcaatgaagacctggagggagccaattgttcca
gttcatctatcacatggccagttggtccattcaacaaatggttattggat
gcccattatgtggcaggcactgttccgggggagaggtacagtaatctaat
aggcttataaatgtgcaattatgaactaagtactttgaagaaaaggaaca
atgattggcattaaagcagcacccttctgttgagggagtaagtcagcagc
tctaggttctgaaaagtgacaatgaaattgtttggctcctgt
>BTKeΔMyc
(SEQ ID NO: 22)
tgttactcggcccccaccagcaagtcctaccatgcttgcgtgagcgctat
cggcgcggaaagaaagaaaccgcgaggcaaacggaagtatataggaggtt
cccgatcgcacttcctcatgggagtcggtaggagcaatcatagagtgtaa
ggctcagcgcagcgccctcgggcggctgagaggactcagttcggagccgc
gggcgggagcttaaggaaggactccgcctaaagggtggtccactcacccc
gacttcctcccgccccgcagctttcaacgtttcgtcactttatctctttt
ggtggactctgctacgtagtggcgttcagtgaagggagcagtgtttttcc
cagatcctctggcctccccgtccccgagggaagccaggactagggtcgaa
tgaaggggtcctccacctccacgttccattcctgttccacctcaaggtca
ctgggaacacctttcgcagcaaactgctaattcaatgaagacctggaggg
agccaattgttccagttcatctatcacatggccagttggtccattcaaca
aatggttattggatgcccattatgtggcaggcactgttccgggggagagg
tacagtaatctaataggcttataaatgtgcaattatgaactaagtacttt
gaagaaaaggaacaatgattggcattaaagcagcacccttctgttgaggg
agtaagtcagcagctctaggttctgaaaagtgacaatgaaattgtttggc
tcctgt

The sequences of gene expression cassettes below are cloned into pRRL backbone of pRRLSIN.cppt.PGK-GFP.WPRE [PGK-GFP removed] Addgene #12252;

The nucleic acid comprising the promoter with GFP sequence is below (BTKp.GFP): (SEQ ID NO: 23)

gcatttcctaggagaatccctgggggaatcattgcagttggagcataatg
tagggggcccctgagaaaacctccaggcttcaagtgacatacctagtctg
ctttaccggtttacaggactcaagagaaaggtggacattgagagttaatc
cctgaggccaaatcttaaatggagaaagtcaacatccacagaaaatgggg
aagggcacaagtatttctgtgggcttatattccgacatttttatctgtag
gggaaaaatgctttcttagaaaatgactcagcacggggaagtcttgtctc
tacctctgtcttgttttgtcctttggggtcccttcactatcaagttcaac
tgtgtgtccctgagactcctctgccccggaggacaggagactcgaaaaac
gctcttcctggccagtctctttgctctgtgtctgccagcccccagcatct
ctcctctttcctgtaagcccctctccctgtgctgactgtcttcatagtac
tttaggtatgttgtccctttacctctgggaggatagcttgatgacctgtc
tgctcaggccagccccatctagagtctcagtggccccagtcatgttgaga
aaggttctttcaaagatagactcaagatagtagtgtcagaggtcccaagc
aaatgaagggcggggacagttgagggggtggaatagggacggcagcaggg
aaccagatagcatgctgctgagaagaaaaaaagacattggtttaggtcag
gaagcaaaaaaagggaactgagtggctgtgaaagggtggggtttgctcag
actgtccttcctctctggactgtaagaattagtctcgaggccaccatggt
gagcaagggcgaggagctgttcaccggggtggtgcccatcctggtcgagc
tggacggcgacgtaaacggccacaagttcagcgtgtccggcgagggcgag
ggcgatgccacctacggcaagctgaccctgaagttcatctgcaccaccgg
caagctgcccgtgccctggcccaccctcgtgaccaccctgacctacggcg
tgcagtgcttcagccgctaccccgaccacatgaagcagcacgacttcttc
aagtccgccatgcccgaaggctacgtccaggagcgcaccatcttcttcaa
ggacgacggcaactacaagacccgcgccgaggtgaagttcgagggcgaca
ccctggtgaaccgcatcgagctgaagggcatcgacttcaaggaggacggc
aacatcctggggcacaagctggagtacaactacaacagccacaacgtcta
tatcatggccgacaagcagaagaacggcatcaaggtgaacttcaagatcc
gccacaacatcgaggacggcagcgtgcagctcgccgaccactaccagcag
aacacccccatcggcgacggccccgtgctgctgcccgacaaccactacct
gagcacccagtccgccctgagcaaagaccccaacgagaagcgcgatcaca
tggtcctgctggagttcgtgaccgccgccgggatcactctcggcatggac
gagctgtacaagtaa
INT4.BTKp.GFP
(SEQ ID NO: 24)
GCATTTCCTAGGAGAATCCCTGGGGGAATCATTGCAGTTGGAGCATAATG
TAGGGGGCCCCTCAAAAAGAATACATAGCAGCACCAATTCTATCATAGTG
TGTCTTGTCTATGATAACTGCATTGAGAAAGATGCTCTGCTTGTTGAGTG
AGCATTTCACTTCCTTCTGGTTCTGACTATCTGTCTAATAGTGGTCATGT
GGGTTGAAAAGATAGAAAAGGGGAGTAGTATTAGGAAGTTCAGTATGAGG
AAGACTTATTAGACTTATGCATAAACCTAAATTCTGTTGTAATCTGGAAG
AGCTGAAGTGCCACATATGCATCTGTTTAGGAGAGCAAGAACTACAAATT
TGGTCTTCAGTTTGGCTTGCTTACATCCTGAGAACTCTGTAGGCCACATG
TCGTGAATATAGCAGCCTCTGCAACAGTGAAAGCCAGAAAAGGAAGTGGA
AAGTCTCAGGGGAGGGGGCTTTCTGTCATGGATTTATGAGCACAGCAAGA
CTAACAAGCAAAAAGAAAAATGTAAAAGGATCTTGTTCGTGTCCCTGACT
ATATCAAATTTACGAAACCTTTGAAAGAGGGGTATTTCAGACACAGTTTT
TACTACCAATGCCTTTCAACAAGATGACATTGTTTGGCTGCACTAGTGAA
CAATGCTGAACAGCAGATAATTTTAAATTAATTTATATTTGACTTCAGAA
TTTATTATGTGATGTCTATAAACTCTGTGAGGGTTGGAACCACATCTTAT
ATATTTCATGCATCTTCGTAACACCTAGCATGATGCCTTGTACATGGTTA
AAAAAATACTATTTGACTTGATGATATTGCTTATTATATACCTTACCCTT
TTTGAAGTAGGTTTGTAGTTCCAAATTTGATTTATTTTGGCTAATATTGG
TGAGGGGAGGCCACTAGGTAGTGGAGGTGGAGGTAATTAGCCATGAATTA
AAATTTTACTCCATATTATCCAAGTGAATAACCCATATGTACTAATCAAG
ACTTAATTTATATAGATTATCACTTCATTTCTCTTCTATACAAAGTCAGA
ATTTAATAATAAGATAAAATACCCTTTGTTGGATAAACAAAAATGTTATC
TCCAGCAAGGGCAGACGGAGACCTTATAAAATAAGTTTATGCATGAATTG
AAGCACAAGTCAGGAGGACCTAAGAGTATGAAAAATTCTAAGAGCTTTTA
AACGCTACATTCCTAGATATGAAGAGTTCAGGAATCCTTCAGATAGTTCA
CATAACCTGAACACCATTGCTGACTGAAGATTCTGCCGGGCCCCTGAGAA
AACCTCCAGGCTTCAAGTGACATACCTAGTCTGCTTTACCGGTTTACAGG
ACTCAAGAGAAAGGTGGACATTGAGAGTTAATCCCTGAGGCCAAATCTTA
AATGGAGAAAGTCAACATCCACAGAAAATGGGGAAGGGCACAAGTATTTC
TGTGGGCTTATATTCCGACATTTTTATCTGTAGGGGAAAAATGCTTTCTT
AGAAAATGACTCAGCACGGGGAAGTCTTGTCTCTACCTCTGTCTTGTTTT
GTCCTTTGGGGTCCCTTCACTATCAAGTTCAACTGTGTGTCCCTGAGACT
CCTCTGCCCCGGAGGACAGGAGACTCGAAAAACGCTCTTCCTGGCCAGTC
TCTTTGCTCTGTGTCTGCCAGCCCCCAGCATCTCTCCTCTTTCCTGTAAG
CCCCTCTCCCTGTGCTGACTGTCTTCATAGTACTTTAGGTATGTTGTCCC
TTTACCTCTGGGAGGATAGCTTGATGACCTGTCTGCTCAGGCCAGCCCCA
TCTAGAGTCTCAGTGGCCCCAGTCATGTTGAGAAAGGTTCTTTCAAAGAT
AGACTCAAGATAGTAGTGTCAGAGGTCCCAAGCAAATGAAGGGCGGGGAC
AGTTGAGGGGGTGGAATAGGGACGGCAGCAGGGAACCAGATAGCATGCTG
CTGAGAAGAAAAAAAGACATTGGTTTAGGTCAGGAAGCAAAAAAAGGGAA
CTGAGTGGCTGTGAAAGGGTGGGGTTTGCTCAGACTGTCCTTCCTCTCTG
GACTGTAAGAATTAGTCTCGAGGCCACCATGGTGAGCAAGGGCGAGGAGC
TGTTCACCGGGGTGGTGCCCATCCTGGTCGAGCTGGACGGCGACGTAAAC
GGCCACAAGTTCAGCGTGTCCGGCGAGGGCGAGGGCGATGCCACCTACGG
CAAGCTGACCCTGAAGTTCATCTGCACCACCGGCAAGCTGCCCGTGCCCT
GGCCCACCCTCGTGACCACCCTGACCTACGGCGTGCAGTGCTTCAGCCGC
TACCCCGACCACATGAAGCAGCACGACTTCTTCAAGTCCGCCATGCCCGA
AGGCTACGTCCAGGAGCGCACCATCTTCTTCAAGGACGACGGCAACTACA
AGACCCGCGCCGAGGTGAAGTTCGAGGGCGACACCCTGGTGAACCGCATC
GAGCTGAAGGGCATCGACTTCAAGGAGGACGGCAACATCCTGGGGCACAA
GCTGGAGTACAACTACAACAGCCACAACGTCTATATCATGGCCGACAAGC
AGAAGAACGGCATCAAGGTGAACTTCAAGATCCGCCACAACATCGAGGAC
GGCAGCGTGCAGCTCGCCGACCACTACCAGCAGAACACCCCCATCGGCGA
CGGCCCCGTGCTGCTGCCCGACAACCACTACCTGAGCACCCAGTCCGCCC
TGAGCAAAGACCCCAACGAGAAGCGCGATCACATGGTCCTGCTGGAGTTC
GTGACCGCCGCCGGGATCACTCTCGGCATGGACGAGCTGTACAAGTAA
INT5.BTKp.GFP
(SEQ ID NO: 25)
GCATTTCCTAGGAGAATCCCTGGGGGAATCATTGCAGTTGGAGCATAATG
TAGGGGGCCCCATCATATGAATAAAAGATAAAATATTAGACTAAGAGTTT
CCCAGTTCTATCACTATCTCTCTAGTGCAATTTTGTAATTTTAGGCACCT
CCATTTCTCTATTTATTAAGAAAAGTCCCATTTTTTGCACTGAATACAAG
GGTGGTGTGGCATTCAATGGTTATGCAATATTTAGAATCCTAAGTATAAA
ATACCTAGCTCCAAAAAATTAAATAGATGATGTATGAGAGTGCTTGAAGT
CAGAGTCAGAGAACCTGGTTTCAAGCCCTCTTTTACCATTTACAAACTGG
GTGATTTCTGGCAAGAAGGCCAACAAGCTTCTTTGATGCTCAGTTTCTTC
TTCTCAAAGGAGAATAATGGTAATTCATGCCACATCTACCTCTGGGGTTG
TTGTAAGGATCAAATGAAGTAATAATACATGTAAAAGCACATGGAAAACT
GTAAATCACTGTATACGAAAGCACTGTACATCTCTTTGTTGTGGATAAAT
ATTTCCCAAGGAAATATTTCATATTCCTTGCTTTGTGAGGACCTAAACTT
GAACAGTTTCAGCATTTCACATCAACCATACAGTGTGTTCAGATAAACTT
GACTGTAAATGCTTGCAGCACAGGATGCATGCACACATATTTGTGTGTAT
ATATAGCCACATGTCAAGTGACTGAGAGATTAACTGGTTGAGTTGACATC
AGGAAAGGCTGCCTAGAGAAACAGGAAGGATGGCAGACTGATAATGGTAT
GGTTGGGTTATTGCATGACAGCCCTTTGTCTTAATAAACATTTAGATTCT
GATACTCACTGGCTGGCTATACCAGCCTTGTACACTCCCTTGAATGCAAG
TTTGGTTGGTCAAAATTATATTAGCCAAGTCTTTCAAACTGAAAGGGAAC
AGGAGGAACGAAGATGAAGGATCATCTATTCAATTATTGCTATGTGCCAG
ATATTGAGCTAAATACCTTTTATATATTATCTCATTTAATTTTCACAACA
ACCTTGGCCTAAAAACAACAACAATGATAACTAACACTTAATGAGCACTT
ACTATGTAGCAGGCTCTGTCCCAAGTGCTTCAAATGTATTACTTATTTGA
TCTTTGCAGTAGCCTTAAGAAGTATGTATTATTCCCCTATTTACAGATGA
GGAAACTGAAGCTCAAAGATGTTAGGGCCCCTGAGAAAACCTCCAGGCTT
CAAGTGACATACCTAGTCTGCTTTACCGGTTTACAGGACTCAAGAGAAAG
GTGGACATTGAGAGTTAATCCCTGAGGCCAAATCTTAAATGGAGAAAGTC
AACATCCACAGAAAATGGGGAAGGGCACAAGTATTTCTGTGGGCTTATAT
TCCGACATTTTTATCTGTAGGGGAAAAATGCTTTCTTAGAAAATGACTCA
GCACGGGGAAGTCTTGTCTCTACCTCTGTCTTGTTTTGTCCTTTGGGGTC
CCTTCACTATCAAGTTCAACTGTGTGTCCCTGAGACTCCTCTGCCCCGGA
GGACAGGAGACTCGAAAAACGCTCTTCCTGGCCAGTCTCTTTGCTCTGTG
TCTGCCAGCCCCCAGCATCTCTCCTCTTTCCTGTAAGCCCCTCTCCCTGT
GCTGACTGTCTTCATAGTACTTTAGGTATGTTGTCCCTTTACCTCTGGGA
GGATAGCTTGATGACCTGTCTGCTCAGGCCAGCCCCATCTAGAGTCTCAG
TGGCCCCAGTCATGTTGAGAAAGGTTCTTTCAAAGATAGACTCAAGATAG
TAGTGTCAGAGGTCCCAAGCAAATGAAGGGCGGGGACAGTTGAGGGGGTG
GAATAGGGACGGCAGCAGGGAACCAGATAGCATGCTGCTGAGAAGAAAAA
AAGACATTGGTTTAGGTCAGGAAGCAAAAAAAGGGAACTGAGTGGCTGTG
AAAGGGTGGGGTTTGCTCAGACTGTCCTTCCTCTCTGGACTGTAAGAATT
AGTCTCGAGGCCACCATGGTGAGCAAGGGCGAGGAGCTGTTCACCGGGGT
GGTGCCCATCCTGGTCGAGCTGGACGGCGACGTAAACGGCCACAAGTTCA
GCGTGTCCGGCGAGGGCGAGGGCGATGCCACCTACGGCAAGCTGACCCTG
AAGTTCATCTGCACCACCGGCAAGCTGCCCGTGCCCTGGCCCACCCTCGT
GACCACCCTGACCTACGGCGTGCAGTGCTTCAGCCGCTACCCCGACCACA
TGAAGCAGCACGACTTCTTCAAGTCCGCCATGCCCGAAGGCTACGTCCAG
GAGCGCACCATCTTCTTCAAGGACGACGGCAACTACAAGACCCGCGCCGA
GGTGAAGTTCGAGGGCGACACCCTGGTGAACCGCATCGAGCTGAAGGGCA
TCGACTTCAAGGAGGACGGCAACATCCTGGGGCACAAGCTGGAGTACAAC
TACAACAGCCACAACGTCTATATCATGGCCGACAAGCAGAAGAACGGCAT
CAAGGTGAACTTCAAGATCCGCCACAACATCGAGGACGGCAGCGTGCAGC
TCGCCGACCACTACCAGCAGAACACCCCCATCGGCGACGGCCCCGTGCTG
CTGCCCGACAACCACTACCTGAGCACCCAGTCCGCCCTGAGCAAAGACCC
CAACGAGAAGCGCGATCACATGGTCCTGCTGGAGTTCGTGACCGCCGCCG
GGATCACTCTCGGCATGGACGAGCTGTACAAGTAA
INT13.BTKp.GFP
(SEQ ID NO: 26)
GCATTTCCTAGGAGAATCCCTGGGGGAATCATTGCAGTTGGAGCATAATG
TAGGGGGCCCGGTAGGGTGGACTGGCCAGTTGCAAAAACTACCTTTGCTG
GCCTTGCCTTAGGGAGTGTCCTTGAGGTACACTGTTCTGCAGCAGCTGCC
TCAAGGACGCTCAAGACAGATCCAAGCAAAAGTTATTCACTGATTTTCTT
CCTCTAGTGGCTACGACTGGGACTGCAAAAACATAGATTCATAAAGGGCT
TTGTCGTTGTCTTGGGTCTTTTTGTCTTTTATTTTTAATTGTGGGAAAAT
TTTCAGTACTATCCCTGAGTTCATTAACTACCATCACTAACATAATCATA
AAGGGATTTGGGGAGGTTGCTTAGTCTATCTTCTTGCCTTATGGCCACCT
TGAACCTAAAATTCCCAGATTCCTCTAACCAATGAATCCCGTTTCTGAGA
TTGACTTAAGCAAAGACAGATTAGTACTTCTAAAAATTTCCCTTTTACTA
GTTTTCCTATTTCTACCCCAGTAGGGATTTTTGTCTATTGTAAGAATTAT
ACATTCATGACCCCAAAGAATCACACCAAGACTTTAGGGCCCCTGAGAAA
ACCTCCAGGCTTCAAGTGACATACCTAGTCTGCTTTACCGGTTTACAGGA
CTCAAGAGAAAGGTGGACATTGAGAGTTAATCCCTGAGGCCAAATCTTAA
ATGGAGAAAGTCAACATCCACAGAAAATGGGGAAGGGCACAAGTATTTCT
GTGGGCTTATATTCCGACATTTTTATCTGTAGGGGAAAAATGCTTTCTTA
GAAAATGACTCAGCACGGGGAAGTCTTGTCTCTACCTCTGTCTTGTTTTG
TCCTTTGGGGTCCCTTCACTATCAAGTTCAACTGTGTGTCCCTGAGACTC
CTCTGCCCCGGAGGACAGGAGACTCGAAAAACGCTCTTCCTGGCCAGTCT
CTTTGCTCTGTGTCTGCCAGCCCCCAGCATCTCTCCTCTTTCCTGTAAGC
CCCTCTCCCTGTGCTGACTGTCTTCATAGTACTTTAGGTATGTTGTCCCT
TTACCTCTGGGAGGATAGCTTGATGACCTGTCTGCTCAGGCCAGCCCCAT
CTAGAGTCTCAGTGGCCCCAGTCATGTTGAGAAAGGTTCTTTCAAAGATA
GACTCAAGATAGTAGTGTCAGAGGTCCCAAGCAAATGAAGGGCGGGGACA
GTTGAGGGGGTGGAATAGGGACGGCAGCAGGGAACCAGATAGCATGCTGC
TGAGAAGAAAAAAAGACATTGGTTTAGGTCAGGAAGCAAAAAAAGGGAAC
TGAGTGGCTGTGAAAGGGTGGGGTTTGCTCAGACTGTCCTTCCTCTCTGG
ACTGTAAGAATTAGTCTCGAGGCCACCATGGTGAGCAAGGGCGAGGAGCT
GTTCACCGGGGTGGTGCCCATCCTGGTCGAGCTGGACGGCGACGTAAACG
GCCACAAGTTCAGCGTGTCCGGCGAGGGCGAGGGCGATGCCACCTACGGC
AAGCTGACCCTGAAGTTCATCTGCACCACCGGCAAGCTGCCCGTGCCCTG
GCCCACCCTCGTGACCACCCTGACCTACGGCGTGCAGTGCTTCAGCCGCT
ACCCCGACCACATGAAGCAGCACGACTTCTTCAAGTCCGCCATGCCCGAA
GGCTACGTCCAGGAGCGCACCATCTTCTTCAAGGACGACGGCAACTACAA
GACCCGCGCCGAGGTGAAGTTCGAGGGCGACACCCTGGTGAACCGCATCG
AGCTGAAGGGCATCGACTTCAAGGAGGACGGCAACATCCTGGGGCACAAG
CTGGAGTACAACTACAACAGCCACAACGTCTATATCATGGCCGACAAGCA
GAAGAACGGCATCAAGGTGAACTTCAAGATCCGCCACAACATCGAGGACG
GCAGCGTGCAGCTCGCCGACCACTACCAGCAGAACACCCCCATCGGCGAC
GGCCCCGTGCTGCTGCCCGACAACCACTACCTGAGCACCCAGTCCGCCCT
GAGCAAAGACCCCAACGAGAAGCGCGATCACATGGTCCTGCTGGAGTTCG
TGACCGCCGCCGGGATCACTCTCGGCATGGACGAGCTGTACAAGTAA
1kb.BTKp.GFP
(SEQ ID NO: 27)
GCATTTCCTAGGAGAATCCCTGGGGGAATCATTGCAGTTGGAGCATAATG
TAGGGGGCCCAAACTGTAGTCTGTTAGCTCACCCTAGAAGAAGGAAATTC
TAGAGTCAAGAGAAGTTAAGTTTTAGACAAGCTGGAAGTTGGAATATACT
GGAGTTTAAGATCATTGACTCAATGAAAAAAAAAAAAGATTTGTCTGCAG
TAAAACCAGAGTTTTAAAAATGATTAGAAATGGACCAGAAAAGAGATAA
GGATAATGAGAAAGGGGGCTCTCTAAAACTATTTGGAACTGAAATTGGGA
CAATGTTTCTGGAAACTATTTTGGCAGTATGTATCAAAATGCAAAATGCA
CATACTTTTTTGACTCTGGAAATGATGCTTGTATAGAGATCCTAATAACA
TGGGAAAATAATTAGGAATAATGTTAAATAAGAACATTAGATCTTTTTTT
GGAAAAAGGTACCTTGAACGGATGGATGGATGGATAGATACATACATACA
TGCATACATACATACATAGATGTTACGAAAAATAGGTAAATTAACTGAAT
GTGCAATATGATCTCAAATATTATAAGAAACACACACAGAAAAAAATGG
AAGGAATATGCCGATATATTAATGCCTCTGGATGAGTTTATGAATGATTT
TTTCTTTTTTATATCCCTGTACTTGACATATTTTCTACAATAAGCATGTT
TTATTTTACTATATTTTGTTTTATTTTGAGATGGGGGTCTCCCTATGTTT
CTCAGGCTGACCTTGAACTCTTGGGCTCAAGCAATCCTCCAATATCAGCC
TCCTGAGTAGCTGGGACTACAGGTGCACCCCACTGCACCCAGCTGCGTGT
GTTATTTTGATATCGATGGAAAAAATAAATAAAATGTTTAAGCCAAGGAA
AACAAAAACTAGGTTGAAAAAGAAGGCCAAAAGGGCACACAAGTCCAGAG
TGAAAGACAGACACCCCAGCAGTCACCCTCAGAGCAGAGGGAGAATATTG
AAAGTATTACCACTGATCTGATCTGGCACTGACTATAAACTTGCAGATTG
GCTCTCTGGCTCTCCTTCCATCATCAGTTGGCCAGAAGGGCCCCTGAGAA
AACCTCCAGGCTTCAAGTGACATACCTAGTCTGCTTTACCGGTTTACAGG
ACTCAAGAGAAAGGTGGACATTGAGAGTTAATCCCTGAGGCCAAATCTTA
AATGGAGAAAGTCAACATCCACAGAAAATGGGGAAGGGCACAAGTATTTC
TGTGGGCTTATATTCCGACATTTTTATCTGTAGGGGAAAAATGCTTTCTT
AGAAAATGACTCAGCACGGGGAAGTCTTGTCTCTACCTCTGTCTTGTTTT
GTCCTTTGGGGTCCCTTCACTATCAAGTTCAACTGTGTGTCCCTGAGACT
CCTCTGCCCCGGAGGACAGGAGACTCGAAAAACGCTCTTCCTGGCCAGTC
TCTTTGCTCTGTGTCTGCCAGCCCCCAGCATCTCTCCTCTTTCCTGTAAG
CCCCTCTCCCTGTGCTGACTGTCTTCATAGTACTTTAGGTATGTTGTCCC
TTTACCTCTGGGAGGATAGCTTGATGACCTGTCTGCTCAGGCCAGCCCCA
TCTAGAGTCTCAGTGGCCCCAGTCATGTTGAGAAAGGTTCTTTCAAAGAT
AGACTCAAGATAGTAGTGTCAGAGGTCCCAAGCAAATGAAGGGCGGGGAC
AGTTGAGGGGGTGGAATAGGGACGGCAGCAGGGAACCAGATAGCATGCTG
CTGAGAAGAAAAAAAGACATTGGTTTAGGTCAGGAAGCAAAAAAAGGGAA
CTGAGTGGCTGTGAAAGGGTGGGGTTTGCTCAGACTGTCCTTCCTCTCTG
GACTGTAAGAATTAGTCTCGAGGCCACCATGGTGAGCAAGGGCGAGGAGC
TGTTCACCGGGGTGGTGCCCATCCTGGTCGAGCTGGACGGCGACGTAAAC
GGCCACAAGTTCAGCGTGTCCGGCGAGGGCGAGGGCGATGCCACCTACGG
CAAGCTGACCCTGAAGTTCATCTGCACCACCGGCAAGCTGCCCGTGCCCT
GGCCCACCCTCGTGACCACCCTGACCTACGGCGTGCAGTGCTTCAGCCGC
TACCCCGACCACATGAAGCAGCACGACTTCTTCAAGTCCGCCATGCCCGA
AGGCTACGTCCAGGAGCGCACCATCTTCTTCAAGGACGACGGCAACTACA
AGACCCGCGCCGAGGTGAAGTTCGAGGGCGACACCCTGGTGAACCGCATC
GAGCTGAAGGGCATCGACTTCAAGGAGGACGGCAACATCCTGGGGCACAA
GCTGGAGTACAACTACAACAGCCACAACGTCTATATCATGGCCGACAAGC
AGAAGAACGGCATCAAGGTGAACTTCAAGATCCGCCACAACATCGAGGAC
GGCAGCGTGCAGCTCGCCGACCACTACCAGCAGAACACCCCCATCGGCGA
CGGCCCCGTGCTGCTGCCCGACAACCACTACCTGAGCACCCAGTCCGCCC
TGAGCAAAGACCCCAACGAGAAGCGCGATCACATGGTCCTGCTGGAGTTC
GTGACCGCCGCCGGGATCACTCTCGGCATGGACGAGCTGTACAAGTAA
CONTIG.BTKp.GFP
(SEQ ID NO: 28)
GCATTTCCTAGGAGAATCCCTGGGGGAATCATTGCAGTTGGAGCATAATG
TAGGGGGCCCCTCAAAAAGAATACATAGCAGCACCAATTCTATCATAGTG
TGTCTTGTCTATGATAACTGCATTGAGAAAGATGCTCTGCTTGTTGAGTG
AGCATTTCACTTCCTTCTGGTTCTGACTATCTGTCTAATAGTGGTCATGT
GGGTTGAAAAGATAGAAAAGGGGAGTAGTATTAGGAAGTTCAGTATGAGG
AAGACTTATTAGACTTATGCATAAACCTAAATTCTGTTGTAATCTGGAAG
AGCTGAAGTGCCACATATGCATCTGTTTAGGAGAGCAAGAACTACAAATT
TGGTCTTCAGTTTGGCTTGCTTACATCCTGAGAACTCTGTAGGCCACATG
TCGTGAATATAGCAGCCTCTGCAACAGTGAAAGCCAGAAAAGGAAGTGGA
AAGTCTCAGGGGAGGGGGCTTTCTGTCATGGATTTATGAGCACAGCAAGA
CTAACAAGCAAAAAGAAAAATGTAAAAGGATCTTGTTCGTGTCCCTGACT
ATATCAAATTTACGAAACCTTTGAAAGAGGGGTATTTCAGACACAGTTTT
TACTACCAATGCCTTTCAACAAGATGACATTGTTTGGCTGCACTAGTGAA
CAATGCTGAACAGCAGATAATTTTAAATTAATTTATATTTGACTTCAGAA
TTTATTATGTGATGTCTATAAACTCTGTGAGGGTTGGAACCACATCTTAT
ATATTTCATGCATCTTCGTAACACCTAGCATGATGCCTTGTACATGGTTA
AAAAAATACTATTTGACTTGATGATATTGCTTATTATATACCTTACCCTT
TTTGAAGTAGGTTTGTAGTTCCAAATTTGATTTATTTTGGCTAATATTGG
TGAGGGGAGGCCACTAGGTAGTGGAGGTGGAGGTAATTAGCCATGAATTA
AAATTTTACTCCATATTATCCAAGTGAATAACCCATATGTACTAATCAAG
ACTTAATTTATATAGATTATCACTTCATTTCTCTTCTATACAAAGTCAGA
ATTTAATAATAAGATAAAATACCCTTTGTTGGATAAACAAAAATGTTATC
TCCAGCAAGGGCAGACGGAGACCTTATAAAATAAGTTTATGCATGAATTG
AAGCACAAGTCAGGAGGACCTAAGAGTATGAAAAATTCTAAGAGCTTTTA
AACGCTACATTCCTAGATATGAAGAGTTCAGGAATCCTTCAGATAGTTCA
CATAACCTGAACACCATTGCTGACTGAAGATTCTGCCATTGAGCTTAGTA
TTCCATCATATGAATAAAAGATAAAATATTAGACTAAGAGTTTCCCAGTT
CTATCACTATCTCTCTAGTGCAATTTTGTAATTTTAGGCACCTCCATTTC
TCTATTTATTAAGAAAAGTCCCATTTTTTGCACTGAATACAAGGGTGGTG
TGGCATTCAATGGTTATGCAATATTTAGAATCCTAAGTATAAAATACCTA
GCTCCAAAAAATTAAATAGATGATGTATGAGAGTGCTTGAAGTCAGAGTC
AGAGAACCTGGTTTCAAGCCCTCTTTTACCATTTACAAACTGGGTGATTT
CTGGCAAGAAGGCCAACAAGCTTCTTTGATGCTCAGTTTCTTCTTCTCAA
AGGAGAATAATGGTAATTCATGCCACATCTACCTCTGGGGTTGTTGTAAG
GATCAAATGAAGTAATAATACATGTAAAAGCACATGGAAAACTGTAAATC
ACTGTATACGAAAGCACTGTACATCTCTTTGTTGTGGATAAATATTTCCC
AAGGAAATATTTCATATTCCTTGCTTTGTGAGGACCTAAACTTGAACAGT
TTCAGCATTTCACATCAACCATACAGTGTGTTCAGATAAACTTGACTGTA
AATGCTTGCAGCACAGGATGCATGCACACATATTTGTGTGTATATATAGC
CACATGTCAAGTGACTGAGAGATTAACTGGTTGAGTTGACATCAGGAAAG
GCTGCCTAGAGAAACAGGAAGGATGGCAGACTGATAATGGTATGGTTGGG
TTATTGCATGACAGCCCTTTGTCTTAATAAACATTTAGATTCTGATACTC
ACTGGCTGGCTATACCAGCCTTGTACACTCCCTTGAATGCAAGTTTGGTT
GGTCAAAATTATATTAGCCAAGTCTTTCAAACTGAAAGGGAACAGGAGGA
ACGAAGATGAAGGATCATCTATTCAATTATTGCTATGTGCCAGATATTGA
GCTAAATACCTTTTATATATTATCTCATTTAATTTTCACAACAACCTTGG
CCTAAAAACAACAACAATGATAACTAACACTTAATGAGCACTTACTATGT
AGCAGGCTCTGTCCCAAGTGCTTCAAATGTATTACTTATTTGATCTTTGC
AGTAGCCTTAAGAAGTATGTATTATTCCCCTATTTACAGATGAGGAAACT
GAAGCTCAAAGATGTTATCCTTATTTCTCTGGGGTAGGGTGGACTGGCCA
GTTGCAAAAACTACCTTTGCTGGCCTTGCCTTAGGGAGTGTCCTTGAGGT
ACACTGTTCTGCAGCAGCTGCCTCAAGGACGCTCAAGACAGATCCAAGCA
AAAGTTATTCACTGATTTTCTTCCTCTAGTGGCTACGACTGGGACTGCAA
AAACATAGATTCATAAAGGGCTTTGTCGTTGTCTTGGGTCTTTTTGTCTT
TTATTTTTAATTGTGGGAAAATTTTCAGTACTATCCCTGAGTTCATTAAC
TACCATCACTAACATAATCATAAAGGGATTTGGGGAGGTTGCTTAGTCTA
TCTTCTTGCCTTATGGCCACCTTGAACCTAAAATTCCCAGATTCCTCTAA
CCAATGAATCCCGTTTCTGAGATTGACTTAAGCAAAGACAGATTAGTACT
TCTAAAAATTTCCCTTTTACTAGTTTTCCTATTTCTACCCCAGTAGGGAT
TTTTGTCTATTGTAAGAATTATACATTCATGACCCCAAAGAATCACACCA
AGACTTTAGGGCCCGTGAGAAAACCTCCAGGCTTCAAGTGACATACCTAG
TCTGCTTTACCGGTTTACAGGACTCAAGAGAAAGGTGGACATTGAGAGTT
AATCCCTGAGGCCAAATCTTAAATGGAGAAAGTCAACATCCACAGAAAAT
GGGGAAGGGCACAAGTATTTCTGTGGGCTTATATTCCGACATTTTTATCT
GTAGGGGAAAAATGCTTTCTTAGAAAATGACTCAGCACGGGGAAGTCTTG
TCTCTACCTCTGTCTTGTTTTGTCCTTTGGGGTCCCTTCACTATCAAGTT
CAACTGTGTGTCCCTGAGACTCCTCTGCCCCGGAGGACAGGAGACTCGAA
AAACGCTCTTCCTGGCCAGTCTCTTTGCTCTGTGTCTGCCAGCCCCCAGC
ATCTCTCCTCTTTCCTGTAAGCCCCTCTCCCTGTGCTGACTGTCTTCATA
GTACTTTAGGTATGTTGTCCCTTTACCTCTGGGAGGATAGCTTGATGACC
TGTCTGCTCAGGCCAGCCCCATCTAGAGTCTCAGTGGCCCCAGTCATGTT
GAGAAAGGTTCTTTCAAAGATAGACTCAAGATAGTAGTGTCAGAGGTCCC
AAGCAAATGAAGGGCGGGGACAGTTGAGGGGGTGGAATAGGGACGGCAGC
AGGGAACCAGATAGCATGCTGCTGAGAAGAAAAAAAGACATTGGTTTAGG
TCAGGAAGCAAAAAAAGGGAACTGAGTGGCTGTGAAAGGGTGGGGTTTGC
TCAGACTGTCCTTCCTCTCTGGACTGTAAGAATTAGTCTCGAGGCCACCA
TGGTGAGCAAGGGCGAGGAGCTGTTCACCGGGGTGGTGCCCATCCTGGTC
GAGCTGGACGGCGACGTAAACGGCCACAAGTTCAGCGTGTCCGGCGAGGG
CGAGGGCGATGCCACCTACGGCAAGCTGACCCTGAAGTTCATCTGCACCA
CCGGCAAGCTGCCCGTGCCCTGGCCCACCCTCGTGACCACCCTGACCTAC
GGCGTGCAGTGCTTCAGCCGCTACCCCGACCACATGAAGCAGCACGACTT
CTTCAAGTCCGCCATGCCCGAAGGCTACGTCCAGGAGCGCACCATCTTCT
TCAAGGACGACGGCAACTACAAGACCCGCGCCGAGGTGAAGTTCGAGGGC
GACACCCTGGTGAACCGCATCGAGCTGAAGGGCATCGACTTCAAGGAGGA
CGGCAACATCCTGGGGCACAAGCTGGAGTACAACTACAACAGCCACAACG
TCTATATCATGGCCGACAAGCAGAAGAACGGCATCAAGGTGAACTTCAAG
ATCCGCCACAACATCGAGGACGGCAGCGTGCAGCTCGCCGACCACTACCA
GCAGAACACCCCCATCGGCGACGGCCCCGTGCTGCTGCCCGACAACCACT
ACCTGAGCACCCAGTCCGCCCTGAGCAAAGACCCCAACGAGAAGCGCGAT
CACATGGTCCTGCTGGAGTTCGTGACCGCCGCCGGGATCACTCTCGGCAT
GGACGAGCTGTACAAGTAA
3kb.BTKp.GFP
(SEQ ID NO: 29)
GCATTTCCTAGGAGAATCCCTGGGGGAATCATTGCAGTTGGAGCATAATG
TAGGGGGCCCAAACTGTAGTCTGTTAGCTCACCCTAGAAGAAGGAAATTC
TAGAGTCAAGAGAAGTTAAGTTTTAGACAAGCTGGAAGTTGGAATATACT
GGAGTTTAAGATCATTGACTCAATGAAAAAAAAAAAAGATTTGTCTGCAG
TAAAACCAGAGTTTTAAAAATGATTAGAAATGGACCAGAAAAGAGATAA
GGATAATGAGAAAGGGGGCTCTCTAAAACTATTTGGAACTGAAATTGGGA
CAATGTTTCTGGAAACTATTTTGGCAGTATGTATCAAAATGCAAAATGCA
CATACTTTTTTGACTCTGGAAATGATGCTTGTATAGAGATCCTAATAACA
TGGGAAAATAATTAGGAATAATGTTAAATAAGAACATTAGATCTTTTTTT
GGAAAAAGGTACCTTGAACGGATGGATGGATGGATAGATACATACATACA
TGCATACATACATACATAGATGTTACGAAAAATAGGTAAATTAACTGAAT
GTGCAATATGATCTCAAATATTATAAGAAACACACACAGAAAAAAATGG
AAGGAATATGCCGATATATTAATGCCTCTGGATGAGTTTATGAATGATTT
TTTCTTTTTTATATCCCTGTACTTGACATATTTTCTACAATAAGCATGTT
TTATTTTACTATATTTTGTTTTATTTTGAGATGGGGGTCTCCCTATGTTT
CTCAGGCTGACCTTGAACTCTTGGGCTCAAGCAATCCTCCAATATCAGCC
TCCTGAGTAGCTGGGACTACAGGTGCACCCCACTGCACCCAGCTGCGTGT
GTTATTTTGATATCGATGGAAAAAATAAATAAAATGTTTAAGCCAAGGAA
AACAAAAACTAGGTTGAAAAAGAAGGCCAAAAGGGCACACAAGTCCAGAG
TGAAAGACAGACACCCCAGCAGTCACCCTCAGAGCAGAGGGAGAATATTG
AAAGTATTACCACTGATCTGATCTGGCACTGACTATAAACTTGCAGATTG
GCTCTCTGGCTCTCCTTCCATCATCAGTTGGCCAGAATATATTGTCGCTA
ATGCAAGACAAAAAGAGATGATGAATCACAGAGACCTAGAAGTTTCCAAA
ACCACAGAAGAAAAATGTAAAATGCTAAAAACATAAGGCCAGGCGCGGTG
GCTCAAGCCTGTAATCCCAGCATTTTGGGAGGCCGAGGCAGGTGTCATGG
TGTGCGGCTGTAGTCCAAGCTACTCAGGAGGCTGAGGCAAGAGAATTGCT
TGAAACTGGGAGGTAGCGATTGCAGTGAGCCAAGATTGCGCTACTGTACC
GAAGCCTGGATGACAGAGCGAGACTCTGTCTCAAAAATAAATAAATAAAT
AAATAAATAAATAAAACATGAACTGCTGTGGAAGAACCAGAGAGCCCTAA
GGCCTCCTGTAAATGACTACAGCAGGAGAGTTTCCCTGGTCAGCATGCAG
CTTTCGAAAATTGAGTTTGGCTTGATGCCTTCATCACAAATTTGGTCCCC
TGTAGCCTTGACTATGAAGCTTATCATAAAATAATTGGGTTTCACTGGGG
CCCGAACAAAAGCATCTTGGGAATTTAACAAGATTAAAGGTTGCTTTGTT
GCTGTGGGTATGATTTTTTTTTCTTTTTTCTTTTTTTTTTTTTTTGAGAC
GGAGTCTGACTCTGTCACCCAGGCTGGAGTGCAGTGGCACGATCTCGGCT
CACTGCAACCTCCGCCTCCTGGGTTCAAGCAATTCTCCTGCCTCAGCCTC
CTGAGTAACTGGGATTACAGGTGCGCGCCACCACGCCAGGCTAATTTTTG
TATTTTTAGTAGAGACAGGGTTTCTCCATGTTGGTCAGGCTGGCTGCTCT
CAAACTCCTGACCTTGCCATCCAGCCGCCTCAGCCTCCCAAAGTGCTGGG
ATTACAGGCATGAGCTACCACACGTGGCCATATGTAAATTTTAAACATGT
AAACCAATACTGTATATCTTTTATAGATATTTACATCTGTAATGATAATA
TGAAAACGTACCTAGGAATGATAAACACCAAATTCAAGATATTGGTAATC
TCTAAGGAGGGAGAAGACTAAGATTTTGGAGTACATAAAGGGGTCTTGAT
TATATAGATAGTATTTCAGTTCTTAACACGTTCTCTTCTGGTCCCAAGGC
CAAGGAATTCTCAATTGAAATCTCAGTTAGAACGTGAAGGATGCTTCTTG
GGACTGGGAAGAGATTTTCCGTAGGCCAAATTTAACCTCCCACTAGAGAT
AGTTGATAATTCTACCACATTTCATAGTGAAGCATCAGGAAGACAGGACT
GAAAAGGCAAGAAGGAATGATGCAAATTGCATAGTCCAAAAGATAAGCTG
GACCTAGGTTTGCCAGTTTGTCTAAGTAACTGAGAATTCATTCTGAGATC
AAAAAATCAAAATAATGTTCACAGCAGCATTGTTTATCACTGAAAGCAAA
CAACCTAAATGTCCATCAACAGGAGAATGAATAAATATATTGGAATGTAT
TCATCATATAGAATATTTTATGCATATAAAAATGAATTAAAACATATGAA
GTAGAACTAGCCAGATAAATAAATCCTAACATATTATATTGAGGGGAAAA
AAGCAAGTTGCTGGAGAATGTGTACCATATATATATATATAAATTTTCTT
TTTTTTTTTTTTTTTTTTTCGCGACCGAGTTTCACTCTTGTTGCCCAGGC
TGGAGTGCAATGGCAAGATCTCGGCTCACTGCAACCTCTGCCTCCCGGGT
TCAAGGGATTCTCCCGCCTCAGCCTCCTGAGTAGCTGGGAGAGTAGCGTT
GGCCCAGCTAGTTTTTGTATTTTTAGTAGAGACAGGGTTTCTCCATGTTG
GTCAGGCTGGCTGCTCTCAAACTCCTGACCTTGTCATCCACCCGCCTCAG
CCTCCCAAAGTGCTGGGATTACAGGCCTGAGTTACCGCACGGGCCCCTGA
GAAAACCTCCAGGCTTCAAGTGACATACCTAGTCTGCTTTACCGGTTTAC
AGGACTCAAGAGAAAGGTGGACATTGAGAGTTAATCCCTGAGGCCAAATC
TTAAATGGAGAAAGTCAACATCCACAGAAAATGGGGAAGGGCACAAGTAT
TTCTGTGGGCTTATATTCCGACATTTTTATCTGTAGGGGAAAAATGCTTT
CTTAGAAAATGACTCAGCACGGGGAAGTCTTGTCTCTACCTCTGTCTTGT
TTTGTCCTTTGGGGTCCCTTCACTATCAAGTTCAACTGTGTGTCCCTGAG
ACTCCTCTGCCCCGGAGGACAGGAGACTCGAAAAACGCTCTTCCTGGCCA
GTCTCTTTGCTCTGTGTCTGCCAGCCCCCAGCATCTCTCCTCTTTCCTGT
AAGCCCCTCTCCCTGTGCTGACTGTCTTCATAGTACTTTAGGTATGTTGT
CCCTTTACCTCTGGGAGGATAGCTTGATGACCTGTCTGCTCAGGCCAGCC
CCATCTAGAGTCTCAGTGGCCCCAGTCATGTTGAGAAAGGTTCTTTCAAA
GATAGACTCAAGATAGTAGTGTCAGAGGTCCCAAGCAAATGAAGGGCGGG
GACAGTTGAGGGGGTGGAATAGGGACGGCAGCAGGGAACCAGATAGCATG
CTGCTGAGAAGAAAAAAAGACATTGGTTTAGGTCAGGAAGCAAAAAAAGG
GAACTGAGTGGCTGTGAAAGGGTGGGGTTTGCTCAGACTGTCCTTCCTCT
CTGGACTGTAAGAATTAGTCTCGAGGCCACCATGGTGAGCAAGGGCGAGG
AGCTGTTCACCGGGGTGGTGCCCATCCTGGTCGAGCTGGACGGCGACGTA
AACGGCCACAAGTTCAGCGTGTCCGGCGAGGGCGAGGGCGATGCCACCTA
CGGCAAGCTGACCCTGAAGTTCATCTGCACCACCGGCAAGCTGCCCGTGC
CCTGGCCCACCCTCGTGACCACCCTGACCTACGGCGTGCAGTGCTTCAGC
CGCTACCCCGACCACATGAAGCAGCACGACTTCTTCAAGTCCGCCATGCC
CGAAGGCTACGTCCAGGAGCGCACCATCTTCTTCAAGGACGACGGCAACT
ACAAGACCCGCGCCGAGGTGAAGTTCGAGGGCGACACCCTGGTGAACCGC
ATCGAGCTGAAGGGCATCGACTTCAAGGAGGACGGCAACATCCTGGGGCA
CAAGCTGGAGTACAACTACAACAGCCACAACGTCTATATCATGGCCGACA
AGCAGAAGAACGGCATCAAGGTGAACTTCAAGATCCGCCACAACATCGAG
GACGGCAGCGTGCAGCTCGCCGACCACTACCAGCAGAACACCCCCATCGG
CGACGGCCCCGTGCTGCTGCCCGACAACCACTACCTGAGCACCCAGTCCG
CCCTGAGCAAAGACCCCAACGAGAAGCGCGATCACATGGTCCTGCTGGAG
TTCGTGACCGCCGCCGGGATCACTCTCGGCATGGACGAGCTGTACAAGTA
A
revCONTIG.BTKp.GFP
(SEQ ID NO: 30)
GCATTTCCTAGGAGAATCCCTGGGGGAATCATTGCAGTTGGAGCATAATG
TAGGGGGCCCTAAAGTCTTGGTGTGATTCTTTGGGGTCATGAATGTATAA
TTCTTACAATAGACAAAAATCCCTACTGGGGTAGAAATAGGAAAACTAGT
AAAAGGGAAATTTTTAGAAGTACTAATCTGTCTTTGCTTAAGTCAATCTC
AGAAACGGGATTCATTGGTTAGAGGAATCTGGGAATTTTAGGTTCAAGGT
GGCCATAAGGCAAGAAGATAGACTAAGCAACCTCCCCAAATCCCTTTATG
ATTATGTTAGTGATGGTAGTTAATGAACTCAGGGATAGTACTGAAAATTT
TCCCACAATTAAAAATAAAAGACAAAAAGACCCAAGACAACGACAAAGCC
CTTTATGAATCTATGTTTTTGCAGTCCCAGTCGTAGCCACTAGAGGAAGA
AAATCAGTGAATAACTTTTGCTTGGATCTGTCTTGAGCGTCCTTGAGGCA
GCTGCTGCAGAACAGTGTACCTCAAGGACACTCCCTAAGGCAAGGCCAGC
AAAGGTAGTTTTTGCAACTGGCCAGTCCACCCTACCCCAGAGAAATAAGG
ATAACATCTTTGAGCTTCAGTTTCCTCATCTGTAAATAGGGGAATAATAC
ATACTTCTTAAGGCTACTGCAAAGATCAAATAAGTAATACATTTGAAGCA
CTTGGGACAGAGCCTGCTACATAGTAAGTGCTCATTAAGTGTTAGTTATC
ATTGTTGTTGTTTTTAGGCCAAGGTTGTTGTGAAAATTAAATGAGATAAT
ATATAAAAGGTATTTAGCTCAATATCTGGCACATAGCAATAATTGAATAG
ATGATCCTTCATCTTCGTTCCTCCTGTTCCCTTTCAGTTTGAAAGACTTG
GCTAATATAATTTTGACCAACCAAACTTGCATTCAAGGGAGTGTACAAGG
CTGGTATAGCCAGCCAGTGAGTATCAGAATCTAAATGTTTATTAAGACAA
AGGGCTGTCATGCAATAACCCAACCATACCATTATCAGTCTGCCATCCTT
CCTGTTTCTCTAGGCAGCCTTTCCTGATGTCAACTCAACCAGTTAATCTC
TCAGTCACTTGACATGTGGCTATATATACACACAAATATGTGTGCATGCA
TCCTGTGCTGCAAGCATTTACAGTCAAGTTTATCTGAACACACTGTATGG
TTGATGTGAAATGCTGAAACTGTTCAAGTTTAGGTCCTCACAAAGCAAGG
AATATGAAATATTTCCTTGGGAAATATTTATCCACAACAAAGAGATGTAC
AGTGCTTTCGTATACAGTGATTTACAGTTTTCCATGTGCTTTTACATGTA
TTATTACTTCATTTGATCCTTACAACAACCCCAGAGGTAGATGTGGCATG
AATTACCATTATTCTCCTTTGAGAAGAAGAAACTGAGCATCAAAGAAGCT
TGTTGGCCTTCTTGCCAGAAATCACCCAGTTTGTAAATGGTAAAAGAGGG
CTTGAAACCAGGTTCTCTGACTCTGACTTCAAGCACTCTCATACATCATC
TATTTAATTTTTTGGAGCTAGGTATTTTATACTTAGGATTCTAAATATTG
CATAACCATTGAATGCCACACCACCCTTGTATTCAGTGCAAAAAATGGGA
CTTTTCTTAATAAATAGAGAAATGGAGGTGCCTAAAATTACAAAATTGCA
CTAGAGAGATAGTGATAGAACTGGGAAACTCTTAGTCTAATATTTTATCT
TTTATTCATATGATGGAATACTAAGCTCAATGGCAGAATCTTCAGTCAGC
AATGGTGTTCAGGTTATGTGAACTATCTGAAGGATTCCTGAACTCTTCAT
ATCTAGGAATGTAGCGTTTAAAAGCTCTTAGAATTTTTCATACTCTTAGG
TCCTCCTGACTTGTGCTTCAATTCATGCATAAACTTATTTTATAAGGTCT
CCGTCTGCCCTTGCTGGAGATAACATTTTTGTTTATCCAACAAAGGGTAT
TTTATCTTATTATTAAATTCTGACTTTGTATAGAAGAGAAATGAAGTGAT
AATCTATATAAATTAAGTCTTGATTAGTACATATGGGTTATTCACTTGGA
TAATATGGAGTAAAATTTTAATTCATGGCTAATTACCTCCACCTCCACTA
CCTAGTGGCCTCCCCTCACCAATATTAGCCAAAATAAATCAAATTTGGAA
CTACAAACCTACTTCAAAAAGGGTAAGGTATATAATAAGCAATATCATCA
AGTCAAATAGTATTTTTTTAACCATGTACAAGGCATCATGCTAGGTGTTA
CGAAGATGCATGAAATATATAAGATGTGGTTCCAACCCTCACAGAGTTTA
TAGACATCACATAATAAATTCTGAAGTCAAATATAAATTAATTTAAAATT
ATCTGCTGTTCAGCATTGTTCACTAGTGCAGCCAAACAATGTCATCTTGT
TGAAAGGCATTGGTAGTAAAAACTGTGTCTGAAATACCCCTCTTTCAAAG
GTTTCGTAAATTTGATATAGTCAGGGACACGAACAAGATCCTTTTACATT
TTTCTTTTTGCTTGTTAGTCTTGCTGTGCTCATAAATCCATGACAGAAAG
CCCCCTCCCCTGAGACTTTCCACTTCCTTTTCTGGCTTTCACTGTTGCAG
AGGCTGCTATATTCACGACATGTGGCCTACAGAGTTCTCAGGATGTAAGC
AAGCCAAACTGAAGACCAAATTTGTAGTTCTTGCTCTCCTAAACAGATGC
ATATGTGGCACTTCAGCTCTTCCAGATTACAACAGAATTTAGGTTTATGC
ATAAGTCTAATAAGTCTTCCTCATACTGAACTTCCTAATACTACTCCCCT
TTTCTATCTTTTCAACCCACATGACCACTATTAGACAGATAGTCAGAACC
AGAAGGAAGTGAAATGCTCACTCAACAAGCAGAGCATCTTTCTCAATGCA
GTTATCATAGACAAGACACACTATGATAGAATTGGTGCTGCTATGTATTC
TTTTTGAGGGGCCCCTGAGAAAACCTCCAGGCTTCAAGTGACATACCTAG
TCTGCTTTACCGGTTTACAGGACTCAAGAGAAAGGTGGACATTGAGAGTT
AATCCCTGAGGCCAAATCTTAAATGGAGAAAGTCAACATCCACAGAAAAT
GGGGAAGGGCACAAGTATTTCTGTGGGCTTATATTCCGACATTTTTATCT
GTAGGGGAAAAATGCTTTCTTAGAAAATGACTCAGCACGGGGAAGTCTTG
TCTCTACCTCTGTCTTGTTTTGTCCTTTGGGGTCCCTTCACTATCAAGTT
CAACTGTGTGTCCCTGAGACTCCTCTGCCCCGGAGGACAGGAGACTCGAA
AAACGCTCTTCCTGGCCAGTCTCTTTGCTCTGTGTCTGCCAGCCCCCAGC
ATCTCTCCTCTTTCCTGTAAGCCCCTCTCCCTGTGCTGACTGTCTTCATA
GTACTTTAGGTATGTTGTCCCTTTACCTCTGGGAGGATAGCTTGATGACC
TGTCTGCTCAGGCCAGCCCCATCTAGAGTCTCAGTGGCCCCAGTCATGTT
GAGAAAGGTTCTTTCAAAGATAGACTCAAGATAGTAGTGTCAGAGGTCCC
AAGCAAATGAAGGGCGGGGACAGTTGAGGGGGTGGAATAGGGACGGCAGC
AGGGAACCAGATAGCATGCTGCTGAGAAGAAAAAAAGACATTGGTTTAGG
TCAGGAAGCAAAAAAAGGGAACTGAGTGGCTGTGAAAGGGTGGGGTTTGC
TCAGACTGTCCTTCCTCTCTGGACTGTAAGAATTAGTCTCGAGGCCACCA
TGGTGAGCAAGGGCGAGGAGCTGTTCACCGGGGTGGTGCCCATCCTGGTC
GAGCTGGACGGCGACGTAAACGGCCACAAGTTCAGCGTGTCCGGCGAGGG
CGAGGGCGATGCCACCTACGGCAAGCTGACCCTGAAGTTCATCTGCACCA
CCGGCAAGCTGCCCGTGCCCTGGCCCACCCTCGTGACCACCCTGACCTAC
GGCGTGCAGTGCTTCAGCCGCTACCCCGACCACATGAAGCAGCACGACTT
CTTCAAGTCCGCCATGCCCGAAGGCTACGTCCAGGAGCGCACCATCTTCT
TCAAGGACGACGGCAACTACAAGACCCGCGCCGAGGTGAAGTTCGAGGGC
GACACCCTGGTGAACCGCATCGAGCTGAAGGGCATCGACTTCAAGGAGGA
CGGCAACATCCTGGGGCACAAGCTGGAGTACAACTACAACAGCCACAACG
TCTATATCATGGCCGACAAGCAGAAGAACGGCATCAAGGTGAACTTCAAG
ATCCGCCACAACATCGAGGACGGCAGCGTGCAGCTCGCCGACCACTACCA
GCAGAACACCCCCATCGGCGACGGCCCCGTGCTGCTGCCCGACAACCACT
ACCTGAGCACCCAGTCCGCCCTGAGCAAAGACCCCAACGAGAAGCGCGAT
CACATGGTCCTGCTGGAGTTCGTGACCGCCGCCGGGATCACTCTCGGCAT
GGACGAGCTGTACAAGTAA
IE.BTKp.GFP
(SEQ ID NO: 31)
GCATTTCCTAGGAGAATCCCTGGGGGAATCATTGCAGTTGGAGCATAATG
TAGGGGGCCCACATCTTTGAGCTTCAGTTTCCTCATCTGTAAATAGGGGA
ATAATACATACTTCTTAAGGCTACTGCAAAGATCAAATAAGTAATACATT
TGAAGCACTTGGGACAGAGCCTGCTACATAGTAAGTGCTCATTAAGTGTT
AGTTATCATTGTTGTTGTTTTTAGGCCAAGGTTGTTGTGAAAATTAAATG
AGATAATATATAAAAGGTATTTAGCTCAATATCTGGCACATAGCAATAAT
TGAATAGATGATCCTTCATCTTCGTTCCTCCTGTTCCCTTTCAGTTTGAA
AGACTTGGCTAATATAATTTTGACCAACCAAACTTGCATTCAAGGGAGTG
TACAAGGCTGGTATAGCCAGCCAGTGAGTATCAGAATCTAAATGTTTATT
AAGACAAAGGGCTGTCATGCAATAACCCAACCATACCATTATCAGTCTGC
CATCCTTCCTGTTTCTCTAGGCAGCCTTTCCTGATGTCAACTCAACCAGT
TAATCTCTCAGTCACTTGACATGTGGCTATATATACACACAAATATGTGT
GCATGCATCCTGTGCTGCAAGCATTTACAGTCAAGTTTATCTGAACACAC
TGTATGGTTGATGTGAAATGCTGAAACTGTTCAAGTTTAGGTCCTCACAA
AGCAAGGAATATGAAATATTTCCTTGGGAAATATTTATCCACAACAAAGA
GATGTACAGTGCTTTCGTATACAGTGATTTACAGTTTTCCATGTGCTTTT
ACATGTATTATTACTTCATTTGATCCTTACAACAACCCCAGAGGTAGATG
TGGCATGAATTACCATTATTCTCCTTTGAGAAGAAGAAACTGAGCATCAA
AGAAGCTTGTTGGCCTTCTTGCCAGAAATCACCCAGTTTGTAAATGGTAA
AAGAGGGCTTGAAACCAGGTTCTCTGACTCTGACTTCAAGCACTCTCATA
CATCATCTATTTAATTTTTTGGAGCTAGGTATTTTATACTTAGGATTCTA
AATATTGCATAACCATTGAATGCCACACCACCCTTGTATTCAGTGCAAAA
AATGGGACTTTTCTTAATAAATAGAGAAATGGAGGTGCCTAAAATTACAA
AATTGCACTAGAGAGATAGTGATAGAACTGGGAAACTCTTAGTCTAATAT
TTTATCTTTTATTCATATGATGGAATACTAAGCTCAATGGCAGAATCTTC
AGTCAGCAATGGTGTTCAGGTTATGTGAACTATCTGAAGGATTCCTGAAC
TCTTCATATCTAGGAATGTAGCGTTTAAAAGCTCTTAGAATTTTTCATAC
TCTTAGGTCCTCCTGACTTGTGCTTCAATTCATGCATAAACTTATTTTAT
AAGGTCTCCGTCTGCCCTTGCTGGAGATAACATTTTTGTTTATCCAACAA
AGGGTATTTTATCTTATTATTAAATTCTGACTTTGTATAGAAGAGAAATG
AAGTGATAATCTATATAAATTAAGTCTTGATTAGTACATATGGGTTATTC
ACTTGGATAATATGGAGTAAAATTTTAATTCATGGCTAATTACCTCCACC
TCCACTACCTAGTGGCCTCCCCTCACCAATATTAGCCAAAATAAATCAAA
TTTGGAACTACAAACCTACTTCAAAAAGGGTAAGGTATATAATAAGCAAT
ATCATCAAGTCAAATAGTATTTTTTTAACCATGTACAAGGCATCATGCTA
GGTGTTACGAAGATGCATGAAATATATAAGATGTGGTTCCAACCCTCACA
GAGTTTATAGACATCACATAATAAATTCTGAAGTCAAATATAAATTAATT
TAAAATTATCTGCTGTTCAGCATTGTTCACTAGTGCAGCCAAACAATGTC
ATCTTGTTGAAAGGCATTGGTAGTAAAAACTGTGTCTGAAATACCCCTCT
TTCAAAGGTTTCGTAAATTTGATATAGTCAGGGACACGAACAAGATCCTT
TTACATTTTTCTTTTTGCTTGTTAGTCTTGCTGTGCTCATAAATCCATGA
CAGAAAGCCCCCTCCCCTGAGACTTTCCACTTCCTTTTCTGGCTTTCACT
GTTGCAGAGGCTGCTATATTCACGACATGTGGCCTACAGAGTTCTCAGGA
TGTAAGCAAGCCAAACTGAAGACCAAATTTGTAGTTCTTGCTCTCCTAAA
CAGATGCATATGTGGCACTTCAGCTCTTCCAGATTACAACAGAATTTAGG
TTTATGCATAAGTCTAATAAGTCTTCCTCATACTGAACTTCCTAATACTA
CTCCCCTTTTCTATCTTTTCAACCCACATGACCACTATTAGACAGATAGT
CAGAACCAGAAGGAAGTGAAATGCTCACTCAACAAGCAGAGCATCTTTCT
CAATGCAGTTATCATAGACAAGACACACTATGATAGAATTGGTGCTGCTA
TGTATTCTTTTTGAGGCCCCTGAGAAAACCTCCAGGCTTCAAGTGACATA
CCTAGTCTGCTTTACCGGTTTACAGGACTCAAGAGAAAGGTGGACATTGA
GAGTTAATCCCTGAGGCCAAATCTTAAATGGAGAAAGTCAACATCCACAG
AAAATGGGGAAGGGCACAAGTATTTCTGTGGGCTTATATTCCGACATTTT
TATCTGTAGGGGAAAAATGCTTTCTTAGAAAATGACTCAGCACGGGGAAG
TCTTGTCTCTACCTCTGTCTTGTTTTGTCCTTTGGGGTCCCTTCACTATC
AAGTTCAACTGTGTGTCCCTGAGACTCCTCTGCCCCGGAGGACAGGAGAC
TCGAAAAACGCTCTTCCTGGCCAGTCTCTTTGCTCTGTGTCTGCCAGCCC
CCAGCATCTCTCCTCTTTCCTGTAAGCCCCTCTCCCTGTGCTGACTGTCT
TCATAGTACTTTAGGTATGTTGTCCCTTTACCTCTGGGAGGATAGCTTGA
TGACCTGTCTGCTCAGGCCAGCCCCATCTAGAGTCTCAGTGGCCCCAGTC
ATGTTGAGAAAGGTTCTTTCAAAGATAGACTCAAGATAGTAGTGTCAGAG
GTCCCAAGCAAATGAAGGGCGGGGACAGTTGAGGGGGTGGAATAGGGACG
GCAGCAGGGAACCAGATAGCATGCTGCTGAGAAGAAAAAAAGACATTGGT
TTAGGTCAGGAAGCAAAAAAAGGGAACTGAGTGGCTGTGAAAGGGTGGGG
TTTGCTCAGACTGTCCTTCCTCTCTGGACTGTAAGAATTAGTCTCGAGGC
CACCATGGTGAGCAAGGGCGAGGAGCTGTTCACCGGGGTGGTGCCCATCC
TGGTCGAGCTGGACGGCGACGTAAACGGCCACAAGTTCAGCGTGTCCGGC
GAGGGCGAGGGCGATGCCACCTACGGCAAGCTGACCCTGAAGTTCATCTG
CACCACCGGCAAGCTGCCCGTGCCCTGGCCCACCCTCGTGACCACCCTGA
CCTACGGCGTGCAGTGCTTCAGCCGCTACCCCGACCACATGAAGCAGCAC
GACTTCTTCAAGTCCGCCATGCCCGAAGGCTACGTCCAGGAGCGCACCAT
CTTCTTCAAGGACGACGGCAACTACAAGACCCGCGCCGAGGTGAAGTTCG
AGGGCGACACCCTGGTGAACCGCATCGAGCTGAAGGGCATCGACTTCAAG
GAGGACGGCAACATCCTGGGGCACAAGCTGGAGTACAACTACAACAGCCA
CAACGTCTATATCATGGCCGACAAGCAGAAGAACGGCATCAAGGTGAACT
TCAAGATCCGCCACAACATCGAGGACGGCAGCGTGCAGCTCGCCGACCAC
TACCAGCAGAACACCCCCATCGGCGACGGCCCCGTGCTGCTGCCCGACAA
CCACTACCTGAGCACCCAGTCCGCCCTGAGCAAAGACCCCAACGAGAAGC
GCGATCACATGGTCCTGCTGGAGTTCGTGACCGCCGCCGGGATCACTCTC
GGCATGGACGAGCTGTACAAGTAA
0.7UCOE.IE.BTKp.GFP
(SEQ ID NO: 32)
GCATTTCCTAGGAGAATCCCTGGGGGAATCATTGCAGTTGGAGCATAATG
TAGGGGGCCGCGTGTGGCATCTGAAGCACCACCAGCGAGCGAGAGCTAG
AGAGAAGGAAAGCCACCGACTTCACCGCCTCCGAGCTGCTCCGGGTCGCG
GGTCTGCAGCGTCTCCGGCCCTCCGCGCCTACAGCTCAAGCCACATCCGA
AGGGGGAGGGAGCCGGGAGCTGCGCGCGGGGCCGCCGGGGGGAGGGGT
GGCACCGCCCACGCCGGGCGGCCACGAAGGGCGGGGCAGCGGGCGCGCG
CCCGGCGGGGGGAGGGGCCGCGCGCCGCGCCCGCTGGGAATTGGGGCCC
TAGGGGGAGGGCGGAGGCGCCGACGACCGCGGCACTTACCGTTCGCGGC
GTGGCGCCCGGTGGTCCCCAAGGGGAGGGAAGGGGGAGGCGGGGCGAG
GACAGTGACCGGAGTCTCCTCAGCGGTGGCTTTTCTGCTTGGCAGCCTCA
GCGGCTGGCGCCAAAACCGGACTCCGCCCACTTCCTCGCCCCTGCGGTGC
GAGGGTGTGGAATCCTCCAGACGCTGGGGGAGGGGGAGTTGGGAGCTTA
AAAACTAGTACCCCTTTGGGACCACTTTCAGCAGCGAACTCTCCTGTACA
CCAGGGGTCAGTTCCACAGACGCGGGCCAGGGGTGGGTCATTGCGGCGT
GAACAATAATTTGACTAGAAGTTGATTCGGGTGTTTGCGGCCCACATCTT
TGAGCTTCAGTTTCCTCATCTGTAAATAGGGGAATAATACATACTTCTTA
AGGCTACTGCAAAGATCAAATAAGTAATACATTTGAAGCACTTGGGACAG
AGCCTGCTACATAGTAAGTGCTCATTAAGTGTTAGTTATCATTGTTGTTG
TTTTTAGGCCAAGGTTGTTGTGAAAATTAAATGAGATAATATATAAAAGG
TATTTAGCTCAATATCTGGCACATAGCAATAATTGAATAGATGATCCTTC
ATCTTCGTTCCTCCTGTTCCCTTTCAGTTTGAAAGACTTGGCTAATATAA
TTTTGACCAACCAAACTTGCATTCAAGGGAGTGTACAAGGCTGGTATAGC
CAGCCAGTGAGTATCAGAATCTAAATGTTTATTAAGACAAAGGGCTGTCA
TGCAATAACCCAACCATACCATTATCAGTCTGCCATCCTTCCTGTTTCTC
TAGGCAGCCTTTCCTGATGTCAACTCAACCAGTTAATCTCTCAGTCACTT
GACATGTGGCTATATATACACACAAATATGTGTGCATGCATCCTGTGCTG
CAAGCATTTACAGTCAAGTTTATCTGAACACACTGTATGGTTGATGTGAA
ATGCTGAAACTGTTCAAGTTTAGGTCCTCACAAAGCAAGGAATATGAAAT
ATTTCCTTGGGAAATATTTATCCACAACAAAGAGATGTACAGTGCTTTCG
TATACAGTGATTTACAGTTTTCCATGTGCTTTTACATGTATTATTACTTC
ATTTGATCCTTACAACAACCCCAGAGGTAGATGTGGCATGAATTACCATT
ATTCTCCTTTGAGAAGAAGAAACTGAGCATCAAAGAAGCTTGTTGGCCTT
CTTGCCAGAAATCACCCAGTTTGTAAATGGTAAAAGAGGGCTTGAAACCA
GGTTCTCTGACTCTGACTTCAAGCACTCTCATACATCATCTATTTAATTT
TTTGGAGCTAGGTATTTTATACTTAGGATTCTAAATATTGCATAACCATT
GAATGCCACACCACCCTTGTATTCAGTGCAAAAAATGGGACTTTTCTTAA
TAAATAGAGAAATGGAGGTGCCTAAAATTACAAAATTGCACTAGAGAGAT
AGTGATAGAACTGGGAAACTCTTAGTCTAATATTTTATCTTTTATTCATA
TGATGGAATACTAAGCTCAATGGCAGAATCTTCAGTCAGCAATGGTGTTC
AGGTTATGTGAACTATCTGAAGGATTCCTGAACTCTTCATATCTAGGAAT
GTAGCGTTTAAAAGCTCTTAGAATTTTTCATACTCTTAGGTCCTCCTGAC
TTGTGCTTCAATTCATGCATAAACTTATTTTATAAGGTCTCCGTCTGCCC
TTGCTGGAGATAACATTTTTGTTTATCCAACAAAGGGTATTTTATCTTAT
TATTAAATTCTGACTTTGTATAGAAGAGAAATGAAGTGATAATCTATATA
AATTAAGTCTTGATTAGTACATATGGGTTATTCACTTGGATAATATGGAG
TAAAATTTTAATTCATGGCTAATTACCTCCACCTCCACTACCTAGTGGCC
TCCCCTCACCAATATTAGCCAAAATAAATCAAATTTGGAACTACAAACCT
ACTTCAAAAAGGGTAAGGTATATAATAAGCAATATCATCAAGTCAAATAG
TATTTTTTTAACCATGTACAAGGCATCATGCTAGGTGTTACGAAGATGCA
TGAAATATATAAGATGTGGTTCCAACCCTCACAGAGTTTATAGACATCAC
ATAATAAATTCTGAAGTCAAATATAAATTAATTTAAAATTATCTGCTGTT
CAGCATTGTTCACTAGTGCAGCCAAACAATGTCATCTTGTTGAAAGGCAT
TGGTAGTAAAAACTGTGTCTGAAATACCCCTCTTTCAAAGGTTTCGTAAA
TTTGATATAGTCAGGGACACGAACAAGATCCTTTTACATTTTTCTTTTTG
CTTGTTAGTCTTGCTGTGCTCATAAATCCATGACAGAAAGCCCCCTCCCC
TGAGACTTTCCACTTCCTTTTCTGGCTTTCACTGTTGCAGAGGCTGCTAT
ATTCACGACATGTGGCCTACAGAGTTCTCAGGATGTAAGCAAGCCAAACT
GAAGACCAAATTTGTAGTTCTTGCTCTCCTAAACAGATGCATATGTGGCA
CTTCAGCTCTTCCAGATTACAACAGAATTTAGGTTTATGCATAAGTCTAA
TAAGTCTTCCTCATACTGAACTTCCTAATACTACTCCCCTTTTCTATCTT
TTCAACCCACATGACCACTATTAGACAGATAGTCAGAACCAGAAGGAAGT
GAAATGCTCACTCAACAAGCAGAGCATCTTTCTCAATGCAGTTATCATAG
ACAAGACACACTATGATAGAATTGGTGCTGCTATGTATTCTTTTTGAGGC
CCCTGAGAAAACCTCCAGGCTTCAAGTGACATACCTAGTCTGCTTTACCG
GTTTACAGGACTCAAGAGAAAGGTGGACATTGAGAGTTAATCCCTGAGGC
CAAATCTTAAATGGAGAAAGTCAACATCCACAGAAAATGGGGAAGGGCAC
AAGTATTTCTGTGGGCTTATATTCCGACATTTTTATCTGTAGGGGAAAAA
TGCTTTCTTAGAAAATGACTCAGCACGGGGAAGTCTTGTCTCTACCTCTG
TCTTGTTTTGTCCTTTGGGGTCCCTTCACTATCAAGTTCAACTGTGTGTC
CCTGAGACTCCTCTGCCCCGGAGGACAGGAGACTCGAAAAACGCTCTTCC
TGGCCAGTCTCTTTGCTCTGTGTCTGCCAGCCCCCAGCATCTCTCCTCTT
TCCTGTAAGCCCCTCTCCCTGTGCTGACTGTCTTCATAGTACTTTAGGTA
TGTTGTCCCTTTACCTCTGGGAGGATAGCTTGATGACCTGTCTGCTCAGG
CCAGCCCCATCTAGAGTCTCAGTGGCCCCAGTCATGTTGAGAAAGGTTCT
TTCAAAGATAGACTCAAGATAGTAGTGTCAGAGGTCCCAAGCAAATGAAG
GGCGGGGACAGTTGAGGGGGTGGAATAGGGACGGCAGCAGGGAACCAGAT
AGCATGCTGCTGAGAAGAAAAAAAGACATTGGTTTAGGTCAGGAAGCAAA
AAAAGGGAACTGAGTGGCTGTGAAAGGGTGGGGTTTGCTCAGACTGTCCT
TCCTCTCTGGACTGTAAGAATTAGTCTCGAGGCCACCATGGTGAGCAAGG
GCGAGGAGCTGTTCACCGGGGTGGTGCCCATCCTGGTCGAGCTGGACGGC
GACGTAAACGGCCACAAGTTCAGCGTGTCCGGCGAGGGCGAGGGCGATGC
CACCTACGGCAAGCTGACCCTGAAGTTCATCTGCACCACCGGCAAGCTGC
CCGTGCCCTGGCCCACCCTCGTGACCACCCTGACCTACGGCGTGCAGTGC
TTCAGCCGCTACCCCGACCACATGAAGCAGCACGACTTCTTCAAGTCCGC
CATGCCCGAAGGCTACGTCCAGGAGCGCACCATCTTCTTCAAGGACGACG
GCAACTACAAGACCCGCGCCGAGGTGAAGTTCGAGGGCGACACCCTGGTG
AACCGCATCGAGCTGAAGGGCATCGACTTCAAGGAGGACGGCAACATCCT
GGGGCACAAGCTGGAGTACAACTACAACAGCCACAACGTCTATATCATGG
CCGACAAGCAGAAGAACGGCATCAAGGTGAACTTCAAGATCCGCCACAAC
ATCGAGGACGGCAGCGTGCAGCTCGCCGACCACTACCAGCAGAACACCCC
CATCGGCGACGGCCCCGTGCTGCTGCCCGACAACCACTACCTGAGCACCC
AGTCCGCCCTGAGCAAAGACCCCAACGAGAAGCGCGATCACATGGTCCTG
CTGGAGTTCGTGACCGCCGCCGGGATCACTCTCGGCATGGACGAGCTGTA
CAAGTAA
0.7UCOE.BTKp.coBTK
(SEQ ID NO: 33)
CCGCGTGTGGCATCTGAAGCACCACCAGCGAGCGAGAGCTAGAGAGAAG
GAAAGCCACCGACTTCACCGCCTCCGAGCTGCTCCGGGTCGCGGGTCTG
CAGCGTCTCCGGCCCTCCGCGCCTACAGCTCAAGCCACATCCGAAGGGG
GAGGGAGCCGGGAGCTGCGCGCGGGGCCGCTGGGGGGAGGGGTGGCACC
GCCCACGCCGGGCGGCCACGAAGGGCGGGGCAGCGGGCGCGCGCCCGGC
GGGGGGAGGGGCCGCGCGCCGCGCCCGCTGGGAATTGGGGCCCTAGGGG
GAGGGCGGAGGCGCCGACGACCGCGGCACTTACCGTTCGCGGCGTGGCG
CCCGGTGGTCCCCAAGGGGAGGGAAGGGGGAGGCGGGGCGAGGACAGTG
ACCGGAGTCTCCTCAGCGGTGGCTTTTCTGCTTGGCAGCCTCAGCGGCT
GGCGCCAAAACCGGACTCCGCCCACTTCCTCGCCCCTGCGGTGCGAGGG
TGTGGAATCCTCCAGACGCTGGGGGAGGGGGAGTTGGGAGCTTAAAAAC
TAGTACCCCTTTGGGACCACTTTCAGCAGCGAACTCTCCTGTACACCAG
GGGTCAGTTCCACAGACGCGGGCCAGGGGTGGGTCATTGCGGCGTGAAC
AATAATTTGACTAGAAGTTGATTCGGGTGTTTGCGGCCGGGGCTAGCTA
CGACGCGTTCCGGAATTCGCCCTTGCATTTCCTAGGAGAATCCCTGGGG
GAATCATTGCAGTTGGAGCATAATGTAGGGGGCCCCTGAGAAAACCTCC
AGGCTTCAAGTGACATACCTAGTCTGCTTTACCGGTTTACAGGACTCAA
GAGAAAGGTGGACATTGAGAGTTAATCCCTGAGGCCAAATCTTAAATGG
AGAAAGTCAACATCCACAGAAAATGGGGAAGGGCACAAGTATTTCTGTG
GGCTTATATTCCGACATTTTTATCTGTAGGGGAAAAATGCTTTCTTAGA
AAATGACTCAGCACGGGGAAGTCTTGTCTCTACCTCTGTCTTGTTTTGT
CCTTTGGGGTCCCTTCACTATCAAGTTCAACTGTGTGTCCCTGAGACTC
CTCTGCCCCGGAGGACAGGAGACTCGAAAAACGCTCTTCCTGGCCAGTC
TCTTTGCTCTGTGTCTGCCAGCCCCCAGCATCTCTCCTCTTTCCTGTAA
GCCCCTCTCCCTGTGCTGACTGTCTTCATAGTACTTTAGGTATGTTGTC
CCTTTACCTCTGGGAGGATAGCTTGATGACCTGTCTGCTCAGGCCAGCC
CCATCTAGAGTCTCAGTGGCCCCAGTCATGTTGAGAAAGGTTCTTTCAA
AGATAGACTCAAGATAGTAGTGTCAGAGGTCCCAAGCAAATGAAGGGCG
GGGACAGTTGAGGGGGTGGAATAGGGACGGCAGCAGGGAACCAGATAGC
ATGCTGCTGAGAAGAAAAAAAGACATTGGTTTAGGTCAGGAAGCAAAAA
AAGGGAACTGAGTGGCTGTGAAAGGGTGGGGTTTGCTCAGACTGTCCTT
CCTCTCTGGACTGTAAGAATTAGTCTCGAGAAAGAAGCCACCATGGCCG
CTGTGATCCTGGAGAGCATTTTCCTGAAGAGGTCCCAGCAGAAAAAGAA
AACCTCTCCCCTGAACTTTAAGAAAAGACTGTTCCTGCTGACAGTGCAC
AAGCTGTCTTACTATGAGTACGACTTTGAGCGGGGCCGCCGAGGATCAA
AAAAGGGGAGCATCGATGTGGAGAAGATTACATGCGTGGAGACCGTGGT
CCCTGAAAAGAATCCACCCCCTGAGAGGCAGATCCCAAGACGGGGCGAG
GAGTCCTCTGAGATGGAGCAGATTAGTATCATTGAGCGCTTCCCCTATC
CTTTTCAGGTGGTGTACGACGAGGGACCACTGTATGTGTTCTCACCCAC
AGAGGAGCTGAGAAAGAGGTGGATTCACCAGCTGAAGAACGTGATTAGA
TACAATAGCGATCTGGTGCAGAAGTATCACCCTTGTTTTTGGATCGACG
GGCAGTACCTGTGCTGTTCCCAGACAGCTAAGAACGCTATGGGATGCCA
GATTCTGGAAAATCGGAACGGATCTCTGAAACCAGGGAGTTCACACCGC
AAGACCAAAAAGCCCCTGCCTCCAACACCCGAGGAGGATCAGATCCTGA
AAAAGCCTCTGCCACCCGAGCCTGCTGCAGCCCCAGTCAGCACTTCCGA
ACTGAAAAAGGTGGTGGCTCTGTATGACTACATGCCCATGAATGCTAAC
GATCTGCAGCTGAGAAAGGGCGACGAGTATTTCATTCTGGAAGAGTCTA
ATCTGCCTTGGTGGAGGGCCAGAGATAAGAACGGACAGGAGGGGTACAT
CCCATCTAATTATGTGACCGAGGCTGAGGACTCTATTGAGATGTACGAG
TGGTATAGCAAGCACATGACACGGTCCCAGGCTGAGCAGCTGCTGAAGC
AGGAGGGCAAAGAGGGAGGGTTTATCGTGCGCGATTCTAGTAAGGCCGG
CAAATACACTGTGTCAGTGTTCGCTAAGAGCACCGGAGACCCCCAGGGC
GTGATCAGACACTATGTGGTGTGTTCCACACCTCAGTCTCAGTACTATC
TGGCTGAGAAGCACCTGTTTAGTACAATCCCAGAGCTGATTAACTACCA
CCAGCACAATTCTGCCGGCCTGATCAGCAGGCTGAAGTATCCCGTCTCC
CAGCAGAACAAAAATGCTCCTTCTACCGCTGGACTGGGGTACGGCAGTT
GGGAGATTGATCCAAAGGACCTGACATTCCTGAAGGAGCTGGGAACTGG
GCAGTTTGGCGTGGTGAAGTATGGAAAATGGAGAGGGCAGTACGATGTG
GCCATCAAGATGATCAAGGAGGGCTCAATGAGCGAGGACGAGTTCATCG
AGGAGGCTAAGGTCATGATGAACCTGTCCCACGAGAAACTGGTGCAGCT
GTATGGAGTGTGCACCAAGCAGCGGCCCATTTTTATCATTACAGAGTAC
ATGGCTAATGGGTGTCTGCTGAACTATCTGCGCGAGATGAGACACAGAT
TCCAGACACAGCAGCTGCTGGAAATGTGCAAGGATGTGTGTGAGGCTAT
GGAGTACCTGGAGTCTAAGCAGTTTCTGCACCGGGACCTGGCTGCTCGC
AATTGCCTGGTGAACGATCAGGGCGTGGTGAAGGTGAGTGACTTCGGAC
TGTCAAGGTATGTGCTGGATGACGAGTACACCAGCTCCGTGGGCTCTAA
GTTTCCTGTGAGATGGTCTCCACCCGAGGTGCTGATGTATAGCAAGTTC
TCCTCTAAGAGCGATATCTGGGCCTTTGGCGTGCTGATGTGGGAAATCT
ACAGCCTGGGCAAGATGCCTTACGAGCGGTTCACAAATTCCGAGACAGC
TGAGCACATCGCCCAGGGCCTGCGCCTGTACCGGCCACATCTGGCCTCT
GAGAAGGTGTACACCATCATGTACAGCTGTTGGCACGAGAAGGCCGACG
AGAGACCCACATTCAAGATCCTGCTGTCCAACATTCTAGATGTGATGGA
CGAGGAGAGCTGA
IE.BTKp.coBTK
(SEQ ID NO: 34)
GCATTTCCTAGGAGAATCCCTGGGGGAATCATTGCAGTTGGAGCATAATG
TAGGGGGCCCACATCTTTGAGCTTCAGTTTCCTCATCTGTAAATAGGGGA
ATAATACATACTTCTTAAGGCTACTGCAAAGATCAAATAAGTAATACATT
TGAAGCACTTGGGACAGAGCCTGCTACATAGTAAGTGCTCATTAAGTGTT
AGTTATCATTGTTGTTGTTTTTAGGCCAAGGTTGTTGTGAAAATTAAATG
AGATAATATATAAAAGGTATTTAGCTCAATATCTGGCACATAGCAATAAT
TGAATAGATGATCCTTCATCTTCGTTCCTCCTGTTCCCTTTCAGTTTGAA
AGACTTGGCTAATATAATTTTGACCAACCAAACTTGCATTCAAGGGAGTG
TACAAGGCTGGTATAGCCAGCCAGTGAGTATCAGAATCTAAATGTTTATT
AAGACAAAGGGCTGTCATGCAATAACCCAACCATACCATTATCAGTCTGC
CATCCTTCCTGTTTCTCTAGGCAGCCTTTCCTGATGTCAACTCAACCAGT
TAATCTCTCAGTCACTTGACATGTGGCTATATATACACACAAATATGTGT
GCATGCATCCTGTGCTGCAAGCATTTACAGTCAAGTTTATCTGAACACAC
TGTATGGTTGATGTGAAATGCTGAAACTGTTCAAGTTTAGGTCCTCACAA
AGCAAGGAATATGAAATATTTCCTTGGGAAATATTTATCCACAACAAAGA
GATGTACAGTGCTTTCGTATACAGTGATTTACAGTTTTCCATGTGCTTTT
ACATGTATTATTACTTCATTTGATCCTTACAACAACCCCAGAGGTAGATG
TGGCATGAATTACCATTATTCTCCTTTGAGAAGAAGAAACTGAGCATCAA
AGAAGCTTGTTGGCCTTCTTGCCAGAAATCACCCAGTTTGTAAATGGTAA
AAGAGGGCTTGAAACCAGGTTCTCTGACTCTGACTTCAAGCACTCTCATA
CATCATCTATTTAATTTTTTGGAGCTAGGTATTTTATACTTAGGATTCTA
AATATTGCATAACCATTGAATGCCACACCACCCTTGTATTCAGTGCAAAA
AATGGGACTTTTCTTAATAAATAGAGAAATGGAGGTGCCTAAAATTACAA
AATTGCACTAGAGAGATAGTGATAGAACTGGGAAACTCTTAGTCTAATAT
TTTATCTTTTATTCATATGATGGAATACTAAGCTCAATGGCAGAATCTTC
AGTCAGCAATGGTGTTCAGGTTATGTGAACTATCTGAAGGATTCCTGAAC
TCTTCATATCTAGGAATGTAGCGTTTAAAAGCTCTTAGAATTTTTCATAC
TCTTAGGTCCTCCTGACTTGTGCTTCAATTCATGCATAAACTTATTTTAT
AAGGTCTCCGTCTGCCCTTGCTGGAGATAACATTTTTGTTTATCCAACAA
AGGGTATTTTATCTTATTATTAAATTCTGACTTTGTATAGAAGAGAAATG
AAGTGATAATCTATATAAATTAAGTCTTGATTAGTACATATGGGTTATTC
ACTTGGATAATATGGAGTAAAATTTTAATTCATGGCTAATTACCTCCACC
TCCACTACCTAGTGGCCTCCCCTCACCAATATTAGCCAAAATAAATCAAA
TTTGGAACTACAAACCTACTTCAAAAAGGGTAAGGTATATAATAAGCAAT
ATCATCAAGTCAAATAGTATTTTTTTAACCATGTACAAGGCATCATGCTA
GGTGTTACGAAGATGCATGAAATATATAAGATGTGGTTCCAACCCTCACA
GAGTTTATAGACATCACATAATAAATTCTGAAGTCAAATATAAATTAATT
TAAAATTATCTGCTGTTCAGCATTGTTCACTAGTGCAGCCAAACAATGTC
ATCTTGTTGAAAGGCATTGGTAGTAAAAACTGTGTCTGAAATACCCCTCT
TTCAAAGGTTTCGTAAATTTGATATAGTCAGGGACACGAACAAGATCCTT
TTACATTTTTCTTTTTGCTTGTTAGTCTTGCTGTGCTCATAAATCCATGA
CAGAAAGCCCCCTCCCCTGAGACTTTCCACTTCCTTTTCTGGCTTTCACT
GTTGCAGAGGCTGCTATATTCACGACATGTGGCCTACAGAGTTCTCAGGA
TGTAAGCAAGCCAAACTGAAGACCAAATTTGTAGTTCTTGCTCTCCTAAA
CAGATGCATATGTGGCACTTCAGCTCTTCCAGATTACAACAGAATTTAGG
TTTATGCATAAGTCTAATAAGTCTTCCTCATACTGAACTTCCTAATACTA
CTCCCCTTTTCTATCTTTTCAACCCACATGACCACTATTAGACAGATAGT
CAGAACCAGAAGGAAGTGAAATGCTCACTCAACAAGCAGAGCATCTTTCT
CAATGCAGTTATCATAGACAAGACACACTATGATAGAATTGGTGCTGCTA
TGTATTCTTTTTGAGGCCCCTGAGAAAACCTCCAGGCTTCAAGTGACATA
CCTAGTCTGCTTTACCGGTTTACAGGACTCAAGAGAAAGGTGGACATTGA
GAGTTAATCCCTGAGGCCAAATCTTAAATGGAGAAAGTCAACATCCACAG
AAAATGGGGAAGGGCACAAGTATTTCTGTGGGCTTATATTCCGACATTTT
TATCTGTAGGGGAAAAATGCTTTCTTAGAAAATGACTCAGCACGGGGAAG
TCTTGTCTCTACCTCTGTCTTGTTTTGTCCTTTGGGGTCCCTTCACTATC
AAGTTCAACTGTGTGTCCCTGAGACTCCTCTGCCCCGGAGGACAGGAGAC
TCGAAAAACGCTCTTCCTGGCCAGTCTCTTTGCTCTGTGTCTGCCAGCCC
CCAGCATCTCTCCTCTTTCCTGTAAGCCCCTCTCCCTGTGCTGACTGTCT
TCATAGTACTTTAGGTATGTTGTCCCTTTACCTCTGGGAGGATAGCTTGA
TGACCTGTCTGCTCAGGCCAGCCCCATCTAGAGTCTCAGTGGCCCCAGTC
ATGTTGAGAAAGGTTCTTTCAAAGATAGACTCAAGATAGTAGTGTCAGAG
GTCCCAAGCAAATGAAGGGCGGGGACAGTTGAGGGGGTGGAATAGGGACG
GCAGCAGGGAACCAGATAGCATGCTGCTGAGAAGAAAAAAAGACATTGGT
TTAGGTCAGGAAGCAAAAAAAGGGAACTGAGTGGCTGTGAAAGGGTGGGG
TTTGCTCAGACTGTCCTTCCTCTCTGGACTGTAAGAATTAGTCTCGAGAA
AGAAGCCACCATGGCCGCTGTGATCCTGGAGAGCATTTTCCTGAAGAGGT
CCCAGCAGAAAAAGAAAACCTCTCCCCTGAACTTTAAGAAAAGACTGTTC
CTGCTGACAGTGCACAAGCTGTCTTACTATGAGTACGACTTTGAGCGGGG
CCGCCGAGGATCAAAAAAGGGGAGCATCGATGTGGAGAAGATTACATGCG
TGGAGACCGTGGTCCCTGAAAAGAATCCACCCCCTGAGAGGCAGATCCCA
AGACGGGGCGAGGAGTCCTCTGAGATGGAGCAGATTAGTATCATTGAGCG
CTTCCCCTATCCTTTTCAGGTGGTGTACGACGAGGGACCACTGTATGTGT
TCTCACCCACAGAGGAGCTGAGAAAGAGGTGGATTCACCAGCTGAAGAAC
GTGATTAGATACAATAGCGATCTGGTGCAGAAGTATCACCCTTGTTTTTG
GATCGACGGGCAGTACCTGTGCTGTTCCCAGACAGCTAAGAACGCTATGG
GATGCCAGATTCTGGAAAATCGGAACGGATCTCTGAAACCAGGGAGTTCA
CACCGCAAGACCAAAAAGCCCCTGCCTCCAACACCCGAGGAGGATCAGAT
CCTGAAAAAGCCTCTGCCACCCGAGCCTGCTGCAGCCCCAGTCAGCACTT
CCGAACTGAAAAAGGTGGTGGCTCTGTATGACTACATGCCCATGAATGCT
AACGATCTGCAGCTGAGAAAGGGCGACGAGTATTTCATTCTGGAAGAGTC
TAATCTGCCTTGGTGGAGGGCCAGAGATAAGAACGGACAGGAGGGGTACA
TCCCATCTAATTATGTGACCGAGGCTGAGGACTCTATTGAGATGTACGAG
TGGTATAGCAAGCACATGACACGGTCCCAGGCTGAGCAGCTGCTGAAGCA
GGAGGGCAAAGAGGGAGGGTTTATCGTGCGCGATTCTAGTAAGGCCGGCA
AATACACTGTGTCAGTGTTCGCTAAGAGCACCGGAGACCCCCAGGGCGTG
ATCAGACACTATGTGGTGTGTTCCACACCTCAGTCTCAGTACTATCTGGC
TGAGAAGCACCTGTTTAGTACAATCCCAGAGCTGATTAACTACCACCAGC
ACAATTCTGCCGGCCTGATCAGCAGGCTGAAGTATCCCGTCTCCCAGCAG
AACAAAAATGCTCCTTCTACCGCTGGACTGGGGTACGGCAGTTGGGAGAT
TGATCCAAAGGACCTGACATTCCTGAAGGAGCTGGGAACTGGGCAGTTTG
GCGTGGTGAAGTATGGAAAATGGAGAGGGCAGTACGATGTGGCCATCAAG
ATGATCAAGGAGGGCTCAATGAGCGAGGACGAGTTCATCGAGGAGGCTAA
GGTCATGATGAACCTGTCCCACGAGAAACTGGTGCAGCTGTATGGAGTGT
GCACCAAGCAGCGGCCCATTTTTATCATTACAGAGTACATGGCTAATGGG
TGTCTGCTGAACTATCTGCGCGAGATGAGACACAGATTCCAGACACAGCA
GCTGCTGGAAATGTGCAAGGATGTGTGTGAGGCTATGGAGTACCTGGAGT
CTAAGCAGTTTCTGCACCGGGACCTGGCTGCTCGCAATTGCCTGGTGAAC
GATCAGGGCGTGGTGAAGGTGAGTGACTTCGGACTGTCAAGGTATGTGCT
GGATGACGAGTACACCAGCTCCGTGGGCTCTAAGTTTCCTGTGAGATGGT
CTCCACCCGAGGTGCTGATGTATAGCAAGTTCTCCTCTAAGAGCGATATC
TGGGCCTTTGGCGTGCTGATGTGGGAAATCTACAGCCTGGGCAAGATGCC
TTACGAGCGGTTCACAAATTCCGAGACAGCTGAGCACATCGCCCAGGGCC
TGCGCCTGTACCGGCCACATCTGGCCTCTGAGAAGGTGTACACCATCATG
TACAGCTGTTGGCACGAGAAGGCCGACGAGAGACCCACATTCAAGATCCT
GCTGTCCAACATTCTAGATGTGATGGACGAGGAGAGCTGA
0.7UCOE.IE.BTKp.coBTK
(SEQ ID NO: 35)
CCGCGTGTGGCATCTGAAGCACCACCAGCGAGCGAGAGCTAGAGAGAAG
GAAAGCCACCGACTTCACCGCCTCCGAGCTGCTCCGGGTCGCGGGTCTGC
AGCGTCTCCGGCCCTCCGCGCCTACAGCTCAAGCCACATCCGAAGGGGGA
GGGAGCCGGGAGCTGCGCGCGGGGCCGCTGGGGGGAGGGGTGGCACCGC
CCACGCCGGGCGGCCACGAAGGGCGGGGCAGCGGGCGCGCGCCCGGCGG
GGGGAGGGGCCGCGCGCCGCGCCCGCTGGGAATTGGGGCCCTAGGGGGA
GGGCGGAGGCGCCGACGACCGCGGCACTTACCGTTCGCGGCGTGGCGCC
CGGTGGTCCCCAAGGGGAGGGAAGGGGGAGGCGGGGCGAGGACAGTGA
CCGGAGTCTCCTCAGCGGTGGCTTTTCTGCTTGGCAGCCTCAGCGGCTGG
CGCCAAAACCGGACTCCGCCCACTTCCTCGCCCCTGCGGTGCGAGGGTGT
GGAATCCTCCAGACGCTGGGGGAGGGGGAGTTGGGAGCTTAAAAACTAGT
ACCCCTTTGGGACCACTTTCAGCAGCGAACTCTCCTGTACACCAGGGGTC
AGTTCCACAGACGCGGGCCAGGGGTGGGTCATTGCGGCGTGAACAATAAT
TTGACTAGAAGTTGATTCGGGTGTTTGCGGCCGGGGCTAGCACATCTTTG
AGCTTCAGTTTCCTCATCTGTAAATAGGGGAATAATACATACTTCTTAAG
GCTACTGCAAAGATCAAATAAGTAATACATTTGAAGCACTTGGGACAGAG
CCTGCTACATAGTAAGTGCTCATTAAGTGTTAGTTATCATTGTTGTTGTT
TTTAGGCCAAGGTTGTTGTGAAAATTAAATGAGATAATATATAAAAGGTA
TTTAGCTCAATATCTGGCACATAGCAATAATTGAATAGATGATCCTTCAT
CTTCGTTCCTCCTGTTCCCTTTCAGTTTGAAAGACTTGGCTAATATAATT
TTGACCAACCAAACTTGCATTCAAGGGAGTGTACAAGGCTGGTATAGCCA
GCCAGTGAGTATCAGAATCTAAATGTTTATTAAGACAAAGGGCTGTCATG
CAATAACCCAACCATACCATTATCAGTCTGCCATCCTTCCTGTTTCTCTA
GGCAGCCTTTCCTGATGTCAACTCAACCAGTTAATCTCTCAGTCACTTGA
CATGTGGCTATATATACACACAAATATGTGTGCATGCATCCTGTGCTGCA
AGCATTTACAGTCAAGTTTATCTGAACACACTGTATGGTTGATGTGAAAT
GCTGAAACTGTTCAAGTTTAGGTCCTCACAAAGCAAGGAATATGAAATAT
TTCCTTGGGAAATATTTATCCACAACAAAGAGATGTACAGTGCTTTCGTA
TACAGTGATTTACAGTTTTCCATGTGCTTTTACATGTATTATTACTTCAT
TTGATCCTTACAACAACCCCAGAGGTAGATGTGGCATGAATTACCATTAT
TCTCCTTTGAGAAGAAGAAACTGAGCATCAAAGAAGCTTGTTGGCCTTCT
TGCCAGAAATCACCCAGTTTGTAAATGGTAAAAGAGGGCTTGAAACCAGG
TTCTCTGACTCTGACTTCAAGCACTCTCATACATCATCTATTTAATTTTT
TGGAGCTAGGTATTTTATACTTAGGATTCTAAATATTGCATAACCATTGA
ATGCCACACCACCCTTGTATTCAGTGCAAAAAATGGGACTTTTCTTAATA
AATAGAGAAATGGAGGTGCCTAAAATTACAAAATTGCACTAGAGAGATAG
TGATAGAACTGGGAAACTCTTAGTCTAATATTTTATCTTTTATTCATATG
ATGGAATACTAAGCTCAATGGCAGAATCTTCAGTCAGCAATGGTGTTCAG
GTTATGTGAACTATCTGAAGGATTCCTGAACTCTTCATATCTAGGAATGT
AGCGTTTAAAAGCTCTTAGAATTTTTCATACTCTTAGGTCCTCCTGACTT
GTGCTTCAATTCATGCATAAACTTATTTTATAAGGTCTCCGTCTGCCCTT
GCTGGAGATAACATTTTTGTTTATCCAACAAAGGGTATTTTATCTTATTA
TTAAATTCTGACTTTGTATAGAAGAGAAATGAAGTGATAATCTATATAAA
TTAAGTCTTGATTAGTACATATGGGTTATTCACTTGGATAATATGGAGTA
AAATTTTAATTCATGGCTAATTACCTCCACCTCCACTACCTAGTGGCCTC
CCCTCACCAATATTAGCCAAAATAAATCAAATTTGGAACTACAAACCTAC
TTCAAAAAGGGTAAGGTATATAATAAGCAATATCATCAAGTCAAATAGTA
TTTTTTTAACCATGTACAAGGCATCATGCTAGGTGTTACGAAGATGCATG
AAATATATAAGATGTGGTTCCAACCCTCACAGAGTTTATAGACATCACAT
AATAAATTCTGAAGTCAAATATAAATTAATTTAAAATTATCTGCTGTTCA
GCATTGTTCACTAGTGCAGCCAAACAATGTCATCTTGTTGAAAGGCATTG
GTAGTAAAAACTGTGTCTGAAATACCCCTCTTTCAAAGGTTTCGTAAATT
TGATATAGTCAGGGACACGAACAAGATCCTTTTACATTTTTCTTTTTGCT
TGTTAGTCTTGCTGTGCTCATAAATCCATGACAGAAAGCCCCCTCCCCTG
AGACTTTCCACTTCCTTTTCTGGCTTTCACTGTTGCAGAGGCTGCTATAT
TCACGACATGTGGCCTACAGAGTTCTCAGGATGTAAGCAAGCCAAACTGA
AGACCAAATTTGTAGTTCTTGCTCTCCTAAACAGATGCATATGTGGCACT
TCAGCTCTTCCAGATTACAACAGAATTTAGGTTTATGCATAAGTCTAATA
AGTCTTCCTCATACTGAACTTCCTAATACTACTCCCCTTTTCTATCTTTT
CAACCCACATGACCACTATTAGACAGATAGTCAGAACCAGAAGGAAGTGA
AATGCTCACTCAACAAGCAGAGCATCTTTCTCAATGCAGTTATCATAGAC
AAGACACACTATGATAGAATTGGTGCTGCTATGTATTCTTTTTGAACGCG
TTCCGGAATTCGCCCTTGCATTTCCTAGGAGAATCCCTGGGGGAATCATT
GCAGTTGGAGCATAATGTAGGGGGCCCCTGAGAAAACCTCCAGGCTTCAA
GTGACATACCTAGTCTGCTTTACCGGTTTACAGGACTCAAGAGAAAGGTG
GACATTGAGAGTTAATCCCTGAGGCCAAATCTTAAATGGAGAAAGTCAAC
ATCCACAGAAAATGGGGAAGGGCACAAGTATTTCTGTGGGCTTATATTCC
GACATTTTTATCTGTAGGGGAAAAATGCTTTCTTAGAAAATGACTCAGCA
CGGGGAAGTCTTGTCTCTACCTCTGTCTTGTTTTGTCCTTTGGGGTCCCT
TCACTATCAAGTTCAACTGTGTGTCCCTGAGACTCCTCTGCCCCGGAGGA
CAGGAGACTCGAAAAACGCTCTTCCTGGCCAGTCTCTTTGCTCTGTGTCT
GCCAGCCCCCAGCATCTCTCCTCTTTCCTGTAAGCCCCTCTCCCTGTGCT
GACTGTCTTCATAGTACTTTAGGTATGTTGTCCCTTTACCTCTGGGAGGA
TAGCTTGATGACCTGTCTGCTCAGGCCAGCCCCATCTAGAGTCTCAGTGG
CCCCAGTCATGTTGAGAAAGGTTCTTTCAAAGATAGACTCAAGATAGTAG
TGTCAGAGGTCCCAAGCAAATGAAGGGCGGGGACAGTTGAGGGGGTGGAA
TAGGGACGGCAGCAGGGAACCAGATAGCATGCTGCTGAGAAGAAAAAAAG
ACATTGGTTTAGGTCAGGAAGCAAAAAAAGGGAACTGAGTGGCTGTGAAA
GGGTGGGGTTTGCTCAGACTGTCCTTCCTCTCTGGACTGTAAGAATTAGT
CTCGAGAAAGAAGCCACCATGGCCGCTGTGATCCTGGAGAGCATTTTCCT
GAAGAGGTCCCAGCAGAAAAAGAAAACCTCTCCCCTGAACTTTAAGAAAA
GACTGTTCCTGCTGACAGTGCACAAGCTGTCTTACTATGAGTACGACTTT
GAGCGGGGCCGCCGAGGATCAAAAAAGGGGAGCATCGATGTGGAGAAGAT
TACATGCGTGGAGACCGTGGTCCCTGAAAAGAATCCACCCCCTGAGAGGC
AGATCCCAAGACGGGGCGAGGAGTCCTCTGAGATGGAGCAGATTAGTATC
ATTGAGCGCTTCCCCTATCCTTTTCAGGTGGTGTACGACGAGGGACCACT
GTATGTGTTCTCACCCACAGAGGAGCTGAGAAAGAGGTGGATTCACCAGC
TGAAGAACGTGATTAGATACAATAGCGATCTGGTGCAGAAGTATCACCCT
TGTTTTTGGATCGACGGGCAGTACCTGTGCTGTTCCCAGACAGCTAAGAA
CGCTATGGGATGCCAGATTCTGGAAAATCGGAACGGATCTCTGAAACCAG
GGAGTTCACACCGCAAGACCAAAAAGCCCCTGCCTCCAACACCCGAGGAG
GATCAGATCCTGAAAAAGCCTCTGCCACCCGAGCCTGCTGCAGCCCCAGT
CAGCACTTCCGAACTGAAAAAGGTGGTGGCTCTGTATGACTACATGCCCA
TGAATGCTAACGATCTGCAGCTGAGAAAGGGCGACGAGTATTTCATTCTG
GAAGAGTCTAATCTGCCTTGGTGGAGGGCCAGAGATAAGAACGGACAGGA
GGGGTACATCCCATCTAATTATGTGACCGAGGCTGAGGACTCTATTGAGA
TGTACGAGTGGTATAGCAAGCACATGACACGGTCCCAGGCTGAGCAGCTG
CTGAAGCAGGAGGGCAAAGAGGGAGGGTTTATCGTGCGCGATTCTAGTAA
GGCCGGCAAATACACTGTGTCAGTGTTCGCTAAGAGCACCGGAGACCCCC
AGGGCGTGATCAGACACTATGTGGTGTGTTCCACACCTCAGTCTCAGTAC
TATCTGGCTGAGAAGCACCTGTTTAGTACAATCCCAGAGCTGATTAACTA
CCACCAGCACAATTCTGCCGGCCTGATCAGCAGGCTGAAGTATCCCGTCT
CCCAGCAGAACAAAAATGCTCCTTCTACCGCTGGACTGGGGTACGGCAGT
TGGGAGATTGATCCAAAGGACCTGACATTCCTGAAGGAGCTGGGAACTGG
GCAGTTTGGCGTGGTGAAGTATGGAAAATGGAGAGGGCAGTACGATGTGG
CCATCAAGATGATCAAGGAGGGCTCAATGAGCGAGGACGAGTTCATCGAG
GAGGCTAAGGTCATGATGAACCTGTCCCACGAGAAACTGGTGCAGCTGTA
TGGAGTGTGCACCAAGCAGCGGCCCATTTTTATCATTACAGAGTACATGG
CTAATGGGTGTCTGCTGAACTATCTGCGCGAGATGAGACACAGATTCCAG
ACACAGCAGCTGCTGGAAATGTGCAAGGATGTGTGTGAGGCTATGGAGTA
CCTGGAGTCTAAGCAGTTTCTGCACCGGGACCTGGCTGCTCGCAATTGCC
TGGTGAACGATCAGGGCGTGGTGAAGGTGAGTGACTTCGGACTGTCAAGG
TATGTGCTGGATGACGAGTACACCAGCTCCGTGGGCTCTAAGTTTCCTGT
GAGATGGTCTCCACCCGAGGTGCTGATGTATAGCAAGTTCTCCTCTAAGA
GCGATATCTGGGCCTTTGGCGTGCTGATGTGGGAAATCTACAGCCTGGGC
AAGATGCCTTACGAGCGGTTCACAAATTCCGAGACAGCTGAGCACATCGC
CCAGGGCCTGCGCCTGTACCGGCCACATCTGGCCTCTGAGAAGGTGTACA
CCATCATGTACAGCTGTTGGCACGAGAAGGCCGACGAGAGACCCACATTC
AAGATCCTGCTGTCCAACATTCTAGATGTGATGGACGAGGAGAGCTGA
ABCD.BTKp.GFP
(SEQ ID NO: 36)
TTTCCTAGGAGAATCCCTGGGGGAATCATTGCAGTTGGAGCATAATGTAG
GGGGCCCGGATCCCTGGGGTATGGCAGGGGCTGGGCAGCAGCAGCAATG
TACCTTGCTTGGGACCCCTAAAAACCAGAGAGACAGCATGGCTGGTGCCA
TTTATCAGCTAGTGGAGGAGGCTGACGGAGGGTGGGAGTGTCATCAGCAC
AAGGCCCTGGCAGTCCCTTCTGGTGATTAGAGAGGCCGAAAGGGTCCTTT
CCGACAAGGGCTGAGGGTGGGCGGAACAGGAAGAGAAAAATGTGACATG
AGGTGACCATCCGAACAGGTAGCAAATGTTAGAAAGGGGTACCTCTGGC
AAACTTAGTGGAAAAGTAATATTGCAGGGAGCAGTCAGATAAAAACAAG
CCCTTCTGTCAAATAGTGCTTGAAGACTCAATAGGGATACATGGGTCAAT
GAAGCCTTTAGAAAAAGAAATACTAAGAGGCAGATTCTCTGAGAACATG
GTAAAAGCTCACGCTCCACGTTATGAAGTTGACCTTTGTGAGCTAGGGAA
AGGCCTGGCTAGGCCAGGGTGTAGGCTACCTGCCTTGAGCTGTACCAGGC
CAAATGTCGCCAGGGTCAGAGCTGGCTTATTAAAGGACTGTGTGGAAGCT
GTGCCAACCTCGTGGTAACAATGGGTAAAAGACTGGGCCAGGAGAAAGC
AGCCTCTGCCTCAGCCCAGACAGTGCGGCCAACCCTTGAGGTTGTGGCAA
AGGTTTCTCCTCTTACCATTGCCCTCCATGTGCATGGCTTGCTTTTCTCT
TGTCTTCATTATTTCTCCTTTCCTTTCCTCGGATCCACGCGTGAATTCTT
TGTAAACTCCTTATGGTGCGAACTAATGTAACTTTCCATCCAGTTATGGG
GGATTGGTGCAATTTTAAATTATCACTATGATTTGCTATTTCCATTTGAG
CAAATTTCCTATAGAGTTTCCTTTCAGTGGACTAGACCCATATCAGGAAG
TGACTTAGGTATAAAGGGAAGATACAGCTTTCGAAAACCAAAGTTTGGGC
GTTCTCCAAAGAGTTATCAGATACCCCCTTCTACACCCACAATGATCTGA
TTGCTGAGATCTGATTGCTAACTACTGAAAATAAGGAAGAACTAGAATTT
TCAGTGACACAGTGCTCAGCAAGAAGCTAGAAAAGAGGCCTTGACATATT
TGACTCCAAAGCTACTTGGTTATGCATGAAGCCATCTGGGGAGGGGAAGG
AGGAGGGAGAACTCCTCTGAGGACCCTGAAACAATTGGGCCACGTGTGAC
TTTCAGTTTCTATGGAGATTCATGTGCAGTGGCTGAGGGCAATCTGAGAG
CATTGGAAACCCAGAAGCTTTAAACGCGTAGGAAAGACAGGGAATGGCCA
GAATCTTCCTAGCCAATTGAGTAGTGCTTTCAAGGAGAAATCAAGAGAAA
ACACTACTTCTTGGATATTTTGGCTAAGTAGTCATTTGAAGTACAGTTGA
CTGGTTATTTTATTTTAAATCATATCTCATAGACTCTTCCCAATCATAGG
CTGGCTGTGTAGTTTTCTGAATTTTGCTCTGGTATTCTTCTTCTTTTTTT
TTTTTATTTTTATTTTTTATTTTTTTTTTGAGACTCCAGGCTGGAGTGCA
GTGTCACGATCTTGGCTCACTGCAACCTCCGCCTCCCGAGTTCAAGCGAT
TCTCCTGCCTCAGCCTCCTGAGTTGCTGGGACTACAGGCGCCTGTCACCA
CGCCCGGCTAATTTTTTGTATTTTAATAGAGACGGGATTTCACCATGTTG
GCCAGGCTGGTCTCTAACTCCTGACCTCAAGTGATCCGCCAGCCTCAGCC
TCCCAAAGTGCTGGGATTACGGGCATGAGCCACTGCGCCTGGACTTATTA
TTCTTAATAGTATTTTATCTTATGAGCGAAGATAAGAGCCCAAGATGGTT
TAGTTTACTGATTCTGCAAGTGCTATTTCTATTAATTCCTTGGCATACTG
CAGTTTGTATGATGGCTGCACTCTTGTTAATAAGCTTCGTCTTTCTGAAT
TCTGTTGCTCCATAGGGAGCTGGGAGGCTGCAAAAGGTGGCCCTGTAAAA
ATCTTTGCATTTATAATTTAATAATTACAGACCCCAGTGGGACAATGTTT
GAAAAATTATATTCACCGTCTAGGAAATTGGGAACTGAAAGTCCAATATC
TGCCTCAGTGGAGTTCTGGCACCTGCATTATCCCTTCTGGGTATATCAAG
ATCAACAGCTGCACAGATACTTTTGCTTTTCACAGATTCTACACATATCA
TATAAAGGTGAATAGTGTAAAGCTACCTCTACACCTTACCAAGCACACAG
GGGGCCCCTGAGAAAACCTCCAGGCTTCAAGTGACATACCTAGTCTGCTT
TACCGGTTTACAGGACTCAAGAGAAAGGTGGACATTGAGAGTTAATCCCT
GAGGCCAAATCTTAAATGGAGAAAGTCAACATCCACAGAAAATGGGGAAG
GGCACAAGTATTTCTGTGGGCTTATATTCCGACATTTTTATCTGTAGGGG
AAAAATGCTTTCTTAGAAAATGACTCAGCACGGGGAAGTCTTGTCTCTAC
CTCTGTCTTGTTTTGTCCTTTGGGGTCCCTTCACTATCAAGTTCAACTGT
GTGTCCCTGAGACTCCTCTGCCCCGGAGGACAGGAGACTCGAAAAACGCT
CTTCCTGGCCAGTCTCTTTGCTCTGTGTCTGCCAGCCCCCAGCATCTCTC
CTCTTTCCTGTAAGCCCCTCTCCCTGTGCTGACTGTCTTCATAGTACTTT
AGGTATGTTGTCCCTTTACCTCTGGGAGGATAGCTTGATGACCTGTCTGC
TCAGGCCAGCCCCATCTAGAGTCTCAGTGGCCCCAGTCATGTTGAGAAAG
GTTCTTTCAAAGATAGACTCAAGATAGTAGTGTCAGAGGTCCCAAGCAAA
TGAAGGGCGGGGACAGTTGAGGGGGTGGAATAGGGACGGCAGCAGGGAAC
CAGATAGCATGCTGCTGAGAAGAAAAAAAGACATTGGTTTAGGTCAGGAA
GCAAAAAAAGGGAACTGAGTGGCTGTGAAAGGGTGGGGTTTGCTCAGACT
GTCCTTCCTCTCTGGACTGTAAGAATTAGTCTCGAGGCCACCATGGTGAG
CAAGGGCGAGGAGCTGTTCACCGGGGTGGTGCCCATCCTGGTCGAGCTGG
ACGGCGACGTAAACGGCCACAAGTTCAGCGTGTCCGGCGAGGGCGAGGGC
GATGCCACCTACGGCAAGCTGACCCTGAAGTTCATCTGCACCACCGGCAA
GCTGCCCGTGCCCTGGCCCACCCTCGTGACCACCCTGACCTACGGCGTGC
AGTGCTTCAGCCGCTACCCCGACCACATGAAGCAGCACGACTTCTTCAAG
TCCGCCATGCCCGAAGGCTACGTCCAGGAGCGCACCATCTTCTTCAAGGA
CGACGGCAACTACAAGACCCGCGCCGAGGTGAAGTTCGAGGGCGACACCC
TGGTGAACCGCATCGAGCTGAAGGGCATCGACTTCAAGGAGGACGGCAAC
ATCCTGGGGCACAAGCTGGAGTACAACTACAACAGCCACAACGTCTATAT
CATGGCCGACAAGCAGAAGAACGGCATCAAGGTGAACTTCAAGATCCGCC
ACAACATCGAGGACGGCAGCGTGCAGCTCGCCGACCACTACCAGCAGAAC
ACCCCCATCGGCGACGGCCCCGTGCTGCTGCCCGACAACCACTACCTGAG
CACCCAGTCCGCCCTGAGCAAAGACCCCAACGAGAAGCGCGATCACATGG
TCCTGCTGGAGTTCGTGACCGCCGCCGGGATCACTCTCGGCATGGACGAG
CTGTACAAGTAA
DBCA.BTKp.GFP
(SEQ ID NO: 37)
TTTCCTAGGAGAATCCCTGGGGGAATCATTGCAGTTGGAGCATAATGTAG
GGGGCCCCCTGTGTGCTTGGTAAGGTGTAGAGGTAGCTTTACACTATTCA
CCTTTATATGATATGTGTAGAATCTGTGAAAAGCAAAAGTATCTGTGCAG
CTGTTGATCTTGATATACCCAGAAGGGATAATGCAGGTGCCAGAACTCCA
CTGAGGCAGATATTGGACTTTCAGTTCCCAATTTCCTAGACGGTGAATAT
AATTTTTCAAACATTGTCCCACTGGGGTCTGTAATTATTAAATTATAAAT
GCAAAGATTTTTACAGGGCCACCTTTTGCAGCCTCCCAGCTCCCTATGGA
GCAACAGAATTCAGAAAGACGAAGCTTATTAACAAGAGTGCAGCCATCAT
ACAAACTGCAGTATGCCAAGGAATTAATAGAAATAGCACTTGCAGAATCA
GTAAACTAAACCATCTTGGGCTCTTATCTTCGCTCATAAGATAAAATACT
ATTAAGAATAATAAGTCCAGGCGCAGTGGCTCATGCCCGTAATCCCAGCA
CTTTGGGAGGCTGAGGCTGGCGGATCACTTGAGGTCAGGAGTTAGAGACC
AGCCTGGCCAACATGGTGAAATCCCGTCTCTATTAAAATACAAAAAATTA
GCCGGGCGTGGTGACAGGCGCCTGTAGTCCCAGCAACTCAGGAGGCTGAG
GCAGGAGAATCGCTTGAACTCGGGAGGCGGAGGTTGCAGTGAGCCAAGA
TCGTGACACTGCACTCCAGCCTGGAGTCTCAAAAAAAAAATAAAAAATAA
AAATAAAAAAAAAAAAGAAGAAGAATACCAGAGCAAAATTCAGAAAAC
TACACAGCCAGCCTATGATTGGGAAGAGTCTATGAGATATGATTTAAAAT
AAAATAACCAGTCAACTGTACTTCAAATGACTACTTAGCCAAAATATCCA
AGAAGTAGTGTTTTCTCTTGATTTCTCCTTGAAAGCACTACTCAATTGGC
TAGGAAGATTCTGGCCATTCCCTGTCTTTCCTACGCGTTTAAAGCTTCTG
GGTTTCCAATGCTCTCAGATTGCCCTCAGCCACTGCACATGAATCTCCAT
AGAAACTGAAAGTCACACGTGGCCCAATTGTTTCAGGGTCCTCAGAGGAG
TTCTCCCTCCTCCTTCCCCTCCCCAGATGGCTTCATGCATAACCAAGTAG
CTTTGGAGTCAAATATGTCAAGGCCTCTTTTCTAGCTTCTTGCTGAGCAC
TGTGTCACTGAAAATTCTAGTTCTTCCTTATTTTCAGTAGTTAGCAATCA
GATCTCAGCAATCAGATCATTGTGGGTGTAGAAGGGGGTATCTGATAACT
CTTTGGAGAACGCCCAAACTTTGGTTTTCGAAAGCTGTATCTTCCCTTTA
TACCTAAGTCACTTCCTGATATGGGTCTAGTCCACTGAAAGGAAACTCTA
TAGGAAATTTGCTCAAATGGAAATAGCAAATCATAGTGATAATTTAAAAT
TGCACCAATCCCCCATAACTGGATGGAAAGTTACATTAGTTCGCACCATA
AGGAGTTTACAAAGAATTCACGCGTGGATCCGAGGAAAGGAAAGGAGAAA
TAATGAAGACAAGAGAAAAGCAAGCCATGCACATGGAGGGCAATGGTAAG
AGGAGAAACCTTTGCCACAACCTCAAGGGTTGGCCGCACTGTCTGGGCTG
AGGCAGAGGCTGCTTTCTCCTGGCCCAGTCTTTTACCCATTGTTACCAC
GAGGTTGGCACAGCTTCCACACAGTCCTTTAATAAGCCAGCTCTGACCCT
GGCGACATTTGGCCTGGTACAGCTCAAGGCAGGTAGCCTACACCCTGGCC
TAGCCAGGCCTTTCCCTAGCTCACAAAGGTCAACTTCATAACGTGGAGCG
TGAGCTTTTACCATGTTCTCAGAGAATCTGCCTCTTAGTATTTCTTTTTC
TAAAGGCTTCATTGACCCATGTATCCCTATTGAGTCTTCAAGCACTATTT
GACAGAAGGGCTTGTTTTTATCTGACTGCTCCCTGCAATATTACTTTTCC
ACTAAGTTTGCCAGAGGTACCCCTTTCTAACATTTGCTACCTGTTCGGAT
GGTCACCTCATGTCACATTTTTCTCTTCCTGTTCCGCCCACCCTCAGCCC
TTGTCGGAAAGGACCCTTTCGGCCTCTCTAATCACCAGAAGGGACTGCCA
GGGCCTTGTGCTGATGACACTCCCACCCTCCGTCAGCCTCCTCCACTAGC
TGATAAATGGCACCAGCCATGCTGTCTCTCTGGTTTTTAGGGGTCCCAAG
CAAGGTACATTGCTGCTGCTGCCCAGCCCCTGCCATACCCCAGGGATCCG
GGCCCCTGAGAAAACCTCCAGGCTTCAAGTGACATACCTAGTCTGCTTTA
CCGGTTTACAGGACTCAAGAGAAAGGTGGACATTGAGAGTTAATCCCTGA
GGCCAAATCTTAAATGGAGAAAGTCAACATCCACAGAAAATGGGGAAGGG
CACAAGTATTTCTGTGGGCTTATATTCCGACATTTTTATCTGTAGGGGAA
AAATGCTTTCTTAGAAAATGACTCAGCACGGGGAAGTCTTGTCTCTACCT
CTGTCTTGTTTTGTCCTTTGGGGTCCCTTCACTATCAAGTTCAACTGTGT
GTCCCTGAGACTCCTCTGCCCCGGAGGACAGGAGACTCGAAAAACGCTCT
TCCTGGCCAGTCTCTTTGCTCTGTGTCTGCCAGCCCCCAGCATCTCTCCT
CTTTCCTGTAAGCCCCTCTCCCTGTGCTGACTGTCTTCATAGTACTTTAG
GTATGTTGTCCCTTTACCTCTGGGAGGATAGCTTGATGACCTGTCTGCTC
AGGCCAGCCCCATCTAGAGTCTCAGTGGCCCCAGTCATGTTGAGAAAGGT
TCTTTCAAAGATAGACTCAAGATAGTAGTGTCAGAGGTCCCAAGCAAATG
AAGGGCGGGGACAGTTGAGGGGGTGGAATAGGGACGGCAGCAGGGAACCA
GATAGCATGCTGCTGAGAAGAAAAAAAGACATTGGTTTAGGTCAGGAAGC
AAAAAAAGGGAACTGAGTGGCTGTGAAAGGGTGGGGTTTGCTCAGACTGT
CCTTCCTCTCTGGACTGTAAGAATTAGTCTCGAGGCCACCATGGTGAGCA
AGGGCGAGGAGCTGTTCACCGGGGTGGTGCCCATCCTGGTCGAGCTGGAC
GGCGACGTAAACGGCCACAAGTTCAGCGTGTCCGGCGAGGGCGAGGGCGA
TGCCACCTACGGCAAGCTGACCCTGAAGTTCATCTGCACCACCGGCAAGC
TGCCCGTGCCCTGGCCCACCCTCGTGACCACCCTGACCTACGGCGTGCAG
TGCTTCAGCCGCTACCCCGACCACATGAAGCAGCACGACTTCTTCAAGTC
CGCCATGCCCGAAGGCTACGTCCAGGAGCGCACCATCTTCTTCAAGGACG
ACGGCAACTACAAGACCCGCGCCGAGGTGAAGTTCGAGGGCGACACCCTG
GTGAACCGCATCGAGCTGAAGGGCATCGACTTCAAGGAGGACGGCAACAT
CCTGGGGCACAAGCTGGAGTACAACTACAACAGCCACAACGTCTATATCA
TGGCCGACAAGCAGAAGAACGGCATCAAGGTGAACTTCAAGATCCGCCAC
AACATCGAGGACGGCAGCGTGCAGCTCGCCGACCACTACCAGCAGAACAC
CCCCATCGGCGACGGCCCCGTGCTGCTGCCCGACAACCACTACCTGAGCA
CCCAGTCCGCCCTGAGCAAAGACCCCAACGAGAAGCGCGATCACATGGTC
CTGCTGGAGTTCGTGACCGCCGCCGGGATCACTCTCGGCATGGACGAGCT
GTACAAGTAA
AB.BTKp.GFP
(SEQ ID NO: 38)
TTTCCTAGGAGAATCCCTGGGGGAATCATTGCAGTTGGAGCATAATGTAG
GGGGCCCGGATCCCTGGGGTATGGCAGGGGCTGGGCAGCAGCAGCAATG
TACCTTGCTTGGGACCCCTAAAAACCAGAGAGACAGCATGGCTGGTGCCA
TTTATCAGCTAGTGGAGGAGGCTGACGGAGGGTGGGAGTGTCATCAGCAC
AAGGCCCTGGCAGTCCCTTCTGGTGATTAGAGAGGCCGAAAGGGTCCTTT
CCGACAAGGGCTGAGGGTGGGCGGAACAGGAAGAGAAAAATGTGACATG
AGGTGACCATCCGAACAGGTAGCAAATGTTAGAAAGGGGTACCTCTGGC
AAACTTAGTGGAAAAGTAATATTGCAGGGAGCAGTCAGATAAAAACAAG
CCCTTCTGTCAAATAGTGCTTGAAGACTCAATAGGGATACATGGGTCAAT
GAAGCCTTTAGAAAAAGAAATACTAAGAGGCAGATTCTCTGAGAACATG
GTAAAAGCTCACGCTCCACGTTATGAAGTTGACCTTTGTGAGCTAGGGAA
AGGCCTGGCTAGGCCAGGGTGTAGGCTACCTGCCTTGAGCTGTACCAGGC
CAAATGTCGCCAGGGTCAGAGCTGGCTTATTAAAGGACTGTGTGGAAGCT
GTGCCAACCTCGTGGTAACAATGGGTAAAAGACTGGGCCAGGAGAAAGC
AGCCTCTGCCTCAGCCCAGACAGTGCGGCCAACCCTTGAGGTTGTGGCAA
AGGTTTCTCCTCTTACCATTGCCCTCCATGTGCATGGCTTGCTTTTCTCT
TGTCTTCATTATTTCTCCTTTCCTTTCCTCGGATCCACGCGTGAATTCTT
TGTAAACTCCTTATGGTGCGAACTAATGTAACTTTCCATCCAGTTATGGG
GGATTGGTGCAATTTTAAATTATCACTATGATTTGCTATTTCCATTTGAG
CAAATTTCCTATAGAGTTTCCTTTCAGTGGACTAGACCCATATCAGGAAG
TGACTTAGGTATAAAGGGAAGATACAGCTTTCGAAAACCAAAGTTTGGGC
GTTCTCCAAAGAGTTATCAGATACCCCCTTCTACACCCACAATGATCTGA
TTGCTGAGATCTGATTGCTAACTACTGAAAATAAGGAAGAACTAGAATTT
TCAGTGACACAGTGCTCAGCAAGAAGCTAGAAAAGAGGCCTTGACATATT
TGACTCCAAAGCTACTTGGTTATGCATGAAGCCATCTGGGGAGGGGAAGG
AGGAGGGAGAACTCCTCTGAGGACCCTGAAACAATTGGGCCACGTGTGAC
TTTCAGTTTCTATGGAGATTCATGTGCAGTGGCTGAGGGCAATCTGAGAG
CATTGGAAACCCAGAAGCTTTAAACGCGTAGGGGCCCCTGAGAAAACCTC
CAGGCTTCAAGTGACATACCTAGTCTGCTTTACCGGTTTACAGGACTCAA
GAGAAAGGTGGACATTGAGAGTTAATCCCTGAGGCCAAATCTTAAATGGA
GAAAGTCAACATCCACAGAAAATGGGGAAGGGCACAAGTATTTCTGTGGG
CTTATATTCCGACATTTTTATCTGTAGGGGAAAAATGCTTTCTTAGAAAA
TGACTCAGCACGGGGAAGTCTTGTCTCTACCTCTGTCTTGTTTTGTCCTT
TGGGGTCCCTTCACTATCAAGTTCAACTGTGTGTCCCTGAGACTCCTCTG
CCCCGGAGGACAGGAGACTCGAAAAACGCTCTTCCTGGCCAGTCTCTTTG
CTCTGTGTCTGCCAGCCCCCAGCATCTCTCCTCTTTCCTGTAAGCCCCTC
TCCCTGTGCTGACTGTCTTCATAGTACTTTAGGTATGTTGTCCCTTTACC
TCTGGGAGGATAGCTTGATGACCTGTCTGCTCAGGCCAGCCCCATCTAGA
GTCTCAGTGGCCCCAGTCATGTTGAGAAAGGTTCTTTCAAAGATAGACTC
AAGATAGTAGTGTCAGAGGTCCCAAGCAAATGAAGGGCGGGGACAGTTGA
GGGGGTGGAATAGGGACGGCAGCAGGGAACCAGATAGCATGCTGCTGAGA
AGAAAAAAAGACATTGGTTTAGGTCAGGAAGCAAAAAAAGGGAACTGAGT
GGCTGTGAAAGGGTGGGGTTTGCTCAGACTGTCCTTCCTCTCTGGACTGT
AAGAATTAGTCTCGAGGCCACCATGGTGAGCAAGGGCGAGGAGCTGTTCA
CCGGGGTGGTGCCCATCCTGGTCGAGCTGGACGGCGACGTAAACGGCCAC
AAGTTCAGCGTGTCCGGCGAGGGCGAGGGCGATGCCACCTACGGCAAGCT
GACCCTGAAGTTCATCTGCACCACCGGCAAGCTGCCCGTGCCCTGGCCCA
CCCTCGTGACCACCCTGACCTACGGCGTGCAGTGCTTCAGCCGCTACCCC
GACCACATGAAGCAGCACGACTTCTTCAAGTCCGCCATGCCCGAAGGCTA
CGTCCAGGAGCGCACCATCTTCTTCAAGGACGACGGCAACTACAAGACCC
GCGCCGAGGTGAAGTTCGAGGGCGACACCCTGGTGAACCGCATCGAGCTG
AAGGGCATCGACTTCAAGGAGGACGGCAACATCCTGGGGCACAAGCTGGA
GTACAACTACAACAGCCACAACGTCTATATCATGGCCGACAAGCAGAAGA
ACGGCATCAAGGTGAACTTCAAGATCCGCCACAACATCGAGGACGGCAGC
GTGCAGCTCGCCGACCACTACCAGCAGAACACCCCCATCGGCGACGGCCC
CGTGCTGCTGCCCGACAACCACTACCTGAGCACCCAGTCCGCCCTGAGCA
AAGACCCCAACGAGAAGCGCGATCACATGGTCCTGCTGGAGTTCGTGACC
GCCGCCGGGATCACTCTCGGCATGGACGAGCTGTACAAGTAA
A.BTKp.GFP
(SEQ ID NO: 39)
TTTCCTAGGAGAATCCCTGGGGGAATCATTGCAGTTGGAGCATAATGTAG
GGGGCCCGGATCCCTGGGGTATGGCAGGGGCTGGGCAGCAGCAGCAATG
TACCTTGCTTGGGACCCCTAAAAACCAGAGAGACAGCATGGCTGGTGCCA
TTTATCAGCTAGTGGAGGAGGCTGACGGAGGGTGGGAGTGTCATCAGCAC
AAGGCCCTGGCAGTCCCTTCTGGTGATTAGAGAGGCCGAAAGGGTCCTTT
CCGACAAGGGCTGAGGGTGGGCGGAACAGGAAGAGAAAAATGTGACATG
AGGTGACCATCCGAACAGGTAGCAAATGTTAGAAAGGGGTACCTCTGGC
AAACTTAGTGGAAAAGTAATATTGCAGGGAGCAGTCAGATAAAAACAAG
CCCTTCTGTCAAATAGTGCTTGAAGACTCAATAGGGATACATGGGTCAAT
GAAGCCTTTAGAAAAAGAAATACTAAGAGGCAGATTCTCTGAGAACATG
GTAAAAGCTCACGCTCCACGTTATGAAGTTGACCTTTGTGAGCTAGGGAA
AGGCCTGGCTAGGCCAGGGTGTAGGCTACCTGCCTTGAGCTGTACCAGGC
CAAATGTCGCCAGGGTCAGAGCTGGCTTATTAAAGGACTGTGTGGAAGCT
GTGCCAACCTCGTGGTAACAATGGGTAAAAGACTGGGCCAGGAGAAAGC
AGCCTCTGCCTCAGCCCAGACAGTGCGGCCAACCCTTGAGGTTGTGGCAA
AGGTTTCTCCTCTTACCATTGCCCTCCATGTGCATGGCTTGCTTTTCTCT
TGTCTTCATTATTTCTCCTTTCCTTTCCTCGGATCCACGCGTAGGGGCCC
CTGAGAAAACCTCCAGGCTTCAAGTGACATACCTAGTCTGCTTTACCGGT
TTACAGGACTCAAGAGAAAGGTGGACATTGAGAGTTAATCCCTGAGGCCA
AATCTTAAATGGAGAAAGTCAACATCCACAGAAAATGGGGAAGGGCACAA
GTATTTCTGTGGGCTTATATTCCGACATTTTTATCTGTAGGGGAAAAATG
CTTTCTTAGAAAATGACTCAGCACGGGGAAGTCTTGTCTCTACCTCTGTC
TTGTTTTGTCCTTTGGGGTCCCTTCACTATCAAGTTCAACTGTGTGTCCC
TGAGACTCCTCTGCCCCGGAGGACAGGAGACTCGAAAAACGCTCTTCCTG
GCCAGTCTCTTTGCTCTGTGTCTGCCAGCCCCCAGCATCTCTCCTCTTTC
CTGTAAGCCCCTCTCCCTGTGCTGACTGTCTTCATAGTACTTTAGGTATG
TTGTCCCTTTACCTCTGGGAGGATAGCTTGATGACCTGTCTGCTCAGGCC
AGCCCCATCTAGAGTCTCAGTGGCCCCAGTCATGTTGAGAAAGGTTCTTT
CAAAGATAGACTCAAGATAGTAGTGTCAGAGGTCCCAAGCAAATGAAGGG
CGGGGACAGTTGAGGGGGTGGAATAGGGACGGCAGCAGGGAACCAGATAG
CATGCTGCTGAGAAGAAAAAAAGACATTGGTTTAGGTCAGGAAGCAAAAA
AAGGGAACTGAGTGGCTGTGAAAGGGTGGGGTTTGCTCAGACTGTCCTTC
CTCTCTGGACTGTAAGAATTAGTCTCGAGGCCACCATGGTGAGCAAGGGC
GAGGAGCTGTTCACCGGGGTGGTGCCCATCCTGGTCGAGCTGGACGGCGA
CGTAAACGGCCACAAGTTCAGCGTGTCCGGCGAGGGCGAGGGCGATGCCA
CCTACGGCAAGCTGACCCTGAAGTTCATCTGCACCACCGGCAAGCTGCCC
GTGCCCTGGCCCACCCTCGTGACCACCCTGACCTACGGCGTGCAGTGCTT
CAGCCGCTACCCCGACCACATGAAGCAGCACGACTTCTTCAAGTCCGCCA
TGCCCGAAGGCTACGTCCAGGAGCGCACCATCTTCTTCAAGGACGACGGC
AACTACAAGACCCGCGCCGAGGTGAAGTTCGAGGGCGACACCCTGGTGAA
CCGCATCGAGCTGAAGGGCATCGACTTCAAGGAGGACGGCAACATCCTGG
GGCACAAGCTGGAGTACAACTACAACAGCCACAACGTCTATATCATGGCC
GACAAGCAGAAGAACGGCATCAAGGTGAACTTCAAGATCCGCCACAACAT
CGAGGACGGCAGCGTGCAGCTCGCCGACCACTACCAGCAGAACACCCCCA
TCGGCGACGGCCCCGTGCTGCTGCCCGACAACCACTACCTGAGCACCCAG
TCCGCCCTGAGCAAAGACCCCAACGAGAAGCGCGATCACATGGTCCTGCT
GGAGTTCGTGACCGCCGCCGGGATCACTCTCGGCATGGACGAGCTGTACA
AGTAA
B.BTKp.GFP
(SEQ ID NO: 40)
TTTCCTAGGAGAATCCCTGGGGGAATCATTGCAGTTGGAGCATAATGTAG
GGGGCCCGGATCCACGCGTGAATTCTTTGTAAACTCCTTATGGTGCGAAC
TAATGTAACTTTCCATCCAGTTATGGGGGATTGGTGCAATTTTAAATTAT
CACTATGATTTGCTATTTCCATTTGAGCAAATTTCCTATAGAGTTTCCTT
TCAGTGGACTAGACCCATATCAGGAAGTGACTTAGGTATAAAGGGAAGAT
ACAGCTTTCGAAAACCAAAGTTTGGGCGTTCTCCAAAGAGTTATCAGATA
CCCCCTTCTACACCCACAATGATCTGATTGCTGAGATCTGATTGCTAACT
ACTGAAAATAAGGAAGAACTAGAATTTTCAGTGACACAGTGCTCAGCAAG
AAGCTAGAAAAGAGGCCTTGACATATTTGACTCCAAAGCTACTTGGTTAT
GCATGAAGCCATCTGGGGAGGGGAAGGAGGAGGGAGAACTCCTCTGAGGA
CCCTGAAACAATTGGGCCACGTGTGACTTTCAGTTTCTATGGAGATTCAT
GTGCAGTGGCTGAGGGCAATCTGAGAGCATTGGAAACCCAGAAGCTTTAA
ACGCGTAGGGGCCCCTGAGAAAACCTCCAGGCTTCAAGTGACATACCTAG
TCTGCTTTACCGGTTTACAGGACTCAAGAGAAAGGTGGACATTGAGAGTT
AATCCCTGAGGCCAAATCTTAAATGGAGAAAGTCAACATCCACAGAAAAT
GGGGAAGGGCACAAGTATTTCTGTGGGCTTATATTCCGACATTTTTATCT
GTAGGGGAAAAATGCTTTCTTAGAAAATGACTCAGCACGGGGAAGTCTTG
TCTCTACCTCTGTCTTGTTTTGTCCTTTGGGGTCCCTTCACTATCAAGTT
CAACTGTGTGTCCCTGAGACTCCTCTGCCCCGGAGGACAGGAGACTCGAA
AAACGCTCTTCCTGGCCAGTCTCTTTGCTCTGTGTCTGCCAGCCCCCAGC
ATCTCTCCTCTTTCCTGTAAGCCCCTCTCCCTGTGCTGACTGTCTTCATA
GTACTTTAGGTATGTTGTCCCTTTACCTCTGGGAGGATAGCTTGATGACC
TGTCTGCTCAGGCCAGCCCCATCTAGAGTCTCAGTGGCCCCAGTCATGTT
GAGAAAGGTTCTTTCAAAGATAGACTCAAGATAGTAGTGTCAGAGGTCCC
AAGCAAATGAAGGGCGGGGACAGTTGAGGGGGTGGAATAGGGACGGCAGC
AGGGAACCAGATAGCATGCTGCTGAGAAGAAAAAAAGACATTGGTTTAGG
TCAGGAAGCAAAAAAAGGGAACTGAGTGGCTGTGAAAGGGTGGGGTTTGC
TCAGACTGTCCTTCCTCTCTGGACTGTAAGAATTAGTCTCGAGGCCACCA
TGGTGAGCAAGGGCGAGGAGCTGTTCACCGGGGTGGTGCCCATCCTGGTC
GAGCTGGACGGCGACGTAAACGGCCACAAGTTCAGCGTGTCCGGCGAGGG
CGAGGGCGATGCCACCTACGGCAAGCTGACCCTGAAGTTCATCTGCACCA
CCGGCAAGCTGCCCGTGCCCTGGCCCACCCTCGTGACCACCCTGACCTAC
GGCGTGCAGTGCTTCAGCCGCTACCCCGACCACATGAAGCAGCACGACTT
CTTCAAGTCCGCCATGCCCGAAGGCTACGTCCAGGAGCGCACCATCTTCT
TCAAGGACGACGGCAACTACAAGACCCGCGCCGAGGTGAAGTTCGAGGGC
GACACCCTGGTGAACCGCATCGAGCTGAAGGGCATCGACTTCAAGGAGGA
CGGCAACATCCTGGGGCACAAGCTGGAGTACAACTACAACAGCCACAACG
TCTATATCATGGCCGACAAGCAGAAGAACGGCATCAAGGTGAACTTCAAG
ATCCGCCACAACATCGAGGACGGCAGCGTGCAGCTCGCCGACCACTACCA
GCAGAACACCCCCATCGGCGACGGCCCCGTGCTGCTGCCCGACAACCACT
ACCTGAGCACCCAGTCCGCCCTGAGCAAAGACCCCAACGAGAAGCGCGAT
CACATGGTCCTGCTGGAGTTCGTGACCGCCGCCGGGATCACTCTCGGCAT
GGACGAGCTGTACAAGTAA
0.7UCOE.AB.BTKp.coBTK
(SEQ ID NO: 41)
TTTCCTAGGAGAATCCCTGGGGGAATCATTGCAGTTGGAGCATAATGTAG
GGGGCCGCGTGTGGCATCTGAAGCACCACCAGCGAGCGAGAGCTAGAGA
GAAGGAAAGCCACCGACTTCACCGCCTCCGAGCTGCTCCGGGTCGCGGGT
CTGCAGCGTCTCCGGCCCTCCGCGCCTACAGCTCAAGCCACATCCGAAGG
GGGAGGGAGCCGGGAGCTGCGCGCGGGGCCGCCGGGGGGAGGGGTGGC
ACCGCCCACGCCGGGCGGCCACGAAGGGCGGGGCAGCGGGCGCGCGCCC
GGCGGGGGGAGGGGCCGCGCGCCGCGCCCGCTGGGAATTGGGGCCCTAG
GGGGAGGGCGGAGGCGCCGACGACCGCGGCACTTACCGTTCGCGGCGTG
GCGCCCGGTGGTCCCCAAGGGGAGGGAAGGGGGAGGCGGGGCGAGGAC
AGTGACCGGAGTCTCCTCAGCGGTGGCTTTTCTGCTTGGCAGCCTCAGCG
GCTGGCGCCAAAACCGGACTCCGCCCACTTCCTCGCCCCTGCGGTGCGAG
GGTGTGGAATCCTCCAGACGCTGGGGGAGGGGGAGTTGGGAGCTTAAAA
ACTAGTACCCCTTTGGGACCACTTTCAGCAGCGAACTCTCCTGTACACCA
GGGGTCAGTTCCACAGACGCGGGCCAGGGGTGGGTCATTGCGGCGTGAA
CAATAATTTGACTAGAAGTTGATTCGGGTGTTTGCGGCCCTGGGGTATGG
CAGGGGCTGGGCAGCAGCAGCAATGTACCTTGCTTGGGACCCCTAAAAAC
CAGAGAGACAGCATGGCTGGTGCCATTTATCAGCTAGTGGAGGAGGCTGA
CGGAGGGTGGGAGTGTCATCAGCACAAGGCCCTGGCAGTCCCTTCTGGTG
ATTAGAGAGGCCGAAAGGGTCCTTTCCGACAAGGGCTGAGGGTGGGCGG
AACAGGAAGAGAAAAATGTGACATGAGGTGACCATCCGAACAGGTAGCA
AATGTTAGAAAGGGGTACCTCTGGCAAACTTAGTGGAAAAGTAATATTGC
AGGGAGCAGTCAGATAAAAACAAGCCCTTCTGTCAAATAGTGCTTGAAGA
CTCAATAGGGATACATGGGTCAATGAAGCCTTTAGAAAAAGAAATACTAA
GAGGCAGATTCTCTGAGAACATGGTAAAAGCTCACGCTCCACGTTATGAA
GTTGACCTTTGTGAGCTAGGGAAAGGCCTGGCTAGGCCAGGGTGTAGGCT
ACCTGCCTTGAGCTGTACCAGGCCAAATGTCGCCAGGGTCAGAGCTGGCT
TATTAAAGGACTGTGTGGAAGCTGTGCCAACCTCGTGGTAACAATGGGTA
AAAGACTGGGCCAGGAGAAAGCAGCCTCTGCCTCAGCCCAGACAGTGCG
GCCAACCCTTGAGGTTGTGGCAAAGGTTTCTCCTCTTACCATTGCCCTCC
ATGTGCATGGCTTGCTTTTCTCTTGTCTTCATTATTTCTCCTTTCCTTTC
CTCGGATCCACGCGTGAATTCTTTGTAAACTCCTTATGGTGCGAACTAAT
GTAACTTTCCATCCAGTTATGGGGGATTGGTGCAATTTTAAATTATCACT
ATGATTTGCTATTTCCATTTGAGCAAATTTCCTATAGAGTTTCCTTTCAG
TGGACTAGACCCATATCAGGAAGTGACTTAGGTATAAAGGGAAGATACAG
CTTTCGAAAACCAAAGTTTGGGCGTTCTCCAAAGAGTTATCAGATACCCC
CTTCTACACCCACAATGATCTGATTGCTGAGATCTGATTGCTAACTACTG
AAAATAAGGAAGAACTAGAATTTTCAGTGACACAGTGCTCAGCAAGAAGC
TAGAAAAGAGGCCTTGACATATTTGACTCCAAAGCTACTTGGTTATGCAT
GAAGCCATCTGGGGAGGGGAAGGAGGAGGGAGAACTCCTCTGAGGACCCT
GAAACAATTGGGCCACGTGTGACTTTCAGTTTCTATGGAGATTCATGTGC
AGTGGCTGAGGGCAATCTGAGAGCATTGGAAACCCAGAAGCTTTAAGGCC
CCTGAGAAAACCTCCAGGCTTCAAGTGACATACCTAGTCTGCTTTACCGG
TTTACAGGACTCAAGAGAAAGGTGGACATTGAGAGTTAATCCCTGAGGCC
AAATCTTAAATGGAGAAAGTCAACATCCACAGAAAATGGGGAAGGGCACA
AGTATTTCTGTGGGCTTATATTCCGACATTTTTATCTGTAGGGGAAAAAT
GCTTTCTTAGAAAATGACTCAGCACGGGGAAGTCTTGTCTCTACCTCTGT
CTTGTTTTGTCCTTTGGGGTCCCTTCACTATCAAGTTCAACTGTGTGTCC
CTGAGACTCCTCTGCCCCGGAGGACAGGAGACTCGAAAAACGCTCTTCCT
GGCCAGTCTCTTTGCTCTGTGTCTGCCAGCCCCCAGCATCTCTCCTCTTT
CCTGTAAGCCCCTCTCCCTGTGCTGACTGTCTTCATAGTACTTTAGGTAT
GTTGTCCCTTTACCTCTGGGAGGATAGCTTGATGACCTGTCTGCTCAGGC
CAGCCCCATCTAGAGTCTCAGTGGCCCCAGTCATGTTGAGAAAGGTTCTT
TCAAAGATAGACTCAAGATAGTAGTGTCAGAGGTCCCAAGCAAATGAAGG
GCGGGGACAGTTGAGGGGGTGGAATAGGGACGGCAGCAGGGAACCAGATA
GCATGCTGCTGAGAAGAAAAAAAGACATTGGTTTAGGTCAGGAAGCAAAA
AAAGGGAACTGAGTGGCTGTGAAAGGGTGGGGTTTGCTCAGACTGTCCTT
CCTCTCTGGACTGTAAGAATTAGTCTCGAGAAAGAAGCCACCATGGCCGC
TGTGATCCTGGAGAGCATTTTCCTGAAGAGGTCCCAGCAGAAAAAGAAAA
CCTCTCCCCTGAACTTTAAGAAAAGACTGTTCCTGCTGACAGTGCACAAG
CTGTCTTACTATGAGTACGACTTTGAGCGGGGCCGCCGAGGATCAAAAAA
GGGGAGCATCGATGTGGAGAAGATTACATGCGTGGAGACCGTGGTCCCTG
AAAAGAATCCACCCCCTGAGAGGCAGATCCCAAGACGGGGCGAGGAGTCC
TCTGAGATGGAGCAGATTAGTATCATTGAGCGCTTCCCCTATCCTTTTCA
GGTGGTGTACGACGAGGGACCACTGTATGTGTTCTCACCCACAGAGGAGC
TGAGAAAGAGGTGGATTCACCAGCTGAAGAACGTGATTAGATACAATAGC
GATCTGGTGCAGAAGTATCACCCTTGTTTTTGGATCGACGGGCAGTACCT
GTGCTGTTCCCAGACAGCTAAGAACGCTATGGGATGCCAGATTCTGGAAA
ATCGGAACGGATCTCTGAAACCAGGGAGTTCACACCGCAAGACCAAAAAG
CCCCTGCCTCCAACACCCGAGGAGGATCAGATCCTGAAAAAGCCTCTGCC
ACCCGAGCCTGCTGCAGCCCCAGTCAGCACTTCCGAACTGAAAAAGGTGG
TGGCTCTGTATGACTACATGCCCATGAATGCTAACGATCTGCAGCTGAGA
AAGGGCGACGAGTATTTCATTCTGGAAGAGTCTAATCTGCCTTGGTGGAG
GGCCAGAGATAAGAACGGACAGGAGGGGTACATCCCATCTAATTATGTGA
CCGAGGCTGAGGACTCTATTGAGATGTACGAGTGGTATAGCAAGCACATG
ACACGGTCCCAGGCTGAGCAGCTGCTGAAGCAGGAGGGCAAAGAGGGAGG
GTTTATCGTGCGCGATTCTAGTAAGGCCGGCAAATACACTGTGTCAGTGT
TCGCTAAGAGCACCGGAGACCCCCAGGGCGTGATCAGACACTATGTGGTG
TGTTCCACACCTCAGTCTCAGTACTATCTGGCTGAGAAGCACCTGTTTAG
TACAATCCCAGAGCTGATTAACTACCACCAGCACAATTCTGCCGGCCTGA
TCAGCAGGCTGAAGTATCCCGTCTCCCAGCAGAACAAAAATGCTCCTTCT
ACCGCTGGACTGGGGTACGGCAGTTGGGAGATTGATCCAAAGGACCTGAC
ATTCCTGAAGGAGCTGGGAACTGGGCAGTTTGGCGTGGTGAAGTATGGAA
AATGGAGAGGGCAGTACGATGTGGCCATCAAGATGATCAAGGAGGGCTCA
ATGAGCGAGGACGAGTTCATCGAGGAGGCTAAGGTCATGATGAACCTGTC
CCACGAGAAACTGGTGCAGCTGTATGGAGTGTGCACCAAGCAGCGGCCCA
TTTTTATCATTACAGAGTACATGGCTAATGGGTGTCTGCTGAACTATCTG
CGCGAGATGAGACACAGATTCCAGACACAGCAGCTGCTGGAAATGTGCAA
GGATGTGTGTGAGGCTATGGAGTACCTGGAGTCTAAGCAGTTTCTGCACC
GGGACCTGGCTGCTCGCAATTGCCTGGTGAACGATCAGGGCGTGGTGAAG
GTGAGTGACTTCGGACTGTCAAGGTATGTGCTGGATGACGAGTACACCAG
CTCCGTGGGCTCTAAGTTTCCTGTGAGATGGTCTCCACCCGAGGTGCTGA
TGTATAGCAAGTTCTCCTCTAAGAGCGATATCTGGGCCTTTGGCGTGCTG
ATGTGGGAAATCTACAGCCTGGGCAAGATGCCTTACGAGCGGTTCACAAA
TTCCGAGACAGCTGAGCACATCGCCCAGGGCCTGCGCCTGTACCGGCCAC
ATCTGGCCTCTGAGAAGGTGTACACCATCATGTACAGCTGTTGGCACGAG
AAGGCCGACGAGAGACCCACATTCAAGATCCTGCTGTCCAACATTCTAGA
TGTGATGGACGAGGAGAGCTGA
AB.BTKp.coBTK
(SEQ ID NO: 42)
TTTCCTAGGAGAATCCCTGGGGGAATCATTGCAGTTGGAGCATAATGTAG
GGGGCCCTGGGGTATGGCAGGGGCTGGGCAGCAGCAGCAATGTACCTTG
CTTGGGACCCCTAAAAACCAGAGAGACAGCATGGCTGGTGCCATTTATCA
GCTAGTGGAGGAGGCTGACGGAGGGTGGGAGTGTCATCAGCACAAGGCC
CTGGCAGTCCCTTCTGGTGATTAGAGAGGCCGAAAGGGTCCTTTCCGACA
AGGGCTGAGGGTGGGCGGAACAGGAAGAGAAAAATGTGACATGAGGTGA
CCATCCGAACAGGTAGCAAATGTTAGAAAGGGGTACCTCTGGCAAACTTA
GTGGAAAAGTAATATTGCAGGGAGCAGTCAGATAAAAACAAGCCCTTCT
GTCAAATAGTGCTTGAAGACTCAATAGGGATACATGGGTCAATGAAGCCT
TTAGAAAAAGAAATACTAAGAGGCAGATTCTCTGAGAACATGGTAAAAG
CTCACGCTCCACGTTATGAAGTTGACCTTTGTGAGCTAGGGAAAGGCCTG
GCTAGGCCAGGGTGTAGGCTACCTGCCTTGAGCTGTACCAGGCCAAATGT
CGCCAGGGTCAGAGCTGGCTTATTAAAGGACTGTGTGGAAGCTGTGCCAA
CCTCGTGGTAACAATGGGTAAAAGACTGGGCCAGGAGAAAGCAGCCTCT
GCCTCAGCCCAGACAGTGCGGCCAACCCTTGAGGTTGTGGCAAAGGTTTC
TCCTCTTACCATTGCCCTCCATGTGCATGGCTTGCTTTTCTCTTGTCTTC
ATTATTTCTCCTTTCCTTTCCTCGGATCCACGCGTGAATTCTTTGTAAAC
TCCTTATGGTGCGAACTAATGTAACTTTCCATCCAGTTATGGGGGATTGG
TGCAATTTTAAATTATCACTATGATTTGCTATTTCCATTTGAGCAAATTT
CCTATAGAGTTTCCTTTCAGTGGACTAGACCCATATCAGGAAGTGACTTA
GGTATAAAGGGAAGATACAGCTTTCGAAAACCAAAGTTTGGGCGTTCTCC
AAAGAGTTATCAGATACCCCCTTCTACACCCACAATGATCTGATTGCTGA
GATCTGATTGCTAACTACTGAAAATAAGGAAGAACTAGAATTTTCAGTGA
CACAGTGCTCAGCAAGAAGCTAGAAAAGAGGCCTTGACATATTTGACTCC
AAAGCTACTTGGTTATGCATGAAGCCATCTGGGGAGGGGAAGGAGGAGGG
AGAACTCCTCTGAGGACCCTGAAACAATTGGGCCACGTGTGACTTTCAGT
TTCTATGGAGATTCATGTGCAGTGGCTGAGGGCAATCTGAGAGCATTGGA
AACCCAGAAGCTTTAAGGCCCCTGAGAAAACCTCCAGGCTTCAAGTGACA
TACCTAGTCTGCTTTACCGGTTTACAGGACTCAAGAGAAAGGTGGACATT
GAGAGTTAATCCCTGAGGCCAAATCTTAAATGGAGAAAGTCAACATCCAC
AGAAAATGGGGAAGGGCACAAGTATTTCTGTGGGCTTATATTCCGACATT
TTTATCTGTAGGGGAAAAATGCTTTCTTAGAAAATGACTCAGCACGGGGA
AGTCTTGTCTCTACCTCTGTCTTGTTTTGTCCTTTGGGGTCCCTTCACTA
TCAAGTTCAACTGTGTGTCCCTGAGACTCCTCTGCCCCGGAGGACAGGAG
ACTCGAAAAACGCTCTTCCTGGCCAGTCTCTTTGCTCTGTGTCTGCCAGC
CCCCAGCATCTCTCCTCTTTCCTGTAAGCCCCTCTCCCTGTGCTGACTGT
CTTCATAGTACTTTAGGTATGTTGTCCCTTTACCTCTGGGAGGATAGCTT
GATGACCTGTCTGCTCAGGCCAGCCCCATCTAGAGTCTCAGTGGCCCCAG
TCATGTTGAGAAAGGTTCTTTCAAAGATAGACTCAAGATAGTAGTGTCAG
AGGTCCCAAGCAAATGAAGGGCGGGGACAGTTGAGGGGGTGGAATAGGGA
CGGCAGCAGGGAACCAGATAGCATGCTGCTGAGAAGAAAAAAAGACATTG
GTTTAGGTCAGGAAGCAAAAAAAGGGAACTGAGTGGCTGTGAAAGGGTGG
GGTTTGCTCAGACTGTCCTTCCTCTCTGGACTGTAAGAATTAGTCTCGAG
AAAGAAGCCACCATGGCCGCTGTGATCCTGGAGAGCATTTTCCTGAAGAG
GTCCCAGCAGAAAAAGAAAACCTCTCCCCTGAACTTTAAGAAAAGACTGT
TCCTGCTGACAGTGCACAAGCTGTCTTACTATGAGTACGACTTTGAGCGG
GGCCGCCGAGGATCAAAAAAGGGGAGCATCGATGTGGAGAAGATTACATG
CGTGGAGACCGTGGTCCCTGAAAAGAATCCACCCCCTGAGAGGCAGATCC
CAAGACGGGGCGAGGAGTCCTCTGAGATGGAGCAGATTAGTATCATTGAG
CGCTTCCCCTATCCTTTTCAGGTGGTGTACGACGAGGGACCACTGTATGT
GTTCTCACCCACAGAGGAGCTGAGAAAGAGGTGGATTCACCAGCTGAAGA
ACGTGATTAGATACAATAGCGATCTGGTGCAGAAGTATCACCCTTGTTTT
TGGATCGACGGGCAGTACCTGTGCTGTTCCCAGACAGCTAAGAACGCTAT
GGGATGCCAGATTCTGGAAAATCGGAACGGATCTCTGAAACCAGGGAGTT
CACACCGCAAGACCAAAAAGCCCCTGCCTCCAACACCCGAGGAGGATCAG
ATCCTGAAAAAGCCTCTGCCACCCGAGCCTGCTGCAGCCCCAGTCAGCAC
TTCCGAACTGAAAAAGGTGGTGGCTCTGTATGACTACATGCCCATGAATG
CTAACGATCTGCAGCTGAGAAAGGGCGACGAGTATTTCATTCTGGAAGAG
TCTAATCTGCCTTGGTGGAGGGCCAGAGATAAGAACGGACAGGAGGGGTA
CATCCCATCTAATTATGTGACCGAGGCTGAGGACTCTATTGAGATGTACG
AGTGGTATAGCAAGCACATGACACGGTCCCAGGCTGAGCAGCTGCTGAAG
CAGGAGGGCAAAGAGGGAGGGTTTATCGTGCGCGATTCTAGTAAGGCCGG
CAAATACACTGTGTCAGTGTTCGCTAAGAGCACCGGAGACCCCCAGGGCG
TGATCAGACACTATGTGGTGTGTTCCACACCTCAGTCTCAGTACTATCTG
GCTGAGAAGCACCTGTTTAGTACAATCCCAGAGCTGATTAACTACCACCA
GCACAATTCTGCCGGCCTGATCAGCAGGCTGAAGTATCCCGTCTCCCAGC
AGAACAAAAATGCTCCTTCTACCGCTGGACTGGGGTACGGCAGTTGGGAG
ATTGATCCAAAGGACCTGACATTCCTGAAGGAGCTGGGAACTGGGCAGTT
TGGCGTGGTGAAGTATGGAAAATGGAGAGGGCAGTACGATGTGGCCATCA
AGATGATCAAGGAGGGCTCAATGAGCGAGGACGAGTTCATCGAGGAGGCT
AAGGTCATGATGAACCTGTCCCACGAGAAACTGGTGCAGCTGTATGGAGT
GTGCACCAAGCAGCGGCCCATTTTTATCATTACAGAGTACATGGCTAATG
GGTGTCTGCTGAACTATCTGCGCGAGATGAGACACAGATTCCAGACACAG
CAGCTGCTGGAAATGTGCAAGGATGTGTGTGAGGCTATGGAGTACCTGGA
GTCTAAGCAGTTTCTGCACCGGGACCTGGCTGCTCGCAATTGCCTGGTGA
ACGATCAGGGCGTGGTGAAGGTGAGTGACTTCGGACTGTCAAGGTATGTG
CTGGATGACGAGTACACCAGCTCCGTGGGCTCTAAGTTTCCTGTGAGATG
GTCTCCACCCGAGGTGCTGATGTATAGCAAGTTCTCCTCTAAGAGCGATA
TCTGGGCCTTTGGCGTGCTGATGTGGGAAATCTACAGCCTGGGCAAGATG
CCTTACGAGCGGTTCACAAATTCCGAGACAGCTGAGCACATCGCCCAGGG
CCTGCGCCTGTACCGGCCACATCTGGCCTCTGAGAAGGTGTACACCATCA
TGTACAGCTGTTGGCACGAGAAGGCCGACGAGAGACCCACATTCAAGATC
CTGCTGTCCAACATTCTAGATGTGATGGACGAGGAGAGCTGA
BTKe.AB.BTKp.coBTK
(SEQ ID NO: 43)
TTTCCTAGGAGAATCCCTGGGGGAATCATTGCAGTTGGAGCATAATGTAG
GGGGCCCTCCATCACCTACTAGATATATCAGTGCAGTGAAAACTTCGCTA
AACTAACGCTATACCTATATCATGAAGTGTGTGGACTAGAGACAAGTGCA
TATCCTTACGGCAATTAACTGGGAAACGTCAAATAGTAACTACCACTCAC
CTTTTTCCGGAAAATCGGCTTAGTTTGCCCACCATAGCCACTCTGCTTCC
TGTCATAACGCCGCTTTCCTGGGAAAACGAATTGGTATTTGTTATAAAAT
ACTGAAGATCAGCAAGTAAGTCTTACAGGTTTTATCTTAATTTCGCAGCA
GAAATATTAACGCTCAAGCCAGGCGTGGAGGGAGAGAGACCCGGACTCGT
ATGTTATTCTACAACACAAATGTCACATTAACACCAAATTATGCGGAATC
CATCTTACCCTGGGCGTACAGAGAATCCTTGCCCTTCTTGTACTGTGTCA
CTTTATGGGGTTGGTGCTTGCCACACTTCTTACAGAAAGTCCGGCGGGTT
TTAGGGACGTTAACCTAGTAAAGAAACAGTTCAGAACGTGCAATGTTATT
TGACCACAATGGCACAACGCCCTACCTTACCCAGCTAAAGCTGAGGCACT
CCAGGAGGACTCCTCATTACTTGCTACCTCTGACTACAGGGTGGGCCAGC
CCCATGTGCTTCAAGCAGAGCTTCCTCCCTCCGTCGAGCCCCAAAGAGGG
AAGAGACCTCATTAACTCCACCCCCGGCTAACTCTACCTCTTTGAACCCA
TCACTTCAATTCCTGGCCCCGTAGCCCGGTCCCTTTAGGGTTGATCCCGG
CAAGATTGGGTTGCTCTGATATATCGAGTCCACACAGGAGCCTGGACCCA
TCCCGGCATAGCACGGGCGACGAAGGGGGGGAAAGATTAAGCTGGATGTT
ACTCGGCCCCCACCAGCAAGTCCTACCATGCTTGCGTGAGCGCTATCGGC
GCGGAAAGAAAGAAACCGCGAGGCAAACGGAAGTATATAGGAGGTTCCCG
ATCGCACTTCCTCATGGGAGTCGGTAGGAGCAATCATAGAGTGTAAGGCT
CAGCGCAGCGCCCTCGGGCGGCTGAGAGGACTCAGTTCGGAGCCGCGGGC
GGGAGCTTAAGGAAGGACTCCGCCTAAAGGGTGGTCCACTCACCCCGACT
TCCTCCCGCCCCGCAGCTTTCAACGTTTCGTCACTTTATCTCTTTTGGTG
GACTCTGCTACGTAGTGGCGTTCAGTGAAGGGAGCAGTGTTTTTCCCAGA
TCCTCTGGCCTCCCCGTCCCCGAGGGAAGCCAGGACTAGGGTCGAATGAA
GGGGTCCTCCACCTCCACGTTCCATTCCTGTTCCACCTCAAGGTCACTGG
GAACACCTTTCGCAGCAAACTGCTAATTCAATGAAGACCTGGAGGGAGCC
AATTGTTCCAGTTCATCTATCACATGGCCAGTTGGTCCATTCAACAAATG
GTTATTGGATGCCCATTATGTGGCAGGCACTGTTCCGGGGGAGAGGTACA
GTAATCTAATAGGCTTATAAATGTGCAATTATGAACTAAGTACTTTGAAG
AAAAGGAACAATGATTGGCATTAAAGCAGCACCCTTCTGTTGAGGGAGTA
AGTCAGCAGCTCTAGGTTCTGAAAAGTGACAATGAAATTGTTTGGCTCCT
GTCTGGGGTATGGCAGGGGCTGGGCAGCAGCAGCAATGTACCTTGCTTGG
GACCCCTAAAAACCAGAGAGACAGCATGGCTGGTGCCATTTATCAGCTAG
TGGAGGAGGCTGACGGAGGGTGGGAGTGTCATCAGCACAAGGCCCTGGCA
GTCCCTTCTGGTGATTAGAGAGGCCGAAAGGGTCCTTTCCGACAAGGGCT
GAGGGTGGGCGGAACAGGAAGAGAAAAATGTGACATGAGGTGACCATCCG
AACAGGTAGCAAATGTTAGAAAGGGGTACCTCTGGCAAACTTAGTGGAAA
AGTAATATTGCAGGGAGCAGTCAGATAAAAACAAGCCCTTCTGTCAAATA
GTGCTTGAAGACTCAATAGGGATACATGGGTCAATGAAGCCTTTAGAAAA
AGAAATACTAAGAGGCAGATTCTCTGAGAACATGGTAAAAGCTCACGCTC
CACGTTATGAAGTTGACCTTTGTGAGCTAGGGAAAGGCCTGGCTAGGCCA
GGGTGTAGGCTACCTGCCTTGAGCTGTACCAGGCCAAATGTCGCCAGGGT
CAGAGCTGGCTTATTAAAGGACTGTGTGGAAGCTGTGCCAACCTCGTGGT
AACAATGGGTAAAAGACTGGGCCAGGAGAAAGCAGCCTCTGCCTCAGCCC
AGACAGTGCGGCCAACCCTTGAGGTTGTGGCAAAGGTTTCTCCTCTTACC
ATTGCCCTCCATGTGCATGGCTTGCTTTTCTCTTGTCTTCATTATTTCTC
CTTTCCTTTCCTCGGATCCACGCGTGAATTCTTTGTAAACTCCTTATGGT
GCGAACTAATGTAACTTTCCATCCAGTTATGGGGGATTGGTGCAATTTTA
AATTATCACTATGATTTGCTATTTCCATTTGAGCAAATTTCCTATAGAGT
TTCCTTTCAGTGGACTAGACCCATATCAGGAAGTGACTTAGGTATAAAGG
GAAGATACAGCTTTCGAAAACCAAAGTTTGGGCGTTCTCCAAAGAGTTAT
CAGATACCCCCTTCTACACCCACAATGATCTGATTGCTGAGATCTGATTG
CTAACTACTGAAAATAAGGAAGAACTAGAATTTTCAGTGACACAGTGCTC
AGCAAGAAGCTAGAAAAGAGGCCTTGACATATTTGACTCCAAAGCTACTT
GGTTATGCATGAAGCCATCTGGGGAGGGGAAGGAGGAGGGAGAACTCCTC
TGAGGACCCTGAAACAATTGGGCCACGTGTGACTTTCAGTTTCTATGGAG
ATTCATGTGCAGTGGCTGAGGGCAATCTGAGAGCATTGGAAACCCAGAAG
CTTTAAGGCCCCTGAGAAAACCTCCAGGCTTCAAGTGACATACCTAGTCT
GCTTTACCGGTTTACAGGACTCAAGAGAAAGGTGGACATTGAGAGTTAAT
CCCTGAGGCCAAATCTTAAATGGAGAAAGTCAACATCCACAGAAAATGGG
GAAGGGCACAAGTATTTCTGTGGGCTTATATTCCGACATTTTTATCTGTA
GGGGAAAAATGCTTTCTTAGAAAATGACTCAGCACGGGGAAGTCTTGTCT
CTACCTCTGTCTTGTTTTGTCCTTTGGGGTCCCTTCACTATCAAGTTCAA
CTGTGTGTCCCTGAGACTCCTCTGCCCCGGAGGACAGGAGACTCGAAAAA
CGCTCTTCCTGGCCAGTCTCTTTGCTCTGTGTCTGCCAGCCCCCAGCATC
TCTCCTCTTTCCTGTAAGCCCCTCTCCCTGTGCTGACTGTCTTCATAGTA
CTTTAGGTATGTTGTCCCTTTACCTCTGGGAGGATAGCTTGATGACCTGT
CTGCTCAGGCCAGCCCCATCTAGAGTCTCAGTGGCCCCAGTCATGTTGAG
AAAGGTTCTTTCAAAGATAGACTCAAGATAGTAGTGTCAGAGGTCCCAAG
CAAATGAAGGGCGGGGACAGTTGAGGGGGTGGAATAGGGACGGCAGCAGG
GAACCAGATAGCATGCTGCTGAGAAGAAAAAAAGACATTGGTTTAGGTCA
GGAAGCAAAAAAAGGGAACTGAGTGGCTGTGAAAGGGTGGGGTTTGCTCA
GACTGTCCTTCCTCTCTGGACTGTAAGAATTAGTCTCGAGAAAGAAGCCA
CCATGGCCGCTGTGATCCTGGAGAGCATTTTCCTGAAGAGGTCCCAGCAG
AAAAAGAAAACCTCTCCCCTGAACTTTAAGAAAAGACTGTTCCTGCTGAC
AGTGCACAAGCTGTCTTACTATGAGTACGACTTTGAGCGGGGCCGCCGAG
GATCAAAAAAGGGGAGCATCGATGTGGAGAAGATTACATGCGTGGAGACC
GTGGTCCCTGAAAAGAATCCACCCCCTGAGAGGCAGATCCCAAGACGGGG
CGAGGAGTCCTCTGAGATGGAGCAGATTAGTATCATTGAGCGCTTCCCCT
ATCCTTTTCAGGTGGTGTACGACGAGGGACCACTGTATGTGTTCTCACCC
ACAGAGGAGCTGAGAAAGAGGTGGATTCACCAGCTGAAGAACGTGATTAG
ATACAATAGCGATCTGGTGCAGAAGTATCACCCTTGTTTTTGGATCGACG
GGCAGTACCTGTGCTGTTCCCAGACAGCTAAGAACGCTATGGGATGCCAG
ATTCTGGAAAATCGGAACGGATCTCTGAAACCAGGGAGTTCACACCGCAA
GACCAAAAAGCCCCTGCCTCCAACACCCGAGGAGGATCAGATCCTGAAAA
AGCCTCTGCCACCCGAGCCTGCTGCAGCCCCAGTCAGCACTTCCGAACTG
AAAAAGGTGGTGGCTCTGTATGACTACATGCCCATGAATGCTAACGATCT
GCAGCTGAGAAAGGGCGACGAGTATTTCATTCTGGAAGAGTCTAATCTGC
CTTGGTGGAGGGCCAGAGATAAGAACGGACAGGAGGGGTACATCCCATCT
AATTATGTGACCGAGGCTGAGGACTCTATTGAGATGTACGAGTGGTATAG
CAAGCACATGACACGGTCCCAGGCTGAGCAGCTGCTGAAGCAGGAGGGCA
AAGAGGGAGGGTTTATCGTGCGCGATTCTAGTAAGGCCGGCAAATACACT
GTGTCAGTGTTCGCTAAGAGCACCGGAGACCCCCAGGGCGTGATCAGACA
CTATGTGGTGTGTTCCACACCTCAGTCTCAGTACTATCTGGCTGAGAAGC
ACCTGTTTAGTACAATCCCAGAGCTGATTAACTACCACCAGCACAATTCT
GCCGGCCTGATCAGCAGGCTGAAGTATCCCGTCTCCCAGCAGAACAAAAA
TGCTCCTTCTACCGCTGGACTGGGGTACGGCAGTTGGGAGATTGATCCAA
AGGACCTGACATTCCTGAAGGAGCTGGGAACTGGGCAGTTTGGCGTGGTG
AAGTATGGAAAATGGAGAGGGCAGTACGATGTGGCCATCAAGATGATCAA
GGAGGGCTCAATGAGCGAGGACGAGTTCATCGAGGAGGCTAAGGTCATGA
TGAACCTGTCCCACGAGAAACTGGTGCAGCTGTATGGAGTGTGCACCAAG
CAGCGGCCCATTTTTATCATTACAGAGTACATGGCTAATGGGTGTCTGCT
GAACTATCTGCGCGAGATGAGACACAGATTCCAGACACAGCAGCTGCTGG
AAATGTGCAAGGATGTGTGTGAGGCTATGGAGTACCTGGAGTCTAAGCAG
TTTCTGCACCGGGACCTGGCTGCTCGCAATTGCCTGGTGAACGATCAGGG
CGTGGTGAAGGTGAGTGACTTCGGACTGTCAAGGTATGTGCTGGATGACG
AGTACACCAGCTCCGTGGGCTCTAAGTTTCCTGTGAGATGGTCTCCACCC
GAGGTGCTGATGTATAGCAAGTTCTCCTCTAAGAGCGATATCTGGGCCTT
TGGCGTGCTGATGTGGGAAATCTACAGCCTGGGCAAGATGCCTTACGAGC
GGTTCACAAATTCCGAGACAGCTGAGCACATCGCCCAGGGCCTGCGCCTG
TACCGGCCACATCTGGCCTCTGAGAAGGTGTACACCATCATGTACAGCTG
TTGGCACGAGAAGGCCGACGAGAGACCCACATTCAAGATCCTGCTGTCCA
ACATTCTAGATGTGATGGACGAGGAGAGCTGA
BTKe.BTKp.coBTK
(SEQ ID NO: 44)
TTTCCTAGGAGAATCCCTGGGGGAATCATTGCAGTTGGAGCATAATGTAG
GGGGCCCTCCATCACCTACTAGATATATCAGTGCAGTGAAAACTTCGCTA
AACTAACGCTATACCTATATCATGAAGTGTGTGGACTAGAGACAAGTGCA
TATCCTTACGGCAATTAACTGGGAAACGTCAAATAGTAACTACCACTCAC
CTTTTTCCGGAAAATCGGCTTAGTTTGCCCACCATAGCCACTCTGCTTCC
TGTCATAACGCCGCTTTCCTGGGAAAACGAATTGGTATTTGTTATAAAAT
ACTGAAGATCAGCAAGTAAGTCTTACAGGTTTTATCTTAATTTCGCAGCA
GAAATATTAACGCTCAAGCCAGGCGTGGAGGGAGAGAGACCCGGACTCGT
ATGTTATTCTACAACACAAATGTCACATTAACACCAAATTATGCGGAATC
CATCTTACCCTGGGCGTACAGAGAATCCTTGCCCTTCTTGTACTGTGTCA
CTTTATGGGGTTGGTGCTTGCCACACTTCTTACAGAAAGTCCGGCGGGTT
TTAGGGACGTTAACCTAGTAAAGAAACAGTTCAGAACGTGCAATGTTATT
TGACCACAATGGCACAACGCCCTACCTTACCCAGCTAAAGCTGAGGCACT
CCAGGAGGACTCCTCATTACTTGCTACCTCTGACTACAGGGTGGGCCAGC
CCCATGTGCTTCAAGCAGAGCTTCCTCCCTCCGTCGAGCCCCAAAGAGGG
AAGAGACCTCATTAACTCCACCCCCGGCTAACTCTACCTCTTTGAACCCA
TCACTTCAATTCCTGGCCCCGTAGCCCGGTCCCTTTAGGGTTGATCCCGG
CAAGATTGGGTTGCTCTGATATATCGAGTCCACACAGGAGCCTGGACCCA
TCCCGGCATAGCACGGGCGACGAAGGGGGGGAAAGATTAAGCTGGATGTT
ACTCGGCCCCCACCAGCAAGTCCTACCATGCTTGCGTGAGCGCTATCGGC
GCGGAAAGAAAGAAACCGCGAGGCAAACGGAAGTATATAGGAGGTTCCCG
ATCGCACTTCCTCATGGGAGTCGGTAGGAGCAATCATAGAGTGTAAGGCT
CAGCGCAGCGCCCTCGGGCGGCTGAGAGGACTCAGTTCGGAGCCGCGGGC
GGGAGCTTAAGGAAGGACTCCGCCTAAAGGGTGGTCCACTCACCCCGACT
TCCTCCCGCCCCGCAGCTTTCAACGTTTCGTCACTTTATCTCTTTTGGTG
GACTCTGCTACGTAGTGGCGTTCAGTGAAGGGAGCAGTGTTTTTCCCAGA
TCCTCTGGCCTCCCCGTCCCCGAGGGAAGCCAGGACTAGGGTCGAATGAA
GGGGTCCTCCACCTCCACGTTCCATTCCTGTTCCACCTCAAGGTCACTGG
GAACACCTTTCGCAGCAAACTGCTAATTCAATGAAGACCTGGAGGGAGCC
AATTGTTCCAGTTCATCTATCACATGGCCAGTTGGTCCATTCAACAAATG
GTTATTGGATGCCCATTATGTGGCAGGCACTGTTCCGGGGGAGAGGTACA
GTAATCTAATAGGCTTATAAATGTGCAATTATGAACTAAGTACTTTGAAG
AAAAGGAACAATGATTGGCATTAAAGCAGCACCCTTCTGTTGAGGGAGTA
AGTCAGCAGCTCTAGGTTCTGAAAAGTGACAATGAAATTGTTTGGCTCCT
GTGGCCCCTGAGAAAACCTCCAGGCTTCAAGTGACATACCTAGTCTGCTT
TACCGGTTTACAGGACTCAAGAGAAAGGTGGACATTGAGAGTTAATCCCT
GAGGCCAAATCTTAAATGGAGAAAGTCAACATCCACAGAAAATGGGGAAG
GGCACAAGTATTTCTGTGGGCTTATATTCCGACATTTTTATCTGTAGGGG
AAAAATGCTTTCTTAGAAAATGACTCAGCACGGGGAAGTCTTGTCTCTAC
CTCTGTCTTGTTTTGTCCTTTGGGGTCCCTTCACTATCAAGTTCAACTGT
GTGTCCCTGAGACTCCTCTGCCCCGGAGGACAGGAGACTCGAAAAACGCT
CTTCCTGGCCAGTCTCTTTGCTCTGTGTCTGCCAGCCCCCAGCATCTCTC
CTCTTTCCTGTAAGCCCCTCTCCCTGTGCTGACTGTCTTCATAGTACTTT
AGGTATGTTGTCCCTTTACCTCTGGGAGGATAGCTTGATGACCTGTCTGC
TCAGGCCAGCCCCATCTAGAGTCTCAGTGGCCCCAGTCATGTTGAGAAAG
GTTCTTTCAAAGATAGACTCAAGATAGTAGTGTCAGAGGTCCCAAGCAAA
TGAAGGGCGGGGACAGTTGAGGGGGTGGAATAGGGACGGCAGCAGGGAAC
CAGATAGCATGCTGCTGAGAAGAAAAAAAGACATTGGTTTAGGTCAGGAA
GCAAAAAAAGGGAACTGAGTGGCTGTGAAAGGGTGGGGTTTGCTCAGACT
GTCCTTCCTCTCTGGACTGTAAGAATTAGTCTCGAGAAAGAAGCCACCAT
GGCCGCTGTGATCCTGGAGAGCATTTTCCTGAAGAGGTCCCAGCAGAAAA
AGAAAACCTCTCCCCTGAACTTTAAGAAAAGACTGTTCCTGCTGACAGTG
CACAAGCTGTCTTACTATGAGTACGACTTTGAGCGGGGCCGCCGAGGATC
AAAAAAGGGGAGCATCGATGTGGAGAAGATTACATGCGTGGAGACCGTGG
TCCCTGAAAAGAATCCACCCCCTGAGAGGCAGATCCCAAGACGGGGCGAG
GAGTCCTCTGAGATGGAGCAGATTAGTATCATTGAGCGCTTCCCCTATCC
TTTTCAGGTGGTGTACGACGAGGGACCACTGTATGTGTTCTCACCCACAG
AGGAGCTGAGAAAGAGGTGGATTCACCAGCTGAAGAACGTGATTAGATAC
AATAGCGATCTGGTGCAGAAGTATCACCCTTGTTTTTGGATCGACGGGCA
GTACCTGTGCTGTTCCCAGACAGCTAAGAACGCTATGGGATGCCAGATTC
TGGAAAATCGGAACGGATCTCTGAAACCAGGGAGTTCACACCGCAAGACC
AAAAAGCCCCTGCCTCCAACACCCGAGGAGGATCAGATCCTGAAAAAGCC
TCTGCCACCCGAGCCTGCTGCAGCCCCAGTCAGCACTTCCGAACTGAAAA
AGGTGGTGGCTCTGTATGACTACATGCCCATGAATGCTAACGATCTGCAG
CTGAGAAAGGGCGACGAGTATTTCATTCTGGAAGAGTCTAATCTGCCTTG
GTGGAGGGCCAGAGATAAGAACGGACAGGAGGGGTACATCCCATCTAATT
ATGTGACCGAGGCTGAGGACTCTATTGAGATGTACGAGTGGTATAGCAAG
CACATGACACGGTCCCAGGCTGAGCAGCTGCTGAAGCAGGAGGGCAAAGA
GGGAGGGTTTATCGTGCGCGATTCTAGTAAGGCCGGCAAATACACTGTGT
CAGTGTTCGCTAAGAGCACCGGAGACCCCCAGGGCGTGATCAGACACTAT
GTGGTGTGTTCCACACCTCAGTCTCAGTACTATCTGGCTGAGAAGCACCT
GTTTAGTACAATCCCAGAGCTGATTAACTACCACCAGCACAATTCTGCCG
GCCTGATCAGCAGGCTGAAGTATCCCGTCTCCCAGCAGAACAAAAATGCT
CCTTCTACCGCTGGACTGGGGTACGGCAGTTGGGAGATTGATCCAAAGGA
CCTGACATTCCTGAAGGAGCTGGGAACTGGGCAGTTTGGCGTGGTGAAGT
ATGGAAAATGGAGAGGGCAGTACGATGTGGCCATCAAGATGATCAAGGAG
GGCTCAATGAGCGAGGACGAGTTCATCGAGGAGGCTAAGGTCATGATGAA
CCTGTCCCACGAGAAACTGGTGCAGCTGTATGGAGTGTGCACCAAGCAGC
GGCCCATTTTTATCATTACAGAGTACATGGCTAATGGGTGTCTGCTGAAC
TATCTGCGCGAGATGAGACACAGATTCCAGACACAGCAGCTGCTGGAAAT
GTGCAAGGATGTGTGTGAGGCTATGGAGTACCTGGAGTCTAAGCAGTTTC
TGCACCGGGACCTGGCTGCTCGCAATTGCCTGGTGAACGATCAGGGCGTG
GTGAAGGTGAGTGACTTCGGACTGTCAAGGTATGTGCTGGATGACGAGTA
CACCAGCTCCGTGGGCTCTAAGTTTCCTGTGAGATGGTCTCCACCCGAGG
TGCTGATGTATAGCAAGTTCTCCTCTAAGAGCGATATCTGGGCCTTTGGC
GTGCTGATGTGGGAAATCTACAGCCTGGGCAAGATGCCTTACGAGCGGTT
CACAAATTCCGAGACAGCTGAGCACATCGCCCAGGGCCTGCGCCTGTACC
GGCCACATCTGGCCTCTGAGAAGGTGTACACCATCATGTACAGCTGTTGG
CACGAGAAGGCCGACGAGAGACCCACATTCAAGATCCTGCTGTCCAACAT
TCTAGATGTGATGGACGAGGAGAGCTGA
BTKeΔMyc.BTKp.coBTK
(SEQ ID NO: 45)
TTTCCTAGGAGAATCCCTGGGGGAATCATTGCAGTTGGAGCATA
ATGTAGGGGGCCCTGTTACTCGGCCCCCACCAGCAAGTCCTACCATGCTT
GCGTGAGCGCTATCGGCGCGGAAAGAAAGAAACCGCGAGGCAAACGGAAG
TATATAGGAGGTTCCCGATCGCACTTCCTCATGGGAGTCGGTAGGAGCAA
TCATAGAGTGTAAGGCTCAGCGCAGCGCCCTCGGGCGGCTGAGAGGACTC
AGTTCGGAGCCGCGGGCGGGAGCTTAAGGAAGGACTCCGCCTAAAGGGTG
GTCCACTCACCCCGACTTCCTCCCGCCCCGCAGCTTTCAACGTTTCGTCA
CTTTATCTCTTTTGGTGGACTCTGCTACGTAGTGGCGTTCAGTGAAGGGA
GCAGTGTTTTTCCCAGATCCTCTGGCCTCCCCGTCCCCGAGGGAAGCCAG
GACTAGGGTCGAATGAAGGGGTCCTCCACCTCCACGTTCCATTCCTGTTC
CACCTCAAGGTCACTGGGAACACCTTTCGCAGCAAACTGCTAATTCAATG
AAGACCTGGAGGGAGCCAATTGTTCCAGTTCATCTATCACATGGCCAGTT
GGTCCATTCAACAAATGGTTATTGGATGCCCATTATGTGGCAGGCACTGT
TCCGGGGGAGAGGTACAGTAATCTAATAGGCTTATAAATGTGCAATTATG
AACTAAGTACTTTGAAGAAAAGGAACAATGATTGGCATTAAAGCAGCACC
CTTCTGTTGAGGGAGTAAGTCAGCAGCTCTAGGTTCTGAAAAGTGACAAT
GAAATTGTTTGGCTCCTGTGGCCCCTGAGAAAACCTCCAGGCTTCAAGTG
ACATACCTAGTCTGCTTTACCGGTTTACAGGACTCAAGAGAAAGGTGGAC
ATTGAGAGTTAATCCCTGAGGCCAAATCTTAAATGGAGAAAGTCAACATC
CACAGAAAATGGGGAAGGGCACAAGTATTTCTGTGGGCTTATATTCCGAC
ATTTTTATCTGTAGGGGAAAAATGCTTTCTTAGAAAATGACTCAGCACGG
GGAAGTCTTGTCTCTACCTCTGTCTTGTTTTGTCCTTTGGGGTCCCTTCA
CTATCAAGTTCAACTGTGTGTCCCTGAGACTCCTCTGCCCCGGAGGACAG
GAGACTCGAAAAACGCTCTTCCTGGCCAGTCTCTTTGCTCTGTGTCTGCC
AGCCCCCAGCATCTCTCCTCTTTCCTGTAAGCCCCTCTCCCTGTGCTGAC
TGTCTTCATAGTACTTTAGGTATGTTGTCCCTTTACCTCTGGGAGGATAG
CTTGATGACCTGTCTGCTCAGGCCAGCCCCATCTAGAGTCTCAGTGGCCC
CAGTCATGTTGAGAAAGGTTCTTTCAAAGATAGACTCAAGATAGTAGTGT
CAGAGGTCCCAAGCAAATGAAGGGCGGGGACAGTTGAGGGGGTGGAATAG
GGACGGCAGCAGGGAACCAGATAGCATGCTGCTGAGAAGAAAAAAAGACA
TTGGTTTAGGTCAGGAAGCAAAAAAAGGGAACTGAGTGGCTGTGAAAGGG
TGGGGTTTGCTCAGACTGTCCTTCCTCTCTGGACTGTAAGAATTAGTCTC
GAGAAAGAAGCCACCATGGCCGCTGTGATCCTGGAGAGCATTTTCCTGAA
GAGGTCCCAGCAGAAAAAGAAAACCTCTCCCCTGAACTTTAAGAAAAGAC
TGTTCCTGCTGACAGTGCACAAGCTGTCTTACTATGAGTACGACTTTGAG
CGGGGCCGCCGAGGATCAAAAAAGGGGAGCATCGATGTGGAGAAGATTAC
ATGCGTGGAGACCGTGGTCCCTGAAAAGAATCCACCCCCTGAGAGGCAGA
TCCCAAGACGGGGCGAGGAGTCCTCTGAGATGGAGCAGATTAGTATCATT
GAGCGCTTCCCCTATCCTTTTCAGGTGGTGTACGACGAGGGACCACTGTA
TGTGTTCTCACCCACAGAGGAGCTGAGAAAGAGGTGGATTCACCAGCTGA
AGAACGTGATTAGATACAATAGCGATCTGGTGCAGAAGTATCACCCTTGT
TTTTGGATCGACGGGCAGTACCTGTGCTGTTCCCAGACAGCTAAGAACGC
TATGGGATGCCAGATTCTGGAAAATCGGAACGGATCTCTGAAACCAGGGA
GTTCACACCGCAAGACCAAAAAGCCCCTGCCTCCAACACCCGAGGAGGAT
CAGATCCTGAAAAAGCCTCTGCCACCCGAGCCTGCTGCAGCCCCAGTCAG
CACTTCCGAACTGAAAAAGGTGGTGGCTCTGTATGACTACATGCCCATGA
ATGCTAACGATCTGCAGCTGAGAAAGGGCGACGAGTATTTCATTCTGGAA
GAGTCTAATCTGCCTTGGTGGAGGGCCAGAGATAAGAACGGACAGGAGGG
GTACATCCCATCTAATTATGTGACCGAGGCTGAGGACTCTATTGAGATGT
ACGAGTGGTATAGCAAGCACATGACACGGTCCCAGGCTGAGCAGCTGCTG
AAGCAGGAGGGCAAAGAGGGAGGGTTTATCGTGCGCGATTCTAGTAAGGC
CGGCAAATACACTGTGTCAGTGTTCGCTAAGAGCACCGGAGACCCCCAGG
GCGTGATCAGACACTATGTGGTGTGTTCCACACCTCAGTCTCAGTACTAT
CTGGCTGAGAAGCACCTGTTTAGTACAATCCCAGAGCTGATTAACTACCA
CCAGCACAATTCTGCCGGCCTGATCAGCAGGCTGAAGTATCCCGTCTCCC
AGCAGAACAAAAATGCTCCTTCTACCGCTGGACTGGGGTACGGCAGTTGG
GAGATTGATCCAAAGGACCTGACATTCCTGAAGGAGCTGGGAACTGGGCA
GTTTGGCGTGGTGAAGTATGGAAAATGGAGAGGGCAGTACGATGTGGCCA
TCAAGATGATCAAGGAGGGCTCAATGAGCGAGGACGAGTTCATCGAGGAG
GCTAAGGTCATGATGAACCTGTCCCACGAGAAACTGGTGCAGCTGTATGG
AGTGTGCACCAAGCAGCGGCCCATTTTTATCATTACAGAGTACATGGCTA
ATGGGTGTCTGCTGAACTATCTGCGCGAGATGAGACACAGATTCCAGACA
CAGCAGCTGCTGGAAATGTGCAAGGATGTGTGTGAGGCTATGGAGTACCT
GGAGTCTAAGCAGTTTCTGCACCGGGACCTGGCTGCTCGCAATTGCCTGG
TGAACGATCAGGGCGTGGTGAAGGTGAGTGACTTCGGACTGTCAAGGTAT
GTGCTGGATGACGAGTACACCAGCTCCGTGGGCTCTAAGTTTCCTGTGAG
ATGGTCTCCACCCGAGGTGCTGATGTATAGCAAGTTCTCCTCTAAGAGCG
ATATCTGGGCCTTTGGCGTGCTGATGTGGGAAATCTACAGCCTGGGCAAG
ATGCCTTACGAGCGGTTCACAAATTCCGAGACAGCTGAGCACATCGCCCA
GGGCCTGCGCCTGTACCGGCCACATCTGGCCTCTGAGAAGGTGTACACCA
TCATGTACAGCTGTTGGCACGAGAAGGCCGACGAGAGACCCACATTCAAG
ATCCTGCTGTCCAACATTCTAGATGTGATGGACGAGGAGAGCTGA
SEQ ID NO: 46: The B29 promoter sequence:
AGGAGGGCCATCATGGCCAAGTTGACCAGTGCTGTCCCAGTGC
TCACAGCCAGGGATGTGGCTGGAGCTGTTGAGTTCTGGACTGACAGGTTG
GGGTTCTCCAGAGATTTTGTGGAGGATGACTTTGCAGGTGTGGTCAGAGA
TGATGTCACCCTGTTCATCTCAGCAGTCCAGGACCAGGTGGTGCCTGACA
ACACCCTGGCTTGGGTGTGGGTGAGAGGACTGGATGAGCTGTATGCTGAG
TGGAGTGAGGTGGTCTCCACCAACTTCAGGGATGCCAGTGGCCCTGCCAT
GACAGAGATTGGAGAGCAGCCCTGGGGGAGAGAGTTTGCCCTGAGAGACC
CAGCAGGCAACTGTGTGCACTTTGTGGCAGAGGAGCAGGACTGA

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

1.-84. (canceled)

85. A nucleic acid for sustained Bruton's tyrosine kinase (BTK) expression, comprising:
a first polynucleotide encoding an ubiquitous chromatin opening element (UCOE);
a second polynucleotide encoding a promoter; and
a third polynucleotide encoding BTK.

86. The nucleic acid of claim 85, wherein the UCOE has a length in a range from 0.25 kb to 2 kb.

87. The nucleic acid of claim 85, wherein the first polynucleotide comprises the nucleotide sequence set forth in SEQ ID NO:01 or SEQ ID NO:02.

88. The nucleic acid of claim 85, wherein the promoter is selected from a BTK promoter, a B29 promoter, or an endogenous promoter.

89. The nucleic acid of claim 88, wherein the BTK promoter comprises the nucleotide sequence set forth in SEQ ID NO:05.

90. The nucleic acid of claim 85, wherein the third polynucleotide comprises the nucleotide sequence set forth in SEQ ID NO:06 or SEQ ID NO:07.

91. The nucleic acid of claim 85, further comprising an enhancer element selected from the group consisting of:
a DNase hypersensitive site (DHS) of a human BTK gene;
an intronic region of a human BTK gene; and
the nucleotide sequence set forth in SEQ ID NO:16, SEQ ID NO:17, SEQ ID NO:18, SEQ ID NO:19, or SEQ ID NO:20.

92. The nucleic acid of claim 91, wherein the DHS is a DNase hypersensitive site 1 (DHS1), a DNase hypersensitive site 2, (DHS2), a DNase hypersensitive site 3 (DHS3), a DNase hypersensitive site 4 (DHS4), or a DNase hypersensitive site 5 (DHS5).

93. The nucleic acid of claim 92, wherein the DHS comprises the nucleotide sequence set forth in SEQ ID NO:03.

94. The nucleic acid claim 91, wherein the intronic region is selected from intron 4, intron 5, or intron 13.

95. The nucleic acid of claim 94, wherein the intronic region comprises the nucleotide sequence set forth in SEQ ID NO:09, SEQ ID NO:10, or SEQ ID NO:11.

96. The nucleic acid of claim 91, wherein the enhancer element comprises the nucleotide sequence set forth in SEQ ID NO:04, SEQ ID NO:14, SEQ ID NO:15, SEQ ID NO:21, or SEQ ID NO:22.

97. A vector comprising the nucleic acid of claim 85.

98. The vector of claim 97, wherein the vector is a lentiviral-based vector.

99. A cell comprising the nucleic acid of claim 85.

100. The cell of claim 99, wherein the cell is selected from a B cell, a myeloid cell, or a hematopoietic stem cell.

101. The cell of claim 100, wherein the cell is a CD34+ hematopoietic stem cell.

102. A pharmaceutical composition comprising the cell of claim 99 and a pharmaceutically acceptable carrier.

103. A method for treating, inhibiting, or ameliorating X linked agammaglobulinemia (XLA) or disease symptoms associated with XLA in a subject, comprising administering the cell of claim 99 to a subject in need thereof.

104. The method of claim 103, wherein the cell is autologous to the subject.

105. The method of claim 103, wherein the administration comprises an adoptive cell transfer.
